SEQUENCE LISTING (504 sequences)

SEQ ID NO: 1 - protein, 511 aa, synthetic construct
MADFVEHIADNGSTLSSDDEFDMETDIEMENDDENDIWTNSSFVENKFEYSLQSRMCIAHIDNMQPIDYFMNFFDETLMNIIVCETNLFQRQNPIPHREHMKKWQDTTLQEMYIFLGIILLTGMIGKRALRDYWTTDFLFQTPVFAKIMSRNRFQDLLRCLHFNDNRKKDNDRLFKLNPIVNYLKQKFRFCTVPSQKLCIDESLLLWKGKLSFKQYIPSKRSRFGIKMFNLVDCSTRVLLDFIIYTGKETNIRIDKSIGFSGSIVMTLLLPFLNKGHILYVDNWYSSPSLFTLLLKKRTGAVGTVRKNRKHLPKLQAKLAKGQTQQLNNGKLLYMKWLDKREVFMLSSIHQPTMAPTGKTDRNNQAIVKPTCIIDYNQNMGAVDQLDMQISFTEVIRKTVKWYKKVFFHLLDLAAFNSYALYRKKEKSKMTHLEFRKTLVRQIFEKFNVFENLPSGPSVRAQHFPGQYTERDNSNRIIKHRCNVCRSLTTWNCETCEIALCLPGCFKTKHS

SEQ ID NO: 2 - protein, 529 aa, synthetic construct
MSNEIADYELLADEGSDIGSDDEIEDFDIINDRAGERIWSKEGNFVPKLHQFQEKPGPKNFRVENEQPIDYFEWFIDSEFMDYVVKETNLYQQQHPLPSQKKMAKWFDVTSNELSVFFAIVMLTGLVSKNELKDYWSVDPLLNTPIFPKTFTRNRFLQIMRFLHFANNEQKTNDKLFKIGNVVNMVKEKFKNAIIPDRNICIDESLMLWKGRLSFKQYIPSKRHRFGIKLFEMVDCTTQCILNFIIYTGKSTKFRFHELLGISGSIVMELMNMHLDKGHVLYVDNWYSSPPLFEILHYKKTGACGTLRKNRKGLPKYSGKMKKGELESYNTDKLLFQRWMDKREVFMLSTIHRNGLVESTKTNFQSFEKIMKPKTVIDYSLFMGGVDYSDMRMSFSECVRKTTKWYKKFFFHIIDMVVHNAYCIYRAKNKSRIQLADFRLILVRQLIEKYGSQRSERVGRPPLSENPLRLQGRHFPDKIPINSGDNGKKCCVVCSTSSDRKRTTIMCQLCDVPLCIVPCFKDYHTKVNL

SEQ ID NO: 3 - protein, 546 aa, synthetic construct
MSDSESEYEYANLSESGSEDSDNYSSDSEDSSDEDIAVDWKQISDSQPNPPPPRFPFVGASGPTFFFDNNFDVLEYFKLFFDDSVMDIIVLETNRYAEQQPGGSTTQWKNVDKNEMMIFFAICILQGLIKKPEERQYWSTNEIWNTPIFPKLMNLRRYTSIKRNLHFCNNETFDRNTHPNPKLYKIWPIYEIINNKCSSLYIPERDITIDESLLLYKGRLGWVQYIPLKRARFGIKLFLLCESKSGYLFSFIIYTGKGTVIDVKYKDMPVTSQIVVSLLDPLLDQGYCLTTDNYYTSPQLADFLVKHKTDTYGTVRKNRKDIPKFIQTKKLKKGEIVAAQRGKVMVMKWQDKRDICLLSTIHNTEKAATNKTDKDGNVVSKPKLVLDYNETMGGVDRLDQHLHDYPIIRKRGKKYYKKIFFHLLDISIWNGYVLYKKNGGGKSNLDFRSELVEKLIQNYHSEQNIKKSGRPKSTGILRLTERHFPSFVPPTPKKEAPTRYCAVCCSKRDSKGKRKRRESRYMCNPCNVGLCAAPCFEIYHTKENFE

SEQ ID NO: 4 - protein, 546 aa, synthetic construct
MPEAATDQDSDASDDEVMCDPGHLPRRILLSKVLNNDVDIDQSEIEFNPPSTSTGRGSKRRKKVVYMWHTDKSKVANLVPDFSETREVVDAPKTNPLEFFEERFWTEDWINVLCEQSKNYAHQKGIPCSDVNADNMKLFLAILILSGYNKVPNRRLYWSESPDTQNKLVMNSMRRDTFDQIMRCLHINDNMKMDGDRFYKVRPLFEHLNNSNKDEKCGEYYSIDEIMVPYYGRHGDKQYIRGKPVRFGFKLWAICTSDGFLHNVEPYCGSHTRISDRGFGLGGNVVLEMIERVNIKPGQHIVFDNFFGSVALLKELSQKKIAATCTLREDRLSGAPLRPRKVLEKEERGTSDEAFTSCISVVKWKDNKVVSVASNKLRSDPAKKAKRWNRVDKKHVEVDLPHSIHVYNQHMGGVDVFDQQVAAYRCRIRSKKWWWPLFAWTVNAQVVNGWKLFREHEKKITLLEFSRQLVISTLTKYGKPKAKPGPVPMPVVNTVRYNSQHHWTYKGKSPYNRCRECGSRTIYFCSVCNIPVHPECMKTFHTKKET

SEQ ID NO: 5 - protein, 567 aa, synthetic construct
MSRRLRSANVLLEKINDIDAECSEDDQEILDMEIDNTENYDPLETDSNTDENEENISNVCRRRKRRRVLVSSDSENGEEVSEIAMDGTVWQEVKTGSNPGRAPSHNIFRETSGPTGYSKRNIMKGEVRTAFSVIIDKNIIELIRKCTETEAFRVLGYKWELSTAKLYAFFAILYARGAYEAKNIDIALLWNKKWGPPFFSNTMSRHDFTEIMRFIRFDDRNQRSQRLQTDKFAMISEVWYKFIDNSQNCYKPGPYITIDEQLFPTKARCRFTQYMPNKPDKFGIKFWLASDVRSKYIINGFPYLGKDESREPSVPLGEFVTMKLAKPYVGCGRNITTDNFFTSLPLATKLLAKKTTIVGTIRANRKELPRLAKLKNDDMALFSTKLYRSNNCMLTVYKAKPRKKVLILSSMHNSVQVEENDTRTPETIQLYNSTKFGVDVTDQMARNYSVKSKSRRWPLQVFFNILDLAGINAWVLYKETTGGEITRQEFLFQLAEELAIEYQQELGKENQPIPIASTSTGSRERKTCQIRYCKDNKANKICLKCKKYVCGKCTVEKPICKKCDEKE

SEQ ID NO: 6 - protein, 570 aa, synthetic construct
MSKQTRQYKFLEDINNIGEEFSQIGEDEFSENEDILVSNESELDSIDTDDEKENTVNICCGRKRIRLLSSFEESEVEINKQHVANDGSVWEEIKVGGTPGRIPLHNIFRQEVGPTGYAKRHIMKGNVSTAFSLIIDHRIMNHIRSCTIEEAKRVLGSDWSLSQEKLDAFIAILYARAAYGANNLNISFLWNNIWGPNFFSETMSRNNFTEILRFIRFDKKSQRSQRLKTDKFAMVSTIWNIFIENSQNSYKPGGNITIDEQLFPTKVRCRFTQYMPNKPDKFGIKFWLASDVDSKYVINAFPYLGKDENRPSSVQLSEYVVLKLMESFTGCGRTVTTDNFFTSKSLATQLLAKNTTLVGTIRSNKRELPLIAKQKKDDMERFSSKIFTTDNCTLTIYKSKPNKKVLLLSSKHKFVTVENNDKRLPETVSYYNKTKFGVDMTDQMARKFTTKSKSCRWPLQVFFNILDLAGINAWILYKQTTGENISRQDFLLKLAVELGADFREAREQPKKRTTTKDSLPKSTTDASQRKRCQIGYCKENKTNKICSKCKKYVCGKCTMDTLICKKCDEQ

SEQ ID NO: 7 - protein, 581 aa, synthetic construct
MSKKAKLSTYLENATNIPLECSDVESESYDSDGDNSSAIIESGDEEIPDEDNIDQSSDSDREKILNKRTRRCMRFPSSSEDECGSNIPNQQTEIASDGTIWTRIEEGGSAGRSPHHSAFKDEHGPTAHAKRNIMNGNLSSAFLLLIDNHILEHIRICTESEASRVLGKNWTITQEKLKAFLAILYARGAYEANNLRLQYLWNNKWGPSFFSNTMSRRDFTEIIRYIRFDKKNQRSQRLQTDKFALVSAVWDKFIENSKNCFKPGAYITVDEQLFPTKARCRFTQYMPNKPQKFGIKFWLASDVETKYVVNGFPYLGKDETRNASTPLSEFVVIKLLEPYTMKGRTVTTDNFFTSIPLALKLRSKNTSLLGTIRANKRELPKICKLKKDSMARFSTMLYQSNGCTLTVYKSKPNKKVLLLSTKHKHIKIDKAAKKLPETVSFYNNTKFGVDVTDQMARKYTVKSGSRRWPLQVFFNILDLAAINSWVLYKNTTGENISRKDFLFQLAEELASEYQTSRQKPHETNIPTINATVPVRKWCQIGYCNNNKTTNICNKCKKSLCGKCTRSKIYTCRNCEQQTVNN

SEQ ID NO: 8 - protein, 582 aa, synthetic construct
MTKQMKIEKLLEKIRNIPEGFSDIEEDKLYDSDGDLIESNNGPYGSDDYMFDSGDNDSDHEESSASDSEYENILNIPKRKRNQQLSTSSEEEVEEDEIELSADGTVWRKLNEGSSGGRLPLHNIFKDTPGPTGYAKRNIMKGSASSAFSLIIDRNIMEYIIKCTEAEAFRVFGKKWTLTEEKLKAFMAIVCARGAYEAKNLKLSYLWNKKWGPKFFADTMSRNDFTEILRFIRFDNKNSRSQRLQTDKFALISKVWNKFIENSQNCYKPGQNITVDEQLFATKARCRFTQYMPNKPNKFGIKFWLAADVQSKYIINGFPYLGKDETRSSSTSLSEFVALKLMEPYTMKGRTVITDNFFTSVSLASKLLAKRTTLVGTIRSNKRELPKFVKIKKDNMQCYSTLLYKANDFILTIYKSKPQKKVLVLSSKHKSVQINKNKKLLPETITFYNKNKCGVDSSDQMARKYSTKSGSRRWPLQVFFNILDLAIINAWILYKKTTAINIARKDFMFQLADELAADFVKSRSQIERHNQQPLDESTNTRKQCQTGFCNKNKTTLMCDKCKRYVCGKCISVKKIICKRCEE

SEQ ID NO: 9 - protein, 590 aa, synthetic construct
MTSKKFLNIEDIETELENFDDNNSFDGFEDDEILEATVSVPDQSNLSCPQSTAIDNLMEVCDLSDNESLSSSLPEENMEVDSDSEESVWNKGELQHERDLPIDFDTVNVTPTYNFSVTDGPVVYFQRFFDAQVITDLVNQTDLYAQQVKIKNWVPVTLEELNAFIGILIGMALHDLPSIDSFWSSDPLFLVKPIADCMPIKRFKKILEALHCNDNSQAPAVSCANRDKLYKVRPLIDILNTKFEIECHSSSSQSIDEAMIKFKGRSGIKQYMPMKPVKRGYKVWARADSNTGYLYQFDVYTGKLEDGVEEGLGSKVIKKLSTSLKNKNCHIVFDNFFTSYTLMEELYEHKIYATGTVRSSRKELPALAKKTQKMNKHESKYRIRNHTGYVKWKDTKIVSVLSTAFRPNIIGEAKRTQKDGSTKIVPCPQSIIEYTKRMGGVDRFDQKRTYYGIGRRSKRWWLRIFYFMIDAAIANANILYNVNHHNATLSGLDFRIRLHRSLISNYTSRKRPSLSNTPFIKKQKHNSTHKDLGVPDEIRLSKVGVHMPEKLTTFRRCRFCSSKTNNKRSNIGCTTCNVALCINCFKKFHK

SEQ ID NO: 10 - protein, 601 aa, synthetic construct
MGKNQKNCLLLKKLIKIKKDQSDNESDAIDIDQCDNEIELEEEVNKIESSSSEDEDLCYIAPNKKRARVISDSETDSEDEVFRYTAPSKKRARVISDSETENDLDAIDTSSDKEISAEKAADGTLWETLNEGRNVGRSPIYTIFKDVSGPTAYAKRHIMLGSASSAFNLIIDEGMMAYIKSCTELEARQVLNNKEWTVTKSQLWAFVAILFARGAYEAKNLKCSYLWSAKWGPAFFAQTMSRDKFIDILRFIRFDKKNERSERLKTDKFALISKIWEKFIENSQASYKPGANITIDEQLFPTKARCRFTQYMPNKPDKFGIKFWLASDVRSKYLVNGFPYLGKDETRGSNIPLSEFVVTKLAEPYLGCGRNITTDNFFTSISLAKKLLAKKTTLVGTIRANRKELPKIAKAKKDKMTQFSTKLYRSENCTLTIYKSKPNKKVSLLSTKHKNITIEKNKKLVPETVTYYNSTKYGVDVLDQMARKYSVKASSRRWPLQVFYNILDLAAINAWILYKETTQVNISRKDFIFQLAEELRSKYREEVENTSIPMTEIHATDARKNCQVQQSCKRNRCTNHCAKCNRNVCGKCVSKTEFICKKCFS

SEQ ID NO: 11 - protein, 605 aa, synthetic construct
MDKRIFSKGKLSQKDLIDFWENFNGFESEDDDTIDDDDLEDPTYHSAVEEDSDDEETTPSATVPTELPCGSSADGIVSKPSKSKDILWQKKNLKPNEEQLHFCGNSDLPGGLLQLQTPYQFFSYFVSEDLIAKIVKQTNLYSVQKHADRPMAFTTSDIKQYIGIVFYMSFVHMPNIRCYWSPELGYPPVNSTMSVNKFEKLRRYIHFNDNSTFVTREEPGFDRLHKIRPLIDHLKKKFNSIPLENHLSIDEQMCSTKVKHFMKQYMPMKPHKWGFKLYILSGISGFAYNFEIYSGQENSMVRPDGEPDLGASSNIVLRLSRIIPRKQNFRLYHDNFYSAIPLMVHLAKEGIFSLGTIRRNRVPNCKLPTEKIMKKDDRGKSYEFVANIDGVDISNLVWKDNKYVTLVSSFAGTHPESVVRRYDRKMKKVIEVNCPYIIQEYNRHMGGVDLLDSHIGRYKIAIKSRKWYMRIFYHMMDLAMVNAWLLYKRVLKQNGCLESSILDQPSFRAEVAHVLCKINTTSAKRGRPNALEKEIQLKKTKGPTQYIPPQDVRNDQFGHWPIQIDIKMRCKYPNCKGFTRTKCEKCGVGLCLNKNNNCFKNFHTQ

SEQ ID NO: 12 - protein, 617 aa, synthetic construct
MARKRQTSQDIRNLFESIIDSDDEELANDNFDSDFSDFDIATDEAAVEMQNIETDEEQEVQNIEAAEEAEVEEEGIWYYAQNKKYKRKWRASPPNVTRVRDFQILRERQGISVETAQNINTHLAAFKKLLPETLINVIVAETNRKARHFYTINSNTARIWYDTNSNEIYALIGILIYAGAHKNWDERLNELYHLDNRPFYRAAMSLQRVQQLLRFLRFDNYQTRAERLKEDKLAAFRDVWNMFLANIKGPYKPSSFLTIDEQLVLTRGRCSFRQYIPSKPGKYGIKIFWICDALNSYPLKAEIYVGKQPNELRAVDYALNLVHRLSAPYINKGRTITMDNFFTSCPLAEQMLQKKTTIIGTIRANKPDLPKEFTNKAEIKNRVVNSSKFCFDDHLTLVSYVPKPNKNVLLLSTLHNDDIVDLDSNKPNIILDYNKTKGGVDTLDKLVRTYTCKRKTKRWPMVLFFNMIDITGIAAYNLYKCANPQLFIQRKSNERKIFLKELAMQLVKDHILNRISNPRAMKKNVKIAVQQIGYNIINLRQSSTAEGTAANRSVSKRPRCYICPRSADKKTGTRCYVCNQPSCTTHIKTVCINCINEEVMDSLEEENNEIMSDDASG

SEQ ID NO: 13 - protein, 564 aa, synthetic construct
MRNLFLDELEIETNGESSDDSDVSENQDPIEPSSSNTFVAKDGTVWNQLPPNFHQTPIHNIVRQRSGPLRSTETMSVCDTFKCIFTDEMIDIIIRHTNKKAKHVYETYNTNNPTKTQLEWKDLTVREFNSFLGILITCGVNRSNTQFSLDMWKTNSYPLYRASMGHNRFRSILRFIRFDDANTREERQQIDKAAPIRDLWSMLNSNLRKYYRPTENLTIDEQLFPYRGRIKFKQYIPSKPAKYGIKIWWICDAENYYPLTGQIYTGKSSTGREVNQGERVVKELVAPYKGSGRNITTDNFFTTLPLAKHLLSWNLTIVGTLKKNKSYIPQEMAPSNSRQVFSTVYGFQNNVTICSYVPKKKKAVILLSTMHHTIATSGEKEKPEIVYFYNKTKSGVDVMDHLLGQYSTQRKTNRWPLALFYNIVDIAALAAYLVYTANNYMIKKRSNARSIFLRQLGEELSLPMIQDRSQNIQVMRHFSSRTGIECMLQKSIVIADETAVASTSRVAEKDKTGRKKVVGSCYICYNKQIRKRRKTRKNCAECCKPICNEHCKMISKCNICYEKE

SEQ ID NO: 14 - protein, 602 aa, synthetic construct
MIHFTDTDDLDLLALTFEGDVSEVEGIDVSDDEDEIEAFHANTESPKCVKPQKDTPVKSGTKVRRNQNKTSAKQAASNQQGKKKGVSTRSKSKKKGTPPKWHEDESVCFDCIEPVVKSSKPPTKPMTPYQYFGLFIDSEIISDICEQTNLYSSQQDETPIDVTSGDIEQHIGQLLLMGVTKVPSYRLHWGSTTRYAPIADVMPRNKFETIKRCLHFNDNTKIKQRDEEGYDKLFKVRPFIDALRRNFLKIEPTPNQSIDEIMIPSKAASPLRQYNKNKPHRFGIKVEGRASSDGILHDFSIYGGKTNNEPSGAWGISGDVVIKLIDTLPQNVPYRIFADNWFSSYALVKEMKSRGLEYTGTVRQNRIPGFEMKKNLKAEGRGSFACNVSNDNIVVVTWMDNKPINLISSCFGVQPIDEVKRWSVSDKTYKSIPRPLVVREYNCYMGGIDLNDFLVALYRTQPGTKKYYMRIFYHLLDVSVVNAWLLYRRHMKQTGQEHMTLLNFRIDVANSLIQYGKKIVRRRGRPTNQPIQKRQREVASVGPSLEARYDGLEHWPVEDRRERCVLCKDRGSFSSFKCTKCDVCLCIKKGKNCFTAFHLPPD

SEQ ID NO: 15 - protein, 513 aa, synthetic construct
MPNKRTRRCMRFPSSSEDEDGSNVPNQQTEIAADGTIWTRIEEGGVVGRLPIHSAFKDVHGPTAHAKRNIMKGNLSSAFLLLIDNHILEHIRICTELEASRVLGKTWTITQEKLKAFLAILYARGAYEGNTLRLQYLWNKKWGPSFFSSTMSRRDFTDILRYIRFDKRNQRSQRLQTDKFALVSAVWDKFIENSQNCFKPGAYITVDEQLFPTKARCRFTQYMPNKPHKFGIKFWLASDVETKYVVNGFPYLGKDETRNASTPLSEFVVMKLLEPYTMKGRTVTTDNFFTSIPLALKLRSKSTSLLGTIRANKRELPKICKLKKDSMARFSTLLYQSNGCTLTVYKSKPNKKVLLLSTKHKNIKIDKAAKKIPETVLFYNKTKFGVDVTDQMARKYTVKSGCRRWPLQVFFNILDLAGINSWILYKNTTGENISRKDFLFRLAEELASEYQTSRQKPHEADISTTAGTVSVRKWCQIGYCNNNKTTNICNKCKKSLCGKCTKSKIYICRNCER

SEQ ID NO: 16 - protein, 610 aa, synthetic construct
MCTNRPNIVSIVVTICTNRPLHRISFAAQLYIFEVICRITNMTRRYTAQEIIDAIPYLSDAEESDFSDSCSEYLPEQPRREDSDSDDSELSSDSEQEDSVSEDNDDAESNSNSGDSLCTGTTNTVLWNNVPPGFQPKLNISQDRPCTIHPDITVTTSILDIFLKLFPYSLLLQIAYYTNKRLSIFQNAKTKRRKLVPTDAHEIMKLFGCFFVMSYNRLPSINNYWSKHQSMGNALMKSAFSRDRFKLIMSKLYAAEPDKPSQASKLYYVEDLLLCLKSTFMKYRQNSPFQSIDESMTKFKGRCSFKQYLPMKPVKRGIKLWMRCDPHSGYTYDVNIYAGKDDQGLSIPLGERVVKTLLSTIPDGDDVTVAFDRFFTSVHLMDSLRYPAVGTVIKSRKNLPVFQDKLSRGDSEFRCNMNGTLAVRWLDTKEVLLLSNFHTNTVGEVRKKQRDGTIINVSCPDAIRCYRQIMGGVDRADQMAGLYDLDRKSTKWWKKVIYRMLAFSAVNAWVIYKELHRHYKKPYLDFLVELAEELIQRGESGSTVKRRSAAGRPSKRASMMQNLGTHLPYEGNTRRRCTNCGKDKKQKRTKLMCSACNLPYCIDCFKPCHS

SEQ ID NO: 17 - protein, 587 aa, synthetic construct
MADRDNIIEEIFADSDSDGEDNFEGFDLEDLNTTELDDESIHANYNVLNSELWNEGDRTPLPLSFEQEPGLQKEVSDSDSPIEYFELFLDDFDYQNISDETNKYADQFLANKQLKEKSRFKKWRDTTSLEMKKFFAIVIAMAIITQMDISEYWTVNPVTSTPFFPSVMSRDRFLLLLAFLHLNDNDKYIPRGVEGHEPLFKLGPLYHRILTKFRSVYKPNQALAIDESMVAWRGNLSFRVYSPDKPVKYGLKAYVLCDSENAYCLRFKLYTGKKSVQPSENGATYDLIMDLMRNYFEKGHILYCDNYYSSPRLFMDLWVLGTGATGTVRSYRKGIPDKLKKAQLSNRGDTATMSYGPLSCLKYLDAKAVYLISTTDTSVNVETRRPDFMRNHEAKVRPSMVHVYDQKMGAVDRSNQMVENYKLGIKTMKWWKKLIFHIINIAIVNSYILYKENCSFNNAMVQRHFRRKLVEQLIQSSGGSISTPLGRPAPRVLERLTGRHFLDKLMEGEKPLHRQCIVCGPAEREMLPPLRPGEKRKRRCGHMTSYKCRQCNVSLCISPCFEIYHTKQEFLLAYKRSRLSSNSSTEE

SEQ ID NO: 18 - protein, 586 aa, synthetic construct
MADSVRNIIDEIFADSDSDGEENFEGFDLEDFGPVEIERNQDNSNYDVLNEDLWAPGDRTPHQLTFTGKSGLCKELDDCDSPRKIFELFIDDSDYENISEETNRYAAQFLHDKVLKEKSRFQKWKDTCASEMKKFFAIILAMGLLTQMDVSEYWTVNPVTSTPFFPSVMSRDRFLLLLAFIHLNDNSNHIPRGMEGYDPLFKLGPIYQKILSRFHTVYAPHQALAIDESMVAWHGNLSFRVYSPDKPIKYGLKAYMLCDAENAYCLKFKLYTGKSSVRPSENGATYDLVMDLLRNYYEKGHILFCDNYYSSPRLFMDLWILGTGATGTVRQYRKGIPKIIKDCKLSTRGETSTVHYGPLSCLKYQDSKTVYLISTTETSNIVTTSNHDFHTNETKIRPSMVHVYDKKMGAVDRNNQMVENYKLNIKTLKWWKKLFFHLINVAIVNSYIIYKECTTRSTPMVQRHFRRRLVEELLEYSGQTNVAPVGRPAPKVLERLTGRHFVDKFIEGGKTLHRQCIVCGPAERKMCDAVRPGEKRRFGHMTSYKCKQCNVPLCITPCFEIFHTKQEPFLAYKRMKSAEANCNTEE

SEQ ID NO: 19 - protein, 597 aa, synthetic construct
MSRKGLTPSQIGSFFNSLDSEDDLEFSADDESDYYPNKEDFETTDDEDLENEVEHPSDLNVSSKDLNDVSSSSKNVQRRNLLWRQRSLILNEDQLQFLTSAALPPELLELSQPIQFFSYLFPDKIFENISNETNLYIHQKDPNVNITFTPLDIQQFVGIVMYMSISRLPKCALYWSASIGIPSIQNIMSQKIFETIRRNIHFNNNDNNLPLTDPRHDRLFKIRPLIQELNNNFGKVPKEQFLCVDEQICSTKARSLLKRYNPNKPHKWGFKIYVLSGNSGFCYKFEIDSGKENVVLLNEPDLGAASNCVVRLAREVPRHQNFRLYFDNYFNSLPLLKYLAESGILALGTIRKNRIPNFKSIENVHLKKNPRGTSVEYVVDYGGVDISCTSWVDNKIVSLASNYAGKVPTDTARRFNKTKKEYIQIERPFAVAQYNSYMGGVDLIDSIIGRYKILLRSKRWQVRIFYHLLDLTISNAWLLYKRVLKANGYSGKILSSSGFRLDIAEVLTKVNTKTKLGKRSTVIENALQLKKQRGPTQNVPNQIVRQDQVGHWPNWADKRKRCKFPNCAGYSQVVCNKCGVALCFNKRNNCFTNFHLN

SEQ ID NO: 20 - protein, 606 aa, synthetic construct
MRPKEKGLSNDDIERIVNFDWDVSSDEESGDEMEDISGMLENNLKSILERGESIEFEHIENLIDEKSIQENSVVADNCFEKVDPKTLKWRNRPFQAPGCIWEEDSNRQIDEVKTPVEYFCSFFDNNVIDLITNQTNLYGLQEHGIELQCTGDQIKRYIGILLYLGVIKVPQFKMAWSKEYKLSAISDAMPRGKFEKIKQCLHFNDNTKQLKKGDLNYDKLYKIRPLLDILKKSFGKLPQEEHQSVDEQIIAYKGQSSYKQYNPAKPHKWGLKMFTRAGTSGLVYDFTLYVGEGTCPNFGLGISSDVVLYLSKGLPKGKPFKLYFDNWFTSVSLLVALKENGIFATGTIRKNRISSCRLLSDAELKKRGRGSFDTKYEVNNNLICVKWFDNKSVQLLSSYEDHQPVGMCKRWSPKEKVYIDVVRPAIVGSYNKGMGGVDLADMLMELYKINHRSRKWYIRIFYWCLGTSMTNAWLLYRKNFHFLNPNQKYIPLIKFQMEVAHELLQCTYSTSFEKKRGRPSNMQRENSIISLGSPTSSVSSNQSKKYKFQPNPTNSMRYDCMEHWVVFTEKGRCRLCKTGTPMSKCIKCKVHLCCNNNKNCFMSYHT

SEQ ID NO: 21 - protein, 505 aa, synthetic construct
MIQGQIMVSDTEEEPSDSTGRTKRRRTYAWRKRDLAKNPVNWPDVQGACQDKRPIEWFENFLDEDVISLLVSESNKYAVKKNLPGDITTEDMKCFIGILLVSGYSWLPRRRMYWENSPDTKNELISSAMTRDRFDFIFRHLHVNDNLDLQDKYTKVRPLVTLLNKKFLEFSPLEEHYSVDEAMIPYYGRHGCKQHIKGKPIRYGFKAWVGATRLGYVLWMEPYQGATTMCNPIYKELGLGASVVLTFCDVLISRGFDLPYHVVFDNFFTGTPLLEEITKKGLRCTGTVRENRTSSCPLITSKLLKKKERGAVDYRTTHDNTFIIAKWHDNNIFSIASNAVGINPKQSAKRFSQSEKRNIVIEEPHMVSIYNKYMGGVDRSDENISHYRIGIRGKKWYMPLLTHMIDLAEHNAWQLYKINHGKLDHLGFRRRVAIALIESNRKNAKRGPSRPSHHEHADSRKDQMNHLVIPQSKQTHCRQCHKKCLTRCKKCDVGVCVKCFETYHS

SEQ ID NO: 22 - protein, 524 aa, synthetic construct
MADDNIELNDSFERLLLQSDDEHDSDLMETIQQDESDESDSADSESTDGGSDDGSEEWSKNVQKPLRWDFDDGTAGMNMDMVMDCKEPVDYYNLFMNNELIDFIVEETNRYGPTKDSNYLPTNNMEMRKFMAIILQMGYVVLPKLDDYWSSDPAIGGKAICGGVMTRKRFYSLMRSLHFADNDNNNGSKLYKIEKFVNLFIARCQHLLVPGRDICIDESMIPFRGRLRFRMYIPSKRHKYGIKIFKLCSNNGYTYNLSIYAGKEDQPRYGSVAERVVLKLMENLLGHGRILYTDNWYTSMNLAKSLLENKTNLVGTARKNRIGFPKHVTSLKLKKGDYVAEQNGDGIMILKWKDKREVIMLSSIHDGSITERGKPAVIVDYNKGKSFNDLSDQLGSYCPYVRKTMKWYLRIFFHIITQVSIVNALHLYKLHSGKNIKIVQFKRDIVSSILQLEKPTSSGRKHQLEKEPGEGTIRKRRCTECYNKLSKEFGATIARNRASQIKTRCDNCLKPFCLACFQKCHTKC

SEQ ID NO: 23 - protein, 662 aa, synthetic construct
MERHKFYDPELMSQDDVFNLLDSIDAGELDTDIEDNFLSDDDPDFVPNAHHFNMENILDNDADETNEILTAMDETNIEPGTSNSEIQLVESEEIEIGPSQSKKPQLEQVENDNFVNTFIGSSTEIDPKNIAFKNILWRKKNIQLEKKQIQFRGNEELPARFYELKTPYECFKYFLEDKLYQEIANTTNLYARQMNISTKFVTTPAEIQKYVGILFYMSIYRYPNTREYWGENSFEPVRKTMTKNRFEEIRRYLHFNDNTKMPAQGDSNFDPIFKMRPIIEYFNIRFQSVPMSQRLCVDEQMCSTKMVSHIRQYMPAKPHKWGMKLFVLCDTYGFSYGFELYSGASDNKIPNGAPDLGAAANVVSRLSQIIPDHMNHIVYFDNYYSTLPLMIYLYSRGIYSLGTVRANRIANCKLPTDKEVAKKPRGFSTEYVGSCYGVDLSTTLWKDNKGVLLASTYVGVLPFKNESNNTLKASRYDRAQKKRIEIDCPNIIREYNAHMGGVDLMDGLLGRYHIRMKTVKWTSRFFYHVLDLAMINAYLLHKRINRQNKACNIQLPQFRKEVAAMLCRFQTDEPQKRSVGRPRSTNAEEDPAPKRLGKRTYLPAADIRFDGKEHFPQWLDRSGKRQCKLPGCKSETQCTCSKCNINLCCTAAKNCFAIFHKQ

SEQ ID NO: 24 - protein, 554 aa, synthetic construct
MASNKRKLSDVELEQLANMDELQWNEFMDEFSDGGSLYEPSSCEHSSENSDADSSECNEDDEQVGTTAATTSNSDEWSDIPRDPPVFVFNENVGLKLDLQNFGINALVDFFFSDEFLTLLCEQTNLYARQEAEKQGGRRSARLSRWRDTDVAEMKVFLGLLLHMGPCCFPSIEHYWSTNPFYNVTFWRSIMSRNRFQLLLRYLHFSDNSLQSNDRLYKIRPVLDHFNNIMSNNYVPDKNLCIDESMVLWRGRLFFRQYIKNKKHKYGVKLYELCESNGVVLKIKIYCGKSEKNAEMGHASDVVLHLMESLLNKGYVLHMDNFYNSVSLTKLLSTYKTYVCGTLRNNRKENPKSLVAMKLKKGQMAWKRNDNVVVCKWKDKRDVLTISNMHCAEMVEVRNRNGKVALKPNIVRDYNNGMAGVDQSDQMLSYYSALRKTVRWPKKLALHIIEIYLQNAHRFYNQTAGTSIKSLAFREKFIQALIGDRMPDTQKKSKTEGSFHYLEAIPPTEKKLRPTKPCRVCTQQKKRRESRYFCPVCEEKPALCVEECFKTYHQ

SEQ ID NO: 25 - protein, 557 aa, synthetic construct
MYRQRGLSDEEVLQLLENSEVESETDFADEDDDYVPETSEDEDIVNEERLVEEDSRADLHQSSHLSSQQAQLAASTKMVARDGTVWETTNFSSSGRRSAVNVLKEKGGPTAYACQRIDDSVTSAFRLIFDEAMLRLMKKYTESNARKVLKNNNWTVSLEELEKLIAIMYARGVICTKGMSVDDIWSHSWGPTFIKDIMPRDRFQELLRFLRFDEKSTRVERLAANKFALVSEIWGKFIENSIRAYRPGENITIDEQLLPSKTRCRFTQFMSNKPDKYGLKFWLAVDVTTKYICNAFPYLGKEDMHREKQPLGEYVVLRLMEPFVNQGRNVTTDSFFTSLQLAKKLKEKKTSLVGTMNKIRREIPDAVRKSNAEIHSTSILHHSGNPECTMTVYQGKKNKNVILLSTLHSEVAISKEGKKKPETVTFYNATKYGVDVVDQMTRKYTTKVACRRWPMHVFYNILDMAAINAWVIYNIVTNKKMERKKFILQLANELKGMKEQEHQAKEEDTGPKSTRKRRKCQISLCKGNKTSENCVKCHKAVCGKCARKTEIICAKCV

SEQ ID NO: 26 - protein, 581 aa, synthetic construct
MTNKDEPLPGPSGMSGSRKRKWWEDKKKLTDEELEKILQESESDIEPLDSDFDFHPSDLSEDESSEHEDDDELEMEQQPATPEPEHRPTRRKTRLESGYVEWSDNSDSIRNFPFRRNEELLIPPPENQPISYFRHILTDSFIEKLVEETNVYAVEVFLSEKTKEKSRVIAWNETTSDELLIFLGLLLHMGHIRLNRLQDYWKKDPLFNIPVFSQNMSRNRFLLILRLLHFERNPEEGEPAPDDRLYKVRGIIHLFNERMNEIYYPGRELSLDESMVLWRGRLTFRQYIKNKKHKYGIKLYMLTNPTGIILKFMVYTGMLDDSGGKGHADKVVLKLLEEKLNVGHSVFMDNYYNSYALARKLSQNETHTTGTLRIDRRNCPTDVKKKKKLMKGDTIAKYANGVMIGKWKDKREVCYISNEYKNEMVDSANKRGQIRQKPLPIVQYNKFMSGIDRQDQMLAYYPSERKTIRWPLKIFIHILQMIMINAHSLYNKYSGKRANLYDFRLAVIRELLGQQPERKPAGKLTHRLTTRKEKTASGKIMRKYCKDCHSRGKRKDTIYVCDSCPNAPGYCLDCAVITHKL

SEQ ID NO: 27 - protein, 583 aa, synthetic construct
MARESLSYSEIYKILYDDDSEVNDSDADPDFEINSDHESNSEVSGDEEEDVQPSASIDAPTVVDSSTAHHSDSGTSPISFYKGRNGATKWRKTCFPQSVRTRRHNIVTMHLPGSIRAAMDVHSPLESWQLFFSKSLIETVLGSTNIYIDSVRDKYNSDPNMARPTSIDELMAYFGLLYLAGTLHGGKMNIEDFWNTDGTGIEIFRASMSLKRFRFLTRCIRFDNIHTREERKAQDNLAAIRTILDMFVSNCNSYYAPSENVTLDEMLVPYRGRCRFRMYIPNKPAKYGIKVFILACSKTYYVKHLEVYAGAQPEGPYKVSNKADDVVLRLVDPVKGSGRNLTVDNWFTSYPIVKRLLHEKNITVVGTLKKKPEIPFEFLPKKSKEVYSSMFGFQEYITLLSYVPKRNKSVLLLSSMHNDDEIDSDSGGKKKPSIITYYNKTKGGVDVVDAMCGEYTVARGTRRWPLCLFFQMLNIAALNGYIVFKANGNVSVRREYLRTLGKSLVTPYLKMRATQANLPRQIRRLASNLSGTQEQGIVEDPQPGRRGRCYECKDSKTKYFCKVCKVWLCLAHAEIVCGECFEK

SEQ ID NO: 28 - protein, 585 aa, synthetic construct
MRILYFRARKRLTSEELEWFLNEDPDFLKDVGSMSDDDTDDEEEVIAQSDHNTESEQEYEFDENEDSNCQSGCTGDWFVGRDKTTMWMKVVSTATSKARSKNIVKTFPGPKAIARDVTTEISGFSKIISMEMIDIIVHYTNKYIQLIPDYQRERDKKETTRTEILALLGLLYLFGIKKANHTNVMEMWNTDGSGIEIARAVMSYRRFLFLLRCIRFDDKTTRAERRKFDKLAAVRFILEEFVTNCKSTYNLSEFVTIDEKLQAFRGRCGFVQYIPNKPAKYGIKIFALCDAKTFFTSNLEVYCGKQPEGPYAVSNSASEIVHRLVSHIEGSNRNITMDNWYTSCTLAISLLQKKLTCIGTLRKNKREIPLEFLPRKDRAPGSSLFGYNKDMTLVSYVPKKNKAVLLLSTMHDSGTIDEDSCKPDIVLDYNATKGGVDTVDQMCGTYSVARITRRWPLVIFFALMDIAGINAQILFYSNGQNHKVSRRIFLKNLAYDLMKAHLVERAKIPSLPKDISLFLQRYQTTSEQLVQPSSNKRGRCIICGRAKNVNTTIKCDSCKSFVCRKHVIISYSCEKCHVATTDSED

SEQ ID NO: 29 - protein, 585 aa, synthetic construct
MAGKAERSMQRILREADVDDQERFGESSDDFDDSDNDPDFEVFSEHDTNSEQEPESDEDVTPDDDEAAYFFGKNRCFRWRKKQPAANVRTRQHNIVLQHPGLRQTAQSLGENPSALEVWNLFFTQDILDEIVLWTKLKISQLRAQYANDRLSYLQDVDVIELKALIGLLVYSAVFKSGHESVYSLFATDGTGRDIFRCTVSKERFLFLLYSLRFDNPDDREKRKEIDKAAAVSQIFLRIIENSQLCYSVGTTVCVDEMLVPFRGRCGFRMYMPNKPARYGVKIQCLTDVSTSYLYNAYIYSGKGSDGHTLSNHEKSHSVPTQAVLRLVKPIENTGRNVTGDNWYSSIELVNELSKKGLTYVGTLKRNKKEIPPQFLPNRKRRVKTSVYGFTEDMTLVSFVPKKAKAAILVSSLHHSENNDPVSGKPEIIQFYNTTKGGVDCLDQRCAGYSSSRRTRRWPLAIFYTLVDVACGVNSYVVHQAYPKAEVMTRMSFMKTLARDLVEPHMKRRLSIGHLPREVSGTIRRLLNIHDEPHTPKQFENRRTCAFCPPHLKRKTRYPCQQCGVPICLGCSRKVCVKCMRGQSD

SEQ ID NO: 30 - protein, 596 aa, synthetic construct
MNVNNCEQIRKWLEEDLISEDGSDPDDAWSDTEVADIPENHVLQESHDSESEQQAGSDSTSDGEEPTRNSQGRTDEISRRGKTYCSKNNFLWHGCPRTTRSRRRLHNIVSETPGIRREYRDKINSESSAEDIWNLLFDENILNKIIASTNQHLEKYKCKYEDKSNCYDVDVVELRALFGMLYYSAIFKENHEDFTNWYSSDGTGRDIYRSIISKNRLKTLLLALHFDDHTTREERRKTDPAAPITEVFDAFITNSRSIYCIGTSACVDEMLVAFRGRCKFKMFMPKKPAKYGVKILCLTDAETGYLHNAYIYVGKDSDGKYLPEEYKIFKKPSQAVLKLAEHIIGSNRNITADNWFTSIELVQELQRKGLTYVGTIKKNQKEIPAAFKASPTRPVGSSLFGFCDDLTLVSYVLKKSKAVILISSMHHDCTVDSTTKKPEIIMYYNANKFGVDLLDQRCANYNTARRTQRWPMAIFYRLLDIGGSNAFIIYNTVHEQKIRRFDFLKQLAGQLARPNMMCRAYNHHIPRELREVIRRILKVPEEKPQGEEKLEKRKTCYTCDPKKKKRKTFHACFMCRKPICVECSQTMCKQCRINLD

SEQ ID NO: 31 - protein, 598 aa, synthetic construct
MSGSASKKRSRKEDSIRFSSPNFESVVTQWVAEDDDFLSDISSASDENHEAVPQAVSYDSLDEEPRSGGGEEDDDDGLDNNISGQISSESANVYIAPNGMLWSKRPPVQNKTPAHNVVKFIPGPARGTITYTHKDAWDLFIPLSIVDEIVRCTNLEARRVCVPRKIIWHDVTRDEMFAFWGILLHIGADKGWDVPIRELFLGKFSNPLYRATMSVSRFEALRRFIRFDDKRTREARLQTDKLAAISYIWKIFIGQCRKVMIPKISVTVDEQLVPFRGRCGFVQYMPNKPAKYGMKIFWMCEAETGYAIDGTVYTGREPGEPPQKNLGLSTVKTLTSGIAGTARNVTVDNYFTSVPLAEHLLSKNLTVVGTMRMNKREIPKEMKPNSSRQVNSSLFAFTKEMTMVSFSPKKNKCVILLSTMHHDNSIDEQHPKRKPEIVKFYNKTKGGVDVMDQLVRNYTCKRQTKRWPMALWCNIMDVCTLNAFITYKAQHPTYHEGLLHKRRLFIKELSMELIQPFMIRRSSNPFLQKSILLDMEKFITIPETQNNSSNSNSTVGRQKKRCSLCPASKDRKTKTVCSKCSKHVCQDHSSVVCNNCKN

SEQ ID NO: 32 - protein, 601 aa, synthetic construct
MEEPSTSTGKGSAATNRQCVNTLDRNFEETVSRWLEESDDSDCDDNPEETEPIVSDHDTLSEQSATENYSDESSESSDPSDDQRRSNYVYGKNQYKWAKAPPTRAVRTPRHNIIMKFPSTTLTTGAPKDPFSLWHLYFDDEVLDHILLWTNAKIDCVRQKYSDKSKAELQNLDLDELLAFLGLLIYSSVFKSNHENVNYIFATDGSGREIFKCVMSRNRFLMLLNCLRFDDFRTRKERLKVDKMAAASFLFRKIVENSQKYFNLGTCATVDEQLVSFRGKCSFVVYMPKKPGKYGLKIMSLCDARTSYFYNGYVYTGKGSDGDTMPDNDKKMMIPTQSLLRLCKPIYGSNRNITADNWFSSIQAVEELMKRGLTFVGTLKKNKREIPQAFQPNKNRPVGSSLYGFTKDTTLLSHVPKTNKAVLLVSSMHHSPAEDLTSQKPEIISYYNATKGGVDELDKKCSIYSCSRRTRRWPMCLFYRLLDISTVNAYVLQKSGSDEAPVDRGIFLKELARSLVLKHMQKRAQNPRLPQELRLTTRRILGPDSPTEEAVEAELGPKNRKTCRICPASKKRKTSHACYVCKRPICMQCCSQVCKHCCENL

SEQ ID NO: 33 - protein, 605 aa, synthetic construct
MCEGYSTIEVADCVQIIVPHYRSQLHFSSVVCRRSSYIYRSNVCGAIMTKRVLSDEQLEALVNASDSEDTISEDENHTSDDNIHPSSESEDISDADISNDNVHAVQFIQSKDKKITWSLQPFPQTGRASSANIINVTPGPTRYAIARVSDIKSSFLVMMNDNIQSIVVEMTNIEGQRVYGDGWEALDKITLQAYIGLLLLAGVYKSYGESTKSLWDPETGRAIFPAVMSLKRFCCISRVLRFDDKSDRRARRVNDKMTPIRVLWDKWVEMLSKLYNPGTNVTVDEQLVAFRGRCPFKQYIPSKPSKYGVKIWTLCDSATSYVLKTQIYTGKLPGQSPEKNQGMRVVMDLTYELKGQNVTCDNFFTSYALGQGLLKRHLTMLGTVRKNKPELPNNISSKEDVHTSKFFFTKDTTIVSYVPRKNKQVVLMSTMHNSVEISNREDKKPKMILDYNASKGAVDTLDQLIGTYTCKRKTNRWPMIIFYNIIDTSAYNAFVLWREINPGWKKNVLYKRRIFLDELGRLLVSPYIVARKMQPRNEQSAAIVRKIQTGNPSTPSFIGGNRNCKRSRCKFCPSSCDNKTPMKCSNCNKHVCKTHVRYLCPNCEK

SEQ ID NO: 34 - protein, 667 aa, synthetic construct
MPVYIRVVIATHQVRCPYIYMGTLSFSYAAPVFKLFSVFLCLYMYRSLLGLVITLISTMELLYCVVLCCSLATVYRMASSSYVGLSEREVSALNELFSTLESEDEDPSFNYVDSDISDDETPAHFRCDNAHLRDNSDAANAARRSAPSPPAEQTEEMFHWVNSEFHPKAHKFSETSSGFKDVFCCVNDASEIIDYFECFVTPELTGKIAFETNRYFQYLISNTDPAVSSRLRKWKDTNSKEIYLFLAVTMLISRNKKLSLNDCWSTDKLLHTPIFGETMPRDRYLLLLRLLHFCDNEQQEPNNRLYKIDMVLNDIRKVCSENFYPFENLCIDESLVLFKGRLSFKQYIKTKRHRFGVKFFVIADVKTDYILDFIIYTGASTRLSVFDKTLGISGSVVLQLMQPFLGKGHSLYTDNWYTSPLLAGFLHRNQTNLCGTVRSDRKKMPAIEKPQVKGESRSKNCGHIMIVKWLDRKVVNMLTTIHADTMVPTGKTNRKTGEPVMKPLCVQEYNKNMGTIDKIDMLLSSVECLHKTMKWYKKVFFHLIDFAVVNSHAVYKLKTGSKLPLAKFQLELIRQLIKKYGNESRERKKTIRVSSDDLPTRLTARHFPQLVPPIPGGKQNPQRYCYVCSNTQLAPKRRRDTRYQCTECNVGLCIINCFQKYHTLQKF

SEQ ID NO: 35 - protein, 671 aa, synthetic construct
MASVRVRRNRQRNLTVDEVLSSVFDENTTDLDSSDSEDGEDELPTLFPAIPADLEDDSVESVSSYEDSSSSSSDSEFDRRQSVTNQRQEKARNLRECSQNARRTIRGVRRGRGARRGRAISVQQRREIQDGGRQQYIWSKEVNRRILKPFTNAVGLANRGQRREKSALEYFELFFDQEVWDCLVTMTNLNAERKGAKGTSGGQWKPITQDEMKAFFGLNIAMGIVKLPEAKMYWQKKIWLLTVPSFSQVMSRNRFFQILQYIHVSDDSNIVPSGQPGYDKLHKIKPLLNLLFPKFENAYNLHKNISIDECMIPWRGRLSFRQFIANKPVRFGIKVWVLADSESKYVYRQQLYIGKNPGERAEVGLAARVVKELCSGLEGMGHHLYTDNYYTSVELYQFLFDNQIYACGTIKGNRKNFAKEVLFENTRGLARGSSKWLMCGPLLAVGWLDNKAVYFLSTIHPPEFPVRAPVHARNVKRRGAGEGGQSGDIPCPPLLKDYNAYMGGVDQSDQMLRYYTCIRKTVKWYRRVLFHEVEVAIHNAFVLECEDREGTPRPIRTSLEFREELAENLIGNMRVAHNAAQRPPRNEELRLTDVGLHLPEMRQDKGNCAVCSKKIRKTHSDRYKDVPLARRPKVPYVPRPSIYCNVCNVFLCLQKDQNCWKDFHTKVEYWR

SEQ ID NO: 36 - protein, 655 aa, synthetic construct
MVFLHPLSAMSLKKGAKTIPAHSLLQCAYVVVFIANVCRKMGDSLTEEEILRLLEESGSEIDDDGFQTENSSSEIDGNYNGQSLCLRITIIISAVSLTYVIYFEGNNEWSENENEPSDVLEECEAPIQKGRGRIQQQQSQNNVVEWVNDDVLQQLPLFEGVSNVLAPLSAESTEFDCWSVFIDANVIKNIKTETNRYAAATISKLRMQNKITSRSLWANWSAVKLHEMYQFFAIIIHMCLVHKPNITDYWSNDLMLDSSLPAKIMKRDRFRSIYFMLHVNNNDNYVRRGQPNHDPIHKIRPFFDNFVLKSKNSFSPGENLTVDEGLCPFRGRIRFRVYIKNKPSRYGIKFFILSDSATGYVLNCEVYTGAGEKDNSADSVVLRLCNSYLRKGHTLYMDRFYTSVKLFEKLFDEGTSAVGTVMRNRRGLPQEFKQNKLRRGEIVFKRKNSVLALHWKDTRDVFALSTKHRMTSSVTKTKSKAGMVEKLKPDLILDYNKNKTGVDHGDQLVTYYYFQRRTMKWWKKVFFHVFMMCVVNSYILYKKLRPVSHKRTSLFTFMVNLGKQILEKSGEVFNENEKQGSAANRLTARHFPTHVPSTTSKKYASRYCIVCSEKGARSTGKRVRRETRWWCEECNVGLCLPDCFKQYHTKTNFTQ

SEQ ID NO: 37 - protein, 605 aa, synthetic construct
MENPGPSSISENSSQKKFIGTDKISEIVNDSDSDNGSFSELSNSETYGANSPFSSSSSEEAEEEIVQPEPDRGRKRTRRAIPKRSNTDLELGWMEKIRKIQKPAFSGVPGINKIYQITQDSSPLDIFEIFFSTDLLKRIQNETNRYAAQQIKKKKQEGPLKSKSVFAQWNPVTLQEIKKFFSVVIHMSVLRKSSLRDYWSVRPIIQTPYAASVGMSRDRFLAILTMLHLNNNDAKAARGEPDYDPLFKIRPVIDTLIAKFQDVYSPEEELTVDEAICPFRGRIFFRVYIKGKPHKYGIKIFELCEAKSGYVCNLEVYTGAHPTSSEHNSAVSVIDRLCDTIKEKGHSVYMDRWFSSPKVFDHLWACKTKAVGTVMSNRKEMPKQAFSVKLKKGEKISRQRDHLLAIKWKDTRDVFFLTSAHEDETVEAPSSRGTHKIKPTAVLDYNKYKTGVDRSDQMLSYYSFERKTIKWWKKLFFHLFDLAIVNAHILHTKTNKKKLSLEIFYEKVAEGLLASAGTEPQVQGQTSSPAGRLVGRDHFLYRIPATHTKLEGKSQRSCRVCAERSKRQTGKTVKKCTTMYCRKCDVGLCIGQCFEVYHTRLNYWE

SEQ ID NO: 38 - protein, 633 aa, synthetic construct
MVVKNPCKLCGGLYYCQNPARRKSSKNISHSFSFTMASRSKCRHVPTDQLESVVKEICADDDSGDEIMIPELNEVDSDNSTVIDNDPQDSNHEHLATTSAVPDTPISWTYGDIVPIIVPFTSSSGIKIDITHKMHPFSWIKFFLDDDLVTHITTETNRYAHQHFNTHELAPHSRELNYKYVTSDEIWTYLGIVFMTGIDKRPEIEDYWSTRPLFHTPWYGQKMSLRRFQQISKFLHFADNDARPADCEDRLYKVRPIIDSLVGKFRNTYIPEKEIAIDEGMIAWKGRLIFRVYMPDKPDKYGIKAYLVCESTSGYIWNFEIYCGKSRPIKELVVDLLGPQLLDKGYHLYQDNYYNSVELCETLLQRNTYVCGTFRMDRGAPKELKNDVKKLKKGESVFSRKGQVLVQVWRDKRDVKLISTLHTAKIVESAKTNRKGEKICRPEVIGDYNKHMRGVDRADQMLHYYPCSRKTVKWTKKLVFFLLQMAALNSFILFKKYTTTEKYKKRNYKFKDFILDCVEKMTEQNKQCGEGEQSRNEVDDESIASDSSAVTPPPATLRRPPVKDPENRLEGGLKVHKMVHVPPSNNKKNGAMRKCRVCAKHKIRKETSVMCASCGVALCKLSCFNVYHTKKNY

SEQ ID NO: 39 - protein, 558 aa, synthetic construct
MDKKRPLTQNEIEYYANLSDSEFDGIFGEDGNDSSDYEPSENSSDSEDEVVSEPEVDPEFEDNDDDSQATTSNANEDLWTEIQQNPELFIFQENVGVKLDTTNFTVQTLVDLFFSDEFLALLVEQTNLYAAQEIEKRKEIKKSNRLSKWEDISAAEMKVFIGLLLQMGPCTFPSIEHYWSTNKLYNVNFWRSHMSRNRFQLLLRYFHLVDNSIPSADRLYKVRPILNHFNNIMHENYVPDKNICIDESMMLWRGRLLFRQYIKNKKHKYGIKLYELCESDGLVMKIKIYSGKSEETDNNVGHTTDVVLHLLEDYLDKGYTVYMDNFYNSVTLTKLLNTRKTYVCGTLRNNRKGNPRDVVNRKLKKGECIWQQNGPVTVCKWKDKRDVLSISNMHAVEMVEVPNRNGKICMKPNIIRDYNNGMSGIDRSDQMLSYYSSLRKTIRWPKKVSLHIIEIYIHNAHKIFQKVTSSKIKLIKFREEFVVALIGEEIQPVSKKIKLQELHYLESLPPTKKKQRPTKPCRICTQAKKRRETRYFCPACKEKPALCVEDCFKKFHKQ

SEQ ID NO: 40 - protein, 559 aa, synthetic construct
MSRRRNQRHTFIQNEQVIQALQESESEGDFSAGSSNEYVPSEHSDESETSSPTSISSEAIEENTIPTVQDQPSCDPENLWSFQSARPQNFIFTGNASFNIPEVSTPGEFIDVFFSDNFVDHIVQMTNAYAKSTPGPVQKNARSLQWKETNRKEMRQFIGILLYLGIVSAPTIAHYWSKDLLYNFPFIRKVMSRNRFQILLRYLHFSENDEEAGTDRLHKITPVLDWFNHIMMTIYQPHQSLSIDESMVLWRGRLVFRQYIKNKKHKYGVKLYQLCESSGLVLKIRIYRGKADRPSSGSLHSTEVVMDLLNGYFDKGHIVYTDNFYNSVDLARKMSAKKTYICGTLRANRKGNPSEVVKAKLKKGEIAAMSSGDITICKWKDKREVMVISNKHKINLIRVRNRNGKIAIKPNIIADYNLGMSGIDRADQMTSYYSCLRKTLRWYKKIAIHFFDIYLQNAHRLYRFQHPESKMRLLKFREEIIKHLIGPLNDVPQSTARPTTQEIHYLDLIPATEKKNAPTKPCRVCTRNKKRRESRYVCGVCPERPALCVTPCFRTYHAQ

SEQ ID NO: 41 - protein, 573 aa, synthetic construct
MEDNRDTAGPSEPKKRKTQDTRNVQKLTDAELLRILEESDSETELASEEDGVESSEESDGAEFVLDETVEMAVNPSDREMAEGVVTRDNAADTTVAWEREPVGMINCPFIKNEGLLIQPTENTPLDYFRLLLTDEFLLTVVEETNRNAIELFLSAGTKGQSRINCWKDVTVGELLVFLGVFLHMGNVKMRNLQDYWKKDALFNVKGIADSISRNRFLLILRALHFSENPKQGKPTPSDRLYKIRPVINFFNERMCQVYYPGRELSLDESMILWRGRLLFRQYIKNKKHKYGIKLYILTTPTGMVLKFAVYTGMLDDLGGRGHAQKIVLHLLDEKLNAGHHVYMDNYYNSFALAKLLLDKKTHCTGTLRANRKDTPKEIQEAKLRKGEAVARFAEGVMIGKWRDKREVCYISNAFTNEMVEVETKRKEKKSKPLAIVNYNKFMAGVDRHDQMLSYYLSERKTIRWYKKLFIHVVEMILTNAHALHNKYCGTKMPLQEFRLSIIRALLPRKQVERPVRNPQHVVVKRESNGKSKTPRKKCRSCASRGQRTDTIYECLDCPNKAGYCLNCCVIHHL

SEQ ID NO: 42 - protein, 576 aa, synthetic construct
MDNQPGPSGIKKRHCSDEKKVYDKKKKLTDTELLQLLEESGGSSDEHYVPDTDDSDVNSSSESEEENFIDPIRPHQTLPSLDEIKWNDRGALKQILFQKTQELLVQIPGQGAPIDFFEMIIDDDFLQTIVERTNSYAVEVLLRPETSTKSRITNFKDLTLDELKIFLALLLHTGTISLSRLNDYWKTDPLFNLPVFRKYMSRNRFLLILRCLHFTSPDMDPADRLGKIRFVADFFNNKMGEIYYPGRELSLDEAMVLYRGRLQFKQYIKNKRHKYGMKLYMLTEPNGLILKFRVYEGATDVYGGSGHTEKIVLHLLEEKLGVGHAVYLDNFYNSVNLARKLLQQNTYCTGTLRAERKDNPKEVMSANLKRGENKSMFCNGVHVGKWRDKRNVQYISTEFGSEMGIFKNKRNQESEKPSAIIGYNAFMSGIDRQDQMMAYYPCSRKTIRWYKKLFMHVLQMSMINAYFLYNKYSRSDQLRLYDFRLDVIRNLLARRPITNLPQIPPGQINVHSISKVEKIVSEKKGTKETKRVSRKECKECRKNKIRKQTVYECKMCPGQPGFCLNCFEDYHKNVQK

SEQ ID NO: 43 - protein, 582 aa, synthetic construct
MDEENPGPSRPKRRHTDFKMMRPLTLSEQEELLYLSESEEDPFVDSGSEYEPTDHESSSDEDELPDVAALEDASLTDIENNETISVQNSNSDIPTWTDVPNQNDFLFSGVPGLRVNLTSDNPIDFFQLFITDDFLEHIATETNNYAEQLFLSKETKEKSRITEWKPLTVPTLKTFLGLWLHMGNIQLNRLQDYWKTDELYNLSCFRNNMSRNRFLVILRCLHFSKNPERGDPDYGDRLCRIRWLQNFFNDRIDKIYYPNKELSLDESMVLWRGRLVFRQYIKNKKHKFGVKLYILSEPDGLVLKARIYTGDEKDMKGELGHAANVVLSLTKNYLDKGHSLYMDNYYNSIALCKELILKKTYATGTLRLNRKGNPPEVSSKKLKKGESITKYSKGIAVGKWRDKREVLFISTEFSGELIKQPNRRNEEKEKPNAIINYNKNMSGIDRQDQMLAYYASERKTIRWYKKLAIHLISMMLLNSYHLYNKYSTQQKLSYYDYRHNIIKNLLSEAKEKPPEPPKVKQNQPSHLPEKCEMDNKGRQMRKRCRVCYTQKMRKNTPFFCPVCPDKPGLCLGNCFRIYHDEK

SEQ ID NO: 44 - protein, 626 aa, synthetic construct
MQRSAVILFILVRPTVVLVYSVCVPCIVFTISQLLVLYKMNYEKEQEELNRLWQECLSDEDDNEPFEDSGDEFVPESSDDSSNDEPISKKLKFVKEPIPSTSRDTPDSVLDTINDVIERCMEEGSDEDNANEHCDNTNELTWEDVSGRNLKLFPFTEINVGVKEEIHAEYYDKGPYEFFKLFLTDELISMMVIETNRYATQCKASATLPKARIREWHDTNCEEMESFIGTLVWMGLCPFSSIEAYWSKCVVYTNKIKDIMSRNRFQLLLKSWHFHNNEILTEDRLQKITPLTNLLIKNFQNVVIPKEHVCIDETLVPFRGRLSFRQYISNKRHKFGIKLYKLCLEKGYTYNLQVYCGKDKVPNQSSSEKVVLSLSDNLLDKGRTIYTDNYYTSISLAHQLLKRNTHLVGTLRTNRKFNPKHITEKKLQKGDTIAAESNTGIVIQKWKDKRDVCTLSTKHTAEMKTVSHFGKDSQKPIVVTVYNKHKSYIDLSDQMKSYTTSLRRGVKWYRKLAVELIVGAAVVNAHILHQEVANEKMSITKFKEELALKLTKIDIRHDTTHHQQAEHVLEDVGTKSRRRCVVCYEKLSKTDRKLAQSKTPQSRYRCITCSKNYCLNCFFEVHKCTK

SEQ ID NO: 45 - protein, 579 aa, synthetic construct
MDYSSDNSDYDPDYYPSDEPSDYSDIGDSSSSDDIPLSSLRLRIPQRADSPSMPSVFGTTANNITEDMIVKQYVDNIKWVPPKDPGPQGEQFTGTLGMKDIYQGILADAQPLDYFELFLTPDIMDLIVDQTNIYASQYLLTNDTSAQSRLHSWKPVDSAEMWKFFGLIGWMGLVKLPAIRDYWRKHKLYGLPFARTVMPRNRFEMILKFLHFSNNEEDTQNDRLFKVRNVMEKFIKNYQNSYTPGEKICVDESLIPWRGRLIFRQYIPNKRHRYGIKVFKLCTDKGYTYNLSVYCGKSLSTVKDMSEKAVMEMTEGLLDAGRTLYTDNYYTSIPLAYRLKHRKTDLVGTFRQNRKHASKEVVRQTLKRGELAGMETDDGKIALTKWKDKRYVTVLSTKHSAKSMETIRTKTGRSILKPSSIVDYNTGKSSIDVSDQMASYSSALRRCVKWYRKLIFELVWGTSLVNAYFLYSKYSRQSKLTITEFRELVIEQMIDKHKGNVTEPEKEPQAASTSAVHKLVDNIIENSPKPKKGRCKGCYAVYGKKGKIIDGKRKYAAQVITICNICKCFYCRKCFNKAH

SEQ ID NO: 46 - protein, 568 aa, synthetic construct
MSDSEDDPDYQPPTDEESESMCTYSDATSSEESVELSTSHQHQHSSEAIPCRSIPNSSWNIPLSDGSRKKQFPFTVDPGMKNIGSDCQTELDFLFLFLDNEIIDLMVRMTNSYAIKTQILKRVTRNSRLSKWNDCDAAEMKKFLGLLIWMGLKKLPKISDYWSTDVLYENNVAKRVMSRNRFELLLRCWHFEDAYYIGMTGTDRLIKIRRFVEILSKKFGQYKTPGEFLTVDETMVAFRGRIAFMQYIPGKRHKYGIKLFKICDDDGYTYDLIVYEGKRTTSTTQNVSSEIVMKLSDQYLNAGRSIVTDNYYTSVTLANSLLQKSTHLLGTLRKNRKGLPKELLKEKLKKGETCVMENNDGVLILRWKDKRDVLALSTRHTPSFVSVRSKRNRNKVTMKPTLIADYNRHKCAIDYSDQMGSYCNPARRSLRWFQKLAIELLFSTSLINAFILFKNSKNLTSKKYTITTFKENICRTLMGLDQQRRQGRINQDCSAGHRFGKSTLVDQRNRIVRRRCIHCYEKKREEGLTSVEACKVTKKVNTVCLLCPSNPSVCSECFANFHSAIVRQ

SEQ ID NO: 47 - protein, 598 aa, synthetic construct
MCNKNVSTIGYSLVVCLLLYLYVLRDPNKALTDSQLEAYWENYESDHDPFSTDSGSNYMPSDSEYASSELDHDISVDEDEEEERQADVSEIDKSDNSNLSWNVYTGNQADFPFIGQAGIQMDLSEYIAPGDVFKLFINDAVLDLIVNETNKYATDMVNSGILSKSSRLHNWQATTPKEIEKFLGIMLWMGLSCRPKLRDYWSSDSLYSNSISKVMSRNRFEILLRMIHFSDNDIPADGNRLKKIQPLLDILQKNFQEVYTIGKKMVIDESMVPWRGRLSFRQYIPGKRHKYGVKLFKICSLEGFTWKIQIYCGKHECSGVGLPEKVAMNLCEGLLDEGRILYTDNFYTSIPLAKNLLTRHTHLVGTLRRNRKYLPKDVINRKLNLNEIYGLESDEGIVVSKWKPKKNKEVIMLSTIHNLEQSCVASRNSKKKVADQPLPQKPNVIIDYNNGKIGIDKSDQMSSYNTSVRKGIKWYRKVAFELLTGVAIVNAHIIYKSITNSKSTITTFREEVCRNLLDLEVRERIKKIKFNHLLKKRDGGGTKDRRNCVECYDNLLKNMTRVEARRKVRKVKTYCDGCENKPTICYECYLRVHGEGSK

SEQ ID NO: 48 - protein, 579 aa, synthetic construct
MDPLSFYGRIRKNSNSDFDNRTGISDDSSDEEDFSSGSDDNYICEDVNDSSDSESDNENNLFDEWPQIASTSTSSVRQEWSNDIAEAQKIPFIAKHGLNKRIVIEDGVPLPINIFEQFFDSEIVDLMVLETNQYAEQFLESNQVRRNSRMKRWSNTTHAEMKKFIGVIITTGIIKYPNIEDYWRMEPLFYHPLFHNIGMSYNRFCLILKNWHVADNKTANPGDRLHKISNFLNTFISNIQEIYTPGEEISVDETMISHSGRLLFKQYNPGKSHKYGIKLFKLCDMTGYVWNFSVYCGKGTSEKLPGLDHAGSIVASLAEDLLNEGRLLVADNWYSSIPLAMFLKNKNTEFCGTLRSNRVGLPNLKSFKLTRNEIAGAINSTGIRVMKYQDKKEIRLISTFHGLNMQETGKKNREGKIVKKPVIILDYNRVKGGIDLSDQMISYYSPARKSVKWYRKVLFECISIAILNSWVLYNKFYSNKQMKLEQFTKSIANSLLGITEERTTMKKTKNTPHELSEIPRKDDGKLVRKRCLGCYKKLAESHNRNTASTKSKQVNTQCIECQKPYCLPCFNEIHNNYTS

SEQ ID NO: 49 - protein, 570 aa, synthetic construct
MADFTYNDYEVILTAESDNSDVEFELPEIYEPNEDNTESAHVDEDNIESANIEEDNIGSTSGVERSPVFHWDSDPFLPQVHNFDSSFSGAKLCTDESMQEVDFFKIFIHQKFVELIVTETNRQYKCIKNKSSSISYRMQQWVDTTVDEIYIFLGLCILMARNSRNTIEEHWSTDILLHSPIFSDVMPRNRFQILLGMLHFCNNEEQPPGDRLFKIKTIVDMITINFQEQMYPYMNVCIDESLILFKGRLMFKQFIPTKASRFGIKLFVICDCATGYVLNFIVYTGATTIYTQHKELGISGSIVMTLMDPYLNRGHTLFVDNWYTSPLLFQSLFDKKTNACGTVRKNRKHMPKFKKKLKKGEIEAYHTDSMAAQVWKDKRDVAMLTTLHPSTLAETGKLHYATNEPILKPESINDYNKNMGAVDKCDMIVSSVHSSRKTMRWYKKIFFHLLDLCLLNAHVIYREKTGKRKALAEFQLEVIRQLFYRHQQSSEPKAGSSRKRKSDLPSRLVDRHFIGKFPATAQKQTATKRCVVCLAKKKRRESRYMCKQCNVALCVEPCFEQYHSLENYNK

SEQ ID NO: 50 - protein, 592 aa, synthetic construct
MPFKPKFYTDDELYQILANSESEDDDVVSNSGDYGWLDDSEPTAGPSTSSKQNYVEEDNLPSIISSSDSESEEEDLVVPTKKFKSDYIWKSSDLSPVVHNFDNSTSGCQIPQLDMSSTALDCFKIFFDENVTQYVADQTNLYSIFVTENTELSKSSRVNRWKNTDSSEIYCFIATNFLMSQVKKNSINSYWTNDKLLATPIFSQIMPRDRFLLLLRMLHFTDNNKKPENDVLWKLRYIIDHLRDVFTKTLYPFQNLCIDESLLLFKGRVFFKQYIPSKRHRFGIKFFLLCDCETGYVLDFIIYVGAATEIKKNEYLDKDIGISGNIVLTFLERYLSKGHSLYVDNWYTSPALFTYLHKNKTNACGTVRKNRRNLPNLSKNLKSGEIEYMSTDRLLALKWKDKREVRMLSTFHNSDMKDSGKVNRTTKEEIRKPVCVLNYNENMGAVDRSDMLLSSVECVRKTVKWYKKLFFHLLDLSLLNSHAVYKMVTGKNISLLDFQGQLIKQILEAHHLPQQHSRSGRRSADGDNPLRLTERHFVSMIPATAAKKFAQRKCIVCSKHGKRSDTRYMCALCDVPLCLLICFERYHSCKYY

SEQ ID NO: 51 - protein, 571 aa, synthetic construct
MDEQQPGPSKALPRPKKGQFYTEKELLEILTRSDSEDEYIENTQNALDSDEEMDVAQEPEDQTSNNTYDFLWKKKDLLPKTFAFDSSEVGCTIDGLTAENKPLDFCKVFFTDNIVNHIVTETNKFYIYVCEHVEVSAKSRLKKWKDTDFNEMWLFFGITYLMPRVKKLRLSEYWSTNTLLSTPAFSELMSRDRFFLILRMLHFSDNNLPKNGDSLVKIRAVIDHVRDAFRNAVKPHKELCIDESLLLFKGRLFFKQYIPSKRHRFGVKFFVLCDAVTGYILDFVVYTGATTDLQTNNCNLGKSGDVVLTLLARYLDKGHTLYCDNWYTSPSLFVWLYNRATNACGTVRKNRKYMPQMAEKLQRGEWSFRSDDKLLSLKYCDKREVYMLTSLHNNTGMETGKKDRLTGRTIVKPQCIVAYNKFMGAVDKTDMLLSSVECVRKTAKWYKKVFLHLMDMVLLNANVLNNIVAKTKTSLAEFQLTLIQQIVEENHKGKMRTSAGRRSVDDNPLRLTCRHFPSHVPPTTSRKVGLRKCVVCAKNKKRKETSFMCRECNVPLCVVNCFERFHTIKHF

SEQ ID NO: 52 - protein, 568 aa, synthetic construct
MAEAKCSSFVIDKDIERLLDESDSDKESLSSQSGFSDASVVDYAQGEVIESDFEDESEEHESPPTPFGWKRVGDAYCPSRIPFVGQSGPQIPVGTILDSFMHIFDAEVIDLMCTETNRYARQFIAKAAPNPRSRVNNWREVTREEMYVFIGIFILMGIIQKPSIKSYFTKDPFLETPIFSQVMSLDRFELLSKFLHFADNEDEKNFAGPKKLFKIFPIVSLLRTKFQTVYVPHGNVCIDESLTLWKGRLGFKQYIPLKAAKFGIKTFELCESKSGYLWNFIVYTGSGSEITCNINVDDNLKSTKIVVHLMEPLFEKGYTLWMDNFYNSPYLSYVLRKNGVNVAGTLRLNRKNVPLSVKTKQLKKGETVAYESNGIMVLKWKDKRDVAMISSFHDDAMIQKKIRGVDIVKPKCVNEYNLAMGGVDLKDQKLQPYLVERKRGMKWYIKFFKRLLNVAVHNAYILYQARNAGVDHLIFRISLVKAIVNINRPQLGSPTVGRPSINPPPRRLVERHFLERIPATGKKAKPQRKCVVCTKHGRRKESIYWCSVCEVGLCLEGCFKTFHTEINF

SEQ ID NO: 53 - protein, 577 aa, synthetic construct
MSSLRGNFANRPEEIQKYLCESDSDVTDDEDFELPTNIVVNDGSEYNSECESDENEDIIRNTYTWEWKNEINRVVEIPFIGHSGVNENIIQKLCNDGTVPKENDAFSEILGDDFFEMIVLETNRYAQVKLDTVDIQNRQKWFPVTVDELKAFFALIVLMSQNPKQRLEMYWTKRIILRTPIFSKVMPYKRFLLLCKYLHFANDYEGDDDRLKKIRPMIEYMNVKYENVYTPDKNIAIDESLMKCRGRLSFIQFNPSKRSRFGVKFYKLNESKSGYCWKFSVYCGQDDHGDMLASEAVVMNLSEPLLNQGYAIHTDNWYSSPTLFRLLLDNKTYALGTVRKHRKNMPAAVVSKKLTAGEVVHQSSNNILCLKWQDKKDVLMLSTMHDSSLMTDTGKTKRVKRKVGGRVQVEEAQVVKPQVVIDYNNAMGGVDRQDQVLSSFPVMRRYSKSYKKIFFYMFDMALYNSFVVWKTLTGKRESFTEFRLNFVEQILEHVTLPQYPSRGRPSLGPSPLRLQTKNWAHFVMKIPPTATKANPTKRCFVCAQKKVRSETVWQCEKCEIPLHIPTCFRDYHTKLNF

SEQ ID NO: 54 - protein, 584 aa, synthetic construct
MAKSKEKKLRLDEICSFLEREDDSESEYSPSESQSDLECSSSDDYESYEELSDAESPDILPQNVSPGTQERNFQWTQYDIDQAEDGYGKPIFAGVAGVCVPLSSDVSPLQCFELFFTDEFVHHVVVETNRYANKCREDALNSGVGSNARIRLWNDTCENEIRLFIALILAMGIISKPELKLYWTRDYILQTPIFYETFSLKRFQLILRYLHFVNDEDTGNTDRLRKIRPLLEMLRLSFRKTYLPEQNISVDESLMLFKGRSLMKQYIPLKRSRFGIKIFFICESQSGYAWDFFIYTGQGEERRGDESASRKLISHASVTKLVMCPQLIAKGYVIFMDRWFSSPNLFQELQSHGFGACGTVDLRRKEMPKELQTAKLKKGDVLCYTSGVYSAIKWRDKKDVTMLSTYHRGKICETGKVDRKTKEAVKKPDAVIEYNANMGGVDRLDQKIKPYECLRKSLRWYRKLFFHLMDISIVNAHIVHGKSGREKCTLLQFRHKVIHGLLEKYAMERKVAKGGRPSKTDPPVRLTERHFPTHIPATEKKKFPSRVCRVCPGRKETRWECKQCGNIPLHPEHCFEMYHTKRKY

SEQ ID NO: 55 - protein, 586 aa, synthetic construct
MNTPQSLTMAGLSRSAIAAELCGEDLSDSSSSDELLSNSSSDTEEGSYSQENEESVIACAVGGDLEVSVQAPLESADWKEVRNFEAPRDFVGDTGVVHDLSPGSSEFCYFKYILGGDNIFKTIAQNTNKYAVHRQNQAKKADQEWFDTTSEEIVAYVGILIYMGVYHLSETMDYWHNPDIECSIIKNCMTYKRYKKLTKYFHVSDIEKEQNAHDEGFDVLQKVRPVLGIMDNFHKKYRPGRELVVDEAMVAFKGRHHIKQYIKNKPTKWGFKVWVLATPHGYVLKGNVYLGKKEVRNKGMLLGSQVVMNLLESYLGLHHHVYFDSFFSSVHLVRYLLQNNTYACSTSQPSRKEWPKELKLTKSLKLKRGEYKCLQSNGVTATVWCDNRYICVLSTNCDPKGSVSVLRKTGKGNERLAIACPLSVAKYTKFMGGVDRADQKRSYYSLGRKTKKWWKYLFGFVMNTAIVNSFILYNETNQPAKSKHGESQKEFRKALYRQMIGNFTSRKRAGRKRSVTPNVLSSQQSHKIIKIEGRKKVCVQCSENKKHTASGKAIQTTWKCRNCDLPLCKIGCFLEFHHKKGVEVDN

SEQ ID NO: 56 - protein, 587 aa, synthetic construct
MFPNSLVFLQMSKKWLSRKELEEEAARLFDDFDDGEDEEDDHFDNSESSDPDYVATDSDSEDNEKENSLGYSDEDLNSNANETNESEDLTEEVQHIQEQETTRELWFSNDDRFVPRLLVPQQKKIQCHLSRDLSEVELFLKMFPTSLFMYIAHCTNLRLAKFEQDTKKKRIPTDYQEIMIVIGTSLIMCYNRVPTFNNYWSNNDSLGNTAIKKAISRDRCKLLLSKMYFNDPKQPENANKLYYIEEVVNCLKSTFAKYRTEATYQSIDESMAKFKGRSSIKQYIPTKPIKRGIKIWVRSDASTGYVYDLNVYTGKDTEETVGTLGERIVNKLCSTIREEEITLCFDRFFSSVSLVDSLPFPAVGTIMANRKNVPKFSTKLDRGQSEFRVNNNGTIICRWKDTKDLLAISNCFSDNVVEVSRTLKDGTQKKFPCPEMLQFYNKHMGGVDLTDQIVGLYDFDRKSGKWWKKVFYKILMMSVANAHVLHNDLHRTKTAFLPFLINLAEGLISLGRKGARVKRKSASSLGRPSAKKSKLYNVGDHLPIEGKTRRRCQRCSLQLKKEKRTTTLCQMCNIPLCKTCFTPFHLK

SEQ ID NO: 57 - protein, 611 aa, synthetic construct
MFMAFCYSYLQKLSIWVIYVPLAFLHGTYVSLPNYIIMIYFRRRKLTENELQYMADHVWDESEEEDPFQDSGSEYTQSEAESSDSEIEGNNLQVLNEIYTAVIDEPVNKIMLDNMSKSNDPLQPQPQIMWQTPSSNFTPRRNLPDGKTRGVPLITVNRSCTELDVFLKIFPKSLLIFISQNTNERIHILKKDKGSKIEPTDPSEIMIVLGCLLVMSYNRVPNMQLYWSTNKSMGNEALKSAISRDRFLLLLSKLYFNNPAKPADASKTYYIDEVMSCLKKTFMAGRSDSNYQSIDESMAKFKGRSVLKQYLPLKPIKRGIKIWQRCDSLTGYVYDLNIYSGKETEQTEGTLGERVVNKLCQTIRNPNVSLCFDRFFTSVNLMNTLQYPAVGTCMTNRKLLPAFTEKLKKRGDTEMHCSNTGLLCFKWKDTKEVVLLSNCHKADIDQVERKNKDGSKTTVSCPEPVLFYNKFMGGVDLADQLAGFYDIDRKSNKWWKKVFYKLLLTSVVNAWVLYQEIRHKNCPLIHFIVPLAEQLIGYGKENASVKRRRQYGRPSQSSLSLQNVGEHLPVEGTTRRRCHSCHKKKKEKRTKIVCCKCNVALCMDCFTSFHI

SEQ ID NO: 58 - protein, 595 aa, synthetic construct
MSDLPFKLVNMTQDDLFAEIENIYLEIEQNHAVAEEIPSDSESLASNKGEEESDTEATIGNNVLIYSSSDSEDDLPLITIARKIQTAAFATPAGRKPPIWSNVETPASPPDFAAQSGLADCVTNMIDPTPCKLFQLFFSENFIKDMVFETNLYAQQKFSKHKPTDVREIKTFLGLNLLMGIKRLPSYRDHWSSAPDLHDSYVSNLMSVKRFSWLLGNFHLNDNSLMAKKGEANYDKLYKVRPLLISMAEAFGKCMLPSREVAIDESMIKFKGRSSLKQYMPKKPTKRGYKVWVLADKNGYCLKFDIYTGKPSGNAEKNLGSRVVKQLCEGLENKNHLVYFDNFFNGVELMEDLKNMNIHACGTVNSSRRHIPNFKADKNMKRGEMEWFTSNSDLSVIKWKDKRSVFLLSNFHDPRDMLEVNRKEKDGSITKVPCPKALADYNANMNFVDKFDQNLSYYKIDRKSHKWWHRIFFFFLDAAVVNAFVLYKGLQLPPLSMKDFRRSIIEGLVAEALVARKRSASSFETIAIKKNKPYVSEEVRFEGSTHQPERGTRRRCANCSTKKNQVRTEWVCSVCNVPLCLTKNKTCFQDFHRNK

SEQ ID NO: 59 - protein, 614 aa, synthetic construct
MSRRREALSFNLIGMTENELYDEIDRIENSCISIPSDCDSEADGDESESEGSSAGDAPAALFDHLDVAEIVVSDYGEDEPSSSDEWDAEDTVPLFTLAKRWKSNLVPTWNKCVSNIKTPDPFTADIGLPNFIRHRASLSPYEAFNLLFTDELLSDIVFQTNLYAEQTFQAKGKAYSQTNVDELKTFLGINILMGIKNYPSYRDHWSSVPDLHDPYISKLMRVHRFGWILSNLHLNNNSIMPVHGSANFDKLYKVRPLLDQLKTNFQSCLQPHEKIAVDESMIKFKGRSSLKQYLPKKPIKRGYKVWVLADETGYCWNFDIYTGKTGDQTEKNLGARVVKSLAAPLTNKNHKIYFDNFFSSIKLMEDLLDSKIYACGTINVSRKNLPYFTNDKDLKRGEYEWYTSDSGLTAVKWRDKKSVHMLSNYHVPTIVTEVKRKEKDGSVLLIPCPFLLSDYNRHMNFVDKFDQMRSYYGLDRKSHKWWHRIFFFFLDTTITNAFILYKLYHKENAKLSLKDFRRQVSEGLVAKALVQRIDKIAMSTELLSVIISSHKPTVSTAVRRAESAHQPMRTTRRRCAYCSSKKKDIRTNWQCSICQMPLCLGKTKTCFQSYHAQS

SEQ ID NO: 60 - protein, 588 aa, synthetic construct
MDPNKLINLAELTSDEAYTLIYSTDLPDDIPSGSESEDGDTDVEENENIDEIGAVPESEDSEDDIPLSAFVRTNSLPQQKGGNVSWEIGNISLIRPPCDFQSQCGLADEVEELDSINCTPYKLFHLLVTDEILNNITFQTNLYAEQEHQLTNKRYSQTTLAEIKSFIGLNLLMGIKRLPSYRDYWSVSEDLHDSYVSQFMVVNRFSWLLNHIHLNDNSSMPSKNNANYDRLYKIRPFISELKKNFKKYYKPHRCISIDESMIKFKGRSALKQYMPKKPIKRGYKVWALADSEGYLCDFDIYTGKTKDYVEYGLGEKVVLRLMEGLEQKHHCLYFDNYFTSYNLLKELKEKCIYACGTVVANRKNLPKLKEDKYLKQGEYDYNISNDGISIVKWKDKRAVHILSNFHDPQAINEVKRREKDGSSKQVPCPNALIEYNKNMNCVDRFDQLKSTYQIDRKSKKWWMRIFWHFLDCSVVNSYILYKLKKLPPITLKEFRRRVIDGLLAEKLVDLKRKEETCGEAIQKKSRKVPPEVRFTSSSHQPLRSTRRRCALCSTKANPVRSDWACSVCEVALCLGKNKTCFQKYHNNN

SEQ ID NO: 61 - protein, 585 aa, synthetic construct
MFRKGRGVFELCENDVLQVLMADNSDDEDDLELDDEDQRLLTQDDDQGIFHVEIEDTTMQQPRSESPQPSTSSVEPSSMSQPQPTFKWNQRTYIPNLFQESDYLFGKVLILETEADSLTPFAIFCAATNLEVLVDILVSETVRYAHQNGREFSVEKEEMKAFLGINLVMGYHILPSLRDYWSTEPDMGVTYVSNVMTRARYEDMRRNLHFCNNEEHRGPTSPDYDRAYKIRPVMVHLNVAFQNALNNTKTQSIDEHMIKFKGHNVMKQYIKNKPVRWGFKLWCRADAETGYLFEFDLYTGKRTSGTEYGLGESVVIQLTKKLDGLGCEIYIDNFFNSPLLQYQLISQNTKVCGTVRTNRKYLPKTAPADKNMKRGDIYTTSYHGITFVKWMDNKAVHLLTNFLSPAETETVKRRMAGSAEKIDVRCPKIVSHYNKNMGGVDLMDQRKVYYEVDRRSKIKYYLRLFFDLFDIAVNNAYIIYTKLHAEKRIEGNLLTCLQYRQVVARSLINDYSMRKRSVPTAATSVVRRSLKRPTPSHPMEKTDLRKRCVNCAKNKVENRTNNRCVVCKVHLCFTNNRNCFAEYHY

SEQ ID NO: 62 - protein, 620 aa, synthetic construct
MSDYVGLSPTEANNLLLLMEGDVSDVDIEDLDSENEEILVSNSQESYEVMQNPVPSHIRRISVEIESQTENYEDKGETETEEPEDPEDLLPLSEIRNKLVRKRIPKWIKKDIDPTSTRYEESFTPISDDEITPLSYFKMFIDDDIINNIVEQTNLYSVQKNFRSVNTNYTEMCQFLGIHIMSGIIQMPAFRMFWANATRLPIIADVMPRNRFEKLRAMLHFNDNSEMKTRNEEGYDKLFKVRPFVDAIRKNFGKVPNGEHQAIDEIMIPFKGRSSLKQYIKNKPHKWGIKIFARAGACGFVHDFEIYVGKGTVTNPTDLGISGDIVLRLCQSLPHNKNHKLFMDNWFTSYQLLSDLKSHGILACGTVRLARMKCNILKNEKELKSEGRGSFDYATDQNKNIIVANWYDNKTVHIASNYTGIYPIGVVKRWSTTEKKYIDVDRPDIIKEYNSYMGGVDLNDMLVSLYRIKHGVKRYYLRIIFHLIDVAIVNAWLLYRKDCNLRGTKHKSLIDFRADIGNALLQKNVCTLFRKRGRPTSTESSPVGSPTLLTAMQNKKKITMPTPVDDVRYDSYGHFPTHLDPKQRCRQCKTSYSRLGCVKCKVALCFTNTKNCFVEFHVKK

SEQ ID NO: 63 - protein, 591 aa, synthetic construct
MDPDDFTPISTKNFYGKKLQPVFHVRNIPDESEDSELSEDEIRESPKPENEDLYTTSSDEEPDDNIPLSVLRQRLNYIDISQIKWTETRSQTDELNTQFLGSEQLPDSILQLNTPYKLFSFFLSEEFFEETARQSCLYSVQKNPNVPFTTGVEELQRFVGIIYWSSLVRQPTTRRHWSPNTRFPQIADVMTVNRFERLKKNIHFADNLTSDKSIDKIMPVLDEIKIACKKLPLEENLSCDEQIIPFKGRISLKTYNPKKPHKWGYKMWVLSGVSGISYNFELYGDKDATQIIQGEPNLGAASNVIVRLCRDVPPNINHKIYYDNYFCSLPLIAYLADKSIQSVGTIRQNRLKNFNPKTEKQMKTNGRGTIEEKSVTLGKSEIRVVQWYDNKIVTLASSYCGTEPVRKVKRFFKTENTKKEIDCPDIVRVYNKHMGGVDLQDSLLGLYPIKMKSRKWYHRIFYHMLDVVAVNAWILDKKIKKQLGQESNIIPLLTFKTELAMQLCNRGFPNSHKRGRPSNEILPIPKRRCQEAGPSRTIQLDSVGHWPNWTVERERCKFPQCNGKSRVKCLKCKVHLCCNSKQNCFVKFHTS

SEQ ID NO: 64 - protein, 610 aa, synthetic construct
MKFFQIMDPKIFYGRKNVTQNCAIRDIPSESDDTELSDDDDTPNEELLVQAGVSSNNIEESDDEFSDEDDIPLIQLHNQAPNPADQKSGGKVTKKPKLNWKNSVTSKDEAGILFCGSELLPDGILKLDTPLKLFGHFLSEDFFTEVVRQSCLYSVQTRPNRPLCTSVEEIQRFVGMIIWMSIIKLPNTRSYWSPQTRISQVSNVMPVNRFEVIKSLLHFSDNSQSASSLDKIMPVLETIKKACLTIPLEEHLSCDEQIIPFKGRISIKTYNPKKPHKWGYKMWVLSGVSGFSYNFELFAGKRGCTIDDNEPDLGAASNVVLRLSRPIPHDKHHKIFYDNYFSSLPLISYLAKRKIHSVATVRQNRLPDYQGQTEKQMKQKGRGSINESTTKFDGIDIHAVQWYDNKIVTLISEYCGSQPVTKVKRFFRSENCRKEVDCPNIITEYNRHMGGVDLQDSLIGLYPIKLKSRKWYHRIFYHMIDVVVVNSWLLHRRIKRQQGSTEKIMPLLGFKTLLAEQLCSTGIRSSNKRGRPSGSNNGTPEPPKKSRRCLEPRPISFVQSDHISHWPEWTIERERCKLQGCKGLSRVKCQKCQVHYCCNSKNNCFLQIHQ

SEQ ID NO: 65 - protein, 590 aa, synthetic construct
MRNICANYTINCIYFAFCCFRPLSLHDIASILEDNDDSYSEGQDIDIFLLPPDTANDEQTDEDSGNEDVVDINNLPANLLRAPAELAASTSSEEPEGPRECNEPLQKRVKNYNWRKKDFQSKPITWQPLYHVPLERSPMEWFSLFFSEDVFELLTEETNRYLSRKLLPADVKIEEIKCLVGILLVSGYCQVSRRRMYWENAPDTRNKLIAAALSRDRFSLIYSNIHVADNLNLDASDKFAKVRPLLKLLAAKFLEHCPHIENHSIDEAMVPYFGRHGCKQFIRGKPIRYGYKLWVGATSGGYIAWFEPYQGASTFTNVRYSQLGLGASVVLTYTDQLEQLGYGKDYHLFVDNFFTGLPLVHEVTERGFRITGTIRENRTGKCPLLGNKEAKAKERGFYDYRVSDDVIICKWHDNSMVSVCSNAIGVDPVHKTERYSQQQKKKISVSQPHLIQQYNKNMGGVDLSDQNISSLRTSIRGKKWYMPLILHCIDMSVQNAWLLYRHNGGHMDLLNFRRRIANTIIDGNKQREKPGPSRTSSITAESRYDGKEHYVTKQEKRTRCGQCHKQCPTRCVKCDIGVHVECFIMYHTKK

SEQ ID NO: 66 - protein, 584 aa, synthetic construct
MDFTPLSTQSFYGHSYANIASALEAVEANQGNGLIDIAIIPPIADYNTDEDDVDSDDITQAQLPNDVPGTIDLFFHECEYDTEDDIPISELKKSLIAEKPSIPQWSDAPCNRDMAATYDFERNMREMKAQLENRNPVEIFETLFDEDVVKLILEQTRLYAVQNNRHNFKFEENDLKIFVGILFYSGYHSMPTERMYWELAEDTASEIISGSMARNRFYDIKRNMHLADNTNINPADKMAKLRPLMNLLCQKFKKWGIMHKDLSIDEAMVEYFGHHSSKQCIRGKPVRFGFKNWMLTSSCGYLYSFDTYCGAKNVNPETACLPLGSRVVLQLLAPIESPTDHIIYFDNFFSSYELFRTLKQNGFRATGTVRENRVKKCPLKPKKEVQKLPRGYYEFKHDTNSGIIFVKWNDNNAVIVGSNFDDVEPLNQVKRWSLAEKKKINVPQPRLIANYNAGMGGVDLHDQSVNAYRIRIRSKKWWWSLFTWMLNATMVNAWKLYRTMGNGKEDLLTFQRYVTRHYLRHYTVPTTSKRSVVPSVAKSTGPHFPKKITNQLRCKVCSMRSRWICEMCNVALCLERDCFKNFHI

SEQ ID NO: 67 - protein, 584 aa, synthetic construct
MSKRQFEVSDNYHGLQEALDAIMAEEDSDVEFDIVAIPPEPSILTDEEEGDEDNIVTNDMPRDIPGNIEVFVNRNEEDWDSSDEEPLSNSARRQRCNVGPRWRKCEPSYDHMPFDENNVQQRCENLITDLKGKNPVSIFEKIFDQEVIDMILQYSDLYASQNNKHDINITVGELKTFLGILSLSGYHTLPRERLFWSLDEDCNVPVVSKAMTRNRFLDIKKYLHFCDNTVAAETSDKMFKVRTLAEVLMKKFMQWGVFHTNLSIDESMVKYFGRHPSKQFIRGKPVRFGYKNWMLASADGYCYNFDIYCGKSQVPTNEPLSTRVVKNLLNKLNTDPRDHVVFFDNFFTSYALLRDLWESGYRATGTVREGRTSKCPLTPVKEMKKKSRADFDYRFDTVNKILFVRWLDNSVCTMGTNYDNVLPLGKVKRWSSIEKKKTDVNIPQVFVNYNRGMGGVDQADQSISTYRTSIRGKKWWWVLFTYMFDLCISNAWRLHILSNDVKMDQLQFRRTIARYYLRQKTNQRSHPSASVVLNLQFDGFGHFPEKLTKQLRCTICHSKARWECKKCKKTLCIEKPCFEKYHTE

SEQ ID NO: 68 - protein, 591 aa, synthetic construct
MDDDFQPITTTSFYGRKIFSTLQEAADAIESHEDEADVIVLPPEPDVLTDQEEFDDDNIMESHIPNDVPGYIEVNFGNTDEWSDSDDEPLSIFVSKRPRAEERNPVWEKIEPVYVFSRDTENGANDRLEKVKESLRGYNPVQIFEKLFDDNVFQLMIEQSIIYSRQKNNHDFFATMEEMKIFIGILLLSGYHKLPQQRMYWSLDEDLGVKIVSGSISRNRFLEIKRYLHFADNDKINKNDKMFKLRPLMDILNKNFRQWGFFHKSLSVDEAMVKYFGHHSAKQFIRGKPVRFGFKDWMLCSSTGYCYAFDTYCGKNSEKKGQSLGLGADVVISLLEHLDNPADHIVYFDNFFTTLRLMNQLTAKGIRATGTIRENRQKNCNIQSSKMMDKEERGTYDYRFNCENNLLIVKWKDNSVCSMASNHDFIEPLNYVKRWSRAKKEITTVQQPLVFQNYNAGMGGVDLNDQATNTYRISIRGKKWWWCLFTHMVNVTMTNSWKLHQICASPEEKISLLDFTRYVARYYLRLNNKTRKTNRPCQVPGAVRYDNVGHFPEKIEKQLRCSVCHLRIRWRCKKCIVTLCVERECFEKYHT

SEQ ID NO: 69 - protein, 571 aa, synthetic construct
MNRKLTQVELEKIVENLWNDDDDDIDHVADSSDCEEEEDHVSSASETDDIQEVNEGENLSNSNTVNTSKFTSKDGTEWSSQPPPTGRSRSCNIIKSKLHKVIIQPGKIIDEPIDAFCLFMEDGIVSEIVLHTNNEANRALKGVFWKPCDNIEIRAFFGLLLTAGHLSANDTSYDVLWSALYGPPIFRGTMGLKRFKNLLRFIRFDNKETREQRRKDNKLAPIREIWEKVNNNFKKHYLPGDNITVDEQLVPFRGRCPFRQYIPSKPDKYGMKIWWACDSQTYYPLNGIPYIGKVGNQVAKGLSSSVVEQLVEPFYNTNRNVTFDNYFTDYLLAQRLLKNGLTCVGTVKKNKRFVPHEFLANKKRDEETSIFGFTPHASLVSYVPKKNKSVIILSTMHHTKGLNGDYKNKPEMIMYYNQTKCGVDTLDQMVHKYMSKRKTNRWPFAFFMNLLDVSGVAAYVIWTNLKPDWNKNKHEKRRLFLVSLSESLVIAHIRRRQSRGLQHSIQNCMNEFVEDQPTSSKKLKLENKESVRRRCYLCPKVKDRKQKQNCSECKQNICKEHSKLLCDNCCK

SEQ ID NO: 70 - protein, 582 aa, synthetic construct
MAFSRDRRYVSASAGDFEPTVEEWLIEEEVLDDVNSDDSDLDPEYHPLVTDIESDDHSTSSCSESDADISVSVTSPPTAGTFISRNGTVWQHDSPVSSRTLAHNIITWTSGPKRGITTSTPKQAWDYFVTQNIIDEILLCSNLEGRRIATEKGKTWRQITVDEINAFFGLLLLAGVEKSWDVPVRELFLDQKQNPLYRATMSVNRFEDIRRVIRFDDRRTRASRAQTDKLAAFRYVWEMFIGNCQNVMTPNAYLTIDEQLLSFRGRCSFVQYMPKKPAKYGIKIFWLCDSKDSYAINGTIYTGRKPNEPVQKNLARGVVNNLAKIVEGSSRNITFDNFFCSVPLAEEMLQKGITVVGTLRPNKPDIPKEMLPSALREIKSSLFGFKGKVTMVSYVPKKKRAVILISTMHHDKAIDSNNMKNKPEIICFYNETKGGVDQMDQKVKNYTCKRQTRRWPFVLWMNVMDVAALNSQILFSAQNSEFYPKRNDTRRLFLKDLSFELVRNLMERRANQPNLPKSLQHALSLCGIVRPEPTPETFRCVQQRRRCSFCPYERHRKSTMVCEKCNRSVCKEHSHIVCSSCL

SEQ ID NO: 71 - protein, 579 aa, synthetic construct
MSSRRFTAEEALLLFFDSDAEEEISEIEDLSDAEDNVIDDPDFQFSDDEEDSEDESAVVSPSDENLGMEQSSSTEGTWASKDGNIKWSTSPHQSRGRLSSSNIIKMTPGPTRFAVTRVDDIQSAFQLFISQPIERIILDMTNLEGRRVFQEKWKSLDQTDLNAYIGILILAGVYRSKGEATSSLWNEENGRPIFRATMSLETFHMISRVIRFDNRDTRVGRRESDKLAAIRDVWDKWVEILPLLYNPGPHVTVDERLVPFRGRCPFRQYMPNKPAKYGIKIWAACDAKSSYAWKMQVYTGKPPGGAPEKNQGMRVVLEMSEGLQGHNITCDNFFTSYRLGEELQKRKLTMLGTVRRNKPELPSEILKIQGRPMHSSIFAFTEKATVVSYCPKRNKNVLVMSTMHTDASLSTRDDMKPQMILDYNSTKGGVDNLDKVTATYSCQRKTARWPMAIFFNIVDVSAYNAYVLWSEINQEWNAGKLYRRRLFLEELGKALITPKIQRRARPARSPAAAAVIEKIKFRPSNQSAMDPVDTDVKKRKRCQVCPSRDDSKTSTSCVKCKNYICRKHTVTFCPSCGEH

SEQ ID NO: 72 - protein, 606 aa, synthetic construct
MSGKRTNVPLTEADLQKLADNFGSYSDESDNEPIDNQSDICSETDDEEYFVPRDEVSESEDDLEIVDEEPFDMEPTDEIAEIEEIGEEFISKSGLKWSTIPKPVTRRLKRNIVTTKPGPTHYVQNAVTPKDFFNLLLTEDIKEIICRHTNEEAERIVSSWNEEHPDRQRKWKNFDTLELDAFLGVLIKAGALRCRRESVREMWTTDKSIKRSFFIAAMPRDRFLQILRFIRFDDKNTRQDRKCTDKLAALRDVWEIFTENCKKAFEPFESVTVDEQLVGFRGKCPFRQFIKSKPARYGIKIWALADVKTSYLFNLQVYTGKPPGGKPEKDQGSRVVRDLTEPIYGTGRGITTDNFFTSVGLANFLLEKKLTLLGTVRKSKPDTPDQLKVAGRPEKSSLFAFCQDVAMVSYVPKKGKLVHILSTQHSDNSICEEDNKKPVMILDYNSTKGGVDNADKMICEFTCARRTARWPFRLFMNILDICALNSFILFVQKNPEWNKQLSHRRRVFLLELGDQFTRPHMIRRLETPIHSIVIESLRDCGVYKKDTLALAEASDEQRKRARCYRCPRMEDKKSRVKCDKCKNFVCDSHRKAAKRVVCNPECSLSD

SEQ ID NO: 73 - protein, 584 aa, synthetic construct
MPFTPRNPNALTDDELRRYLEEDDEEVDNVLGGVEGEESEEEDLIEDESITDSENENDSDSEPITTVVDSTNAYISRDGSIWAKTPVRTVRKHPGNIIKISEGLTKYSSGFSNITEVFTLFLSEEIINIIVQETNRKARHYIDLWNNKHENASNRIWKDMDSVELWAYIGLLVSMGALKSRQEPIEMLWTTDNAYCRPIFPVAMSKHRFKELTAFMRFDNFETRIERRQQDKLAAFREVFEIFVENCKRAYIPGSHLTVDEQLVGFRGRAPFRVYMKSKPDRYGIKIWALVDCATLYTMNLQIYLGKLGNKPEKNQGVRVVLDLIQCVPSGRGITTDNFFTSKDLALELQKRKLTLCGTLRQNKRFIPKELLPHKEKPQFSSMFAFQRDVTLVSYVPKPGKSVILLSSEHHEDSVSTEETDYKPDVILHYNSTKGSVDAVDKMAKQYSARRTSNRWPMVLFGHLIDMGAINAYQLWLIKNPSEKQVLDGRRKFLLVLGKQLIKPNALRRFQNPKIRQSIKNQISGVYPEATTQPLQNMTMAMQGRCHLCERGKDRKSRQRCSKCHVFICTTHSVKSIVCLNCNK

SEQ ID NO: 74 - protein, 586 aa, synthetic construct
MEYYNLTIYISVIMDESERFRILGMLDEVSSEVNSDLAESDDDSDADPDFEILSEHDTDSEESGNEECMVESSAEVESRVYLGKDKTTIWKKEMPNRRVRTRAHNIISHIPGVKDTAKQKRSFDGVFSLFLSDDILNEITRCTNLYIKDASSRYSSRQSFIQETNVDEIKALLGLLYLGGVFKAGRVNVEEFWNKDGTGLEIFQVTMTVQRFRFLLQSLRFDEKATRAERKRFDKLAPIRRVFELFNSNCKSHYCLGEYVTVDEMLVAFRGRCSFRMYMPKKPAKYGIKIFNLTDARTAYSSNLEVYVGEQPDGPYKVSNKAHDVVVRLSSPIHGSSRNITMDNWFTTYTLAISLVKDFKLTMLGTVRKNKREIPPEFTIAKSRQCYSSIFGLQENCTIVSYVPKKNKCVILMSTMHHDTKIDENTGEARKPEMITLYNATKGGVDLVDQMSATYDVARTTRRWPLALFFCIMNVAAINSLIIYKSNNKSSIRRRDFLKNLGKELIRNHLVKRTEVNSVPPTMRKRVRGMLGEEEPEKQDVGEDGRPQKGRCYLCKDNKSQFSCAKCHKFLCLRKHANFLCKDCLG

SEQ ID NO: 75 - protein, 569 aa, synthetic construct
MSYEEEQDRLSRLMEECLNEDTENISAVEYDGSNDEGEVDELEINDLNTDTEQETSDSEDFNVIYGPTFTGKDGTEWAKHVPNQKVRTRAENLIKHLPGVKGPAKELKSEIDIWKHFFPEKTLILIVRWTNQHIQSTRENYERERDAKYTDIVEIQALFGLLYFAGVLKSNRLNTEDLWNVNGTGVEIFRLTMSIQRFRFLLVHLRFDDSSTRIERKNVDKLAPIRDVFEGFVNQCKSAYIPFEYVTIDEKLEGFRGKCSFRQYIPSKPNKYGIKIFAMADAKTFYTCNMEVYVGKQPDGPFSQSNSPKDVVQRLCQPIKGTSRNVTTDNWFTSMDLLYTLYNDFKLTLLGTIRKNKRELPKEFVNSKGRPILSSMFGFRDKCTLVSFVPKRNKNVLLVSSMHSGDKIDKDTNKPEIIMDYNSTKSGVDSVDQLCASYNCARNTRRWPMVIFYSILNVAGINSFIIYAMNNQVNVQRRKFLQRLSFQLVETHTRRRINQNIPRSMSQRIREIFAIEAAETTPAANNQRGRCSFCDRKKNRPTRYSCVDCRTFICLEHCLFMCQECKSKY

SEQ ID NO: 76 - protein, 593 aa, synthetic construct
MDNNVVAERAMNYEKECEYLQRLHDEVETDIEPDDDDISLTSEGEEIQDHDTDDEQTIETIDINLHVPLAEHETGQQFAPYYIGKDGKTNWRKHPRNRQMRTRAENIIKFLPGVKASAGEKKSIIDIWGLFFTDDILGVIVENTNIYIGLGNFNPSNRTAKPTDIIELKALLGLLYISAMSKNNRVNAADLFRTNGTSMEIFRLTMSLQRFKFLLRHIRFDNKTTREERQKHDKLTPIREVFDAFNSNLSKYFCLSEYTTVDEMLISFRGKCGFRVYMPNKPNRYGIKIYSLVDAKMFYTAKLEIYVGQQHPGPFKVETDNVSLLSRLCQPISGSRRNVTMDNFFTSMAVANKLSNDHKLTILGTIRKNRKELPLEFTQPITRPEKTSMFGFQDKATICSYIPRKHKNVLLLSTMHFEEDTIDEETGKPEIILDYNRTKGGVDTVDKMCEAYNCARATRRWPMVIFYSCLNIAGINSFIIYKNNAGYMDKTPRRLFLESLGMELLDTHLRRRVLNERIPRTIRIRLKEILGINDQQDGQTHNPEVQKGRCFQCDRKKNRPTRFKCSNCQKFICLEHSKYICHHCANETEYCED

SEQ ID NO: 77 - protein, 573 aa, synthetic construct
MSYEQRQRRLEALLQEAISDTEELRRSSESETGSDLEPENFEPDQLQNSESEEDLSDRESSGNESLSFYLGKDKSTRWSKEPHRQSVRTRRFNIISQLPGPKRAIAGEKDHFQIWKYFFDNNIIDIIVKYTNQYIDSVKHKFVRETYARPTNFNEIQCFMGLILLAGVKKSNHLNVDELFRTDGGAVEIFRLSMSAQRFKFLLRVLRFDDCNTRQQRKAQDKICHIREIFEMFVQNCKDNYTLSAFVTVDEKLEAFRGRCSFRQYIPNKPNPYGIKIQALCDAKMFYTFSMEIYPGKQPVEGPFNLDNSGLAVVQRLCEPIFNTGRNVTTDNWYTSLPLADALLEKGLTTVGTIRKNKKEIPPDFNVLRGRRVKSSMFGFRDNMVLVSHVPKAGKNVLLISTMHNDDKVDSATQKPDIILSYNDTKGGVDVVDRLCANYNCARATRRWPMVVFYAMLNVSTINSQVIHTANNQDSKLLRRHFIENLAMNLIEPHIRERQTQLNLPRSLRQRLSEILKIDDAPDVAGPSRNGRCFECGWQKNRKTRFTCHKCKKYLCLEHVVPTCSSCLAVDYE

SEQ ID NO: 78 - protein, 568 aa, synthetic construct
MDDDHIRLLLEELSSSSSYEDENDSEVEDNVEVQEETYDTEQDDDNDEISEEEHNERSHDFYLGKDGQTRWNKSPASNPTRRRARNIVTQLPGARGEARNLKEPGELWRLFFSEGTISLIVKYTNEQINRMKHNFTRERDCADTDNIEIEAFLGLLLFAGVRRNARLNAKDFFRTDGSSPEIFRLTMGLSRFFLLMRCLRFDSKDTREERIQLDKLAPIRELFDIVVGTFKKYYSVSQYVTIDEKLEAFRGRCNFRQYIPSKPNKYGIKIYALTDAKMFYTHTMEVYVGKQPDGPYQVDNSALALVLRLSENIHNTGRNITCDNFFTSIPLIDKLESQHKLTVIGTIRKNKRELPKNFTEIRGRAEKSSLFGHRGNCSLVSYVPKKGKNVLLVSSMHDDAQINEDTGKPEIIMDYNRTKGGVDTVDKMCETYNVARGTNRWPMVIFYSLMNVAGINTFIIYLQNNPNKKISRRVFLEALAYDLINPQLRRRALISSLPRSIRLRLTEICKLDGPESAATSYNNKVGRCGYCSSKKNRKTRYNCATCKKYFCLEHAVTVCPDCYGEAQN

SEQ ID NO: 79 - protein, 585 aa, synthetic construct
MASVSQEDYIRQLLDESLHSEGDLENLANDSPDEDEVDHIEVFQEDTDTEQEGEDDEKEANNDQPQQQVQPLPGMPCYTGKDKSTEWKIHCTRTKTVRTPRQNIVIHLPGVKHYAKQAKTPIDCWGLFFDEEMLNIIVTNTNLHIEEVSRTYSRQRRGKLTDIVEIKAVFGLLYLAGHLKTSRLNTKQLWSRDGCGVERCWLTMSEDRFSFILRHLRFDRADTRLDRKELDKFTPIREIFQMFTNNCKKSYSVSEFTTVDEMLLAFRGRCMFRQYMPQKPNKYGLKVYALVDAKTRYMLNWEPYVGQQPEGPFRQSNSQVDLVTRLIAPISGTGRNITSDNFFTSFPLIKKLKQDHRLTMVGTVRKNKRELPPEFVNTKQRPIKSSMFGFQDEVTLVSYIPKKNKNVLLASSMHFDDAIDENTGDTMKPEIITFYNSTKGGVDTLDQLCATYDVARNTRRWPMVIFYSLLNVAGVNSQIIYIANNPTTTCNRREYLKELALSLTSEELQRRSLLTNINPEIRLRRQEVAGTSTTAEQADQFPPGTRKRCFFCKKDSKTKYSCKYCHKFICLGHAQTVCPNCPNIE

SEQ ID NO: 80 - protein, 605 aa, synthetic construct
MSFQLKLSNMDYEKEQARLLSLLEDAFSEDEVVGDESDHEEDIIEVREENTDTEQSGEDDVTEFRRENAGREELQEEIDNSIIRNKRSKKDTTFLAKDKKTIWKKHVPPRNVRTRSENKVIHLPGVRPPAKQAKSPIECWELFFSDVILDIIVEETNKFIETISVNFTRESSARPTDKTEIRALLGLLYMAGILKSNRLNFEDLWDRKGCGVEIFWLTMSQQRFLFLLRCIRFDDLATREFRKATDKLAPIRSIFELFVNNCKINYTVSEYTTIDEMLLGFRGRCGFRQYIPSKPNKYGLKMFSLVDSKMFYTQNLEVYVGQQPEGPFQVDNDAASIVLRLIEPISKTGRNLTCDNWFTSIKLIDSLLRDHKLTYVGTIRKNKRELPIEFLNIKNRPVMSSMFAYQENITLVSYIPKKRKNVILASSLHHDDSIDPTSVDKYKPEIITFYNSTKGGVDTVDLLSSNYDVARKTRRWPMVIFYALMNIAGINAQVIHTANNRASKLLRRKFLKTVALSLVDDHLKRRSLAANVPTNVQQRRREVAGTVAGHEEQPDIPHGTRKRCSECHKDRKTKYYCVTCKTFLCLSHVNIICSNCLGKSQNPNK

SEQ ID NO: 81 - protein, 611 aa, synthetic construct
MNYEKEQERIINLLESVPDIASSELGDDLSDKSDVEDEGENARNNEGETASEESFDEDQEEGNMPNASIININNQATQNNGLTNPIYVGNDGPNPTVWSDRPQRRTSRTRRHNLITQLPGPVRHAQNAKTEIECWNLFFPEETINKIVTYTNQKITEVKANFARERDARDTDLVEIKALIGLLYLAGVSHSNHQNVLDLWGNDGMGVEIFRLTMSISRFRFLLRNLRCDDRRDNREQRKSEDRFYCMREFFSEFVRLCQQNYSHSEFVTIDEMLPNFRGKCPFRVYMPQKPGKFGIKVWAMTDSKTFYTSNMEVFISKQQPGPYVLDNSSRAIVNRLTANIHNTGRNVTCDNFFTSLPLARDLLEKRLTLVGTIRKNKREIPIQLKSEPAGSSRKVNSSMFAFTHQETLVSYVPRKKKFVLLLSTLHETDEIDETTGESCKPAIITFYNQYKCGVDVCDAMQKQYSVSRISNRWPLTIFFYTLNVGGINAFVINKANNVSIDYETAQPRRKFLTNLGRRLVLEHAQRRYSEASNIPRPMKVRIKEIFNLQPIQPPVVEVPNQGQYSHCVICPRRKNRKTTVKCTSCQIPICKEHTSPRCTKCVDNEAMDTD

SEQ ID NO: 82 - protein, 596 aa, synthetic construct
MDERQCQRILRWLEEEDVEAVGGEEDEEENIDYPDEVSEHDTDSEQDCEDGNPQEHLLQSDSSNSDEEDTSTANLSSDNFYIVKKRRRDGSYSVICKWKKTPYPQAVRTRLHNIVTEQPGPQGAAREVCNPLESWLLFMDTEMLQLIVDCTNIKITSMQIKYGRERDCELTNLIELKALFGIMYIIGLHKSSHTSIDDIWITDGTGLDFCHAVMSKQRFQFLLRALRFDDIQTRAERNLTDKLAPIRKLYEAFVNNCEKYYRNNEYVTLDEMLESFRGSCRFRQYMPSKPAKYGVKLFSLVDAVTFYTRKFEIYVGKQPEGPFHQDTSTNALVKRLISPISGTGRNVTMDNWFTSVPLAEELLRDHKLTVVGTVRKNKAEIPNEFKQKGMQVCTTIFGYQPTKTLLSYCPKKNKVVILLSTIHHDGVIDDSTKKKLPEIIRFYNKTKGGVDVVDKLIGSYSVARICHRWPLRLLFTFLDVGALNAHIILDTINQVNKMERKVFLKTLAFELCRHQLQLRATIMTLPRELRQTIRRFTPHLQDMLPDDNDFVPMRKRCIHCPRQKDRKFKSVCHFCKGNICPNHTKFVCTDCIKTEN

SEQ ID NO: 83 - protein, 580 aa, synthetic construct
MSRRQLSQSAIEDLLREDEEESESELSDHGSENSDHIVAEEPRSAEESNCSDEEEDDLPLSVINSYFTGRDKVTKWQKSNNNRVRRQVQNVLTEDAGIRRNGVGATTFTQAWSLFISENIIEKVVRYTNEFIQKKAQSYNRIRDCKPTDAVEIRALFGLLYIAGSLKTSHANLEDLYASDGTGIPIFPTTMSVKRVKFLLNSLRFDDATTRQARRTLDKAAPIREVLDMFTQNCLLPYSVGENVVIDEMMIGFRGKCPFRQYMKSKPDKYGIKLYPLVDAATFYVLNIELYAGRQPEGPYQLSNAAADVVKRLITPISGTGRNLTIDNWYTSVPLINDLAKDYKISVVGTMRKNKREIPPCFIDKNQPEYSSQFGFSDTSTLVSYVPKKKRSVVLISSLHCAAEIDESTNEKRKPSIITFYNKTKGGVDEVDKKAKMYSVSRKNNRWPLTLFFRLLDIAGINSMVILRANNVILRKRRMYLRALGIELIKEYVQRRSTMSGLPRKLKRNIREHLNMEDSAADAEAGSSGTCRGTCKICPAKKRRQSKTQCNLCMKNICRSHTTFICNQCYMNEETDSNEN

SEQ ID NO: 84 - protein, 597 aa, synthetic construct
MSRMRIDTSSIEKILQEDEIEGEDCSEIEDNLEINDHDSNSCQSSEEESAEFIASDPVTTPGDLFQRRTDLNSVPLIYIRQGYYLSKNGAVKWNIEEPPRSVRTRSHNIIREPFGPINEAKDAQTPLDCWTLMYPDNVIEQIVVYTNRYIAAIQNKFQRERDCLPTDLIELKALLGLLYYFGKLRGAHLNTKDFWATDGTGSDICIATMSRQRFHFLLRCLRFDDINTREQRRQSDKLAAIRDFFVTFVDTIKKHYSLSDTVTVDEKLEPFRGRCPFRQYMPNKPARYGIKIFVMADSRTYYTAGMEIYAGKQNDGPFDVSNSPLDLVKRMVEQIRGSHRNVVMDNWFTSFPLITDLFENYGLTVLGTLRKNKPEIPSAFTTTKGREEKSTYFGFQPNCTLLSYAPKKGKVVLLVSTMHHDAMIDPETGDKLKPKMITDYNMYKCGVDVVDEMCGTYSVSRVSKRWPLTIFFGLMNVGAINAYVIYNENMKRLQKVTVERRHFLKDIALGLVMPQIQKRSSITTLPRLTRSKMFQILGKEEITNRSQPSVVSSNVRGRCSLCPPKKDRKTKTQCQYCQIFICRDHTTFVCKNCEQKD

SEQ ID NO: 85 - protein, 597 aa, synthetic construct
MSDSKRARLMDTDILEQLQHPISSDEEEFAEGDSDDGEEDNLEVEVHSEDEEEVQSANDNSNEAVPLSDSDCDETPLGNIYFSRDQHKNVMKWRKLPPDPPNVRTRASNIVLRMPGPKGEAKIVKTELECFNLFIDNTVIRMIVTGTNIYISQIRRSFARDRDAKDTDDTEIRALIGILLLSGYLGSGRTNTKNIWDNSRGTGVESCYLAMSENRFHFLLRCLRFDDIRDRPQRKELDRLAAVREMFELVVRNFQRFYTPSEYCTVDEQLVKFRGRCPFRIYMPDKPAKYGVKVFALCCAKTMYALNLEVYPGQQPNGPFKLSSSSADVTIRLCEPIAGTNRNVTTDNWFTSIPLAEALLEEKKLTLVGTIKANRIGVPKDFKADKEREVRSSIFAHHKNKTLVSYCPKRNKAVILLSTMHNDQRIDNESGDLKKPEIVTFYNKTKVGVDVLDQMCAKYDVSRNSKRWPMVVFFDLINIAAINGSRIHSFNNIEHSPLPRRKFLEHLSWELIHPQIRKRIESPFLPVDLKTRAKKLLGIEENPCKPLPQDNKVGRCFLCGRARDKSTRKCCQLCGQKICPEHSCLVCRCCINGTTAD

SEQ ID NO: 86 - protein, 618 aa, synthetic construct
MGDLSFRVNMGDSERDREQIRLWLRETQDSEDVESVLGIEDPDEVESNPPDIETVDTNIVESEQENEPMDVDNIQENETPGRLPAEHFPETIAEEVCGPPFDLREFYIGKDKTTRWYLEPHMANTSRTPRYNIIPTFHRPGPKGLAKNAKSHLESFQCIMNDLIIEKIVSYTNIYINKKKGQFSRERDARLTDVCEIKSLIGILLLAGTLKAGRKNVIEMWDNSNGTGVEAIYVTMSEHRFRFLMQCLRFDDVRSRDERRALDKLAPIREIFEMFLKNSQNSFNPSDYLTVDEQLVAFRGNCRFRQYIPSKAAKYGIKIFALVSASNFYTTNMEIYVGTQPEGPFNLSNASKDLVLRLVEPVSGSNRNITADNWFVSLPLAVSLIDEKSLTLVGTVRGDRRIVPRNFLADRDRETYSSIFGYHEKCTLVSYAAKKKKVVIALSTMHSTGEIDPSTGDQRKPSIITTYNDTKVGVDILDKMCKHYDCARNSRRWPLTVFFHLLNVGAVNSLNIFRSNNNSAFINRCDYLRTLALELMKPEIHRRINIPTLPKELRRRGRCLLKLEEEQRPVVQPRGNSTIGRCVFCGRSRDKKTRRHCAKCDQWACADHLRDVCVRCYE

SEQ ID NO: 87 - protein, 601 aa, synthetic construct
MDAQSQRVARWLEETDEEEENDLEDLPSSEDEEDCCLEERHDSESEQESDVGEQIIPDTPSALQPLSESSSDEDVPLANLRIYKSKSGLEWQATPYRTSVRSQASNIRSRICGPKNDASACKTPISCFELYFNEETIDIITRCTNIYIDKIKTKYQRQRDAKNTDYTEIRAMIGMLLMMGVEKSGRRILKDFWDNRFGTAVEAIYLTMSQNRFAFLLKCIRFDDIHTRDSRKEIDKLAPIRQIFDIFVSNLQKYYTPSEYLTLDEQLVAFRGRCGFRMFIPNKPDKYGLKLFALVDVTFPYAYNLEIYAGAQPPGPFQVSNKVKDIVHRMLKPLEGMGSNVTMDNWFTSIPLLMELEMKKITAIGTLKSNKPDIPLNFKSSKGRPINDSLFGFQKKCTLVSYVPKPKKVVLVASTMHNDGTIDLSTAEKRKPEIITEYNRTKCGVDVLDKMCKHFSVARNSRRWPLTLFFDMVNIAGINGMIIDTMVNHNYQRKSRRDYLRSVAIELIKAQMRQRLMTKNLPRTIKEKIMMFLEVKESNLVPEDEVIPKKRTKTARCYDCGRAKDRSTRTFCQKCQKPVCNDHKYPVCLGCYNKYVTEPKE

SEQ ID NO: 88 - protein, 584 aa, synthetic construct
MSERSTNPEVWKQWLRELSEDETGDTAIESDPDDPEVDHVNKSDDDTDSEFDGDECNSENEDVLSHIGRDGTTKWRNTPVLSSHSRTRRENIIIHLPGTKGHARNALTPLESLKCILDDKMLSMITNYTNLYIEKIKGQYTRERDAQETNELEIWALIGLLFLAGVKKSGRQSVRDLWDRSGTGIEIFYMTMSYNRFFFLMRCLRFDDHRDRLQRSEIDKLAPIREIFEIFIQNSQNSFTPSEFLTVDEQLVAFRGRCSFRQFIPSKPAKYGIKIFLLVDTKNMYTINAEVYVGLQRDGPYKQRNTPDEVVKRLVNPVSGTNRNITFDNWFSSLQLAIDLQKYFKLTCIGTLRKNKREVPASFVNVKGREVSSSIFGYLKNGRCTLVSYIPKKGRIVLALSTMHFDNAIVTQPKPKPQIIIDYNSSKFGVDIVDKMCSGYNVGRNNRRWPLTVFFNILNLAGINSQVIYNANKPLGGSNESRASFLQSLALELIKPQLKKRLFTKAVPRSIKTKIKIFLGTQEEIPSEKDAAGSSGRCHICRRTRDRKSRFVCAKCRNYVCRDHYKVVCEDCMEYECYSESEEN

SEQ ID NO: 89 - protein, 586 aa, synthetic construct
MARRPLTQNELEEIARDLFEEPEQDSETYFPSSDSDFSEEEIEQQAEESASEQENTSGESEEEDETMEEFFIGKNKSTKWRKATYRVSGKTKGINVVTVPTGLTQKALGIENELNAFFQIIDVSMIDDVLRYTNMYIDQLRVDRQYSRDRDCKNIDRHEFMAYLGLLYLIGIKKSHHANVKEIFSSDGTGIEIARAVMSYKRFLFITRCLRFDDKNTRIERRKVDKLAAIRDFFQTFVNNCKSSFNLSEYTTIDEMLHPFRGRCSFIQYIPSKPAKYGLKMFALCDAKSFYTSNLEIYCGIQPDGPFAASNTPTDIVKRLVTPIENSNRNLTIDNWYTSVPLADYLLTKKLTVLGTIKKNKAEIPKEFLPDKSRAVGSTLFGFQKEKMMLSYVPRKNKSVILLSTLHDDNEMDPDTRKPLVILDYNETKGGVDTVDKMCAAYSVSRITKRWPCVVFYTLMNIGGINAQILYKFACPRKAPKHRRIFLKNLAFIMMKEHLIFRSSVKHLPQDISAFLKVNYGREVEPITEGENRSPPKRGVCRSCALEKKRSSASMKCCRCHSFTCKNHSAVEVVCNNCKDENIDSD

SEQ ID NO: 90 - protein, 577 aa, synthetic construct
MSNFLITDEEIEAELLQDDDDSSVFSEGSEDEDNLHISEHDSESDLVYTSENEAIPITGNYFLSKDKKTKWSKEPVSQMGRPKSHNIISHLPGVRTTAKLAKKPKEIFSLFFDDQVMDSLVLYSNMYIMKVRERFERARNARNTDRVEMNAFLGLLLYAGTYRASHLHFKDLWDQDGPEIFSITMSYDRFLFLLRCIRFDDTDSREVRKKNDKLAAVRDFISALQTNFLKVYTPAEYLTIDEQLIPFRGRCPFRQYLPNKPKKYGIKVFAMVDSRTFYLLNFEIYSGNQPEGPFKKSNSPIEIVQRLVIPISGTGRNITTDNWYTSLPLAKNLLKKKLTIVGTLRKNKPDIPLEFLPSKQKEVNSSLFGFTKEVTMTSYVPARSRSVVIISTIHKDKKIDESTGPQQKPEIVTFYNCTKGGVDTCDQLCSNYSVGRRTRRWPLAVFFHFVNVSALNSYIIFKANTGQKIMRRVFLKDLAKDLVLKCVQRRMEDLHIHRSLRQKIKLYLIERGVTSVTANVRPENDRSRCTTKSAKRKRCHICPSTNDNKHSVVCSLCSQHICKMHTKLICISCEGDD

SEQ ID NO: 91 - protein, 603 aa, synthetic construct
MANQSQDRYRPLTQAELEQIAEEIMTNSDDDPWSADEGEDNNHYIVSEHESESEMSGHESDEDTNMSEDDSAVAGNSSTFFYGREKSNKSKWSKIEPPRNTRTRTHNLIRNLPGLKPAAKQLGTQPQPEEVWRLLFCDKIIQEVLKWSNVNLAKRRALNKYQDNSNLREIGITELYAFLGLLFYTEVFKSSKESLDSFYATDGTGRDIFRATMNRKRFEIILSSLRFDDPATREARKTDDPAAPISTIFDKFFLNSQHYYTIGEYACVDEMLVPFRGRCRFRMYMPKKPAKYGIKIMGLTDARTCYLLNAYIYTGKDSDGRTLSQEEKRLPIPCQSVIRLSKPIHGTNRNITADNWFSSMPLVEELRKRNLTFVGTMKKNKAAIPKEFLPNKHRNVETSLFGFQKDTTIVSFVPKKNKAVILVSSMHHSKEIDQITRKPEIIMLYNMTKGGVDSMDQKIANYTSNRRSRRWPMKIFFTLLDISGVNSYILYSSYKDITPISRLDFVKSLAKALVHPFLRERSYLTNLPQAIKLKISEILQIPLEEEASVTGAEGKLATRKYCKTCPPRIRRKTAYVCACCSMPICLSHARKICQNCITKNKLN

SEQ ID NO: 92 - protein, 610 aa, synthetic construct
MANDAPGPSKKIRFCDTDFESTGLRWLDEIENEMLTAIESDEDSASESIYEPSEHDTDTEQEGEEEEFANTDEQEDSEDYEEPVNQASKKSRNFYGKNRFKWSSESFTSRSRTPKHNIVLRLPTLRGSASLLGHEAQPFAVWNLLITDDMLKTILHWTNEKIKKQRANLKNSSRSEHQDLDIVELKAFIGLLFYSAIFVSNHENISRIFATDGTGREIFRCVMSKQRFAFLLACLRFDNSLDRNIRKQENPLAAISELFDQFIKNCQTLYSLGVNCCIDEMLVSFRGRCRFKMYMPNKPCKYGIKIVCMTDARTHYFFNGYIYTGKDSDGFGLEEHLKKFNKPTQSVLRLVQPIAGSNRNITADNWFTSLELVEQLKNRKLTYVGTVKKNKKEIPPDFLPKKDRKVGKDGTLFGFTKDVTLVSYVPQKNRAVILVSSMHHSTSFDDGTEKPEIVTYYNSTKGGVDAIDEKCAKHSTSRRTRRWPMVIFFRILDISGVNAYILHQSYKNNKVMEKTDFIKILADELVRPHLERRANNPRIARDLRSTIKRILNIPEERLEQVEMEKLEKRKTCYTCPSKLKRKTFHLCYMCTKPICVECSTKLCINCRHNL

SEQ ID NO: 93 - protein, 633 aa, synthetic construct
MDIPSTSSGHVSKCSRKQTVQFGDENFERRVTEWYLESDDELEENEEIDDSDIDPDYVLSDDEQQATQEEVEISEGSESDTEDNLLNAEQRTTDDDNHKQHGRETNEETQFFIGRKNKKEYKWLKNAPSGVRKTPKHNIIRQLPGLTRKARQLAKPGQLEIWKCLFDQQIIDKIVLFTNAKLVSTRDKLKPESNKSNYRNTDSVEIHAFIGLLMLTSIFKSGHENMKTLFAKGLTGRPIFRATMSVKRFEILGMALRFDDSTTREERKKVDRAAPISELFNAFIQNSQEMYCLSDNVTVDEMLVPFRGKCGFRMYMPKKPAKYGIKIQCLTDSYSSYLFNAYIYTGKDSDGIGLSEEERKLLKPTQSVLRLCKPLEGSNRNITADNWFSSLELVEELRDRSLTYVGTLKLNKSIIPPEFRANKSRPVGSALYGFATEMPITLLSIVPKMKKSVVLISSMHDSVQTDSDKNKPEIVCYYNKTKSGVDLLDMKNALYSSNRRTRRWPLVIFYTLVNVSCVNGFILYLCYRQNPTMKRSTFIESLAMLLIEPHLRRRLAISNLPRDIKSLIREVIPEGLENSTEEVPSDKLQKRKTCSICPPRKERKTSYKCIKCGNPICLECSRKVCTNCVTLSF

SEQ ID NO: 94 - protein, 615 aa, synthetic construct
MALKNNALTDKDIEDLFEDENFWNDQDITTQAALLPSEFNDIVVSNDQSDQEEEDNEVLSDHDTESEFSWSSGDDSDFDQDEEIIEVPPSISTSEKEKRSGSWHGKDKTIWSKQEPTRSRTTSENIIKILPGLAGEARQNPPKNVLEAWNLLFTDNMIQKIIEFTNQKIKIMAANYTVSAKNRLSFLSTTTSEEIRAFIGLLYLQGIFKSNNENLRSMWATDGTGRDIFNCTMTLARFNFLLSTLRFDDETTRAERRAMTKLAPIKDLFEEFVERSKTNYIPGEFLTVDEMLVAFRGRCGFRMYMPNKPAKYGIKLQILADSKTHYMCNAEVYLGRDNGPVQKSVFLQPTQVVLRLITSLPKKSNRNITGDNWYSSVELVNELLKNNLTYVGTMKKNKRQIPPEFQAIRSRPESSSLFGFQSKTTLVSHVPKKGKSVILISSMHHDAKIDKSTLKPDIILFYNDTKSGVDALDQKCVHFSTSRRTRRWPMVLFFQMLNIAGVNSRVLYQCSPKGTEIERYSFLKKLGMALCKDYMEIRMNNQHIRRQLRESIALRLGTSLPVYPTPIPEQSRSKRKRCAFCPPKKDRKTSAFCKKCNKPICFSCCNILCEPCLNK

SEQ ID NO: 95 - protein, 593 aa, synthetic construct
MTGFLNENQISQLLEDEDEESDLDSARATTTAVCDDEPISDCEVAETVSCSEESVLGTDSESSSSSSEEDEETVRTPGYYRGKNRFKWSKNPPSSSRVRQHNIITHRAGLAGEAREKMNMKETESWQLLIPDNLIEIVCLHTNKKITEFTEKYSSQGAHTDHVSLLEMKAFFGLMLLAGVFKSGHEDVESLWATDGTGRDVFRVTMSLKRFLFLLSALRFDEIDTRAARKETDRAALISEIFNKFIENCQKNYSCSEYMTVDEMLCPFRGRCLFRVYMKSKPARYGLKVLCLCDAKTHYLYNAFIYTGKTETNRRDGLSIPTQTVLTLTDPVKGTHRNITGDNWFSSIELVDKLKENGLTYVGTVRKNKREVPPEFLPHKTRPIGSTLFGFTNDKTLVSYVPQKNKSVVMISTMHHNQSTDTNSGKPEIIIFYNSTKGGVDTLDQKCANYSVARRTQRWPCVIFSAILNISMVNGFILWKAANPNKRLKRKNYIKNIGINLVTPFVKERQKESSNFGADLKRRIENFMKEHQEENVEPEPDIPIAEPQINIRKRGRCYLCDRKKDVKCTKKCSKCLLFICKEHTAKAITCTKC

SEQ ID NO: 96 - protein, 587 aa, synthetic construct
MTRFLRDEDIARLLEEGTDSEYEDTELNDPDEDVDDISEHDSIHSEHVSGSEMELSEGSSTSDEDEESSGNYFYGKNRFKWSKSAPHSSRVRQHNMVRERTGLRGPAASKREMSPVESWELLFTDDVIETIVHCTNKKLEDKYSPPLLRPYYGNNIDCCELRAYLGLLLFTSIFKSNHENVRSLWATDFTGRDIFRGVMGINRFLFIHFNVTFDDISTREERKSTDKLALIRGIFEKFLSNCSANYSCSEYLCVDEMLVKFRGRCSFRMFIKSKPGKYGLKIQCICDAKTHYLCNAFIYSGKPLTQRNIPFAVPTQDVLKLAEPFYGTNRNITGDSWFTSVELVDELLKRGLTYVGTVRKNKREIPRQFLPDKTRPTESSLFGFVRDKTLVSFVPKRNKSVVLLSSMHHEVSVNQQSKKPEIIDFYNATKGGVDALDLKCSNYSCSRRTRRWPNAIFAAMLNISLANGYVLWVGSNPNKKVSRREYIKSTAMSLVKPYVCRRLENNPKLAKDIREIMCKLVGREQMFSTPNEDEPAPSASKKRRCYICPRSKDKKSSLTCNVCKQCICKPHSKTINFCLSCESKGND

SEQ ID NO: 97 - protein, 594 aa, synthetic construct
MAFNRLFSTPEEVLAEFERQLESDDEELDLQLDFEESDLEDGDVLKSDDFEDEFSVGDEDLDPSAVMDLNSDERVFELGKDGETIWTNQPINSKFSRTKACNLITDRPGPKGNAKNLTDELELLSLFITEDMISKIVRHTNEQIEVTQIRFQTPQSYIGPTDEVEIRATIGLLYLSGVLKNSGLSVDDMFSTTYGLPLFRLTMSKNRFVYILANLRFDNRATRKERQLSDKFAPFREIWSMFEEKCSKYYSPTECVTVDETLLGFRGRCCFRMYIPSKPDKYGLKIISLCDANTFYFLGGIPYIGKEQRRKNEDLPLPAQYVLRLTNSLSGSNRNVTCDNWFSSVELADALLEKKLTFVGTLRKNKRQIPQSTLSKRPVGTSEYFFQDNKMLICYTPKKNKKVLLLSTMHSTGETDEISQKPEIVHYYNQTKGGVDVLDKLCHDKTTKRKNRRWPMRYFFGILDISAVNAFVLYKWNKNKASYPRQERSNFLKSLAFLLIKPTFEKKWVNTLVPRENRIFIGKLLNKQEILPAVDSPTTPAASKKRKRCAFCDLSRDRKGNAICSKCSNSLCGEHKIIVCPSCDNEDKENDNFM

SEQ ID NO: 98 - protein, 597 aa, synthetic construct
MAYLNDGAIYEELMLEDPRDPNEHSSDEDDVVITDNVQEDLNSVHSDTELQDDLQNSELISEEETPPEKQPRHSDPSSKPSSSTACHVLVPSRTSVKGKNGYRWSTVAPVKFGRTPKRNIVVSIPGPKGNAKHVKTPLSSFLLFLDNDILETIIQCTNEEIERQRLNYSDDQRNLGIITYDELLALLGLYIYFGAHKSSQVSTELLWHKMRGLRITETVMSQRRFRFLTNCLRFDEKSTRSERKQLDSFAAIRDVWNRFEEKCRMLYNPGSYCTIDEQLLAFRGKCPFRMYIPNKPAKYGIKTVMCCDAKTGYMLTAIPYTGKTMNTNGKPLAEFFVEELSKPIHGTNRNITVDNWFSSIPLFKAMAQRKLTMVGTLRKNKPQIPKEMIDIRGRNENTSMFAFDNELVLLSYVPKKGKYVLLLSNMHQGASVSESGLPEIIEFYNSTKGGVDMFDQMCSTYSCNRKTQRWPLCVFYGIINSSCLNAYVILRHNMTTVGAHVQPREMFLLSLGEAMMTPWMEKRLVMQNLPRLIRENIADQLGKNVPETSTTLQGEGAKRRCHLCPRSKDRKSKIQCSECKRTVCQDHKVSLCLQCKR

SEQ ID NO: 99 - protein, 613 aa, synthetic construct
MDLIRDQDKILQILNESDNDDDDSPFFGPSGVPSDSEPDDNLEEDLVEDTDEIEDDSSSSSDEDLNVSRRSRNRNRILDSDDDSLECLQPCPPIASHQDLVIQPERPYIYGKNKHKWATTPRSSNTRASARNIIHFVPGPNSDARDLVEPLPIFHLYLSDEMIDQLVIYTNAEIEIKKVKYKELTHTISPTSAIELKALLGLLMQSAAIKSNHLPTRILFDDRRSGNIFKACMSAERFDFLLKCLRFDDKATRQDRRASDKFAPIREFWEKFIGHCKQWYKPSSYLTIDEQLVGFRGRCPFRIYIPNKPNKYGIKIVMVADSNSKYVYNATPYLGKGTNCDNMPLATHFVKQLCEPVYGTNRNITMDNWFTSVPLASELLQDPYKLTLVGTIRSNKREIPPEMKNKKSRKIHTSMFAFDGEKTLVSYKPKSNKTVFLLSTVHSQPDINSTSKKPEIIHFYNSTKGAVDTVDQMCSAICVNRKTNRWPQCVFYNVLNLCLINSYVIYVSNMVRERQKPMRRRSFVQSLADKLIEPWLRERYNTVTLRRDIKASIKDILKIEDATPSTSQAQQNKRKICSFCPYKKHRMTKYVCSRCKTAICGEHTVPVCADCQE

SEQ ID NO: 100 - protein, 611 aa, synthetic construct
MDLNEQQIANCLEDDTSDSETEDNLEVADYDSDDSVVDPTFVPYRLENELQNIVLDDPDPQDRSSPIPSSSGSTLPNPQTSPTSETQTTEELIVLPSTPVVRGKNNYIWARDTSGRPRSNRTVARNIVHIFPGPKLDGRDVIKPLECFEKFFTPDMWDEILLRTNEDISVKASKYKQQSSTIAPTRQAELQALLGLIIFSGAMGNNDLPTKELFNPVLCGSKYIAAMSLDRFEFLLRCLHFDDRRTREDRKKHDKFAPIRNIWEIFIQRCRDLYKPGSYVTIDEQLLAFRGRCPFRMYIPNKPAKYGLKIILACDVNSKYMFDACPYLGKTGEITPTEPLGTHYCKLLTKTIYGSNRNITADNWFTSVPLAEQLLEDPYKLTFLGTIKANKKEIPAEFKHDPSRRVGTSMFGFHDKLTLVSHKPKPQKTVLLLSTMHDEPTVDPVTKKPAIILSYNKTKGAVDTFDQMCGHLSCNRKTRRWPLCFFYGMINMGVLNSYILHISNSKSCGAKPMNRRDFMKELSDELCRPWCAKRAEERPKLRKRVRDAIHEVINTETTENNEEPSIKKGKRSMCSYCPAKKRRMSVYYCAKCKKKYCLEHRSPNCYSCDKK

SEQ ID NO: 101 - protein, 619 aa, synthetic construct
MTARLITDEDIVRALDDEDYVNTNDVSSDSENEDSLLVDDVESDFHDEPSDTEDAQVSPPLDVEQEQERSPSDAHRLREDSPSVSSSIIHLPQPNIRGKNRHVWATHKGHTSSRTSAMNIVRTARGPARSVRNITDPLVLFRSFMTDEICEEILQWTNAEIAIKKQSYETKTSTQNETCKEELNALFGVLLLSAALKDNHLTSDELFNTSFCATRYISCMSRERFDFLIRCLRFDDRAVRIGTLQSDPLTPIRKIWDLLMIQCRNNYVPGTNVTIDEQLLAFRGRCKFRMYIPNKPAKYGIKIEMMCGSGTRYMIDAMPYLGKATNTGGVPLGEYVVKELTRSIYGSNRNVTTDNWFTSIPLAKQLLQQPYKLTIVGTLRANKREIPEEIKNSRGRAIGTSMFCYDGPLTLVSFKPKPSKMVYLLSSCDEEGTVNPTSKKPHIIEFYNATKGGVDTFDQMCSNMSCNRKTNRWPMCMFYGMLNITSINSYVIYCHNVLVSGGKPLARRSYMKLLHEKLVEPWLKRRLEIPTMPRRMKDKIRDVLGLNHEDRAESQGQNLEQSGSESSPILENASTRKRKSCSLCSYKKRRMTKNHCFSCRVPICGEHTVDFCPTCAKKI

SEQ ID NO: 102 - protein, 604 aa, synthetic construct
MERGRRSRRILRGNRNPRLLQRQLNVNEIPGILMEPESTDDEDFSSGSDNNYVPEREARSSGSSYETSTSDSESQPPPATLTEASSCDPEFQWNIGSARPQQLAFTGESGIKIDFRPTPAALADIFFSNDFLLSLVQATNNYARRTAGPLQKHARFRYWKDTNIEEMRKFIGMLLYMGLVQLPSISHYWRLDPLYNMPLFRTLMSRNRFQLLLRNFHFSEDDSSATSRLHKIQPILERFNNIMIDLYYPKADLSIDESMVLWRGRLIFRQYIKNKKHKYGIKLYELCESSGIVMKIRVYRGKSEKPPPGTLHSTEVVLDLMEGYLDKGHVIYADNFYNSPDLTRRLTQRKTYICGTLRKNRKNLPVDVTNAKLKRGELAAKVCNDITVCKWKDKRDVLVISNKHNLQLVRVRNKNGRISLKPNIVADYNLGMSGVDRGDQMVSYYSCLRKTLRWHKKLGLHIFEVYIQNAHRILRLQDRTSKMRLLQFREGFIRHLLGMNNNDPTIATGSHSTQAGPSQEPQSILSPASTSQLSAKTRVPLDNFHYLEKLPPTAKKAVPAKPCRVCTKNKIRKESSFVCATCPEKPALCVNNCFKIYHTDPKFA

SEQ ID NO: 103 - protein, 577 aa, synthetic construct
MADEPGPSKRVKYNKREERLTDEELLALLEESESENDPFENSSDEDPDYSTENEVSESEDEDLPSFPKVLPPPPAPVNSPPPSLPNIQGKSMDFWTFAEVPSQKFSFTKTPGLLVNVNSNEPIDYFNIMAPDVFFDMLCSRANNHAIDLISLGRGDEARITRWVDVTPKEMRTFLGLLFHMGTIKINRMNDYWKKNYLFNLTCFSSFMSRNRFLLILRSLQFENTDSNEPRTQLGKIMPLINYYNNKMVEIYYPTNELSIDESMVLWRGRLKFRQYVKGKRHKFGIKLYVLCEPKGIPLKMLVYAGSADTELSGERHTEKVVLALMAEKLDNGHSLYMDNFYNSVKLAEELLTRKTYVTGTLRSNRKGNPQEIIKKKLKKGELVVQHNSQGICVVKWKDRREILAISSQYGGQLEKVTTQRGQEKLKPAMINKYNQFMAGVDHCDQMLSYYSCEHKTLIWYKKLAIHIFQMMLLTSFYLYKQGHPNNKKNLYDFRLSIIEKLLGPLPTEPLPKIRITHLPELCPKPDGENTRVKRRRCAVCWEQKKQRKDSIYYCPLCPNQPGLCLTPCFRVFHSTK

SEQ ID NO: 104 - protein, 577 aa, synthetic construct
MNMEPNRKRRRVCTNSEIDRELIDSDSDTEISSLFSSDGNSEYIPTSNSSDDSSSEVDRNEVVTEVEAHEYVQNQNNFTDDVNVQFVANEVHNDHISINGCWTDTNKVPLIHDFVGEEKVNIQTTDPVEVFGNIFDTALLEKIVTWTNTRAGIMRGTPLSKHAHLNFWENVSVEELKKFLGLCMLMGNVNLPSIKHYWSKDGLYEHPIFGRTMSRNRFECILRCLCFYDPNIDSTDNRMHKIENVLNDILANSVRVYSPGKSLSLDEAMVLFRGRLSFRQYIKNKSHKYGIKLYELTTSDGFILNIIIYLGKGTLIDKEKGHAFEVVRKLMQNYLGKGHIIFLDNFYNSVDLAEYLMTNQTNMCGTLQSNRVGNPQVVVQTKLKKGECVSRQRGDVTVMKWRDKRNVLTISTSNGPEMEETRTKRGEVIQKPSMVVRYNKGMSGIDRSDQMISYYSTPRKSLRWYIKIFFHLLDVSLWNSTFIFSKLTTKTTYLNFRDIIIKQYIHVEPIPRKPRPIRSSQTTHAPVKLEKRLRCHVCREAHNTRKQTFYACSICKDSKDKPFGLCMPTCFNLWHEK

SEQ ID NO: 105 - protein, 581 aa, synthetic construct
MCEPLDMDLAMEDFEFTQDEWKQINALEISFSNASINNDCLLESDSEPMQLPKKKRRLVIASDSDSDSESAENFVAANISQNDLISAKWSRPKRRQPSVIPFTEFEGMKPPYSNLLKEAGPDQYYDLLVPEEILEMIAEQTNLFASQNITARQTKPGSRSHSWKPTNKTEIKQFLGLVLFMGVVKLPKLAYYWSKDKILGQTFPATVMSRNRFELLLQYLHFSDNLSADRNDRIAKIIPLIDALNATFQKYYSPKEDICVDESQVPFRGRIIFRQYNKSKRHKYGMKLFKLCTVPGYTCKLNLYSGKNIDAINTTPTNVVMALCENIFDKGHTLATDNWYTSLELAYRLLEKQTHLIGTLRKNRRGLPKAVVDSKLQKGETMAMENEDGVTVLKWKDKRDVLMLSTKHSDKIAVVVKKGKQIRKPKVILDYNKSKGSVDMSDQMGAYSSPLRKTVKWYRKLAIELLLNTAVVNAWVMYNENKQSKSSIVDFRRALIDYLTRPADSQEIIINERPKRLKHVLKLKEGKVRDTRRFCVQCYKDSVQEFGRKVAKNKAKKVSTYCVQCKGEPHFCLKCFNYVHR

SEQ ID NO: 106 - protein, 616 aa, synthetic construct
MDYESDDSLYDPDYYPSDGNATASSESDSANESWNPPLSFRRASTFTESALPPLPEVQRRISARRIGNTDLDAYNMPSTSSATISMSSTSATTLTTGNNEPSLSGTTSEWLKQKLDSIEWHMPTQEGPTPMEYTGPISGFKEIYSGILANASPIDYLDLFMTNDIIQHIVDQTNLYATQQLLSNDIPSGSRQHAWVPVTLDEMKKFLALIGWMGLVKLPSIKDYWRVHKLYNIPLARTVMPRNRFELILKFIHFSDNTLADPNDRLYKLRDILDKFINNYKKTYTPGEKICVDESLIPWRGRLLFRQYIPNKSAKYGIKVFKLCTEKGYTWNLSVYCGKSRDPQTEVSEKTVMSLAEELLDEGRTLYTDNYYTSVPLALRLRNRKTHLVGTLRANRKYLPKEVVQAKLKRGEMAAKQSNEGIVVLNWRDKRDVRMLTTKTSALEMVPNNSRNTNAMHTKPKCIADYNKGKSSIDISDQMATYGTALRRCTKWYRKLAFEIIWGTSVVNAHFLYNEYSLQKKLTITEFREQVIDALLERISSTNVDRPSRSRPDDKHYLVQNIVNDKKVRGRCLECYKIYGRTGLQIAGKNKKAPQIYTKCSVCNFFLCRQCFNKTH

SEQ ID NO: 107 - protein, 630 aa, synthetic construct
MSTRTRKILFLSQCEIENVDKKTVNSVKDKSSVCVSEEEEINDVVGYPVECEEPSSSKNISTNKPSNDPISDEEIDSDLDVFDSDDSIVDKNYVPSSSDSESDENRSNDEDSYMDYTENDNVVLDEIPVRNLILNQDEWHDITADNGCVYQKQESCTLNFDTTTIKNPIHAYQLFLTDDIIKTIVEETNRYARQMLISQNSSKSRLNLWVDSDEAEIKRFFSIIFVMGLNDLPALNLYWSRDSMFRNEFISNTMPRDRFLLLLRCIHFCNNDTADKSDRLYKIRSLLEAANKNFQQVLTPGRVIVVDESMIPFQGRLVFRQYIPNKKHPYGIKLYKLCTVDGYTNNIIVYTGKNVTPPEVTHSEQIVHDLLECVEDKRGHYLYADNFYSSLGLAKSLFNQEVIYCGTLRSNRKGIPKSLPSKMKKGDIFGTQNGPLRIIKWMDKRPVLMLSTDPTHTATCIPTGKRTRQGDDITKPKAVTEYNKAKKGVDLSDQMSSYHSVLRRSLKWYRKLAFEILFGTFVVNSWIIHNKINSSKLSITEFRRRLAQELVFTPVDTREKTPKRVSHTFVKPSGPGKKRRRQCQSCYSNLRETCSSREASNLVTKVTSFCNDCPGQPGMCLPCFNIKHKK

SEQ ID NO: 108 - protein, 596 aa, synthetic construct
MSSGKKKLTDEEVREMIEDSDDEDFSDGSDEEFVPSGEELEDSEMEDVIEDIEYDRNYVCESQPNENDRNRASSSPSESTDEESDPVRGSPRQLPFEQQENLIWLSDVLALGRENFSGNPGVRNIEEILDEDRKVNIIKIFEYFLDDSLIEEMVTHTNNYAQTYQNVRSGLHSRMKRWKPVDKGDLKKFFGIIMFMGVKILPSIEHYWKLDILYYCPLMHSIQMSYNRFVTILRCWHFADMQVDNSNITHKIDSLVQKLVGKFRSLYVPPEVIVVDESMIKFYGRLRIKTYNPSKASSYGIKVYKLCTVNSYTWSYEIYAGASIQYEGLDKPGTVVAKLCEPLLDQGRVLIADNYYTSVPLAKYLKDRKTDLCGTLRKNRVGLPLNVTKSKLKKGQCVARQKDNYITVLKWHDKRDVLMISTCHGDEMTSVRTRLGETLKPNAVIDYNDAKKGIDVADQLSSYHSPLHKSLTWFKKVAVDLLFRVAVVNARCIYNEYSTGAPLTMLEVQEQIIRHLLGPSANVTTPARPPSQGLSTHKLTEIPRRNNMLVRKRCTNCYTKLRKEGMPAASKAKQVHTECIQCQKAFCLDCFNNVHC

SEQ ID NO: 109 - protein, 588 aa, synthetic construct
MSKRKQSGGLSPDDIHNILTNEESDDEYSSGSDYDISGEITDSSSNDMTSDPGPATKVKRSRLEPDKPPVLRKWMPVGKFLPTISPFFDDSKSGISECFSQKLEVTDDDNGGNRLEISIFKEIVDEGFLSKIAIEMNKYHTFILENTDVKENSKLHKWKGITVDDLHVFLALVMLMGHIKKNRLKDYWSKNSLIETPIFGKIMSLDSFQLTLRLLHFCDNNTQPQGDRLYKIRAVVDHFRRKYQSILYPFRNLCIDESMVAWKGRLQWKQYIPSKRHRFGIKMFLLCDCETGFVLDHIVYTGKETEIEYDESVGISGSVVKTLLTPYIGKNHNVYLDNWYTSPSLFEYLHRNRTGACGTVRKNRKGMPEFPKNIKKGECVSRHTTVMLAQRWRDKREVNMLTTIHEDLMVQTGKSDYTTGQSITKPLSVVRYNENMGLIDKADMQISFTGNVRKTMKWYVKLFFHEIDIAVLNTYIVYQMKTGRKPQLHDFRLKLIDQLLEEFAKERKSPRGGRPSPVGDCLRLSGRHFPKPVPATASKEKPQRKCFVCQHTTRRESKRKDTRVMCIECNKPLCLHPCFEEYHSLKNF

SEQ ID NO: 110 - protein, 567 aa, synthetic construct
MASSSRELSDEQVLDVLEDESAECSDTISEDSIGYSLDSSSEEEEPPQLSVNTLDNNWEEIDSANPPVPFACDLLGKQIVSVQCAANPLDYFELFFDDDLIAIIVTETNRYAEQFLAREKEKILAQKKSRFQKWVPTTPREIRVFLGLVMLMGIIQKPSLRMYFSRQHILETPFFPNVMSEERFSLLNKFLHFVDNTNEELVKRDPKLYKILPISEYLKNKFQKVYVPGKEVSVDESLLLWKGRLSWKQYIPMKASRFGIKMFELCDSVGYLWNFIIYSGSGTKLGCEITNDKDLFSTKVVLSLCEKLLDSGRCIYMDNYYSSPDLFLRLVQRTTDAVGTVKITRKGIPANFKRKLKKGEVVSAKCGKLVALKWKDKRDVTMLTTKHSDQMHPVSRKGGLEFVNKPACVIDYNKYMGGVDLKDQLLESFLLERKRTKVWYKKLFNHFVNIAVLNSYILYCKAGNKKDHLLFRVELVNKIIVKYHGQNIEKRKGRPSDMDTECTRLTGQHFIEENPRTESLKVGRRKCVVCTKNGKRKDTRYRCKNCPGAPSLCLDPCFEKYHTQVHY

SEQ ID NO: 111 - protein, 643 aa, synthetic construct
MKRANSPLSDNTISRVINEEENFSSDESFLNNFGSEVDSSSSEVDDTDNDPTYDPDQPGPSSNILPIVSRPRILSESESESSIDGENVRSRLTTIHQQPRQPSRPRPRHTNGNSTDDSSGENNEGWIDVTAENDPGYSHSFSFLELPGLKHCPPMNTPSIAYFRLFFTVTLMEMFVKYTNMYAEEVVRKYNSNLGPNNSLRTWVKVTTAEIQAFICILINMGLNHKPTYASYWWKSNSQNCSWFGRMMSRNRFQEILAYFHMVDTRNLPKPKEPNYDPCARVQPLIDHMNRISKHHYSPNMCLSIDESIIPTKCQNPLMQYLPKKHHRFGIKLWVLCDAVTHYCVHFFVYKGAKNTDRQHVRETGLAYNVVMKLLDVAGCLYKGFHIFVDNFFTSVRLAKALYTKCTHLTGTIRMNRKGVPQEIKTNYTIGERKYKRKNSMLLLGFRQRKTQKKPVMLLSTVYKAGEQQKSKKRGNKIYITTKPKIIREYNNFMGGVDGHDQMLHQYMGDRRSAKFWKKLTFNLLSRMILNSYILYKQNTTNPMTRLKFLVTLVDSLSAEWFDFKEQRHPIPDTDVSDDENQGGGDAGILPRFFEKLPNKKERNCCVCSAASTRLGGKRKKSSYICKKCKKGLHTKCFPLHKC

SEQ ID NO: 112 - protein, 619 aa, synthetic construct
MSRSPVSDNTIHNIIVEDEFLSDSEINNLQNSSSSEVSESEADDTDLDPSYTPTPSTSCNFQGTLASNRPVNRSVISSSEDEDRSAATTTTPRPRGRPRLPENISSSESNSDDDQWENIEENNDPGYNHSFQFNEIPGCKHCPPRNSAPITYFDLYFPMTFLNILVTYTNLYARNFIQQNLHKLGNHSRSRAWRPVSFVEIKAFLAVVINMGLNKKPTIESYWSTKSSQNSPWFGKMFSRNRFESILKFFHMLDTRHLPSPKEPGYDPCARFAPIVDHANNVFKHYYAPNKQLSIDESLVGTKNHTQLLQYLPNKHHHKWGVKLWLLCDSVTHYCLSFFCYKGKRDTEQSNGRVKHGLGYTVVMKLLEMGNYLNKGFHVFVDNFFTSFPLANDLYAKLTHLTGTLRINRQGIPPTLTQKFGVGQTKYCRKGKMLLVAARLKKSQKKQVLLLSTNAEAKSELKSKKRGNKVFITSKPSVIRQYNSYMGGVDTSDQMLYCYLDERRTLKYWLKVTFHIFGRMIVNLYVLYKNNTDNPISRLDFTVALVEGLAAEWLGDQAPVQAQRAGNEPLDLLPGGKERNCSVCSRLSTAQGGKRKKSRYICKNCKKSVHPLCLPKHTC

SEQ ID NO: 113 - protein, 586 aa, synthetic construct
MSRRKSLSQDEIYRLLFEEIPSDQDSVVDSDDSATDYTYLNQNVNKRQFCDKVLSSSESEKSDCETTNVSAAQPSTAALNVTVTWTQDTRVLPVPLYIQESGVAAKIKNKANHKPLDLFCEYFDEALIDHIVFHTNLYATQSGKKFVPTDTCEIKTFIGINLYMGITKKPSYRDYWSTEPDLGENYISKLMTVKRFSFLLSHLHLNDNSLAPDRSSANYDKLYKIRPLLDHLGEKFLECYKPHQKLAIDETMVKFKGRSSLKQYMRGKPINRGYKIWMLCDESSYNLKFEIYTGKNLGATEVGLGAKVVQTLMSGFEGHHHIVFMDNYFSSYELFKNLKSKHQVYACGTINHRRKFLPTLKDEKQLKRGEFDSRVSVDGVAVYRWKDNRAVNLISTCHDPSDVSSVNRKMKDGTITTIKCPQVLKDYNSYMNFVDNFDRLKSDYSTDRKSKKWWMRLFFHFIDCCVTNAFIMHKELPEEQFTNKDFRRQVYKGLLAPRIVASRGTTTSGGQGNSQSPITIKAHKPFVDKSIRHESSKHQPVHSTSKRCAACSSRKNPVRTVWMCSVCKVPLCIRAGKECFRTYHTK

SEQ ID NO: 114 - protein, 654 aa, synthetic construct
MDPKKIKWLSRKPLTPSELEDIAEQIFSEDLPLDNNFMTDEEMSDSEDADFQSDLFIDYLPNLSSTGNSNSESCLAGADKLAECVAENQDAIGTDFDPVPPCSSKRSPQSAVLEEKTLDDMGKNVGTTKKRKETRSKRNFEEIMIEETSRQRGSETKAERRWKRTPAEEIIPDYHISEGPVESQLIGCNSPTDVFLRVMGDSVEDIVFQSNLYATQRNKVLNLTKEELFAFIGINFFMSYHRIPAWKNYWSTSEDLGVPLVAKCMNRNRFEEILRYLHCNDNTLIPKDNKDKIYKIRPLSDSLNRQFQKCYETTRRISIDESMILFKGRSSLKQYNPMKPIKRGYKLWCVADQNAYVSRFEVYQGKNEKLEEEFKDFGLGERIVLSLSKPYWEKYKIIYADNYFISINLLEQLYMKKTLACGTIRSTRLGFPMMGEDKQMKRGDYDYRVSNTKIRAFKWKDNKVVNFASNFHGTEEITVKRTQKDGTRVDVKCPSIVADYNKHMGGVDRADQLRSTYGMIRRSRKWWHRLFWGFLDIAFVNAYIIYNKMDFEKKTTLEFRRCVSMGLMAALNSTKSRRSGPARKRRKHNYSVPEDVRLGNRGSHWVIFQENRGRCEMCSAQAVQSRPYSKCSACGVFLCSNKNKNCFLEFHEMD

SEQ ID NO: 115 - protein, 597 aa, synthetic construct
MPFPWESKQRLTDKELLEIVDDDEFFAEFDLDEDNNVQTDVESDAESGNEDQNEHVFISQTNNSEQREPKDLCEEVESEPTQEIEGNETEDPIREEPARTWQKVERSTTILPEYQPGSWEETYFRDCITATDVFVKLLGNVIPDITFQTNLYGTQNNKTLNITQNEMLAFIGLNFLMGYHVLPNLESYWSSADDLKLEIVPETMSRNRFQTILANLHLNDNALIPNNNTDKLYKVRPFVDSLNVVFSNYVGEQKMCVDESIVKFKGRSSIKQYNPMKPVKRGFKFWMLSDMSGFVKKFEIYQGKNEKVNENFKDFNLGERIVLELTQQDWNKGKVLYFDNFFTTVRLLEKLKLENTLACGTIRSNRKGLPKGMIIDKNLKRGDFDHKTSNTGITFFKWKDNKVVTLASNFHGTKSVPIKRKLRDGTISYIPGPEIVYDYNKFMGGVDYHDQLRQCYGIDRRSKKWWHRIFWGCLEIAFINAFVVYKKIREEPITVLAFRREVTLGLCSRSPKREQVKRKSVASGPASKKRRYNYSVSDEIRLGNRGNHWPQFVDRRGRCEMCSAQGIQSKPHSTCSLCGVFLCVNEKKNCFVQYHNI

SEQ ID NO: 116 - protein, 604 aa, synthetic construct
MATSLTTKEIVYILEGDISDIDISDDEDISEENPDWLLNREAVGPDVMNTEVQGFPNIPDVGLAFEVNKCSDDSDDDDMPLAQRYPHLMNQSQRKVNRSLWTYEDLGYVDSSCDSQFSPPPDEISTPFSYFKLFVDNDMISNIVEQTNLYSVQEKCQNVNTNDKEIHQLLGIHMIMSIVTMPSYSMFWSEKSRYGQIADVMSRNRFKTLRGNLHFNNNDNMLSRDDPAYDKLFKIRPFLNALRKNFLKVEPEEHNSVDEFMIPLKAHTALKQYMKSKPHKWGVKVFARAGVSGFVYDFEIYLGKQTNVKESGLGISGDIVIRLAENIPKHKNFKLYCDNWFSSPKLFSQLRKDGILAAGTIRRNRLGQCTLKSDKELKKQGRGSFDYRLEKSENTFVLKWLDNKPVYLISNFVGAHPVENVRRWSVAEKRYIDVPQPNIVRAYNKHMGGVDKQDMLLELYRTNLKGKRYYLRVIFHFIDLSVVNAWLLYRRHCEQLKEKYMPLIEFKLSVAHALLYFGPNISNKRGRPSSSPSVEGKGVAKRTFTPRPVADVRFDGIDHWPLHVNTKNRCKLCIKSYTRVKCEKCNLPLCFSNEKNCFKTFHVK

SEQ ID NO: 117 - protein, 594 aa, synthetic construct
MRGQYDVIREDEIILLLHGDESEISDNEIISDDENEDSDPSVLLPLPEQKQDELHRENILEDPLTDDETPLAERLENLDSGNASRTSYTWVCKDLEAVNVPCYVKFSDPPSEEHSPLNYFENFIDEDIIDNLVTQTNLYSTQVLCQSINTNAGEIRQFIGIHLLTGIMSVPSYKMYWANETRYSQIADVMPLRRFYQLRRYLHVNDNAKILPRDDPHHDKLFKVRPFLERLRENFLKTEVEEFNSIDELMIPLKSHTSMRQYIKSKPHRWGIKVFARAGVSGYIYDFEVYVGKGTKTVGDAELGMSGNVVMRLIENVPRHANYKVFMDNWFTSLNLAATLKNEGIFIVGTIRKNRLSGCQLKSDAELKREGRGTCDYKINEENNLIVLKWYDNKAVYLTSTYKGKEPVENVQRWSQKKRSYIQVPRPNIVQEYNTYMGGVDLHDMLVQIYRINIKSKRYYLRIIFHFIDMACVNAWLLYRRHCGQRNTKYVPLLKFRSMIASALLQKGITPSARKKGRPSSESPRPSTPKRKPKAIRPVADVRFDQMGHWPEHLDSKSRCKLCINAYSRVKCSKCNVALCFNKHNNCFKTFHCK

SEQ ID NO: 118 - protein, 644 aa, synthetic construct
MDGSSVRKRPLTSKELAELAENFAETVNWSESEDGLDFSDTDSVADPTYLPDSPNPIEYFTSEDLLENLSSEDENENNTEDNEEKNKESGENNTENDNANLNDNEETTRKNTSRKNKSLPKRDVLWRHRGLHLNDLQLEFHGNNQLPPDCMYLDTPYSFFKYFFSNELIDRIAEESSLYATQQNPNNSAHISAEDIKQYMGICFYMSLVHLPSVRAYWSVDLGYDPIRNVMPVNKFEKIRRYIHFNDNSKHLPRENPDHDRLHKLRPLIDHLNETFRSIPLEQFLAVDEQMCATKVKHFMKQYLPMKPHKWGFKLYVLAGVSGFAYHFEIYSGQENLQTRPANEPDIGASANIVVRLARIIPTMQNYRLYFDNYYTSLELMLYLAKRGIYSLGTVRRNRIPNCKLPSEKDLKKEKRGTSSEYLTTIDGIDISSLVWKDNKYVTLLSTYAGKEPITSVKRFDKKEKRMVDVDCPSVVKEYNHHMGGVDLMDSFIGRYRIALKSRKWYIRIFYHLLDLGMVNAWLLHKKVLTLKGQSQNILPQSKFRSEVAHCLCLVASQNTSKRGRPSSNLEQAIQAKKHRGNAQHVPPKDIRLDQVGHWPVFAEKRTRCKYPGCRGFTDINCEKCGVTLCMNKKNNCFKLFHNN

SEQ ID NO: 119 - protein, 681 aa, synthetic construct
MSLRKEHYNLSAMSEEEIDALMNSIDYGDDTGSEEDFDSDDDVADPNFALQGIDNVIDDCLNSTETMEAMAGTLNLSNINVSEVEHEISDTSEILPCTSSGATATLLDSVSVPKLTLKNRLFCKKRKHSHELKQETGGPSIPSSAQFIGIVSDIKNDSSEFKSMLWKKKNLQLHVNEIVFRGTREMPAELTNLKTPFECLNYFVTDELLEFLVEQMNLYARQKHIESRFTTNAEEVRKFIGILYFMSVYRYPSIREYWARHSFEAIRQTMTRKRFDEIRKMLHFNDNSSDPGNDKLYKVRPLVKHFNKRFESVPKLQRLCVDEQMCGTKMGGNPTRQYLPDKPHKWGTKLFALCDSSGFLYSFEIYSGAGDNVIPENAPDLGASSNVVIRLSSKIVNNVNHIVYFDNFYTSLGLVTYLKSRGIYSLGTVRANRVPNIKLSNEVELASKKVQRGYSEEYVANAYGIDISNVLWKDNKTVRLLSTYVGIKPFVSRNIDEQPQKAKRWDRKTKQQTEVDCPFIIREYNRHMGGVDLMDGLIGRYHIRVKTRKWTNRIFFHLLDVAMVNAYVLFHRLHKGDRSTQLPKFRTEVAESLCAINSSPENRQVGRPVTTQPPKAKKAYTPTDDIRYDNMSHWCAFADRSGKKMCKLPGCKSETQAYCTKCNLNLCNSTSKSCFLTFHTK

SEQ ID NO: 120 - protein, 626 aa, synthetic construct
MRPEEIENILKRLENGEISEDDSTDNEDDIDYYSSQRDRLMELDDEEDVGNFPTDPNLDADPPAANVDTDMQHNLETVERNPTPTTSSQNNLQTAPFNSRNLVWRVKNMDLVQNAFQFAGNTEYSQEIMNLDTPFQFFSYFFGEDILRFLVDESNKYAFQKNPNFTEPVTVLELRKFIGILIFTSVYHYPSVRSYWSNITKFEPIVQTMNRNRFDKIREIFHMNDDSKHLPIDHPQHDRLHKVRPVIDYLNKKFITLPFEHRLSLDEQMCSTKMKHFMKQYLPNKPHKWGFKLYVLCSLSGYAYSFEIYSGAKDMDRLPGEPDLGAVSNTVIRLLRPVPRHVNHIIYFDNFYTNIPLVHYLTNEGIYCLGTVQRNRLGKSCKLPEKREIMKSNVPRGTYHENVASHEGLELSVTSWKDNKQVLLLSTYVGAEPADTITRYEKKLKANIQISCPRVVKEYNAHMGGVDLMDSFIGRYRIRIKSRKWTTRLFYHLLDMTVINAWVLYKKVNTRKGKLQKNIMKLADFRTELADTLCRYQSHSKNKRGRPSTNSQQDNTVPSKKPRTGAQVLPPTDVRSDCIGHEKIFMESRNKCKFSNCRKLTSWFCKKCKVSLCDNKNNRCFALFHA

SEQ ID NO: 121 - protein, 617 aa, synthetic construct
MNNRDIERYLASLEDGLLSEDDMEASDQEEEQVPDYNDKEQMLNMLESDEEDTENPQSAEDDPPLIENEIIDIQVENISVPTGDTFSGLLDKRKLIWKKRNMEFDDSKIKFLGSSDLGPEILELDTPYTCFNYFFTEHFMKSIVEQTNLYATQQNPENSTAYNLSDLRKFFGVLIYMSVQHFPSIRSYWSPKYGYEPIYSVITNNRFEQIKKSLHFNNNDLHKAVGEPGHDRLFKIRPVIDELNNKFSSVTMEQRLSIDEQMCATKVAHFLKQYLPNKPHKWGFKLFVLCSLQGFAYKFEVYAGQDLTNKPENEPDLGPTSNIVLRLARGIPRFVNHIVYFDNFYTSVPLVYYLANQGILSVGTVQQNRIPNVKLPDKKQFMSKSTPRGSYEERVTTFQNVDMSVVAWKDNKIVTLLSSYVGALPLNNVSRYDKKIKEKVQIPCPKIISEYNAHMGGVDLMDSFLGRYRIRAKSRKWYIRIFFHLLDLAVINAWILYKKNLIAKNLPKKDILNLGAFRNELAFTLCNLGLVQENKRGRPSSSALEHEIQSKKKRSAVAVAPPKEVRRDGQGHWPLVGGSLRCKYPNCKGYSTLHCSKCNVNLCLNKNNNCFFKYHNE

SEQ ID NO: 122 - protein, 621 aa, synthetic construct
MSRKSQEEMLMEWLEIPLSSDDDLECFSDDSIQDETYVAPESVDCDSDSSDEESQVPVIRTEDVSGTRQSDVIMKESTSQLSDNALKLPCSSKNVTKNSRSVVWKDKQLIIPELQSKFHGNTSLPEEVINLNTPYQFFKHIFPSKLFDLIVEESHRYSVQCNPDRPIDLSCDDIKKFIGICLVMSIVHVPNTRDYWGEVTGTHLIKTTMTVNQYEQIRKFLHFNDNSAMIPRGEKGHDRLFKIRPIIELMRSRFQTIPVEECVSVDEQICSTKARSYLKQYMPNKPHKYGYKLFVISGISGYAYDFEIFTGDENEPEKRVTGEEDLGASANVVVRLSRILPRNMNHKLYCDNYYTSLPLLVWLHKQGIYSLGTVRRNRVPDCKLPSEAELKKMARGASVERVATVDGVDISNVVWKDNKSVMLLSTLAGQQPIHEAGRYDKKTKSRITIPCPQIIKLYNKHMGGVDLLDSIMGRHKILVKSRKWYIRLFYHLLDMGVVNSWLLYRRVAETKGIRRTMKLSEFRMEIAHCLCRSGQTSAKRGRPSNELENQIQAKKTKGPTAHVPPQDVRLDQVGHLPSYLQVRQRCKMPGCKGFSWVQCNKCAVALCLHKQKNCFQTFHVK

SEQ ID NO: 123 - protein, 619 aa, synthetic construct
MDPKRFYGSNKLANPIPGDGLSDDELAPDSDSELPRSSIPFRRPIIVPETDDEFTDEDDIPIANLPSTSKNNVQARRRKKLVEKTKWTSAHLASYNPNQFVFTGETELPSYIKQLDSPADFFLFLFTDDLISMIVEQSNLKAIQDNVNKPAHITKEEMEQFIGTVIFMSIVKLPASRLYWNTTIGQHQVNNTMTCNRWETIKRFLHLSDNTKFIPFGTQGHDKLFKIRPLLDSIRQRLLLVPKEEYLAVDEQMIPTKGRHELKQYNPAKPDKWGYKNQVLSGASGFSYDFDLFAGEQSNKVPDGAPDLGVSGNVVTRLTQSVPKHKNHKIFFDNWYNSPKLQVYLTKSGLLPLGTVRLNRVPNAQMPSEKDLKEQGRGHMIEKTAKIDGVQVSLITWFDNKPVHMLSTYVGSEPVTSKRRYFRKERKYKHIDCPQAVEVYNRHMGGVDLLDSMLGLYRIHLRSKKWYKRIFFHMIDLCVVNAWILWRRKWTDTYMSLFDFKHAVAEHLCKAGKCLPRKRGRPTTTSVTGSPSTCYQESPTNTSNFGTPTRRNGMRKAPAKHQELPLNSVRIDNIGHLPEWHKYRQMCQVCARKTYTKCPKCGVFLCYNDKRNCLFKFHQ

SEQ ID NO: 124 - protein, 632 aa, synthetic construct
MDAEELLNYLESLSSDDECYEDVKNANEIQLVLYPPEDGLDSDIDDAPSDDEGAFSLRDVGKGVLKQPMDVICVTQDRQKYNIQDSKANCETEEDKDLPDTSEDRAEDNIEGESASKIVYKSDDIFSSDDEIPLAKRFKIKFSAKKKAKWHSINLEQIKEKKKIPKFSEPDIVKRIRDENLKPIDLFKMMYDEAFISYICIETQKYAASKGNHDFVVSTQEIYKYFAILLLSGYVKVPNRRMMWETKADTFNPLVSNSISRNRFESIHRYLHFNDNSKIDKENKVYKIQPFIDQINNVSQKLFQPLGSSFSLDEAMEPYYGRHHMKQFIKGKPIRYGFKFWCLTTSEGYLLKFTPYCGAGDKMEGKSLGSSVTEKLCVGYLPGGSTVYIDNFFNSLPLLRTLKSNNINCIGTIRADRVEKAPLQDLKKSARGSSYAIHEASDKITVMRWHDNSQVTVATNVEDREACLTKGTCRRWSKKDKSVVTVEQPTLIDMYNQGMGGVDLFDKMRGLYRTRIRSKKWYWPFIRFGLNGGVVNLWNLYKIVFPKMGLLEFIRSIVLAILTAPPYLVGPKPKCSRNVLLEVRYDRLDHLVESQATQRRCAYCGKCAKFSCSKCQVGLHPNICFRSYHTSP

SEQ ID NO: 125 - protein, 626 aa, synthetic construct
MRRTLVTIRQEIILTLYTSRNMNSRQHSSVFLTLKEVEDIINDPEFIVSDEEDDGATIDIVELPPEKVDDLTDEENIEENFLEDTTPTETPGLIEIHHTYSENCNEVNCIKDKQSSSNRKKRKPNTVSDWDKTEPHFGILCLNIEEEKIQNKIKATLMGMTPTEVFEKLIDDDIMNYIVKESVIYATQKNNHGFVLSTDCLKKFIGILVFTGYHCLPQEQLYWSEDEDVNIQIIRKCMPRNRYLEIKKFLHFNNNANLQGQPLESRDKLFKIRPLIEKFNSNIVKFGIFSEHLSIDEQMVKYFGHHYLKQFLRGKPIRFGFKQWVMCCSKTGYCYHAEVYEGRKEDRNSEFSYGLGGDVVMRQVAVIEEPQKHKFYFDNYFTSFELIKDLGERGIRATGTCRYDRMNKCPIMKEREMKTEERGAYDYRFDKQNGVLGVIWKDNSCVKMLSNHEDILPLSQAKRWSRAEKKQKTIQQPKLISQYNKYMGGVDKLDWNIQKYRIRIRGKKWYFPLFTNIMDIAVVNSYVLYCMVNGPMSLLDFRRKIACAYLCVSSCSDPKNKGRPSFTKSSISRVPVELRFAGAHYLERTEDGKQRKCGVCKKKVRKQCPLCNVGLHIECTKEWHTK

SEQ ID NO: 126 - protein, 623 aa, synthetic construct
MVSKILVSNIFLLYSIDGRRYRVDDARRILEAMLDDVPDDSVVGLPTDDYDEVAQISEDEDTPPLLENNYENDGVTEEEDVVEEEILENDVITESDTDEEEEDHDDEAEEYYLAKSGMKWNKSSNKKVQTLACNIVREKPGCKGTRPATIKDSFKTFMTDEMLDIIVRETNREAKRVYDEWNNFPGQEKNKIWKEINVTELEAFLGLLICAGAYRSGREDLNELWSEEHGRPIFRATMPLKRFKAIMRFIRFDNKNTREDRRLEDKLAAIRDIWEMFIQKISSSYVPGFDMTIDEQLVGFRGHCPFRQYIKSKPAKYGIKIFWLCDSDTAFPLNGQVYLGKKAGQPREKNQGARVVKDLARKWMNKGRNIVADNFFSSIPLAQDLLSVKTTYVGTLRKNKTEIPKEMQPHRSREVYSSIFGFSGNMTLVSYVPKKSNAVVLLSSMHQEARVNNDEEKKPEIILHYNSKKGAVDTLDKMVATYSCKRNTKRWPLVLFFNILDVAGVAAMITWLFHNPHSFGNKKQKRRLFLLQLGRELVEEYIQLRITNRRCIQKGAKSGLIALGYEINPHSQPQTRQRNSRKRCQLCPRSNDKKTKIECKICGIPVCTGHSETVTTCSNCKDY

SEQ ID NO: 127 - protein, 590 aa, synthetic construct
MSTPRRNRESDAVPPTSTNFETVISEWIAEDEYLSSGESMFSDSDESYMPLEAEKNTSESNSYEEDSSCEDEGETSLFQSQQSSTDDVFVSPNGSVWSKATPSQGRARVHNVIKFQPGPSRGVTTANLKDAWDYFIPQVIVEEIQRCTNLEAKRVSLEKRKKWQCISIAEMNAFLGILLHAGADKNWDVPIREIFLGNYANPVYRATMSVNRFEDIRRYLRFDDRRTRQDRLQTDNLAHISYVWEVFIQQCRRVFVINEHATIDEQLVSFRGRCKFKQYMPSKPGKYGLKIFWMCESSSGYAIDGMIYTGRKIGEPPQKDIAMNTVKRLVETITNSSRNVTMDNFFTSVPLARYLLDRRLTLVGTMRENKRDIPAEFKSNNAVDVYSSKFGFQNDMTLVSYVPKRRKSVVLLSTMHHNKIIEESTMKKKPEIVIFYNKTKGGVDVMDQKVNNYTCKRQTKRWPMALWCNIMDIAALNAFILYQWQHPNFYPKISHKRRLFLKELSMEMMKPYMMERSTKASLQRSIRMDIEKFLGEAIPANSDNKEKRLTMKKRCCVCPSSKDRKVMTTCCKCNKHVCKEHSILICENCR

SEQ ID NO: 128 - protein, 590 aa, synthetic construct
MASTRKARESVRANAANFESVVAQWFEEDDSCEEGNIFEDASSEESANEPADVNIEADDSPSDNITSSESENEEPPQKGIEDHTYISRNGTIWKLEPPATFRTPVHNIVKKAPGPARGLKTCKPKDAWDYFISKEILEEIINCTNIEGRRVAALRGKTWKNVSLAEMEGFLGLLLLSGVEKSWDVPIRELFLDEKANPTYKATMSVNRFEDIRRMIRFDDRRTREARSADDKLAAVRYVWELFLDKCRNRMIPNDSLTVDEQLVPFRGRCSFTQYMPSKPAKYGIKIFWLCDATSAYALDGIVYTGRKPHEPIQKNLGLNVVKDLAKSIEGSSKNITVDNFFTSVQLAEEMLQKQITVVGTIKQNKPEIPNEMKPSASRAIHSSLFAFRGDITMVSYVPKKKKSVVLISTMHHDRNIDETHAKKKPDIIKFYNSTKGGVDQMDQKIRYYTCKRQTRRWPFALWMNMMDVAAMNSEILFSAQHLTYHSGRSDKRRLFLRDLAEEMVRPLMELRIQIPNLPKKIVDAMQRCGVQKDVILPNQLPVSGKRRRCNYCPYNKHRKSAMTCIKCKTNICKEHSGVLCASCLSHVEN

SEQ ID NO: 129 - protein, 555 aa, synthetic construct
MAMRPLNDRELEEIVNASPDEGSLSEFEDHISNASESECSDDSYDSPQPIQNSVETFLSKNGNIEWQLHPPAQHGRLPASNIIKSTPGVTRYAVSRISDVKCSFEAVFHTALQNEIIEMTNIEGQRVYGEQWTDIDGSVFHAYLGLLLLAGVYRSHGESTKSLWDKDTGRNIFRATMSHETFCKISRVLRFDKKSTREERRRTDKLAAIRSIWEKWVEVLPKLYNPGENVTVDEQLVAFRGRCPFKQYIPSKPAKYGIKIWTMCDSKTSYVLKAQIYTGKVSGAAPERNQGMRVVSDLTSELRGQNITCDNFFTSYNLGQLLLKRKLTMLGTIRKNKPELPHKMTNKEVHSSSFYFTNDTTVVNYIPKRHKNVVLMSTLHHDAEISDRADKKPKMILDYNSTKGAVDTLDQLLGTYTCKRKSNRWPMIVFYNILDVSAYNAYVLWISVDPNWNASKLTRRRIFLEELGKSLIKEHIASRTHFPRTEDSLRMVTSIQNPNDVGGVSESVTTRKSTKRARCKFCPSSNDNKTNMVCGKCSKHICKKHVTYLCPQCKQ

SEQ ID NO: 130 - protein, 607 aa, synthetic construct
MKRTKHLGAPQPSTSRYAPLTEEQLRYYAENSDDDEEFLKAISSDSDDDDQVECDEKHDDEIATEDVTESSSGDDESTDDVLGNYMIAKSGFKWSTTPVKTSRRQLANILVNERGLTGYSSNVSTIESAFMLFFTREMIELIAMETNRKANFVYQEWNKNHPEKIRDWKPTDSIEIYAYIGLLFTQGALKAKKESVRSLWESNELYCRPIFPATMSRTRFALLTRFIRFDNFQTRVERRSTDKLAAIRDIVEMFVDKCKIAMNPSSHVCIDEQLVGFRGKAPFRVYMKSKPDKYGLKHWILADTEIAYVHNLQVYLGKTGQNPEKEQGKRVVLDLVDHLPAGHGITTDNFFTSMSLADCLLEKHLTLCGTIRKNKVDIPHQLLPSPQKEKFSSQFCFTKQHTLVSYVPEKRRAVILLSTEHHDNKIAMEEEHKKPDIILHYNATKGGVDTVDKLAKEYTTKRKCNRWPMAIFSNLLDLGAINAFAVYRTKFPDWQKQRSDNRKIFLLELGKSLITPYIKRRSENPAGLSRRILSCMKLLNPDLQERENRQAFPVEDSPERQQGRCFACARKRDRKVRTICGTCKRFVCAEHSTSVTEKLCNPCREKQ

SEQ ID NO: 131 - protein, 548 aa, synthetic construct
MPKVYSAQQALDMILDHVNPCDSDGDDVVLQMSSDSEVSSDEETSLPPVKRGRVETHTGPVEKAKDGTVWCEEQVGRPFNQSPVEPYATDGEPTALVRRTVSSRLQSFLCFITLDMLRTVQEWTVQHGRRTEESSWFMAVPELMAFIAILLLRGVIRLPSLRDTWSSKLGHPLITRIMARNRFQDIMQHLRFDDMDTRSERAETDRFAAVADIWRSFVSNCISSYNPGRHITIDEQLFPSKTRCCFLQYIATKPDKFGIKFWVACDLKSKYVCNVIPYLGKDPSRPSGERLSENVVMKLMEPFMDKGRTVTTDNFFTSLSLAQRLLNRKTTLLGTVNKIRRELPGSAKQAMNRQEFSTQVFSTSGVTLTVYAPKRRKTVCILSSMHSVVETEDTRKKKPNTVTDYNSMKCGVDVMDQMVREYSVRSGTRRWPVAVFYNMIDMAALNAYVLYQACTGVQERRVDFLVELASELAQSHLGAKKVNKENLLRQQPPTPDQGKRAMCQVNCRCKRNHATVRCVDCYRYTCGKCRKEVAWQCQVCSDNAGQNH

SEQ ID NO: 132 - protein, 632 aa, synthetic construct
MAKRRSNFNLEREDDVQGLNEILFCDSESDYENVNEFGDESDTEGEDNVEDRVENSDTEQSDEEYVSSAILENSRLLEQYSSSSFEEVLPQALTQQQQVNRNENIFITANFGKDNVLKWYLRPRKENQKTLPQNIIRRLPGVIGPAKQSKTPLECWSNFFTDDMLEKIVKFTNQYIESVQEKFSRIRDAKFTDIREIKACIGLLYLLGVNHSNRQSLEEVWATDGTGIEKCRLTMSLRRFKFLLRCLRFDDLTTRAVRRQTDNLAPIRELFEDFVSNCKKSYSLGENVTIDEKLEAFRGRCRFRQYIPSKPAKYGIKIFALCDSKTFYTSTLEIYCGKQPEGPWKTSNKSEDVVKRMSEHIYGSGRNITADNWFSAVDLVDFLKSKKLSYVGTLKKNRKGIPAELKNIKHRPLNSSFFVFSDNSSMVSFVPAPKKNVLLISSLHHDDEVSEENNKPEIILHYNKTKSGVDCVDKLCSTYNVARNTRRWPMVVFFAMCNVGGINALVVSFGNGMENTKRRCFLRKLSEELIVEHIQRRSEITVRGGMSCALQKSLKRFKPNPEEDRPANAGQNQLEKRVKCRPCSEEKRSRVTKYICYQCNRAICLEHAEKVCIECAPKLKINQLDEEDDSDN

SEQ ID NO: 133 - protein, 621 aa, synthetic construct
MDSKEQERLRRLLDSVEKEDRQEEVTALADRERLRRQLFVHDDDEFEEIEEGIIQRYDSDVSEDHESESEHDTASEATLEDLGDQELLREESWIVGKDNVTQWGAHMNEANLRGRVRQHNIIRGERIGVRLPCPRNEALAATTPIDAIKLFLPDDLVNSIVEFTNIWIDKNRPNSTRERDYRPTDGPEIRAVFGLLYLAGFLKSSHTNLIDLWATDGLGVEYFKATMSLKRFRFLLRAMRFDDIRTRRQRRAQDKLAPIRKVFEDFVSRCKKYYALSEYVTIDEMLEAFRGRCPFRQYIPKKPARYGIKLFALCDSRTYYTANLEVYTGTQPEGPFQKSNSAADIVKRLIEPISGSGRHLTTDNWYGSIPLAEELLNDHKMTFLSTLRKNKKEIPPSFINSRIRKECSSLFAYQPNITLLSYVPRKNKVVLLISTLHHTDTIDPDSGDKMKPEILTCYNKYKGGVDTVDQMQSAYSVSRKTNRWPLTLFFGMMNIAGINSYLIWRHNTQDQTKNRRDFLKSAGIALCQEHMMRRASMPMLPVELRHTLRRLLGQPVRPTLPEEDNEPRTGRCYLCGRKKNRKSQTRCSQCRYFICREHTRSSCVECTGQEVEHGSSSEDNV

SEQ ID NO: 134 - protein, 615 aa, synthetic construct
MEKANEDEIRCWLEENDVSEGNISFQGNLSDTDNESEHDDCDVDTEQSGDNSPRYYTAQHVDEAIDIVQTIPINTNTVEPGPSTQKQRPIERVSASYTSKDGVTNWNVHKPQSTKRARQNIIKKLPSVKRSFRDLKEPVDCWKIFFPNDMISDIVHYTNQELQRMRPNYTRARDCNDTDFVEVQAFFGLLYLIGVKKANHLNTHELWSNDGTAPEIFAATMSERRFHTLLQAIRFDDRHTRAQRKEIDNAAPIRDIFERFVSLCKLSYNVGELVTLDEMLEGFRGRCKFRQYIPNKPAKYGLKIYALVDAKSFFTCNMELYAGKQPPGPYATANDATSVVLRMVEPIDNSGINVTMDNYFTSVPLLNDLYANHRITVVGTIRKNKPELPQELLAIKDRKRCSSMFAFGNPPNKCTIVSYIPDKKTKKNVLVASTMHDDDEIDADTDDAAKPTIITFYNMTKGGVDVVDRLKSEYSVTRISNRWPLTVFGGLLNVGAINAQIICKTNTGELKLRRLFLTDLAKILIKPHMIRRGSIETLPTELRGTIRRLTGQPALLAPAMPQEQKGRCAFCPVRKNRFTTRACSSCSQKICGEHTKKTVYMCFRCTEEPVSDSDE

SEQ ID NO: 135 - protein, 587 aa, synthetic construct
MGTENTSNICKWLDEDLDVGIENESDDESNKEDLLNEVHDSNSEQDNEEEDDDYGDGDERLMSINDGNCYTGRDNTTIWQEEPVSLRGRRRAQNIVIHLPGPKRAAKNARTHTECFDCFIDQTIINVIVRCTNIYIEKVKRNYGRDRDCKLTNDVEIRALLGILYLAGALKSGRLNVRELWDKNGTGIESIYLTMTYNRFQFLLRCLRFDNIHDRQERKEIDKLAAVREVFELFVQNSQRCYIPSEYVTIDEQLVAFRGRCPMKVYIPTKPAKYGIKIFAMVDVKTYYTHNLEIYAGIQPDGPFHQSNSAHAVVKRLVHPIKGTGRNVTTDNWFTSIPLCLDLLENDKITLVGTLKKNKREIPPHFTTTQDREVNSTLFGFNENCTIISYVPKKNKTVLALSTMHYDKAMDEETGGSQKPEIISFYNRTKCAVDVLDQLCSAYDVSRNSRRWPLTIFFDLLNIAGVNALVVYSANKQKILRKNFLKTLALDLMKPLISERITQQTIPREIKRRGEQLLGIPQTGQEEGTRPEGIGRCYMCGRTRNKSTRQSCSQCLKRICPTHSDVICLNCVEEMHRPTQIIESDVD

SEQ ID NO: 136 - protein, 659 aa, synthetic construct
MEGPKYVSNRSKRIVALARSQNYLEESDSEVDSDESCIPDEDLTIQDPRNETDNDDDSPNDDDRDDETIREIFEVENTAVREETGEQPQSDITSGGSRLITPQKPADVASSSTRSDDTLDESRLPVSYYGRNKCFTWSSTPVRSKTRTAASNIIKVRLSKIQGPALTVGKNPLPGEVWRLLFTDDMIETIVKHTNEKLAKIRSVIELKESVAYRDTTVQEVNAVIGVTVLCSIFKSAREPMTSLFSTSVLGRPIFRSIITEKRLGILLRAFRFDDSSTREQREKYDPAAAITDIFNKFIWNCQQIYSPGAHLTVDETLVPFRGRVKFRIYMPNKPAKYGIKVMGLADAHNAYIYNAYIYSGKGCDGATLSTAEKKKSIPTQSVIRLSKGLYGTNRNISCDNWFCSVELAQELLAHNLTLVGTLKRNRPQIPAEFQARSDREIGSSLYGFTKEMTLLSYVPKRNKAVLLLSSMHHSNYTDVSNDKPEIISFYNATKSGIDTLDMKCSVYSTNRKTRRWPLAIFYHMVAMAGSNASILYGLFGEKKTVSRFDFLKRVSLYLVYDFVKSRLAISNLPQELQSIISSLEEEAAKEKQIVSQRNKFVVRVDECPTDTLGKRKTCRFCPYQKKRKTAYKCISCEEPTCLECSRKVCKTCAIKNKS

SEQ ID NO: 137 - protein, 632 aa, synthetic construct
MDMPGPSRRRREEAVLRTDDPNFAAQVRALLQELSDGEDVTDNESENDEVPEARSLCNAFDSELSDEDEEQDQKLKHYDHTPVMNVEDSEVPEAQVHTNPLHYTGKDGSKWSSTEPTRSGRVPAHNILKLPSLPGISNYAKQKLGQAPSEMEVWSQFFDNTIINEILKWTNVHLAQKRKKINNDEMLLYHKDLSKEELLAFFGLLMLCAIMKSGREDSKFLFRNDEFGRPIFHLVMSRNRFVTIKSALRFDNPDDRVERQKSDKGAPISYVFDQFISNCQLLFVPGASCTIDETLVPFRGNCGFRVYMPKKPAKYGLKILSLVDSGTSYFYNGYLYKGRGSDGLTLTEDEQRLSIPTQSLIRLTKPIYNTNRNITGDNWFSSLEAVHELRKRKLSYVGTVRKNKRFLPPEFVAHKGRPLYDDIYGFSNDSITLLSRKVKNNNVVVLVSSMHHKKQIDEETNKPEIIAYYNSTKGGVDTLDFECSVYSSNRKSERWPYVIFSALLNISLVNSFIIHRCFLQNEMKPRNIFIKKLATHLIKEQLRQRCENPHINKTLLASIKKFLEPSVVLMNMDICILRDDKMDKRKYCALCDYRKHRKTAYKCVKCQKPICLECSRKLCITCVTEMSDNMST

SEQ ID NO: 138 - protein, 605 aa, synthetic construct
MDQQPSSSCDQRILASDVDFDTRIQRLLNDDDNLSDVSSEADDSDQDPNYVPSDSDSSNCDYNDSSDDEDIMLEDAVGVVTVDNYLDSQPQVLHGKNGYEWSTVEPPRNRRTPAHNIIRGLSGLTAEAKAVGENPTKKDVWKLIFTDSMIDTIVKETNRKLTELRGRISDHTSKYNYNPCDQVEVRALLGLFLYTSIFKSGHEDMRSLFNKDPTGRPIFLATMSCKRFEILISCLRFDDSTTREQRKAEDKAAAISDLFNQLISNGRGLFCPKANVTVDEMLVPFRGKCRFKQYMPKKPAKYGIKIFCMADSGSSYLCNAFIYTGKNCYSVGLTRDEQQLLKSTQTVIKLTKHIERTNRNVTADNWFTSIELVDQLKLRGLTFVGTIKKNKKEIPPEFLPHRQRRVGSSLYAFHNNTTLLSVVPKKNKAVLLISSMHHDIETNSEINKPEIVGYYNKTKGGVDTLDMKTSIYGSNRKTRRWPMAVFYAIINIASVNAFILFLCYRGSPVLTRFQFIKQLAFELVEPHIRRRLEMPNLRADLKKTILSVLGQEQERGEVRDDRLQKRKTCSICPYEKKRKTQYKCIECENAICLECSRKVCLNCAR

SEQ ID NO: 139 - protein, 594 aa, synthetic construct
MSRERVIFRSDDPQFARLILEELNRPLGDELPISDLEDSDNDGGDADFVLSDHQSDSEQSEVSDSESTDEDGESVPRDVYRGKGKNRIPYVWGKQKPRHRGRAPAHNIVSHLPGLRNEARALSPGDINPLVMWELLMPNTMLEKIVNFTNTQLEAYRSNFQDPNRTDLRNVDLVEMRAYLGLLLLSSISKSGRESMSSLFSDDGHGRDIFRCTMSMKRFFILTVCIRFDDKNTREARKQEDPTAAVNEVWAEFLLNSQRMYCISQNACVDEMLVPFRGRCKFRVYMPNKPAKYGLKVLCLTDARNSYLFNAYLYGGKNSDSIGLTHEEKKLSIPTQSVVRLVKPIEKSNRNVTCDNWFMSMELLNELKNRGLTVLGTLRKNKPEIPKEFLPNRGRAPGSSLFGFTNAATLVSYVPKKNKAVILLSSCHHDDKCDEVSGKPEMIIDYNSFKGGVDNLDKKCAIYSCNRRTRRWTLALFFALLDISANNAYVLYSDSRERSQMNRFCFIKKLGCFLIKEHLERRAVNLRLPRELRQKIQAISGIKPEDPVQHGGPPKKKRCYTCPRNKDRKTANFCVKCNQNICGECSHAICKNCV

SEQ ID NO: 140 - protein, 659 aa, synthetic construct
MALRSSEIDKLIDEPLAIESDSFSENESGSELENSILSYSGSDDYQPSSDEYPSDDEYVEARNKKRKISCVGLKKSVGDLLQSLNTHSNIPSTSTQTSSPTVKQSSSSLLSPPVLQPGLYATIQGPLPATTSNSGTQCSDNTLPRTIRVIKGKNGHCWTQEPRTSSSRTRAANIVYTAPKVKGRARDTTNSSGSFLSFITNEDINLIVNHTNSEILLKQRNYKEITASFEVTSSLEIKALIAVLLFSAVHHDNHLNTVEMFDTTTSGNIYKATFSEKRFNFLLDSLRFDDKQTRPQRRETDKFAPIRELWENFIQRCTENYEPSPYVTIDEQLLAFRGRCPFKMYMPNKPSKYGIKLVLLCDVGTKYMLKGEPYLGKGSTTNNEPASHYYVKTLTAPYHGSNRNVTMDNWFSSVPLAHEMIKPPYNLTMIGTIRKNKRDLPDCMITTKNREIGNSRYLFDGTVTLLSSKVKKNKVVCLISTMHDNKSTSSSGKPTIIDHYNQTKGGVDAFDQMCSGVSCSRKTKRWPLCIFYGILNMAGINSWITRNCLLTLKKEPPISRKTFLREVYRELVTPWLDRRNHLPTIQRQLKSIISEVLNKEMGRNTPEDQVQHPPNNTRTNCKICPSAKRRMTTHHCSKCAQAYCNEHRGLVCKQCTEHN

SEQ ID NO: 141 - protein, 657 aa, synthetic construct
MEEERENQRIRQLLDEELSDSDESEREVRPVDIFEESDEGEDDEVERDAVEDTDDTDADPNYELDDFDDESDDCVTPPKRKRQRTDYLRLAAAPPTAAPPADDPSATTPLLNPPADDPPSADPPVATPPTDGPPVAVLPATDPHIILLRERVVRARNNQIWSTRPRPQNTRTPARNIVVGRRKVLGQARNASTARQCFTQIIDEHILDKVVLHTNKQINRIRQNYVNQSNTVSPINNEEMLAFLGILVNTALLKDNHLASRDIFDHRKSGTQYKCVMSSERFDFILNCLRFDDRATRLQRKSTDPFAPIRELWEMFTNNCHSSYSPGSFITVDEQLLGFRGKCPFRMYIPNKPSKYGIKIVLACDTTTNYMINGIPYIGKKTPTGGLPASEYFVKEVTKPIHGTFKNVTTDNWFTGVQLADDLLKPPYNLTTIGTIRLTRKGIPSHVLSNKKERPVNSSIFLFDKEKTLVSFKPKKNKTVTLLSTMHDTAQVRTDTHKPEIIHHYNETKGAVDTMDQMCKNMSCSRKTQRWPLCVFFGMLNIAFVNSYVIYCLNCFNQGRNPKNRKNFMSELMMDLTSPHMQTRLRNTSLQRNLRMMISENLGIPLNANGRPPAGTRKICFFCPSAKRRMTTNYCNHCHSAICAEHRAMSCVHCDEN

SEQ ID NO: 142 - protein, 651 aa, synthetic construct
MAAKKIYTDPDEILALLSDVEIESEEEEDCTEVEENDSDSGDDSDYIPVDHREPSNDELVIEDDIRRIEKELRPTAGTSKKLPAKRPAPAKRPAPAKRPAPAKRPDPVKKKTTLVRKSKESSLPAEDGNYILATDSTEFEAKNGDKWSTIPNSNPSDPTKTSKKNVIHIRPGPVGDAKQANEPIECFRLYFTENIMAKILKHTNEKIEETAKRYKRQEGSLQPVTMEELEALFGILYLAAALKSNHLTAEMLFQTEFCGYKFVATMSRRRFEFLISCLRFDDGSTREERKSETKFAAISEIWEMLIENCKTKYKPGSYVTIDEQLLAFRGRCPFKMYIPSKPCKYGIKTVMMCDNANKYLIDALPYWGKGSIPKDVPAGEYYVKTLVASIKGSNRNVTMDNWFTSVPLFSDLLKDYNLTAIGTVKKNKSELPSSFVDVKFNNRRIETSFFLFNHDMTAVSYKPKKDKVVCLLSTMHDDNDLSKEVGHKPEIVLAYNETKGAVDTFDELCRGINTGRKTRRWPMCVMYNMLNIAAINSYVIYLHNFFRNPVNDDAKPMPRLQFMIKLHEQMARPWQKFRLDNNPRFSKELQHQIRSVLGEKDVAECVAGEQTGPRKYCSRCPSQIRRRTSVYCFSCSKPICGEHQKKFCLDC

SEQ ID NO: 143 - protein, 690 aa, synthetic construct
MDTDQEQRILQCLNESLSDDFISEEEETTDQRYYSGIDDSDDTDADPDYSPSGTSSASDNEDLLEIPTAKIATQGIIMDTIDETINAVIAAYCHGHPPPTDIPQVGNAPLARPASPPMSRPQEALARPASPPVPRPRAAVARPASPLVPVPQAAVARPASPPTVAHGPPPVPRDPHHFICPNQRMRGRNGHLWSNIPSRPTNTRASARNIVHIRPGPTVNARDKTNPLDVFSLFCTDNMKDSIISHTNSAIRRKQVNYTNFTSTISEITREEFDAFLGVLILTASQKDNHMPSNELFNVEFCGNRYLATMSCARFDFLVEALRFDGYETRNERRRQDRFAPIREVWEMFVASCRDNYKPGSYLTIDEQLLAFRGRCKFRMYIPNKPSKYGIKIVMVCDTGSKYMINAMPYLGKGTTPRNIPQAEFFVKELTQCVYGSNRNLTMDNWFTSISLATQLLNPPINMTLVGTLRRNKPEIPPEMKEAGDRPVGSSMFCFDGHKTMVSYKAKSKRVVLLLSTTHEQPSINPRSKKPEIIECYNATKGAVDSLDQMCNNMSCSRKTRRWPLCVFYGMINIMLVNCYTIYAHNMVAQGSKPVSRRDFAKELHSELVKPWLQHRNRITTLPKRLRESIASILEVDIPAAQIDPQLVQGRKICAFCPSKKRRMTSHFCRRCSKAMCGEHQGKWCVDCTC

SEQ ID NO: 144 - protein, 603 aa, synthetic construct
MNFTCVLLTIMTRRMKESDIEKILINFEDSDLSDIDDDDPEDIDVLQDLIETLDQNDSFVRKELTRENIYEVNLANTEIEIEEPVEDEFVVKKTEDFRWRKCPWATPETDFNSEHPLQCQKTPVKYFLEYYTEDFIDEVVLATNQSVMIKNGTSFNLSREEFLKFLSLEIMTGSIGYPRLEMYWKSPTTIPAFTENMARARFHSIRSNLKFSVNDVANDKLYKVRPIINMFSRKSYMIPKDENLCIDEMMIPFRGKTELRQYMPKKPIKWGIKVFCLCSSSGILHDFEIYQGKNTILSGTKLGFCSDIVIHLAKTIPEKQNFKLFFDNYFSTLNLILLLRQKGIWSTGTIRANRINRVPLKSEKDLKKEGRGSYDFRIERENNLFIIRWFDGGIVQVVSSIYGDEPLNEVSRFCAEKKRKITIPQPHVIAMYNKSMGGVDKLDFLLSLYRISIRSKKWTLKVFDHFIDMAICNSWLEYRKDYEYTNGSNKGYMDLLSFRNDIADGLLAQSKQNTCSRKRGRPKNSTGGEELDSPSTSKKAYERKPSSSVRFDGNQHWPAYIENKSRCKNNGCQGYSRYKCSKCNVHLCMPNKDRNCFYDYHNL

SEQ ID NO: 145 - protein, 592 aa, synthetic construct
MFIHLQLCTYVFSVLFRGKCLTTAEIIEECERLSEDDIPDSIYILPPIQTCDETDEDSGDEDDFDPNRLNRNQLTAPAEVRFSDREDRSDDIPHDESASAFKKLKVCEWVHGDISDVESIPVPEEKCFLPPLTISEHSEAIDFLKLFLNEEIVEGLVKHTSMYSSQKNSSLLVNHDDMYAFIGILILSGYNSCPRRRMYWENRADTENALVKNTMRRNQFDELFRHFHACDNLNLDANDKFAKVRPLVSKVNELFIRFAPLESFASIDESMIRYFGKHSCKQFLKGKPVRFGYKAWSLALKSGYCLQFMLYQGKDTSNGENKKEFGLGGSVIFSFVDLLESSYPGFKFSLFFDNFFTSSKVISQLSERGLGATGTVRENRTEKCTITSKKDMKKMERGTIDKRVEKGRNIIAVEWKDNNAVLLMSNQFGVEPLSKATRYSLKEKRKIEIPEPNIIKMYNRYMGGVDQMDNHISNYQIGMRGKKWYMPILFWTIDVCVNNAWLLSKSYGCRWDKLEFLRQAVDSLCKTHGTKRKVSGPFKKNRVSQCQKELGRHLVVTAQPRRNCVHCKNKTVTACETCNVPLHDKCFIPFHT

SEQ ID NO: 146 - protein, 573 aa, synthetic construct
MAGNFERNNELIRLMLEQEIDNPLSDEEEEVGSDPEIFSEHNTDSEGGGEDEEEPPIALPQIRPPTPTEFYLGKDKKTRWEVNEVPPGQAPCRNVIRARIPALSQTANQASTEYEFWSLFMDENIMRRIVTFTNQYIMNISQNYADQSKVMVTNEIEMNSFIGLLVLAGVLHAGRLNVEELWEKDGTGVEIFPATMSLQRFRFLCRSLRFDNAETREERKSVDRLAPIRELFDSFVNNCKANFSVGMFVTVDETLVSFRGRCPFRMYMPNKPAKYGLKIFSMVDSNLFYTSNMEVYVGVQPNGPYEVSNSSKDVVLRMCEPVYNSGRNVTLDNWFVTYALVEALTQKRLTVVGTVRRDKPFIPVDFVSTSHRAVNSSLFAFQKDKTLVSYIPKRNKCVVLISTMHLDSKIDESTGDLRKPEIITFYNHTKGGVDSIDQMCTEYNVMRNSRRWPLTIFFHLLNVAGINSHVLHTIKNQTKLPRRKFLKNLALELVKPMIEQRATNLRLPTQTRQLCKRLAGIQDPPRIAPEAGRRGRCHACKDRKTQYYCQKCHTWICLAHATMLCRDCAEAQN

SEQ ID NO: 147 - DNA, 244 bp, synthetic construct
  source 1..244: mol_type = other DNA
ttaaccctttgagtcccgcttgtatctccggagatcatttgatattaaaaaataactgtttgcctcatccttgaacgcgatacctagatcttttctactaaacgagagctatcttttgacaaaataaaaaagtaaaaaagttgtttagaaaaattttggtgagttggagaaaagtgaaatttgacaccattttctttctttgttgaatataaaataagtttatctaaaaaagtgatcttgaatt

SEQ ID NO: 148 - DNA, 1242 bp, synthetic construct
  source 1..1242: mol_type = other DNA
ttaaccctttgaggaccggctttttttaaaatttatttaaatttttaaattttattttttttataaatatttatttatttcattaaaaaccggaaaaaaaaattaattttataatttttaatataaaatttatttacatgaatataaatattttataatatattttttataaaatattgttcaatattcacataggtttatttcattcattcattcattcattcgaatcattcaaacaaatgtctgatccgaggcgagtaggaagaccacgcaaaagaattccaccttcaagggctggttttactttggtgtgtcctgataaaggttgctatcaccctcctacaagagaagtgtatcctccttatttggaaaatgcaccatcttcatgcaaatcaaaggaagagaaagcgatgtgggtaagtatggcccgacatttgcctcctttcagagattgcaaagatgaaaaaagtagaaaatatatatgcgcaaaggataccccatgcttgccatatatatttcaggaggtaaaaaatcattacgattttagttacctaaatcctgaagaatggacaaattcaataaaacctgattttatgtctgatgatgattggaatcattttattgaatcatcatcgagaaaaggaagagattctcaaaccgggccagctggcgaaggtttgaaaagaggtcttgaccaacaagaaacaaaaaagaaacgaaataggcaaatataaatccttttagatatgaaaggtaggtcaaaccagcctcgaaatggtaatcctttcagatatgagaggtaggtctaaccagcctagaattggtaccattcttctggaaaggccttgtcgtggcagaatggcttttcactaacttaccaaatcattttttgttaaaaatttaattcaacgttttttgaattatctcacaactgagaattttatgacaaaaactattatgattttcagaaccagcataaaaaactctataaaataggtattttgttttttctaattttccaaatttttcacatatcttttttatattcaacaaaagtcgcatacggatcacgtccgattttttttcgattatcttctaaactacgctatttagaggtgagttgatattttttatgaatataatatacgaaaataaagattttgacatcaaaaaaattgaattacgtaaaggattagaaaagttagcaaatttattttattttttatacatttttatagagaa

SEQ ID NO: 149 - DNA, 382 bp, synthetic construct
  source 1..382: mol_type = other DNA
ttaaccccttaacgcgcatactcgagttaattcgagcgcgctaaaccggccaaattgtgcacactcgaattaactcgagcggcgtcacattttatgccgctaaaatatttacactcgtaaataatcgagcattgaaaataactcaataaaaactagttaaaatattcgtttttctgatatccatcattttaactctatattgcaagctataaagttaataaaatatggccttttaggacgatgtttataaacagttgcattttgtttaccaaaaaaattgtgtaaattggatatttagtgtataaaaggtatgttttctaatgatcaagtaaggaatattattctatttgacattttattctagtaaaatatatattgaaaa

SEQ ID NO: 150 - DNA, 551 bp, synthetic construct
  source 1..551: mol_type = other DNA
ttaacccattaatgccgcttggcaagtatacttgccacacaaaaaaatgtgtaattttcaatactatgaatgtgagggtttggattgaagtctaacggtaacgagacgagtactgtacgtgttcagaaccagttacagcggtcggctgtgcttcacgcctgctcagttgaccttggactttgctgctgtgaacaatggcgttactacgtcagaaaaaaacgtaagttactgcttatatctgttagctatgatttactactatactgaaaactgttgttaaaaagttgttctaatgatatatgtatgcacttacatgattctaatctcttacgaccactatattttactatctatatttgtggcaagtgtacttgccactcggtaaaaaaattatggaatcgctatttttccattatatcacttgacgattattggtaatatattttaacccttatttcaggttgtcacttgacgaaattcatgatttcctgttagaagaagatgaagaagagcataatgaagtccgagtagcaatagagccccctgaagag

SEQ ID NO: 151 - DNA, 787 bp, synthetic construct
  source 1..787: mol_type = other DNA
ttaacactattcctaccgggaccggtcaaatgaccgtttttaaattttcaattggaatttcctacattcaacgtaagcgaatcgcctttaaactttatgacttttcctcatatatatgtatgatacatttttcaatattcacttatttttttattgtttattttctgagaaaaatttgtagtctgtcttacctaccacgaccggtcatttgaccggtggtacgatttatacatttttgtaatagtttttctctaagactcaatgccaaaactataaagtcgttacaaacgaaaggtaatgtagttgtggcatgattttatcctataagtgccttccagaaaatggtttacggcacttccaagtgtttgcagttctcgctcgactatcgggttagcggtgtttatttgcttgattgatctgatacggtccacttcctcgaattttctcacgcaatcttatctactatgacggaaaagaaatgaactcataactgaaggaaatgtatagacgtcttctcacgaattcaaatcagtagagtcttgcaacacgaacagagtgcatcgaaacgtgaaagtcacaaagagcatacttggcagtcttttattattattttataatattttatatcgtattacgtgttgtaagtgtcattttcaaaatagtgaacaagactaataatacgtcgcagtaagtttcacattttatcttttattgatttttttatctatattgatttacattttcatattttagtttttattttttccgttgaatatttgtccccaaa

SEQ ID NO: 152 - DNA, 449 bp, synthetic construct
  source 1..449: mol_type = other DNA
ttaacactagacataccataacgggtcaaatgacccattttgaacttttgcattgaaaatgcacgtatcattttattgcttttgcacatttgacttcatgactttttcgaactaattaatctacagttaatatagtattttttttatttttttttgttttgtttatgttgagtaatacttgtcgtatactgttatctaccacagcgggtcatttgaccctgagagcattcgtttgttttaagcagttttagttgcacatttctgcaaaaactgttgcgtaaagtagttcaatcaaaatagctgagaataatttcaagaaaatatcaaaattttatattggatttagctttttagtgtttgaaaaggtagcaaaaattgaaagttttggtaagtagcttatattttattttgatggctttagaaatacaggtcgctttttacccagaaaa

SEQ ID NO: 153 - DNA, 480 bp, synthetic construct
  source 1..480: mol_type = other DNA
ttaacactagatttaccagaccagtcatttagactggtcgtgcaatttcaatccaaaattcctactatattacacttttttttccagaaatgatgttatgagaaatgatgttatgacttttgtagctataattagagagtacgttttataaacttaatgttgttttatatacattagtaacaaatataatttttgttgttgacatttataccagtaccagtcaaaatgacaggttgtgtttgtatgaaaaaatgtatgattttgggaatcatgagaccgatgcagtacatatagcactgaagcgtactttgttatcgtcttttgttttctttcactatcgtcattgagttgatgacgtcattttgatgagcatttgttttgtaaatgcactagacatgttcagtcttgtttctagctttgtgcaacgcgcagcatttttcttataaacaagatttttatagtgattgtgatcccctaaaa

SEQ ID NO: 154 - DNA, 598 bp, synthetic construct
  source 1..598: mol_type = other DNA
ttaacactagatttaccgagaccgtcaaaacgacggttttcaattttcttgctaaaaaattacataattacaattaatattttttcgaaataattgcatgacttttctacatatgtagcggtaattaattttataaattgtttctatttttaaatgtatccaaaatgcaaatttttttgtgctatacctatttctaccaacacccgtcaaaatgacggctactaatatttaagacaaatttgatatttgttgtacggtttattgctatattcgccagtatgattttagaagttgataaataataccttgttatctcaaaaatgaatcatttattctatatactacatattttctgttgaaaaagaaaacaaattggttgtcacatgatgagtgatctcacaggctccatatgatttcgttgaaaactgaggttctcaccgcatatgctcagtcagtaattttagccattggcagatatagtagtatttatccgcgccagttcatctccaccctcacgatttcaacagaggtaagataatttagaaaaaataaaaaatgtttttggttttatatttttgttatttatcaggccgtaaaa

SEQ ID NO: 155 - DNA, 479 bp, synthetic construct
  source 1..479: mol_type = other DNA
ttaaccctttctaaggtggtggcaatatactgtacaccattttaaactaataaaaatatctaagtgtaacgctgtcggtaatcatttttacttttggtacaactactgacatatattgagccacaagagaacaaatttatagtatttgaaaccatagatgtgttttcccgctatatttattgacagctgacagacaagtgtgtgtgacctactacataaatcttcatttatataattgatcttcgattcaaaaggtatattatttattattttgatgcgatacataatgcagtattcctatgaaattattattttccttctactatatctatattttgaaaattttgtagcaaaactttgggtggcaagatagtgtacattgcccgtttgatggtagcattggtggcaacatacttgccactgcctttgaatgccactatatattcaaccttttatttcagctattcaattttttagaa

SEQ ID NO: 156 - DNA, 587 bp, synthetic construct
  source 1..587: mol_type = other DNA
ttaacactagatttacggaccgagccaatttgactcattaaaaatttgatcactatatatatatatatataaaactttaatattactttttaaaaacatacgttggtttttagttaagtaatgaaatatatgtaaacgttcattcctcctccccacagaccccttcatttcgcattatcgaatgtggtatacctattttcacggttgtgagccaatttgactcatagaatggtttaacagttaatcgcataacatgaaatttattggttctatcaggcctgggcaacagtcaagtcgcacgtgatttgtttcgcttctcatttggcattcctcacatacaatatttgtgtgtatgtgggagccattgtcgcttgttttcgctactccgctacatcattacccacttctgtcatcatttcgatttatacatcgcgtgtgagcagtgcagtgaagtagtatataatttataattttataaaattcattttttgccagaaaaatttgatccacaaaggtaattttgtaatttatttgtaaaaggtattacaattgatatttattttatctggttatctagggtttctatt

SEQ ID NO: 157 - DNA, 426 bp, synthetic construct
  source 1..426: mol_type = other DNA
ttaacccttaaatgcatcgtgttgccatttggcaacataccaatattgaagctcatacattgcacagaaataaaatattgatgcaatacatgttacagtggacaggctacatagttctgcgcatgcgcatgatgtgaccagcgtgaatatagcagctataggttatttcttcttattcgtttacaggcgtttaaacttattcgcgcgctcgtcatacgcgaaagttattcagtttttttaaatagtttacaaaacatttttatttggttttaaatataattgacattaacaaaggtatgtattctttattgagcatttgtaattataaataaactttgtattactgcatataaaatctatatttcgctttgttgccgtttggctacagcatgcatttacttataaatttttgtccctaaaatttag

SEQ ID NO: 158 - DNA, 461 bp, synthetic construct
  source 1..461: mol_type = other DNA
ttaaccttcagtctgtgcattggggtcaaatggaccccagtctttgtaataagtgatataataagtttttacaccaactaactatgtcaagcaactagatttgcctcaactaaggttttccacacacattacaaaagaaataatcaaaaatacattatatatgacgtagctacgatagttttaagcaatttaattatattttggtgcattggggttaatctgaccccaatttttttcccgcctaacaatctatagaagttgtctctttttcaaatgtaaaaagtgaaattcagtgaatattatttttgaatagaagtgttgttattataaattaatttgtttgtgataattatatattcatataaaaaactatttggtgactatttttataaggtgagaatatttatatatttttgtggttgttgtgtttagtctaattttctaggcatataataatttgg

SEQ ID NO: 159 - DNA, 713 bp, synthetic construct
  source 1..713: mol_type = other DNA
ttaaccttcaattgaccaaccaaaagttgcacgctatctgaccaaccggggtacccgggtacccactatagaatttcttacattgcaggtagaaaaagtctgtaaatgggttcttggccttcctttctggcttcccgcgttttcgaacaatagaatgcgcagccgttagggctcccattccctgtcttgcctgtcgaaacttgttttaacgtgtgtatagtcgcgcctggtcgtgtcgctcagtcttaaaacgttcgttcgagaaggcgttaggaagtttgatgcgctcgaataattacagcagaatatattatattttagtatttattttggtgctattaaatttggtgttatatcatatttgttataacaaatataaataaagtgacaacaaaacgttacaacaggtaagcgtaatatgtatgaaaaattattaattagtattacggaataaaaagatatattgtttttatagattgagcattaaaaagaaaattatacaataaaaagatatggaaactattgaaaatatgtctttttcggacgacagtaattattccgatagcgaagatgacgaatggataccacaatcggcgaatcaattagaagaaatttcagatgttgattttaatggtatgcttaaaaatttgtctgttaaatttccaataaattatatataatataaattatacgatatttatgtaatattatga

SEQ ID NO: 160 - DNA, 1246 bp, synthetic construct
  source 1..1246: mol_type = other DNA
ttaaccctttccttcccatggtagcacaggtgatccaccactttgcataggtcgtatacaaactggacaagttagatcatacccattttttcaccacatgttcacatatgtttcatgaactactgtacaaagtttcactagaaatagacacttggatttcaatttaaagcataacgactgaagtaaagtcggcattttttcaaatttaaataaagtggattttcaaacatccatattcagtaatatgaacataattttcaccaaacaaaaataagaacaagctcgatttagttatttttccaagattcaataaagtttttataatattctttacacaaaactattgattttgaggaaaacgatgtggatcactgatgatccattgggaacaaaacgagattttcatcgattctattctaacctatctttctgaaaacgaaactgtcatcacatttgtttacgtctcagaaagttgttctaagttcctcgtttcgaacgtgttttaagtgaaaagtgatttttaaagaaacaataattgtttatggacgctaaaatggaaacgaatgaaccagaaaaggctacactcacggatgaagaggatttcttcggatttgaagcagatgatgaaatatttccgggtatcacagacgaggaggatgcttctggactgaaattgaaacgccgccgcaagagtaaattatcttccgacaccaatcttcctacaaaatggaccgaaaggatgccctgtggaacaatccaccgtagatcccgagaaggtgaagaaagaaagaatgtcccgttcgaagatacttcgtacgaacacgaacaagtacccagtgatgatccgattccgctggttgctgggagcgatggtagtatttgagtttttaaaatttttatcaatcgctaataatacaggtatttgtagattgcaacaataagccaataacattcgagggagatacttccgtgcttgaagaacttagcgaaccggagaaaattattgaaaccggaacgagccgacttcgcacatctcttgaaactgccgatggacacaaaggtaccactgagcaatcaacgattgaaaccgataacttcgatgacgatagtagttctgaagatttgttcgaagatcgtgatgaaacatatgatttttctaaattaaaaccgaaaactacaacagagctggatgagtgggatggtagtatatcaaaaattgatttattacgaatctataatataaata

SEQ ID NO: 161 - DNA, 728 bp, synthetic construct
  source 1..728: mol_type = other DNA
ttaacactagatttaccagaccagtcattttgactggtcgtgcaacttcaattcaaaattactactacattaaatatttgctttccaaaatgatgttatgacttttgtaggtataagtagagaatatattttatgaacttaattttactttatataatgttagtaacaaatatattttttgttataatgatttatacaagaaccagtcaaaatgactggtgcatgtttctaggaagaaatgtatgatttcgggagaatcatgagaccgtgaaaatacaaatagtgcccatgcttcttttgttcattacgttatcgttatcgagccgatttgggcgattaccgtatctatacgatttttttacgagtacttagtccaagcgatctccaaacgtaatcattcatctttacccatacagtgacacgtaataaacatttatgagtatttgaaaaatattcaaatatatgttcttcagcaagtagcctgcgtatcgttaccccctaaaaatgtcaaaacgaataaaagtttctacatatttagaaatgcaacaaacatacctcaggattgttcagatgtggaaagtgaattatttgatagcgacggtgaattattagatacacaaattgctccacaaggcaatgatgaaaatagtgctaatatcttgagtagtgatgaggaagttcttgatgaagacaatataaatcagtcaagtgacagtgattgcgaaaat

SEQ ID NO: 162 - DNA, 374 bp, synthetic construct
  source 1..374: mol_type = other DNA
ttaaccctttcaagactaacggtacatatatgtaccaccgacatgatgctccggtctgagaggccattattgtaccataaatgcctttattttgacgttttccgacttttcaggaactacgcatgcttttttcgctttgtgtcatgggatttagctatcacgtgacctattttcgcgcctttagcttcatttaccatgtggtgttgagacagatcacagtcttgcaggtgttttacaatgctgtattgcagttacattggctagcattagtttggaaagacttgtaagtgttttagaagttagattaccgttatattggctagcattagatcgaattggaaaggctgcatttactatcagtataactggtacac

SEQ ID NO: 163 - DNA, 1111 bp, synthetic construct
  source 1..1111: mol_type = other DNA
ttaacccttgctctgctgcgcgcacatatatgtgcgcattacagagtgtgcgctaccatgcggggcgcacatatatgtgcgtttcaatgaaaaacagacgatcacattttgtctggatgatttttttttatattgctcttattttccggtaaaagtctctgaaatatataatactggtccataattttaagatttattaaaataaaatgattacatacaggtttggttacattttttttgcatttttctccagtaagtaatgtgttgtatcggcgccatttttaaaagtgaaagcgatagtagacgccgacattcggtaccctgatatgaatcaaccgataaattttatcgcatgtttatatccctcttgtatttattaatgttcatatcaagtacacttatatttctttttcttcattatataatttttacgcatttattgatagtatttttctaatgttatatggaacattttgtatacagttaaaaatagatgtaggtggattccacctcgtattattcttataattttcaatactaaaagaagaaagaatcgataaacactagcttaattgcctttcacgtttaaaatttgtattaccataacaaaacaatggtgtaacacctatgctttattatttggaaatttggagggaaatttagttaatgatataggcgtttaaacaacgcgacagaaatggctgctgcacacgtgttttcgttgttggtgtctggtgatctaattacgtaaataggagataatactatagcaaggatagtaaatctaacatttgaaaggtatatataaacacaccattctttgtataaactagtttaaaggaaataacgccgcaatcttaggaagcacttgatctaacggcaccggaagttgagatatgacgcggcgatcagctgttttgtatcgtttggactaccgatgatatattggcaaaaatatccatttttataaattaaacaatgaaatctattgcttgttttcgttttttcagtgttaatacaaactgaattgagtggaatatttgtttgtttcatgagagatatacaattaaaccctgttgaaaatatgtaaacaatatgccctatagacgtt

SEQ ID NO: 164 - DNA, 1110 bp, synthetic construct
  source 1..1110: mol_type = other DNA
ttaaccctcacagtgcgggacgcacatatatgtgcgtttgtagaactattagctgtcctgcgggacgcacatatatgtgcgtttgcaagctcatctgcatctcgcatgttttgaatacgaaactttttaattgatggctgattttccacgtaaaagtcaatgaattatgttcagagaataaaaaccctttttcttaaaaagtactgaggcttaagatgggatatttaaagaaaaaatataattagtaaaaacttagtgaatctcattaccgtacgtcgtcgatcatttgggcgcgaatggataaatcccttcaaatttgcaattgtgtgaatttttgtggaactttcatatgcatttaaattttttagaatataaaatgaaacatactgaggtacaagagaagtatgacaaatcttgacagttttttaataaattctattgcccaacgagtccaacatgataaaggggaattccagtttataaatagctttcttaatcttaccatgttctcgaaaaaaaaaataaatttagatttctccatgaaaccttacttttcagatagtgatagttaatcagcataattaaataggcctaatttgctttaatttgcaccaaaggagaatcttgaatatttggagggaaaatgtgaccgtgatctaagcggttacaaaatggctgcctagcttgaacacgcgttgattgaatactttgtttgtgacagtattatagttcttaaatatggcatgactttgatgtaagtattttagaattaaattttttttcttagtaagatgttactttaacatttttaatgactaggggtaaacttcaatcgtgagataggagcaatcaccggctccggaagatttgtttcgaatattatgtagaacaggaagttttgaacagctgttagttgtcgtttggggttgatatctttaacatcttgatttcttctttattatgaataaaagtgtctcttttgttatacttttctctttgtcgtaatatcggagaacaaactttgcagcatacctacttgttttttctcaggcaattttatatattccactatatattataacacgtactgtatataaacatgttgataca

SEQ ID NO: 165 - DNA, 609 bp, synthetic construct
  source 1..609: mol_type = other DNA
ttaaccctttatacggccgtgttgcatatctgcaacataccgtttgaattcaaatatttcataatgtatatcaagaaagtttcatgtgttaatgtgatatcattattcaacaatgcttttttttaattgtaagacatggtcatactcaactatgtgttttcacaacatatttttacttttcccgccaatttttgtgaagcactcacgtgacctccgttccaaactttgtattacaaatatttcctaaaatacaagttatttgtctaaacaaaactgcacagttgaaaaggtatttatgtttataatgcatatgcagtcaagttattatgttatgtgttatgtgttacgtgaaaatagtgagtttcgttatgttgcagatatacatcaaactgcttttctgacttgatttgtgcagtctcttatttgatgcatatatgcaacatacatgtgtttgtgttcattattatgttgtgtgttgtgtttttatatatatttatttggtatttgttgtcagtgtcgtctgtgttatattttcatctatgggaccacagttgtttttttcaaatggtatttagtaaatactattgacttttgtattacataggaata

SEQ ID NO: 166 - DNA, 419 bp, synthetic construct
  source 1..419: mol_type = other DNA
ttaacccattcgaccccgaagttgcctgtgagcaatttcaaaagtttaacggctatcggtagtcggtgcttttgtttttgtctgcttattcttcgagataacggtgctttgatgtgtatacttagaatgtaagcaaaaaaattctagtttgcgcatcatttgacgaaattatttggaatgtgctggacgtgcctcgcgtaaaaagtgattgttgtattgtgaggtatattgaaaagaaagtgatttactttttaattacgcattaaaagttgaagaatgtgagtgaaatttgttaataaatgaattctttaatgcatgttgtagttcaatctaattgatttctattaaaaaacagctgaggaattttcaaagtaataaagttgcttgtgagcaaccttgggccattgcaccaccaaaat

SEQ ID NO: 167 - DNA, 628 bp, synthetic construct
  source 1..628: mol_type = other DNA
ttaacccagataagcctactgtcctatatttaggacagtagaaataaaaaaaaatatcagaataccctctttatctaagattatttagtcttatgatttgcagtaagaaacgtgtcagctttattagaaaaaataaaaagacagttattttaacagtttttaccttatattttaccttataagtgtgtaataaaagtgcgttgcagacatggcaaataacaggtatgtataaaaagttatattttactcgtgagttgtttttttctgtgttaatatctagttgataaccttaacttttgtactaaataaatattctagcaaaataatataaataaattacgtataacttgttacaaatactagcgtactatatataggacagtaggataatatacatgattttagtacaataatacaactttttctttacagaccgttagctgcgcatgaaattttagatgctttagaaaatgtttctgataatgaagaagattatagagaacgactgatatgtattctacctcctcctgttgatcctgactgtctcactgacgaagattcgggtgaagaagataatgtaactttgaataatttgccacgaaacattctgcttcaaccggctgaagta

SEQ ID NO: 168 - DNA, 474 bp, synthetic construct
  source 1..474: mol_type = other DNA
ttaacacgttgaccgccttgcctttttttgaaaaaatcacattgccaaataatctttttttttttgaatatttcgtgtatacatatgtatacacaaataagtcaaagaggagttttttttttttaataatttttagtttttgtttgaacattcgaacaataattttctcaaaacctaaactatttaacttttaaatatttaaaaatatatttttccaaaatgttccagaaccttccagcacgccgataaacgggtatataaacctagcgttttctgctaaacaccagttcagcaattgtatggactttaaatattgttgaataaataattgtgatcgactgaaagaaaacttgactgtcgaaacttaacagttttcaacttttttcctcaaatacttgattgatattactaattttttgatacaatcgtaaatttttattattttctagctattaatttttcttatttattaga

SEQ ID NO: 169 - DNA, 467 bp, synthetic construct
  source 1..467: mol_type = other DNA
ttaaccctctagttcccaaaaccgcctcgaggcgggtattataaaaatccatgtaaaaattacatcgctagaaattttagcgtaatgctcgtgtccgcctatagacaatagcatacgaaccaagttacgtgcgccggcgcacccgcaaagcacgtggttttgtttgtataatccgtgcagtgttatttcgcagcgattactacgattagttttgtgtcagtgattggaaaattgttaaaaaatatttgtagtgcccccaattgaataaaaatattattggtaagtttgtactttatttagcattgcaacaaaatatatttgaatgaataattgctcatacaaatattatacttgccctattcatgatacccgtctccaggcggacttgggcagcaagttatatttttttgatataacttgtaattgagaaacattttttatacattgctaaatatttcatttcagag

SEQ ID NO: 170 - DNA, 370 bp, synthetic construct
  source 1..370: mol_type = other DNA
ttaacacgttgactgccacggccaaaatgagtaaactgctcccagaagccatgttaagcagttcatacagtttgattcgtcatctcatttgaagtccatctgtatcatcgaaactttaaaaataacatttttggcgggaaaatggggcgtgcgagttatctcgtagttggcgtacggagtagaaagttcagctcagacgccagctacgagataactcgttgtcaacaatcagagacgagcttgagtatttcgaagtacatttgacaatacgacacatgtcattgttgtaaccgcgtcagtatttattcagtcttcattgtagatacttcactgacgtttatcgttatctgtgttcaattttcctcgtaaa

SEQ ID NO: 171 - DNA, 394 bp, synthetic construct
  source 1..394: mol_type = other DNA
ttaacactagaacggcggaaggggtcaaaatgacccctgccatacttttttccttcattgtcatttcagtttatttactttcctaatgaaatcatatgacttttcctaattttttctcctctttatgatgatgtattagaatttaaaaagtatttatattttctttgacatttctatcgatatacctagaacggcggatggggtcaatttgaccccaatgtaatttccttctgaatatatgtaagtaatccaacaatgtagcggcaacagtcagcagtattgcagagtagttgcctgtcattgttgcaacttggcacgtgttcgttccagtctgccacagtttatatcgtgtcattctggtgatagtttttgtcctcgaaacacgcttttgaac

SEQ ID NO: 172 - DNA, 542 bp, synthetic construct
  source 1..542: mol_type = other DNA
ttaacacgttcgctgccgttcacgctctggcgcgtgtaaacgttacttgagtcaggtgccgctcacgctgtggcgcgtgctgcgacctagcatcctattatgggttgttgtaaactggcatcgtctaataaagctgtatactctggtaggcactcagggaagagtataagtgatatgcacacatacgtggttccagatttccgcgtttgcattaccggctacgagccacacacgcttctcatgatgtcatgatagatttattggattcttatgtaaacaagcatcgtttatgggggaattacgatcacagcggtgttttggaactctttgtatcacatgttgaattatgcattcgatatttcacgtctctgattcaagtacttacactatatcctgctaatttccttgccagctctcagaaagtatagagtgtaaaatattttccgatttcagtctttgactatcacgtatactgcaaggttgcagatgctttatattcagcagaagaaatacgtacaaaacacttatttcttggtgaaact

SEQ ID NO: 173 - DNA, 345 bp, synthetic construct
  source 1..345: mol_type = other DNA
ttaaccctagaacggtcgggctgggtctgtgagacccactagtttttgtttcactgattttatattcttataattgctgatttgactcgagcggtccgctagcttcctgaaacacccgagagaaagcgttgacagcttgaatgaggtttttcattgtttagtcattgtttggcagtctttcaaaaagcgcgcgggtctgtcagacccagcccgaccgtctacgtaactaatttctgccagtgcgatcccgcttatattttgttgtatcgttgagttaccagttactttgcggactatttgtgatctcctaacaccaagctatcagtcaatagtttatagtataaa

SEQ ID NO: 174 - DNA, 302 bp, synthetic construct
  source 1..302: mol_type = other DNA
ttaaccctcgagcggtcgcgctggcgcactgagtgcgccatagttaaataaacatggttttcgttatgttggtaacaatggagacgtttgaaactctagctattcctccactgttatcctcactattgttgtgtgcagtctggtaattgtcggtgctcaagaaaattcttaacaggagaatttgtgaagcgctgtcataactggcgcactcagtgcgccgtgcgactggtcatgtaagtatttttttactttctcaaacgtagtgttgtatttcagttacgttattcctatggtccgtgttc

SEQ ID NO: 175 - DNA, 336 bp, synthetic construct
  source 1..336: mol_type = other DNA
ttaacccttaactactatctgcggtctctcagaccgctacgactttttgagtcgagatatttcacacacccctgccaagctaaaaggactcttccgtgtaccttcctggtggccgacaaggtccacgtgccagcattcttgcagtgttgctgctctttgttccgtatttatgagccttgcgcggcctcactgaccgaccatagtgtttgcgtgccgctggcgcaggctctgcctcgtagcgcctggccagtcggcttcgtgagcgcggcctgctaagcgacagcgctgtggctgtggcctctctctcgctgttgttgttccgtgtcgtgtttctcc

SEQ ID NO: 176 - DNA, 295 bp, synthetic construct
  source 1..295: mol_type = other DNA
ttaaccctctacaggccaggtgcggtctctgtgaccgatgcaggtttctttttcggttcattccagttcacactgtagctcgacggcttcgaccctccccctaccttcctaattctgtctgttattgctgccagtccaattgttgcggttgtgcgcatactcattgttcagttatttcgtttctgctcagttttcggtcaccgtgaccgcacctggcctgttgtcgctcggttttcagtcagcatgaccgcacctggcctgttgtgtacttagttttcgttttacccaaacagaa

SEQ ID NO: 177 - DNA, 621 bp, synthetic construct
  source 1..621: mol_type = other DNA
ttaaccctagtatgcataccctgggtccaatggacccgggaacaattttaatttctaatttgtgtattaaagacttgaaattgagtcccaaccgttcaggtatgctctctggcaatattcagtaatacattacagatactactagaattgtgtgtcattagtttaagtgccactttaattttcttctttatactacacatttacataacctgggtccgatggaccctgtggtttatgttaaagtgtcgttaaattagattgggttgtaatcaattacattttgttggagtaccgagaatacgttactgccacacatatgcattgttgtgatcgcagaaaacgtattgttataacttggcatgttcaactgtcagacagcatacttctgtgttgtgtaagaagcaaaatatcgcagtgggtggttattagtgtgtgacgctgtacagaaagactgtaggagtttgctgcacagcagtattgtttgacattcgatactcattcggaagaaaggtaagggttattggtaacactaaaaaataacatgaagagccgctatattcctacactccataggagaagagagtactgaggttatttattcatattattatttttcagaca

SEQ ID NO: 178 - DNA, 291 bp, synthetic construct
  source 1..291: mol_type = other DNA
ttaacctttaactaacgtggtgcggtctatctgaccgcgcgctttcatcaacaattgcgatttagccagttccctacccattattgcgcccgtcggctcagtagcttcgagtttagttgtgagtggaccggcaaagcgtgtggtgttgttccagagcttctataacgtgagtgacgtccgtttgaaaataggtcgcggtcattttgaccgcactacgttgtttgtgtaattttttctgctgtgactttccttacatattctcgatatcttccatttcacagcttttgaaat

SEQ ID NO: 179 - DNA, 328 bp, synthetic construct
  source 1..328: mol_type = other DNA
ttaaccctgctgttatcttcgggtcattatgacccgaaaaggaatttgttctttcatagctttttaatatttaaatattatttcttcaaacttgaagactttgtttttcgagacattataaatacttcaaatatcaaattaaaatcaatacttttctcccccttcctaaaacttaatttgttacagttgtgttcttcgggtcaatatgacccggggcaattaaaaattcacagaacgtacagatatcatagtcaagttttatttcgtcatgtgtgaagtatacacacgggtttcatgacaaagtaaactctgattctttattgttgaa

SEQ ID NO: 180 - DNA, 17 bp, synthetic construct
  source 1..17: mol_type = other DNA
ttaaccctttcactgct

SEQ ID NO: 181 - DNA, 234 bp, synthetic construct
  source 1..234: mol_type = other DNA
ttaaccctttccggaccaggtagcactacagtgctacctgaccacgttgtcatataattagggagtcctgtggtacccatttgccgtgaattagaacttttaggggcctgggcactaaaatatgtaagaggaagccattttcttttgagacattcaaagatcatcgatttctcgtgttttggagtgatatttttcaatttttcgacctgatgtttacagctggggtatttaaaa

SEQ ID NO: 182 - DNA, 185 bp, synthetic construct
  source 1..185: mol_type = other DNA
ttaacacattccggtcgttgcccgtagaaactacgagcgcgcatttattgccgtaacagtcggcgctcggagattctccgagcgccgccttctcgattattatttcaggttgcggcgcattatatccgtgcagtgacatctggagttgagtgtttcggtcttgtagtacatcggacgaccgctag

SEQ ID NO: 183 - DNA, 401 bp, synthetic construct
  source 1..401: mol_type = other DNA
ttaacactttccgtccgcgcggcgtactagtactccgcggcctcacacgccacctgtccggccggagtactagtacttcccgactttcataaattcgttaaaaaaaaactattggagatactggcttcccgcttataccattcgactcctccatgactgtagtttcagtggctatgtgcagcttcctgcagcactgcgtagtgtgcacgctggactcttgagaccgaacggttgcgcaacaaactgttttgacgtctgactgaaagcaggcctttgttttaccgcggctgaaacgcgagaccagatgcaatgaatgacgtaccccctcccagcatgtggttcggaaaagtatcaatgagctgtggttgtagttagttgatgtttagcgcgcctcaagcaac

SEQ ID NO: 184 - DNA, 152 bp, synthetic construct
  source 1..152: mol_type = other DNA
ttaactctttgactggcgcgggcgtgttttaacgccagggcgatgccagccgttccctggcgttggcgtgttttaacgctttgaaaatctcttcccttatttgctctcttggctctcatcaaccaaagacatatatatgatagtttttgtgc

SEQ ID NO: 185 - DNA, 394 bp, synthetic construct
  source 1..394: mol_type = other DNA
ttaacacgttgactgccacggcaattttcagaaatctggcctaaaatgacaatttgatttaatcacattatattattaaaaactctaattctagtgcatagttatcatcaaaaagcataaaaaatgtgtttttttttaaaaaaaaaggtcttacgaattatctcgtagttggcttctgtaacagaaatctcttcgcggaagccaactacgagttaattcgcacgcagacacgcacagacacgcttcgacctgtcgcttctctaaagctgaatcatagatacgcgtccatattcgccagtagtatctcagaaattgtcgagtgcatcacatttacgtctttcttgagtgttttgttttataattattgttataattaaattatcaagaacgcaat

SEQ ID NO: 186 - DNA, 323 bp, synthetic construct
  source 1..323: mol_type = other DNA
ttaacacattgaatgccaagattttggaggagccgtattcagttcccaccatacattgtaagcacatacgagaaaattcgtagctggcgtcttcattagtgaagttaactaaagctgccaatgtacgagatatctcgtatacgggatgtctcgtactttggcaccagcaatgttatgcttagattaggtgccatctacgagagaactcgtatgtgtcttccttgtctatcatgcgtatttcttgtgcaacaaagcgatttcagatcagtcacgcggcaggagccgcactagtttgacgtgtgattccacgctttggcgccaaa

SEQ ID NO: 187 - DNA, 233 bp, synthetic construct
  source 1..233: mol_type = other DNA
ttaacacgttcactgccatcggcgcaccagcgcgtccggccgatactagtgcatatgccgtcgacgcgcctgcgcggcgagacactcagctgtttctcagcgccacagtttagtcgggcacagccattcggtgtggttcattgcgcaggcactctaagaacgtattgttttcaatttttgcgggtgcggttcgtgtgttttccctacagtttttctctgttgtgggactgaaa

SEQ ID NO: 188 - DNA, 232 bp, synthetic construct
  source 1..232: mol_type = other DNA
ttaacaggttgactgccgaccactccgatcggagtgcgtcgggtctcttcgccaacgccgtctactccgatcggagttcgacatgcgttcttacgaaacgttttagtattccatcggcgttgtatatgaaacgtgtttctagtggtggcgttgcttaaaaacgcaagttctaacaggtgcttaagtggttccgcggatttgcttttggtttggttgtaatctgccgtacaat

SEQ ID NO: 189 - DNA, 221 bp, synthetic construct
  source 1..221: mol_type = other DNA
ttaacacgttcactgccaatgactcaacatatgagtcacggcggtacccctcctatggccagtggctcaactgttgagtcatgtgcatggctttagtatatgagtttagtattgtttcggcttccgcaggggatacgtctcgtttcaccggtcgcaagtgtacttataaacacgtgttttttcgaaatcggtttttgttggtgataagtagttttcctgac

SEQ ID NO: 190 - DNA, 252 bp, synthetic construct
  source 1..252: mol_type = other DNA
ttaacacgttgactgccgtaatagtcacacaaatttcgatcctgctggccgaaagttttctatgttaaaatacgccagcaatatatcccttgacttgaaataaaagtatattttcataaaaaaaacaatttttcttttgttttgaggtgagaagcattatatgattcacgcggcccatatagttcatatttgcactaatggccatgtgaatcataagccgatgcacatgaaaacttgtttattcttgcagaa

SEQ ID NO: 191 - DNA, 449 bp, synthetic construct
  source 1..449: mol_type = other DNA
ttaacacggtcagcgccaaagccatttctgtaaaaatgtgtggattttcatatttttttttattgcaaatttggattctactcgtgtcagtcattacatatatatatattttttatttatttattatttttttaccccccttttttaaggtgtatactttactatacaccggcctgggaatgtagaatcgacggatttgtattcagcaggccaggtgtatactacagtatacacctaggtttttcctgtttacttttaattttgtgttgaaaacgtttatttttggattgtttcagatcataagacgaacctacgaaacaaaagaagaaagaaaatagaagaaatatagaaaaaaattacattttcaatgtatttttttctatatttcttatattttttttctgattgttatagtttgagtgatttgagacattagtgtttacgaaaaa

SEQ ID NO: 192 - DNA, 301 bp, synthetic construct
  source 1..301: mol_type = other DNA
ttaacactctcaccgccatacggtccgccggcgggacgtatcttttcgagtagagtacgccatctatatgttgcacttggcaagatcagcaggccatacgggccgccggcgtaccgaatctttcgcgccaaaatctaacaaaatttccgttataacttaaaacaattatcgactctgtcacgccacactagtaaacaatcattagcccgccggccgcccgcacggcttgtaacggtcgcattttggcgcgcgttttgtggaatttcatcgttttattgtgaatttcttccatttaattatt

SEQ ID NO: 193 - DNA, 283 bp, synthetic construct
  source 1..283: mol_type = other DNA
atttaacactctggctgccatgtcagtcatattcgactgacagcggatttacaattgaggccaggcataaaaatgtatataactcgtatcaggccacgtatgtagaaagacaactagtggtcataccgcaaatttattggtagggatttattcaccaaaataaagcgcatgcgcatatgcggtgtatagtgcgtcagtcttttttgactgacgcggctctgttaaagatttttttctttgggcgccatgtcagtcattttgaggttatagtatagtgttgtgc

SEQ ID NO: 194 - DNA, 347 bp, synthetic construct
  source 1..347: mol_type = other DNA
ttaaccctttgactgtccccgtacaaaattttttttttatagtgtacgtccacagtaattatttggtaaaaatgacgtgcatcacatgtgatacacggagcaatatttaaactatttattattcaaacgttctgtgcatcacatatgattcatgtcatttttattcgttactatttatttattttgccgtatagtgttagcaacattgaaataaatacgttagcaacactgaaataaacatgtgtgtagagtgaaataaggggaatcccattttttgcaattgtcggtggattgttgtacaaccatggtaggagcttctagcagtcagtatcaatttattagtgagt

SEQ ID NO: 195 - DNA, 479 bp, synthetic construct
  source 1..479: mol_type = other DNA
ttaaccctttccctgccgtaagcgaatatattcgcgctagctcgccagtcccgccacgccgtgagcgaatatattcgcgtcgatctttacgtcgtcatgagagcgtaaataaacacgaattttaactgtcacatattgcatctcctactgccatctgtggggataaaccttaagaccttataatcatatcattgtgtatttaaaatgacgaaatctgtatttttgtgaagtgctccatttgacagccggcagaacgcgctagggtcatgacttaagagtttctatagggattcccacaggcgtcagtgtttgtttcctttgttcggagatcgcgaggttttttatcttacatttgttttattttcgctttatagtattgagatatttgtgtagtgattttattatctcggagttgcttcgtttgtttgttgcattttgtcaaattttataacattatttttgagttttagtgtattaca

SEQ ID NO: 196 - DNA, 293 bp, synthetic construct
  source 1..293: mol_type = other DNA
ttaaccctttaactgctccaggcgtatatatacggtatgcataaaattgctcctgacgtatataaacggttgcgtcaaagaaattatttaacgacaagctgctctggccgtataaacacgattaaaaatggtgatatttaaagacgcattggaaccgcctagagccatccacgcgccgaaatatttcagtggggggtggggttacttttcaacttgtgcgaaccgtctcgatcttgtttgtttagtatgctgattgaattattgaagggaggttattgttttgctgtttcaaa

SEQ ID NO: 197 - DNA, 327 bp, synthetic construct
  source 1..327: mol_type = other DNA
ttaaccctttgaatgctgcggacgtcatttgacgtctttgatttgccgtgcctacagtgctatggacgtcatttagttattttgatttgccgtgaagtgctgtggccgacaattgcagtttcctgcgcacgtgttcatttgtgggtaattcaaggcggggctagcgcttgctgagtagtgggcggtctctttacgtctgcgtttagatgttgttattcactcattgccgatgcttgtcccaacacgaaaacattgcgatttctatcagttttactttagtattgcatcatttttacatcagtatcgtcctattagtttttataaata

SEQ ID NO: 198 - DNA, 271 bp, synthetic construct
  source 1..271: mol_type = other DNA
ttaacctctccagccggttcgacgtacacgtgtacgtccccggctggtcgcttccaccagcgtttcgacgtacgccgtacgccctctcgcctcattcagcattagtcgcttcccttatgccatctcttgttctatttagtcaattctttcggtcctctgctaggaagctttgcatgatgctgccatcttaattgactcatcataacctttttggaaatattttttttctcttgatgaagtttgagaattagtgcctccagacctcggagaa

SEQ ID NO: 199 - DNA, 369 bp, synthetic construct
  source 1..369: mol_type = other DNA
ttaacccattcgctgacattgacgagtgctactcgccatggatttacaatacaacagggactttgacgagcgtgactcgcggcatatgtccgtccactgactttgacgtatctttctcgccgtgttatttcaatacaatgaattgtttcgtagtagttctatgaatgtccaatagatgtcagagaaatccagttccttctagttatggttttatttttctctaatagatgtctagcgtaatgtgagctctaacatctgtgtcattattatccaatctctgttagtatcctttattagtgacaaaagtatagtgtttattacttctaccttgtatataattgtttattcacttggctttttcccaacaaa

SEQ ID NO: 200 - DNA, 273 bp, synthetic construct
  source 1..273: mol_type = other DNA
ttaacctcttcagtggtacgcccggaaaagttccgggccttgccccactgcccaaaacgttctaccccggaagatttccgtggtaaacttagaatctagttcgttcactcccgaaacctttccggggcaatggtactattatgctagtagtttatatgttgcccattagatgtctcactcttattaggaagcttataccacctagcggtgtttttgggtattgtttagtgatatgtatagtatggtgtgcactgtgcagtgtaaccctgaacg

SEQ ID NO: 201 - DNA, 264 bp, synthetic construct
  source 1..264: mol_type = other DNA
ttaaccctttcccgaccaaagccctttaaaccgctcgacgggctcccgacctaagcgcccgaaaccgcccgtctcgaaatttccgcatcgggtccaagcgcccgagaccgcccgtaatcttgtatcccctaagaggcgccttcagcaagagttatgagacatctctactaaacaactcctttcatttcatctgtgaacggcctggtcatcctgcacacatggccatatgtccctatttttttttttttttgggccttcgttgtt

SEQ ID NO: 202 - DNA, 361 bp, synthetic construct
  source 1..361: mol_type = other DNA
ttaaccctttcgagcctattgggtcatatgtaacccaacaagttttttcagctgtctgttgggtcaaattattcccattttaacgtatattgggaagagtttcgtccttctgaaacccgttgataaaacaaagtgtgttataacctcacatctcagtttattcgttattttaccgcgtaaacacgtgtgtgtcggttgtttagagtgttttgtagttggtaaaagtgttcattggttagtctgtttgtcgagaacgctttagaaacaaggtaagatattgttgttgctcaccttatattgaaattattgaaaacaaaaatccgggacatagatgtcccaccagtctttttacttgggttaa

SEQ ID NO: 203 - DNA, 299 bp, synthetic construct
  source 1..299: mol_type = other DNA
ttaaccctttcgcgtctaatgggtcatatatgtcccaccaagaaattgaaaaattctagagggaacaattcgtacttatagagcatataaacaagtcccactcgcatcggggaacttcctgcacgtattcccgcgcaaatttcggcctaataaccttcagtatgtttacgttagcgaaatatagcacgtggcagcgggtaatttggggattttatgctgttaattaccgtaaacaaagattataactagttcaatttgattttacaatggcgaggtaagtaatacaaagtgatattata

SEQ ID NO: 204 - DNA, 468 bp, synthetic construct
  source 1..468: mol_type = other DNA
ttaactctttcaagggccctggtgtacaaaatgtaccaccttaatgcatgctctagttaggcggtggtacatatttagcaccactggtataaaatttatttacgaatggagcagtggtttagtttatgaaccaccattagaaataagttgtttacgtccttataaagaagtgcgccttgcggtaacggcgccggacatagagccactgcctgaaaaataaattaaggttataattaagttgtcaagagtgtaaactaactgtgagtgagaggaagactaaaaaagtaagtatatttgtatatttgcgttgtaattatattataaagctattttcttatgatggagactgaatatttatttgaaaaaatcctgtaatatttgatatctgtctggtactctatgcgtaccaccgcacacattaggttttttattttttgcgacagcttttttttattttcaggttccaac

SEQ ID NO: 205 - DNA, 436 bp, synthetic construct
  source 1..436: mol_type = other DNA
ttaactctttgcagtaccttggttgacaaattgaaccacttttttgctgaccttttctgtatggtggtccataaaacgtaccacttaaatcaaaattatttttattgcgtacgtcggtttaataatcgtccctccatagcgaaacatctcattggcctatgattagttgggtgatacaaattcgtcgaacgctggcgtttatttttacggttttaggtatttatttacttctcgcgtgccctgctgtgatttgaggttattgtttttaaaagaaaagtgttgtttttattcatctggtgcattactttatgaatttcatataaaaacacataataaaggtaggttaatatccatttttaatgttcgtgaagtttgaaacctgaattttgttattttttttagtgtttaaaaattgtaccaccgtactgttagaaac

SEQ ID NO: 206 - DNA, 472 bp, synthetic construct
  source 1..472: mol_type = other DNA
ttaactctttgctgcatatgggattgcaatttgaaccaccaaattcaagcagtctgtgacacatcggtccacagaatagaccacttacaaatatgtatgtaggaagctgaacagcgacgggaattatgaaccacctatcctaacctctttttcatagttccattaccaactgaaagagatcccgctctttagttatcaaacattcaacgtcacatgcagtgtttttgtgagaaagtgaggttattgtgtatattaatttcaatattgaacagaaactaaggtatgtttatttattataatatcattgtatagaattgtttatttatatcactagttagtatttattatgattgtaataaaaatttacattcaattaattgtaattatttatttttggtgagtcccaattatgaaccactatgccgctaggtacaatttattctaaaatttattgttacagatagactgtatt

SEQ ID NO: 207 - DNA, 463 bp, synthetic construct
  source 1..463: mol_type = other DNA
ttaaccctttcacgaccatagggacatatgtgtcccaagaaataaaagttatctatacataacaaatgaaaatttatggctaacgttttattaattatgggttccggaacgtaccttctttttttttctcacagccattacaatctaagtgacatctgtcaaatttagttgaaggttgtttattgcagtttgttcggcgccatcatcacgagttttgaaagactacaaatataactgaaagtaactttctaattctggaagtgtgaaataattacagcctggtaagtaaacaactactccataatacaataaatctgattcttacgttttgtgccgtgacaaacctataaatatgttatgggacacctgtgtccctacggtcggtagcggtaaaaaaatgtagtaaaataagagttatttttttcaatctacccttgcttttcaagttattttgttgcagatt

SEQ ID NO: 208 - DNA, 434 bp, synthetic construct
  source 1..434: mol_type = other DNA
ttaaccctttccttcccagtgatgccatatggcgtaggtaactaaatttgttaatatcaggcagatttcttttcccattatttgttttttactaaacctacactttatcgttaccaatagatggcactagagtatcaaaacaaaaacgcaggcataacctcttttttttattacatgttcaattgtcagtagttgtgcgcataaataataggcggttattttgtgagtcttttttgaagaaacgtaagtatatagttgtaatttttgcttatttacctttagttagatctaatcaagagctctatgcaatattatggtgatttccaaattgttactgtcattacacgactttgaacttgatgattccatatggagtcattgagcgccaaggttaaaattttggttaaaatcaattactgttgcaggtttttatt

SEQ ID NO: 209 - DNA, 475 bp, synthetic construct
  source 1..475: mol_type = other DNA
attaaccctctattgcattttgttcccaaacgggaacaatatcatttttcaataatttctattaaataaaacttacaaaatattgtttttatgcatggagtgacttctcgggaacattattatttaaaactttgtcattctcccactagatggtaggccaagacaaaatattccatattttggtttctactgttctgacgagacgtaacgtgtttttgtgcgagttttagtggtaaataattatatttttaggtaagtaataattactatttttttaaaatatcattttgtagtatataaattagtataaggtacatttatttcaaatacttcttgaaaataaagtaataaaaaacatatttatatttttgttcccatccgggaacattatgcgtagaaattacttcggtactagatatttttgtttttttttgacaagtatattttttagctaaattattgtttttttttcaga

SEQ ID NO: 210 - DNA, 447 bp, synthetic construct
  source 1..447: mol_type = other DNA
ttaaccctctattgcatattgttgccaattggtaactcaaactttaaaatttcctatctatttcgcattactataaactgttccttgtatgcgtggtgttacctcctggtaacataactgaatgcatagctatgcgcgtaaattgataaatggtgctaggtgttgtaaatgtgatttttgtttgttgacagcacaaaagcaaggttatagttgtaatcttggtgtgttgaactagaagtttgaagtttttatagtgaaataaaggtaagaactgctatagtaaaaatcaaaaatggtcacataaatcatcactatgtcatgttgtatttctcacatatgacttttgcatgaggaaatcgaaacaattacgtgctcaacgaatgttgcctttcggaaacgttatgcactgaccaaacattttcgtaaaataaaaaattatattgatac

SEQ ID NO: 211 - DNA, 386 bp, synthetic construct
  source 1..386: mol_type = other DNA
ttaacccatatagacctaccgtaccctattcggtacggtaacttcataaaattattttgacgaacctgtgggtagaacaatttttttttattgtggctatgaggctcaagtgtcagtcttacgataaaaatattacaaactggttacgtgtaggtgcattcgagttttaattattattttctggcagaaaaggaaaaaaaatatttaccatgtcttcaagaaggtaagttccaaattatttattataaattatattatttatcactattattgtgaagtttagaatatagtagatactattacaagaaaaaaagttaggctaatattatttttcttaggtatattcaaacctcaattagatgtaccgtatatggtacagtaggtct

SEQ ID NO: 212 - DNA, 437 bp, synthetic construct
  source 1..437: mol_type = other DNA
aattaacccattaccgcccattgtactcatctgagtacaaatttttaatgaatcattttgcacttcacaaatcttacatacatacctaaaatgtcttattgcgcgcaaatgcgtagtttcatgtgtttacttcgaattcgtgctagcaaagagtacaaaaaacataacggtttatttatttttgttgtttttttctgttgtgttttgcaatttttcatttttttgagctcagtttataattgtcaacagtgtgagatgcatacgcaaatatacgcagcgatattataaataaacattgaaaacatatctagaaagtgatttgagtacataaattcataattagaaattattttggttcaggtaagtgtgtttgaaattaggtgtactcgattgggtacaatgggcagatctgtactcagcaaaaaaatttttttaga

SEQ ID NO: 213 - DNA, 437 bp, synthetic construct
  source 1..437: mol_type = other DNA
ttaacccatttacgcctgccgtgcccaaacgaggacgccttcaaatctacccaccattttgttattaatgtggtaagttttgctaacagtgcattattgtcgaggtgattgatgtgtcttacattttatatcaaacattttggataacactatttataaaaagtattttagtgtcataaataaagtgaatctcagagttgcagtgattaccaaatgtaagtatttttttttatatgtttactttcgtaaattcttaattgttctacataatcttattgaacactttgtttactttggaataagaatttgataaggtaaaatatataatttactgaactgtgaactacttttttttgttggtgtccccaaatagggacagtgggcacataatgagtaaaatattctttttttttacagagttttgactgcacataaat

SEQ ID NO: 214 - DNA, 425 bp, synthetic construct
  source 1..425: mol_type = other DNA
ttaacccattaacgcccgccgtactcaaatgagtacagttattttggacaaactctgccatctgatgggcaatatattaagttgttagctatcgcgccatattttattaatctctgggcagtgtttactgatagttgacaggtattggtgtgttttgaaaagattagttacagttttaaaatatgacttatttttgaacctggtaagtaaagtgaatataatatatttaggaggctatttgtataattattgacaaattacgccaaatatctattttatatgtattaaacaagtggtaaaaattagccatagattcataacctcatttcctgaaacgtgaatcagcgccaaacacggatgtactcaaatgagtacagtgggctcaagtatgaatttttaatttcaagctacgttttgtttcagaa

SEQ ID NO: 215 - DNA, 462 bp, synthetic construct
  source 1..462: mol_type = other DNA
ttaacccttatccgggcaaggggggtctagtaggcccgaacaagaaataaaacttaaatatttctattaataaatggaatttgagttaatgctttctagaattcctccaaaatggtatttactaattagtcatcaagagattatggaatttatttcctgcatattaattttatcagcatttttcaattaaaatcacattaccggtcaagcaggcctgatgagccctaaattgtttaacgaaatataattaccttttaagattatatttatctaatgatatacctacataattacaccgtcatttgtataaaggtatttagtgaaaattattggtttttacaatagaagaaaactttcccaccactcctgaaggggtactacttatattatataaatgcatgtcaatattagcttgaagttttagtttgtatgtaattgtgaatctaattgtgctgcgtaaga

SEQ ID NO: 216 - DNA, 428 bp, synthetic construct
  source 1..428: mol_type = other DNA
ttaaccctaggatgcatatgcagggcctccgaggccctatcggtttttattttgcttgttactctgtaaggcttgaaatatgagaactgaccggatgtgtaatcttcaattgattattctactttcatttgatcttctcgcaaaggaagaattcttagaagttttatagatagttactaaaatacaattctactacgtcaatgcatatgcagggcctttgaggccctatcgtgaaaaggtcagatttcaaagcgtggggcgataagcaggcttttacaatgcgtggttcattctgagaaacaaagttatcgtgtttttcctttgttgacaagcaatcttggagaatcttgcgtatagttagtaagatttcagctgcgaaacagtttggcaaggagtgggaagtgtgccgtgtgctttcaatttgaaat

SEQ ID NO: 217 - DNA, 423 bp, synthetic construct
  source 1..423: mol_type = other DNA
ttaaccctcatattatgttaagggtcaatttgacccatttcagtttttggtttgaccaaagaactggttatcctttctttttcttcatgaaagttggtgacttttcctcatctagggtcatgaacttgtgtgtaaaatctggatactgtgatgtgtcgtggaatgtctgtgaacagtttgtatacaaagatgatgttgcgggtcattttgacccacacactttgatgtgggcaagtagctgtccagatccgaaataaacatgtctctttgatgtactttattttgattgctaaattatttatattttgactgtctctgaatagaccttcagactcagagacccaggtgtgtgtgggggaggagctttctctcccttgtccttgtcactgttctcgtgtcatctctttgagaaacacagcaaaa

SEQ ID NO: 218 - DNA, 276 bp, synthetic construct
  source 1..276: mol_type = other DNA
ttaacctttagaggcctaagtgggggacattggggaccccagtctacatgttgtgagtggcaacactgtttaagttaccagtattgcgtttagcttctagctatgtgtcaacagtaatttaaggttatgtgtcaccaattgtcgttgactaattgctataaatttaaaagttattacaatttccggtgagtgggcccacaatgtccccctctcaggcatttacgtaaccaatatatattttagataaactggttttgtgacattgttttttgaaaa

SEQ ID NO: 219 - DNA, 425 bp, synthetic construct
  source 1..425: mol_type = other DNA
ttaaccttcgtaaggtcaactggggtgcaaatgcaccccaggtgattatttatgttcgtattttttgaaataaacactattgagatatagtaatttttctaatcgtttattgaacattgtactacaatgtgctgtgctaagaattaatgtagtatgcgtatttttattgatataattttatttacgctctggggtgcatttggaccccagttgaccatctacgttataatatttcctataccggatgcaagttgctgttcgtcttgccgcttctgggaatgcaagcttgcagtattgcaggcttcgacatgtttcaacttgattttaggtttcactataaatataaacttttctgtttgtgtgcagtattagtttgtcttgaagttgagcagtgctagtgtgcaattctcgcgtttgtgttgaaa

SEQ ID NO: 220 - DNA, 310 bp, synthetic construct
  source 1..310: mol_type = other DNA
ttaaccctccgttgctactcctgggtttggcagacccactcgcatatcgccgttaattttttttaatgttgccattactgcacgatcgtcatatgtcttgtagcttcctgtcttagtcctaacagtgttttgacacagtaatttgtattaattttgttgtgttactccatcccagccgtaattttgattgttacgggtttcgtggacccagagtagcaagcgggtgagtgaaatacgggttcggcagacccaggtagcaacgctgcgtgcgtattctttttacgttcaatgcaagtatattgctatcaag

SEQ ID NO: 221 - DNA, 488 bp, synthetic construct
  source 1..488: mol_type = other DNA
attaaccctgcagtagtcgcgcgcggactcccgctccgcattcaaatatttttgtctataattcgagtgtttttacgcgcagcggctcgccccacctaataccttcctgtttgggtccgagatacccgaagtccgacgaagaaaacaatcagtgatgattcgaacgtgctggaggctattcgttgtcgtaataattattttgcggagtgtcagtccgcgcgcgactgtatacgaggtactttctttacgtgttttttggtgtgtattctgagcagatgtaagtatttctttaattttttcctttagtataggtactttgacagttttattcttcttggtaattttaagagccaatatttcttttttataaaaacataatagtacattattatacttttcccaatctgactttaactgattttgttctgaataatttattcttctaaaactgattttgtgcatattttataaaacaagtttttctttag

SEQ ID NO: 222 - DNA, 264 bp, synthetic construct
  source 1..264: mol_type = other DNA
ttaaccttcgacagataacgtgcggagcgagactccgcacgctccgaatttactcgatatttaacgaattattccgactacaccgctctgcggcacagtagctgttacatttggggtggtgaaattagtgttaaatagtgagttcgggagagtgcttgattctggtttttcggcctcttgcggagtgacactccgcacgttatccttcttgcgatatatttttagacgtgagtgattctgtttattattaattttataatcact

SEQ ID NO: 223 - DNA, 403 bp, synthetic construct
  source 1..403: mol_type = other DNA
ttaaccctacattggtgacgtatatgtctgtgagaccgggtagtttaaaaacaaagaatattttttacaccgtttatcgaattgcgttgcgatttcgagtagctgccttaatgcacgccccctaccattagtccaccccgcgacagcgacgcgggcgcacgttcctgagttttggcgccaacctggtccgtgagaccggtgtcaccaacgaagtaagtcaaatgtttgttgtgtatttttttatttcacttattttgataggaatgttgctgttatttatagtgtgattgtaagattttatgtttagtaagggttttacccgtttttattgatattttactcgatattattagaagtgtttatgcatttttcaaataaatagtgtttttttttttgttttagt

SEQ ID NO: 224 - DNA, 382 bp, synthetic construct
  source 1..382: mol_type = other DNA
ttaaccttcctatactcgcgcgttggtctgtgagaccgacgaaacacattttcgtaattatttttatgcaggtaaatcctaagaagttgagttccttgctagctgccggatttgtggctaacttcgttttagtgcacgccgcgcgttcgggcgaaacacacgtgttgtagcactcgtcaccaatcggtccacgagaccgacgcgagtataggtgtaactttttgtgtaagtattaatataatttatttcgtgcatttgtatattttttgcggactttgattactttataaagtatatatagtaaaatatatgtatgtatgttgctctatattatattttgaccaattttatcattcttttttaggttttaatccatatcaaa

SEQ ID NO: 225 - DNA, 381 bp, synthetic construct
  source 1..381: mol_type = other DNA
ttaaccctggagccggggcgccaagtctcatagacccggagtaggataatttcgttggcacggttggtaggagtgcgccagaaacctaccgctttgtctagctgtctatttttatgttccagtcaaggtcgaagtgtcgttggagtctcttctgtgcgtatttacgattcgggtctgacagactaggcgccccggctcgtgttacagtgatttgggtctgagaaactaggcgccccggtttgtgttagtaaattttgtcattttaaattaaattaaatcttgtttttttttacttttttcagtgtttatgaataattttctcaatttgtaagtgtagatagtttatagactataacgaattattttgtatgaactagaaca

SEQ ID NO: 226 - DNA, 318 bp, synthetic construct
  source 1..318: mol_type = other DNA
ttaaccttgcactagtggcgccgggtatgtgagacccgagcgctcctactccaactaatcattttggcgtcatacgagctacgacgctaggctcctcagtaccttcctagctcttgcgtgtgccacatgtgctgtattgaccacttgatcggatgtccgtaaatacgtttatgttgcttgaaaagcctgacagacccagcaccaccagttacgttgcgtgaacaaatataatttattatcattgttttctacatttttccagaaagtgtgagtattagaaattttattcatatcagaattgaattattgaatgatgtt

SEQ ID NO: 227 - DNA, 305 bp, synthetic construct
  source 1..305: mol_type = other DNA
ttaacacgcgagtagtcgcgcccgcggcaatttgccgcgcattctaagtttttgtaagttgttgagggaatttttgttttatgaggttgtcatcccgtctaatcctcggcacgacatccctctacgtgaccttgggctgtttccgtcttcagtgtgaacctttcgcttgccgtgatcgttatactgaacgtatgtcaagtctgtgagcaaattgctcacgtgcgatcactcgtgttagttgtatttttagttgctaattttgtatatatttttgtaaatagtagtgtagttattgttgtacagaa

SEQ ID NO: 228 - DNA, 320 bp, synthetic construct
  source 1..320: mol_type = other DNA
ttaaccctcgacccgtcgcgtggggtctttgaaagcccagcttttacgaaaatgcaaattacgtacctttataatctttgcagtggtctgttactttaggtagcttcctaaaaaggtttattgttgacgctcgcactctcaacaatcagtctacgcacgtgcgccgttagatacagacgtgtttttcggcgcgaggctctgagggaccccacgcgacgagtggcgtaagtaaaatttcactcatactttttttctgttttattattatatagcttacattcaaatttcttttattttagatacgctacaagactgttatc

SEQ ID NO: 229 - DNA, 337 bp, synthetic construct
  source 1..337: mol_type = other DNA
ttaaccttcaagcgggcgcgcgtagtcgaatcgaccgcgcttagtacatttttgtttatattattgttactacccaccacattaatttctcattcctagtagcctgtcgctatgtcattgggagtaaaataaacaataaaccgtcgctcgaggcgctcttcccactccgcagatcgcaattgttttagtcccggcgtagtcgtcgaaaagattacacgcgcctgtcgtcgaaaagattacgcgcgacgctcaacgcgacatacactcgttcatgcttgtaatagttcttcggacattattcaatattatctaatttattttggatttttatatagca

SEQ ID NO: 230 - DNA, 279 bp, synthetic construct
  source 1..279: mol_type = other DNA
ttaacccgccagtagtcgcgcatgggtcgaaaagacccagaggtgcaaaatttgatccgtaattcttttaatttcattagtgttaggttgccacgctaggtaccttcaagtgactttataatgtactcgcaccccccacctcagccattttgcctgcgacgtttgaacaggacggacgtgtcaagtaagtatgggtcttttagacccttgcgcgacgttttatgtgagtattttttgtaattgttcaaaatatatattattattattattgtttacaga

SEQ ID NO: 231 - DNA, 226 bp, synthetic construct
  source 1..226: mol_type = other DNA
ttaacccttcggtactcgcgcgggctacatgtgtagcccagctgtgttgttttgcacacacctcggaataccgtggtgggggaaagggcggggcctcagtaccttcaaatcaatatttgttctatcgggtcagtgtgagttggcacgtctaggagagtggatttgcattgcttttacgttcaaagttgtttgggctacaactgtagcccgcgcgagtgttgttgca

SEQ ID NO: 232 - DNA, 349 bp, synthetic construct
  source 1..349: mol_type = other DNA
ttaacactccagcggtcgcgcggggtacaattgtatcccagtgttttgtttttacgcgccattgtgagttggcagcctgtgtgaccttgagcagccatctaccttttttgtgaagtgtctagagtgtgccagttgtagcttgaccgtcttcgtgaacacacagtgagtgttattcttgttttcctagtgacagtcactttttggggtacaagtgtaccccacgcgaccagtcgtgtcccggtctgcatttaaactgcttatagttatgtaataatgtgtttgtgtaaatattaaatataattacttttttgacactttgctggttagtaatttaagttttagagtaaat

SEQ ID NO: 233 - DNA, 431 bp, synthetic construct
  source 1..431: mol_type = other DNA
ttaacactccgttacacgcgcggggcacaggcgtgccccagctgcatatttttgtctgtaaatcacaaaacttaacagccaggattttgattttttaagtagctgttgcatatatcttcgcgtgagcgtcagtgacgcgtcgactgtgccggcgctcggacgtgctctgtgtaagtgtttatgcatctttggggcacgcctgtgccccacgcgtgtaattacgtaactagtggtacgcggcttatttggtatttattatgtttttttttattattttcaagataattatgttaaaaatctgtgtaaatttgttaaattcatattactgtttttatttttctttattggtggtatgattttcgtaattaaattggacatttttcttgttttaggtattttatcttgtaaagtttgtctgattatctgctaag

SEQ ID NO: 234 - DNA, 312 bp, synthetic construct
  source 1..312: mol_type = other DNA
ttaacactccagtactcgcatagggaaaaaagttaccccactcataattttcctagaatttttctgaataggcttaatctaataaagctgttttttatagtaccttccttctaagaactgtggcccatatctaagcgtaaacaagctccccaaattcagtattcgcgttgccagagccagataaagttctctggggtaaattattaccccgtgcgagtaaaagtgcatttgttttagacaatattgtgttatatttattattattatttttattattatttgtgttaaattagtgttatattttttgtgacg

SEQ ID NO: 235 - DNA, 307 bp, synthetic construct
  source 1..307: mol_type = other DNA
ttaaccttccagcgggcgcgctgtcgcaaattttacgacaaatgcaccttatcgcttcccccacttttaccacttagtcgcaagcgtcgagctccgagctattcgaaggtcagtgtctctctaaactatagacgtaccggttgtgcttagtagtttttgttattcattgatatttgggaatttcctttaatcgtaaattttacgacacgcgccttaaagtgtaagtatattaaataaactattttttctcatttatatcgtttcttttatagctaaatgtatttttttgttttagacgtgtttcatc

SEQ ID NO: 236 - DNA, 363 bp, synthetic construct
  source 1..363: mol_type = other DNA
ttaacccttaactactggcgtcgggtcaatttgacccgacggcattttctttttttattttccggtaacgcgacgagctaccgatgatgcgggttccacgtaatctcttctgataacgttctgagtcagggcatgctgtgaagaaagccgttgacatatcggtttctctgaattgccctttctgacccgacgccagcgtcagtgttcccgtgtctttcgagagtggcggatgtgcaagcttggtgccttagcgataattcgcgactcatcttaggtaagtggtgaatactgcttccactgggtgtcggcatatgttttgaaaacgagtaagataacttgcatttgttcttatcctgttaggtc

SEQ ID NO: 237 - DNA, 297 bp, synthetic construct
  source 1..297: mol_type = other DNA
ttaacctttataccgacacgaggagtctcccagactcctcgaccaatagaaatctaaatattttcatttgtatttgattttaaatttcgcgctactagataccttccaggcgttataatgcccatatgcagctgaacgctcgacagtcggcgtgcgacagctcttgagtaaacaggtgctaaaatccgtaaggagtctgtcagactccgcgtgtcggtttaggatacaaacattggaaaaaagttttttcacatttttgacatttttttgtattaaagtgtattttgtgagagttta

SEQ ID NO: 238 - DNA, 266 bp, synthetic construct
  source 1..266: mol_type = other DNA
ttaacccttaactactgtggtgcggtcaccatgaccgcagcgcttatcaagatctctaaaatttgccccctctcaagttataatgccttgggcgtccttgtacctttaaattagtacaattcaagaaagcagtgcgagtacgtcgtgttttatgtctctatttttttagttacgccgagttaaattttttttgcggtcatagtgaccgcagtacagtaacggtgaaatattttattctttgcttgtaacgaatattcaaatttatt

SEQ ID NO: 239 - DNA, 399 bp, synthetic construct
  source 1..399: mol_type = other DNA
ttaacccttaaaccgaaacaccggtctctgagacctgtggggtcacatttggctaacttataacaacccagtttgatctgtgtctctcccctcccgaatatgcttctatcaactccttacctatgcaaagcgggcaatagcgtttagtatatatcttgttactgtaaggtgcacgtcggttttctaaagtgttccggtacgagagaccgggtgtttcgcgttacgctagtaataatacaggtatgtatgaccgtatgtttcgcattatgtacaaattatacctaagctattattgtattgcaatttttttttcagttggagagagattataaaatattttacttagtagttgtctggacaaatcaaaaagtgttcttattattttcagatcccgttatc

SEQ ID NO: 240 - DNA, 297 bp, synthetic construct
  source 1..297: mol_type = other DNA
ttaaccctcaagtactatgcgtgggtcctagggacccatgtaaataagttttcatgcagattttggcaacgccgcgcgccactggcgtttccgcttgtgtaatctccagtaacattaaacacttctcagtcgtgaatctgttgttaaagtggacgtatcgaatttcgagttattagtgtcgacgggtctgtgggacccacgcatagtaattactttacaatttctggtttggtaagttttaattttttttgttttttgtttaatttttatgttttttttttaattttagaaagaaat

SEQ ID NO: 241 - DNA, 275 bp, synthetic construct
  source 1..275: mol_type = other DNA
ttaacccttagctggtttcgggggtcaataattaccccaggcgcacacattttcaaagatatctttcctgtttaaaaacgtatactcttgcgcgtcggtgtaatctcactacgtcgctgggtgcggcgggtgacggacgcacgtcgctctaccattgctggaagcggaggtacgtacactacaggggtcaaaaattacccccgacaccaaaagtgctgttttttttgcgcgcgcatgtaacggtttttttgttattttaggaaaatatcgttgca

SEQ ID NO: 242 - DNA, 278 bp, synthetic construct
  source 1..278: mol_type = other DNA
ttaacctttaactacacgggtggggtctcagcagaccccacgcgaaattttttgtttataatttccattgttggcgcactacagactcgtcattgtaggtattctcagtgtgccgctgccgcttgcagtaggtggacgtgcgccgcgctctcgcaacagtaagtgcctgtttttcattgttggggtacctgtagaccccacgcgtgcttttacgttgtatttatttgcaaaatgctattatagaaatttcctcttatttcagtatattttttacaaca

SEQ ID NO: 243 - DNA, 309 bp, synthetic construct
  source 1..309: mol_type = other DNA
ttaaccctcaatcaacactaggccgacaaatccgacccagaccgttgtttttaacgtttttttcccccttccgttgtcttttgaggattaggacttcaggtaccctttcacccacttattaagagcacattgtgacagaagaatcgttagaggtaagtaatccattctttttattttaaacgtttttccaagttctatgaacccgggtcggtcccgacccgctagtgttggaattgttaaaatatttttttaattttttaatttttttttttttttttcgtttcttttagactctataagtattcaact

SEQ ID NO: 244 - DNA, 410 bp, synthetic construct
  source 1..410: mol_type = other DNA
ttaaccctggatgagtaacatggggtatttgaataccccactgaattttgttttctacatatttttgtgttggttgcactggcacatctggctttccgttacttttcctaacaatgtgtcataagttagtacctcctttgtgtttgtggtgagtggttgtgttgtggagttaccaagtgattggggtccggaggaaccccacgttactcttagcgtttttaggtgagaaattccattactaaaatatatatcattgtacaagtttattttatgtgactgatatacttacaatttcctaatttcattccaagtgatatattatattatattatattatattatattatattattattattaattatattataatttagaatgtattacgctattttccaggttttcctaat

SEQ ID NO: 245 - DNA, 417 bp, synthetic construct
  source 1..417: mol_type = other DNA
ttaaccctagatcactaaacattagtctcacaggctacacgactctgacgcattcgctgtttatgtatgttcccgttaccgcacccccctcagccgctcagtaccttcctaattcggccaatcagtgacccgtgaactcgcattgcgtacgcacgcttcctacttccggagatattacgcaatagtagtctctcaggctaatgtttagtaattgcgccacaaaagtgaagaaaggtaagttatgaaatatttctttttatttacgattttatctataataactaattactgattaatgtaaatgaataataaatatgtgtgatactacgagcaatatttattttatgaacttattatcattttttatttttttatgagattttgaacattttgttttagcagtattttctgttacag

SEQ ID NO: 246 - DNA, 307 bp, synthetic construct
  source 1..307: mol_type = other DNA
ttaaccctagagtactaatcatcgcctgagaggccgcccgcggtcggttttttctggttattttgaaattgcggggacgtgtcgcgccgtgctttcgcgccaactcagtgtagcccacgacgcggcggggagagcttgcgcgctcggcggggatacagcgttcccgagatattgcctacgtgatcgcctgggaggcgatcattagtattaaaggtagggtttttactattgacttaatttacatttttgagttttttctttgtttactcactattataagatataaaatacatgttttatttcatag

SEQ ID NO: 247 - DNA, 289 bp, synthetic construct
  source 1..289: mol_type = other DNA
ttaaccctagttagctaacgttcgtcaaaatgacgagcatctaataaatatctccatctccctttctcatcgttttacctacacattcacggtttcgcgccaacttagtgttcgctagacttcgtcgcgtggtggttgcgtttagccggtaagtacttttgtttttgagatacctactgcatactcgtctttttgacgaatggttagcttatgcgtaacttttagcttacgtgctttgtgttgatttttgcattgttttttttttcagttatttcgttgatagtgaaga

SEQ ID NO: 248 - DNA, 410 bp, synthetic construct
  source 1..410: mol_type = other DNA
ttaacccattcaatgccgaaggtaagcgtcagaccaaacctggtggccaccatacaaatggtctactacgtaattattcgtagttggcatcacgtatataaggatataggtaggatgccagaatacgtaatattacgtatacgcagtattacgtagataggcacagatagaaaaataaatgttagtagtgcctacctacgcaataatacgtatacgtaaataacataccttagctccctaataaaacacagactattgtctatctcgttttaacatgtataaatggcaatcacgtgttctccccacttatttttgcatcaatagtacatttattacctgtgtgcgcattcagtgtcgcgccgcacgcgctgagagactgtcgctagataggtgtaggtagttattgcaat

SEQ ID NO: 249 - DNA, 443 bp, synthetic construct
  source 1..443: mol_type = other DNA
ttaacacattcactgccagcaacgcctcagaggcgttcatgcgcacttcgtaaaaatgccgggaccgcccgagcggcgttctcgtctaacgcttattcatagggagataaataagcacggtcagtttgaattcgttcactattatgatcggatgttttgtgtaccttatttttactacgtgacggctttggtttctgaaagtgttaattctgtgtgttttttttatcagttttttgtaaaaggtaggtttttaatattcttctagtttttttattattttattagtgcaaatcatagaaaaataccaaaatcatgttattctagtaattttttgttagatatatgaatatataaacacgaaaattaaataatgaccgcccgagaggcgttcaagggcacaggcaaatttatgatagttttaatgaattttcttacagattatt

SEQ ID NO: 250 - DNA, 463 bp, synthetic construct
  source 1..463: mol_type = other DNA
ttaacacgttcgctaccgatatgttaaataaaaaacatatatatagaccagctgtcccatatatgcgccgtacggtttttactcctggaccagcgtcccacatatgagccgctcgggcccgctgatacagaccgacggcgcatatttgggacagtgttctattgttactaataatatcctgacaaatcactggtctattttaacggtttaatattttcgtatcggtctctgggggtgtctggcgcaaatatgggacggttggtctctggataagtcaaagtcaatgacctgtatttacggtatatatttcactggtctcaacagtattagtttgtgaaatgctcgtccggccgcgccactgcagcagagcacgtggttcactaagtttgaggttatgttcagtgaatgaggttacgttctgtgagtaagtttacgtactgtataagcttctatctgatttttt

SEQ ID NO: 251 - DNA, 338 bp, synthetic construct
  source 1..338: mol_type = other DNA
ttaacacgttaaacgccgatagaaaaaattaaaaatcgtaaaaatacttttggtcgtaatttgtttattatgttacgatacgtatagaaaataagttttattcaattctgtacatgggttacatagttacagtcaaatacatagctgaggtcaccggtgcaccccatggcgcacaaatacagaaaatatattttccggtaccaggcgccgcgccgcccaccccgccggccacccgctacaccagtagtgcgctcgcgcttacggtgatagaacgtgtgttactagttcaattacgttcctactatcttagctgtcatcattttcttattgaatagtta

SEQ ID NO: 252 - DNA, 405 bp, synthetic construct
  source 1..405: mol_type = other DNA
ttaacaccttcaccgccaagcccaatctcttaaaatttcatttttttaataatgtattttttttgcttaaatatgcatttttttatattttttgctgttatattttatccagctgtcacccctttcatttttcaggatttcaggaatgtgtatcaaaaatgatacactggcctatgaaagacatttctctgttaaccaatatcgtaggccatgtgtatcattttggatacacggtatgttttagtttatttgattattttatatttgtttcagatcaaacacgaagaaatcacaaaggagttttagaaaaacaacaactgttgtgtttttctataactccttttttgttttgttataggatattctataaaacgtaaataacgtgagtgagaaaagtgaatcaaa

SEQ ID NO: 253 - DNA, 345 bp, synthetic construct
  source 1..345: mol_type = other DNA
ttaacaggttgaatgcgtcaccggtcccgggggaccggtggcgtactttggtgtcttgcggttgcgtatatcggtattttttcgcaacttgcggatgttgtattttcgttaatatagcttcccttagaattttacaaatacgttttggtttatcgctacgtttaggaacttatagattatgaaaaagtttaccggtcgccgaggaccggtaacgcactgatctaatactatattacttgtgtgcggtaccaatcgcccgggaccggtgcggttttcaatgtaaaacggtggggatgcctatgaatgcagtattagtaaagaccacgtgttgtagtgtgttacaaa

SEQ ID NO: 254 - DNA, 482 bp, synthetic construct
  source 1..482: mol_type = other DNA
ttaacaggttccctgtccccgtactaatctgtagggtgtccctccgcgctcaaaattttttccatgaaaataaatttagaatacgatgctataacaaatgtaatatacacaatttagaaaattgtattctaaatttgaaaatttaaaatttgaagttttgatcgtatatgattcatgagcgtgacatgaaagtgcattatattcacacgctcaatgaatcgtatatgattcatatgaaattttatcttattggacgcactggcattgcattttatggtgaaatgcagtgttgttaaatgtagatagcgtttataccgttattcttttgtttgaacacgattacacataacatttgtgcatttgggaatttgtgttgcatcgatcagtattttagcagttgatcgcaaaggaacgtcagtacagagataaaaaggcgaaagtacgggtattttgaagaaaaagttggttgtgagagcttcaaa

SEQ ID NO: 255 - DNA, 465 bp, synthetic construct
  source 1..465: mol_type = other DNA
ttaaccctttgagcagtacgaacgatcatgtacgtccttgcgccacccggccatccggagtacgaccgtacatttacgttttcaatctttgcgctagagcagtgaaggcgtgaatgcacgcctttctttcagaatcattgtagattgtgttgccatctatcctcgagactggtgagcccagtgatcactatgatgtatctctgaagagtgctaccacctattctatatcactagtaactacgttcactccgagtacctgttaccatgtatgtagctgccctagttctgttgacagaactgtttgcatttactgttttttattgttattattgttttcttctggcgaaatattcaggatcttagattgataatttaggtacaattgttgtttttatatttgtttgtaatttctacataatatacatcattcattcgtcaaacattttcagctcgtcatcaggaaag

SEQ ID NO: 256 - DNA, 662 bp, synthetic construct
  source 1..662: mol_type = other DNA
tcattaacccctcaagcgtgactttaaatggaggcatttaatccctcagcgtgtgcgacgcaccgtctgcgtccgctagaatccatgtctcaagcgtgtgcggcgcaccgcttgcgtccaggattttcattgggaattcttatcgctcaggctcaaatatatggttttttcagtgagtatatatgtggtccaatgcctcctgccttcgtcctggtcctcctacgtatctgtgttaatgtttatgcatcaaaaaaaattttttttcaaatgatgcctgggaaagggaaggctctctcgcagcccagcgtctggtgtctccatacgaacgtattttttccctctcacgatctcctttatgagcttccagagagatgatacggacgtcctgatgctgaatgctttcgtctgtggttccttgttatctgtttttctgtgtcaacatttcctttctctttcatcgtggtaataattacgaagccacggtccttccaggcatggtcactcaatgcttctaggaacttcgaagacctgtcagctccggtatcttatttacgatgtttgaagataagggtcgagttaccctgacgcctatggttgagtcgggggcttagttttttttcttcgttgggaccgtcccgtcgctgacccctctttagttgacc

SEQ ID NO: 257 - DNA, 431 bp, synthetic construct
  source 1..431: mol_type = other DNA
ttaacccttttgatcccagcgtccgattttacggacgttttgtaagatcgtgttcgatcccaacgtccgaattttcggacgttttaaaaaatcacgttcgatcccaacgtccgattttccggacgttcacaaaaatgttttttttaatacaatataatcggttgaatacttcttatgttatattaaaccatttgtaatgaacacctttacaaagttatagtatgtaaaggaacttttagttcaatttggcaacgatgttttcgttcaaactagaaggctgtggctgctcggtgccatgtgctgtctgctctcatcgtcgtcagtcgtgagtgtatcgtctgtttgttatgagctgtagttaaccttattgtttggaagtttattggttacgttatttacaatgagtttatgattctttgtagttatttaca

SEQ ID NO: 258 - DNA, 405 bp, synthetic construct
  source 1..405: mol_type = other DNA
ttaaccctttgaccgccaacgtccgattgatcggacagtgtaggtttatgccaaaaccgccaacgtccgactgatcggacagcctaggttcgttccaaaaccgccaacgtccgaccgatcggacgttcaggaaaaaatattttttaaatatacatatgaactttaaaagtttgtgtcgacactgttcgacttccttttgactaaataagatgatatgtgaacaattttaatgttggcagtcctggagaccttgactcagctgtttgtttacttctctaactcgccgagcgtgcgctagtgtttacgactaacctatttgccaagttcccgcctttctcatagatgcctacttggttttttgtttagtttttctctgaagttagtgatctgtattatttgtgcaag

SEQ ID NO: 259 - DNA, 499 bp, synthetic construct
  source 1..499: mol_type = other DNA
ttaactctttgaagggcgtgtgtcactataactaaccaccaaattgatgctgttctatgggcggtggtcattgtagctaaccaccaacttagtgctgttgtatgcgcggtggttacctggtgtaaccacattcttttattgtgctcccttacatggaccgctacgactctttctaaccacatgttatagttgtcctgtggcccaatagatggcagaaagagtactctgcggcatttctatgatgtatttattgtttaagcggtcgctttattcagatgtgccattgtcacttgcagtgttacgtttctgactgctgttttatttcagattattgtattttattggtggtaagtatttcatttgatatttaattcatttaaaaagctattgaaatattattagagtctttacaaagttcatttgttattccaggcaacatatttacaggtggtcagtgctgctaaccaccgcccttataaccttataacgtacaacagat

SEQ ID NO: 260 - DNA, 394 bp, synthetic construct
  source 1..394: mol_type = other DNA
ttaaccctttagtgcccgtcggtactcctcagtaccaaccattttaattccatataactttgtattgcaggcaagtaaacgctttgtaccccgacggtacacgggagtaccatttataaagcaatgtaaacgttcatccgggccatgcgcgcagtttcaagcgttatcccgccatttgtgtatcgctgtgttttgggttatgacgcaattttagtgctctgtatttgcactacaattttactggttattagtgcttttaatttgtcttctttgttttgtacagttgctaatacatatactgggtaagtttatatttgaaacaattaaaaattgataagttttgacataaaaatgtctcttttattcataggacttcagagtaccgttaggaatt

SEQ ID NO: 261 - DNA, 463 bp, synthetic construct
  source 1..463: mol_type = other DNA
ttaaccctttgtcttccaggcagtttttttgtgtaggcctatttatctcccgacggtacttttaagtccgcaggttttgttccccctatactccactattattgatcaaactcctttaaaacgccaagggtacttttaaggactgtagaaacaacataatgtttgtttggtaaatttggtaacactgatttttgtcactgttttcatgcttgttgtgacagcttggtggttagaaagcaacgtgttcaattatattgaaaatatcgtggtatttgttgttacaattgaaatccagcaagcaattaatgtaagtaagacctttatttatgttgtctattaaaatatttgtatgtttagtccatttcaaacaagatacacatctttatctatatggtactcataagtacctttggcggttagtgtataatttatcgaattttattatttttcaaatttttacaga

SEQ ID NO: 262 - DNA, 635 bp, synthetic construct
  source 1..635: mol_type = other DNA
ttaaccctttccggtccagtgagtccaaatggaatcacgtcgctttcctgcttcgagcatcaattcaaatcccccgcggactctatttagcgttagcggccgtagtacatagtgccaccaccaggttttatgactcgccgttcaagaataaatacgatcttcagctattgatgaagattgctgcacatgtgcttgtgagtattcttcggagttcatcggcggagttttacatgaatgaaaaacggaggtaagtgcagtattttgaccataaaaattaagagctctttcgacattgtttgacattgattataagagattttaagcttctgcgagaggaaatgtctttgtaattttataaaatataacatgattccatttggaatcagtggcccagatgtatgtgttaagtttgaccgagtttgcaaagcatcattttactgaaagtaagctcattagttgagatccaacgaaatagttggtattttaaatacgaattagaattgacggaacgtgttaattgaatatctcatgaacggaattgtctaattgttttttcagtgtatttatgtttagtaaagccacagggattttggaaaatagattttattttcaccgaatcatttccatttcagacg

SEQ ID NO: 263 - DNA, 513 bp, synthetic construct
  source 1..513: mol_type = other DNA
ttaaccctttctgcgtccacgattccacttggcatcgatactttctcccggcctgtaagggtataagaatgacaacaattacttcgtttggcgtcgctacacgtagttctacgcttggccaacagatcgcattgttgtattttgaatttcgttgtcgttagtttggaatatttaacggcgaaagttttagaacgttacgcgttggtttgttcgcaaacagcctcgtgtgttaattattctgtgcttcatattctaaaagcttcagtgatgaattcagcatcatcttattcctcaagaaatagggaaagaaataaggtaggtttttggtattgttacatatgcattttcgtcagtaattattgttttatttctaatgacgccatttggcgacaatgacgcattagcacatatgctatatgcaaacatgaatttaaaatgtttgaaatattgagcttgttttacttatataacactttgtaatttaattcttgctttatatttctattataggtc

SEQ ID NO: 264 - DNA, 477 bp, synthetic construct
  source 1..477: mol_type = other DNA
tattaacccttaaatgcataaggttgcaaatttgcaacgaccaaatttcatctatataaagttgctttaaaaatgaaatagtaagtaactgcatactgttgcagattcatgacactctgtttaacgtaagtttaaggttttgtatcatagatggcagacgtgtcaggcattgtgtttggtttattggttagttggttatgaaagttacccggtgtgcttatagttaaaacaaaagtcgttatatcttgcttattcttgtgataaattgaattcgaagactgtgtccgtgtgtacaaaggtatgtagttactatgtatttgatgtagattgagcaaaaatttgaatttctttgtatttttgttaaattaccgctgttgtaaatatgcaacaccatgcacatttacataattttgactttttaatggtgttgtagatttgcaacattgtgcatttggagaacggaaatttgtaatccac

SEQ ID NO: 265 - DNA, 549 bp, synthetic construct
  source 1..549: mol_type = other DNA
ttaaccctctaactccccagtccgcctctaggcggggtttttctgtagagctttagcacccaggggttaactattttttgcggctaacggtattttcgtgtgcaacaggaatgtgtatgtatctattgctatgcttggctttgctctagcacgtgttttgtagcagttatgttaattgaaagtggaagtttgccaagaaaaggtgtttttttaattttcgcgaataagaagtttttaaaaaattcataaaaaatcgaaaaattatccaattgaataaaaaaaaattatcggtgagtagaaaaaactgttatcttttagaaaaagatgcaatttgtatacttaggtgcatttagcagtttacagtgttaaattcgttgaggccgcctgtaggcggacctgggcactcaagtgaaaatttttttgcggtgcattttagttattcaatagcatttacatggctatttttgtaatttccatttataatttgtaaattttttgttagttttgtaattttcatttgtaatctgtaaattttttgtattattagtc

SEQ ID NO: 266 - DNA, 525 bp, synthetic construct
  source 1..525: mol_type = other DNA
ttaacccttaataacctagagtttcaattttgatactaatattttaaaccgaatattcacattactattagtttttttttatatgttggctgtatgtattttactgttgtatagtattctatccaacgaaattaaaaaaaaaacaaatgtcattattgttttattttatctgtgacatattacttgcgcggcaaagtgaacaagtaaacacattttactaaaagttttgttgaatataaatttgatatattttctttttctgaagaatataaatacttttgaggtaagtgaatacattattttcgctatgatataattatttttagcaaatgcatgttttacttgaagaaattagtgcttttttttttggtatcaatattgatacttcaggcattccgccctacataaaacctacctactaactctttagctataaaactttttgaactttcgtttcatattttttctgcatagcttggtgtggggattacttttgtttttatttattatagaaatttatccaaa

SEQ ID NO: 267 - DNA, 486 bp, synthetic construct
  source 1..486: mol_type = other DNA
ttaacccttaaatgcatcgtgatggaaatttccatcaaccgattcaatgccattttttcgtttctacagaggtcggtagtcacatattttatacatggcaacttttatgatacattaacaatgtttctgaacgaaaaaaaaaagtttatccctcaatgtcagctgacaaatgtcattcgaaaatgtgaagagccgctatggtgatttcgaaattttcgatatatgtaaatgtgaaaaagttaaataaatgctcaatgtttacattctattttattcctaattgatagatttgaggtaagtttagcgatttatatctttatttttgttcgtcaagaaagaaataggtaaaataaaactcaaaatagtgttgatgtaaatttacatcatcatgtcattacggctatttagtttgtatgaaaagctagtaactagtgttttttattggtttcttgttattttctgttttaggtgttgatattttacgaa

SEQ ID NO: 268 - DNA, 421 bp, synthetic construct
  source 1..421: mol_type = other DNA
ttaaccctttaatgcatactgtagctgataagctacagacctcatttcttcgatttattccatatggagaaacattttcgatcaaattcgttatgtagctaagtgtatacactccgctgcagtttctagtagttcttcatagcgatcatagatggcaggacgagctgaagcagttcgacagtgagctgtgtggacatactgccaagtgtgtgttttggcggaaagttgaggtgtgtttttggtgtttgtgcactgatttctgggtttgaaaattactttttcattttgaaaacgtaagtagatatggtgtaaacagttaattcgagtgtctttgcgatagatttgaaatgaggcctgtttttgtgtatgtagcttatcagctacatttttcatttactgcatttcaggcgctgacgtaaaa

SEQ ID NO: 269 - DNA, 646 bp, synthetic construct
  source 1..646: mol_type = other DNA
ttaaccctctaccgcataggaacccatatatggtttcccaactttaaagtcacctttgttacataatttcgaattatttataggtaaaaactatttttatgtgttcgatagatacataggaataacgaaacgcaattaatttgttcaatgtcaaagggtttttgaagttaccctcgtttttgtgcgaatggtcggagcaaatttagcgacgtgagtaaagtagtgcgaacctcaacttcaaataattattatatagtgataaggtaagctttttgaactataaaaaatattgtattatgtgccacaacctcttctttgcataacatgtgtcgcattttcactattcgaaggttttcagtaattttcatactttcaaatgtcgaaaccatatttggttttgtatgcattcgaacgctttttcactgagacataaatgggttcgtatgcattgaagcttgatactttgtatctacaataaaactgcaaaatatgggttcgctttatttctttccttcataaacagctaataatagtccactgctgaacttgaacctcctcttcatcagtggagagttttgccaaaatattagctttgctttgatattcgagagctttgctatatacagcttacctatttattttcaga

SEQ ID NO: 270 - DNA, 545 bp, synthetic construct
  source 1..545: mol_type = other DNA
attaacccaataacgcccagcgtatgatttatcatacaaaggtgacgtcgttttgttaggttagaatactctctccagttgcaaataaacagtgtccgagtgtttatatattatagttaaaaggtcatttaaactatctaaaaacaaatttaaagttaattggcaattcagtgtttaagagtttggtttatttaaaagtgcattcttcaatttgatgcaaaatgagttacaggtaagttcaaacgtattttttgtaacgtagtactaaaaaaaaagtatttgtaggcgaaatactgttgtttaagatattacctataattataaaaacaagtaggtactcgcttttgcaacctttttttgtaacagctgaaaaaatatgcggtatgaaatatcatacgttgggaactaccgtgtaataaaaacagtaaaaaatttaaacctaaaagtggcaatgcgtagttcgtatttttatattatataattatacaaatactaatattctaaatttctcgcttcgcaggaaaaagtatgatttggacaatcta

SEQ ID NO: 271 - DNA, 392 bp, synthetic construct
  source 1..392: mol_type = other DNA
ttaacccattaactgccatggtcctcatttgaggacctaagagctttttcattgttacgtcatactgtatgccattgttggtcatataagtagcttacatagtggtatcgtgatttacgatcagtgttacattgtcagtgacacttgacggagctcagtattctttatattgaagtaaaggtaagactctgcagcaaatttgacttcaatttttctaacagtatttgtgtttttgtttagtaatgaattattaatatttaaaagttaaaactattaaattaagtgaaaatggatataattcaacaaatttaaatgttaatattagttgtgtttttactcactgactttaaaattcatcttgttgagtggtcctcatttgaggaccatggcac

SEQ ID NO: 272 - DNA, 429 bp, synthetic construct
  source 1..429: mol_type = other DNA
ttaaccttcgactgagcacgtggggtcttgtcggaccccactctcagattttctctaaaatcttttgatagagaatttatagtgactctattttttagaacatctctttttagatgtttatttatatatctgtatattattcgtacaacagaatcaaatcaaattcgacaaaaaaaatactgaatccaaaacaagcacggatgtgcacttggggtctgttgagaccccagacaaacaggactctcagtcgtctgtaggtgttccgtggtgttatatttgaggtgattgtttgtgttttattagtgcaatactatggatgattatatgtaagtataatttttacataaatttctgttagctatgttgtgttttcataaaagtttagatacaaacattacatttttttgagatgtttgaaattgtgaggtt

SEQ ID NO: 273 - DNA, 643 bp, synthetic construct
  source 1..643: mol_type = other DNA
ttaacccttggatgcatgacccgggtcttttggacccgacggcatatttgtatgtgaatatctttatcggaattgaattttagaatcccaccttcctttaatacctcaattaaagctttgcgttccatattatactttgtttgaagcaaatgggctttttgttaattttattttttaatttttttgtttgtatacctcttttacatgacccgggcactttagacccggtgacatattttcgcatttcgcagcatgacacgggccataaaaatgcttcatgggtacctataggccatctactggccattttaattattagagatgattcattagtttcggcactagggtgcagcacatactttatttgttgttcgtagttagatgtggagcgaggctggattttctgtttcgtgcaacgtgttcacgcaacgttcattctcagaataagtgtttcctgttgttttgttgttgtgagcactgttgatattttacggtaagtgctgaactattacttgcaatttaaatttattcattgatcgtaatacagttctgagtttcacattttgaatttttaggttagggttattttattgtctgtataatacaaaatgacaaaactaattgtagcttatttcttgaagat

SEQ ID NO: 274 - DNA, 580 bp, synthetic construct
  source 1..580: mol_type = other DNA
ttaaccctaggatgcatatacagggcctccgaggcccttgtatgtcttcattttttttaaaaatctgtaattttttgtttgatttcaaactgctttctagtactctttcactaacactgtgacattcctcctaccaagtctgaacgagatacctttttaagttttctgtataattctatacgtttacccatacaccgtgaatgcataagcagggcttcagaagccctcacatatttataaatgttctttagcctatattgtcttcaacatttgtttgggagcagttattaattcaacaataactgtagtagtatttccagcaactggagtgccaacagcagcagtagctggagtagtaactaaaaaataatacacactgctttcacatattggcgatgtcggtgtatcattgatctttttgctatcaaatgttttattattgcatagtattgttatcctgtgctgctcttgtcagcctcattgttactgtagtttgtttgttgctgcaaggcccatattgttacgagtatgactcgtttagtgctgcacaagctgctgttcgagggacacgtcttttgct

SEQ ID NO: 275 - DNA, 450 bp, synthetic construct
  source 1..450: mol_type = other DNA
ttaaccctgctgttctgttcgggtctatatgacccgaaacgaatatgaacgttattttacattatgtaaataccaatatatatttatgaaactttgtgactttgtctgaaaatggatgagcagcatgtgttttaaaaggttttaaaaaagtttaagaagtttgggcttcaactgtaatagttgcatatgtaatgttcgggtcataatgacccgacacaaatatgtttagtgtaactcgtatttatttctaaaatctggaaatggcgaaaattatttttgttgtgttgtgtgggaatagtgactgcatcattccaacttactctcaacaatcacctaaagctccatgcgttcacaacaatgggcttgtttgttgctttccccaggaaataataattggcagttctcaataattggaatgctttgtttctgcccagtttgacttgtgacacc

SEQ ID NO: 276 - DNA, 467 bp, synthetic construct
  source 1..467: mol_type = other DNA
ttaaccttcatcgaaccaagggggggtgcaactgcaccccagaattggttttttcgccgtagcaaagtaaatattttttttttagttttgatacttcatgactttgttaatcaatgctttttacggcctgtgcgttattttgtaatttttaatttatttcacacacacggataaaaataaaataggcactggggtgcgcgtgcaccccctccttaacacttaacgttacaactttattgtaggctgcgttgtcggtttccaatgttatatgtcatttcagatatagcaataataattataagattttattttggtcaaaacgtctacaatatttcagtcaggaaacgtttcagattgctgtaagttattgtgtgctatttctaattattagatttctaaaaattatattctcttgtgtaatataacctcaaatatttgttgtgtagaaactgcaattgaaaaaaata

SEQ ID NO: 277 - DNA, 622 bp, synthetic construct
  source 1..622: mol_type = other DNA
ttaacactagaaaacccagagatttcttacccctctaccatgcccagagtacagccaaaaggctgcccggtttgaaattaacaattcaatggccaacaattagcacctttacatttgtaaacacagccattacctgccgttagctgttcaagcatactccttggggggaggagtgtgcttgaaaagagccttgtagtctgctttcaactgtgctgtatagtttcactcaccacaaacggtatatgtgcttttgaccatttctcacattttcatgtaccctttattgagcattggtcatgtgagttttcatcgtcatcacactattgtacgcccagcttgctttcaagtgagagattatcacgtttctgtttccacaaaggccaaaaggaaagcataatcaagtatttcaaatgagagataattacattccttttgacctgggaacagaaaagcaaaatcaaaactggtttcaccacattgtgggattgttgtgggtgcttcacaaattggtgaaatggtgggcgtcggtgtgaacgactaaactagggaactgaggtggataaatagctcactcacagcttcaagattttcatggtgaacaacggtaagccaagaagtgaag

SEQ ID NO: 278 - DNA, 287 bp, synthetic construct
  source 1..287: mol_type = other DNA
ttaaccctccgtcggtcgcactatatctgacagataccagcacatgtttttttcccgcttctttgacgtttcaccgccaaatgcccccaccacttcagtggtagcttccctccacccccccaacgagtcagtgtcgaatccggtttagtcgagataatacctgccttgctagacgttttgaatctcttagatacggtgcgactcttcccgtgagttttttgttagtatctgacagatactgtgcgactcttcttgtttgtaatacgttacatatttaactttaaaat

SEQ ID NO: 279 - DNA, 639 bp, synthetic construct
  source 1..639: mol_type = other DNA
ttaaccctagagtggtcgcgcggcgtcaaatcgaccgcagcaaacactaatattaagttggcaccactgttatttgcttcccgcgcatgacagtcaaggacatctccctccctacctccttctattccgtctggctcggggaaccggtttgtttcactctgccagtcgtaatttgtctgtcattgtgaggtgcggtcgatttgacgccgcgtgaccacttgtgtaactcaaaatcccgtgtttatcgccttgcgctagctgtgttgtgaatacatatctttgtacatttgacgtgtgtctatcatttttacaccggttattttatagttttggtatttttgtacatttcacttgtgtttatcatttttacaccggttattttattagttttggtatttttgtgcattttacgtgtgtttattatttttacaccggttattttattagttttggtatttttgtacatttcacgtgtgtttatcatttttacaccggttattttattagttttggtatctgtgtacatttcacgtgtgtttataatttttacaccggttattttattagttttggtgtctttgtacatttcacgtgtgtttattatttttacaccggttattttaggtattttttagtgca

SEQ ID NO: 280 - DNA, 368 bp, synthetic construct
  source 1..368: mol_type = other DNA
ttaacccgcgagaactcgcgctattagattgaacctaacattgttagttgttgtaaaggaattttttttaatttttgtcgaatgggtctcaggttctaaatatgctatctagagtatattcagcagcttccgcacttgttccgagaatgtagagttcggggtgggggtagagggagtgggggaagtgaggcgccgcgctgttgttagtctgctcctaactacgcgagtggtcgtgtcagtgctggtgttagttccattctaacgacgcgagtgatcgcggaatattccattttagttttattatttgcggtgttttgttgttttagtgtttttgttagtaaaactttgctagtaggctaagtttagtg

SEQ ID NO: 281 - DNA, 359 bp, synthetic construct
  source 1..359: mol_type = other DNA
attaacccttcagtactcgcgtggattacaatagtaatccactgatatttaatcctgtattacgtcaccgcctgcagcactgcgtagctgagcgtcaatgcaatttctcgtcacagtctaagtgaggcattactggtgtttagacgtatttgtcgcaacgctttggaaagttatacggagttttatcaagttatacgcagtttttccgagtggattaccattgtaatccacgcgagtactgacgtcagtttcttgctcaggtaagtgaaatagtttttatatggttatgagtgatgcaatgcaggaactttatgaaaaaagtgtgtgatatcgttccatcctctttgtagttggctcca

SEQ ID NO: 282 - DNA, 605 bp, synthetic construct
  source 1..605: mol_type = other DNA
ttaaccctttgaacggaacatcggtctgagataccgccatagttgacattaacaatgctaacggcgctgcattgttcgtgagggtggggagaagcccagtaatcttcagtcgggtcccgaaccgcagctggccaacaacgtattgtcggcgcatgcattgttttcaagtaagtagaatttttgttacgtattgtcggtctgtgagaccgtgttccgaaaaaatgcattcccctcccggtctgacagaccgggtgttcctaaaagtgtcaaatttttgccggtctcccagaccggtgttcctaaaagtatcacgcatttgtcggtctgtatgaccgatgttccactctttgaactcttctgttttctgtgtagcaacatttcatctatttgattgaaatttttgccaccacacgatgcttaatacttttgtgttcatagaagaacgagaaaaaagtgtaacaactcgtatttaacgttttaatttttcctctttccgtttttcacacacattctccaatttaccgcgcaaacttattacagcgatagaattatccccattaaatttcgttaaaactcatttttccctttttccttgcaggtttaca

SEQ ID NO: 283 - DNA, 357 bp, synthetic construct
  source 1..357: mol_type = other DNA
ttaacccgtaccgccacatgtggatcccccagactcacagggaaatttcgattggttacgttggccttcctgcacattgcacagttacgtggtggccatgtaaatctcaaccagtatctcaaggacagcagaggatagtagaagatttattgccgctcttcatttcacgtaagtaacctttttcccaaaaagttgtgggtctgtgagactcacctgtggcagtggattcttgacactgtgggtctgtgagactcacctgtggcactggattctgacaaatttatataaaattaatataaatttttgtttctatttctaatctattttctgcctttttacccatttacttatttcaga

SEQ ID NO: 284 - DNA, 432 bp, synthetic construct
  source 1..432: mol_type = other DNA
atttaacccttaaatcgaaataccggtcttggagaccgctagggtattttgacttgtgaatgttggtagtactacttgaccttgaggcgtacgcgcctgtgtaccttccccccactcttttttcggtcgatcaacactcttcccgctcagttccagtccagttgtgaagccagttagttgtgtcttttaaaagtctggcggtctcttataccggtatttctcttacgcgctgcccgttttagcggtcttacatactgatatttctggaatgagctcaaaatacattttgaacttttcttatttctttgtttatctttacttgacatgtattaatagagtgaacatttgtaaatacttgtaaattgacttgtatagtgggtgtggaaatagtgaaatagtggaagtagtggaaaaaggtctggttcattcact

SEQ ID NO: 285 - DNA, 261 bp, synthetic construct
  source 1..261: mol_type = other DNA
attttaacctttaaatggtgaggtggggtctaggagaccccagcagccctttaaatttctgtgaatccccttcccctcatgctaacatcttgtccccacctgacatttcctgtctcaacatgctctcactgtaactcacttttccagtcagtttccttcgtgtgtttggtcaggaaagacgtcttggggtcccacagaccccacctcactgctagtgttcggattttttcaaatatagttttttctaatattctggtataa

SEQ ID NO: 286 - DNA, 326 bp, synthetic construct
  source 1..326: mol_type = other DNA
ttaaccctggaatggtaagctttcgtatttttgactactcatctttgtttttccccggtaaatatgttgtgcgtcggttggcaaacctgtccatccgtgccatccccccaccctgcagtgccaacacaagtgttcactcggctgtcgtctgtcactatctatcaacgagttatttatagtgcgtagtaaattttacgaaagcttaccgattgcgttgttttttttgaggtgccgtattttttgatttctagtttataagtgtatattttgtatataagaagtttattttattatatttgcagtaaatgttttaacattttaaaatt

SEQ ID NO: 287 - DNA, 351 bp, synthetic construct
  source 1..351: mol_type = other DNA
ttaaccctggattacaaacccttcgtaattctgacgaccggccatttgcaaatacgctagaactcgtaaaccacgccccctagcgctgcccctaccatagtaccttccgctattgggctacaaagctcagtactgccgtgacagccaagacggtctagtctaacttattgttttatgtataccgtagtaaattttacgaagggtttgtaagagcgtgacctttttaatgcgagttaaagttttatttattccaagtttttagtgttgtaagtgtaagtttttaggatttgtggagtgtgtttgtacagattttgtttaaaaatagtgtagtatactttagaaatagcgaat

SEQ ID NO: 288 - DNA, 344 bp, synthetic construct
  source 1..344: mol_type = other DNA
ttaaccctggaacgctagcgtacgtcagactgacgcatcgtgcgcgcgccgatcgcttccccttcggtcccttccctcctgcacctctgtacccccactctatctctcagcgactcagttgcctcgcagccgccgcgggagaaacatcacagcataggaagcgtcgcgctctgtgaggttagttctgcaacactgtacgtcaaattgacgcatcgctagtacagcgagcatattttcctgggagtcattttgacgtacgccagtaacccatgtatttcttatggactacttttatctgttcgcacgctgttgttaactggtatgtttacttttgttttagtgca

SEQ ID NO: 289 - DNA, 365 bp, synthetic construct
  source 1..365: mol_type = other DNA
ttaaccctagaaagctaaggcgtcgtcactgtgacgacgcgatacagtttttcgagtgtgccgcgctaagttattgtttgaacgcgccccccctccacgtaaggtcagtcaacgctccgccgggtccgtgagtagtttggcagccacctcgctcgtatagtttgtgcaatatatcgaatgcgtagtcgtggagacgaaacgttagcttgtacgttgtttttacaatagcattgtgtcgtcttgccgacgatagtttagtgttttacgtgctcgtcatatgtacgaatgtttagtgtttctatgtgtttttgtatgtgataaagttgtaaatatttatgtaatatagttattggtgaactctcaaa

SEQ ID NO: 290 - DNA, 441 bp, synthetic construct
  source 1..441: mol_type = other DNA
ttaaccctttcgctattttgttgccatatggcaacagccacttttatgtctaatatttttgttcatatttaatatatctgaattgtttcttgttgattattattttttaatatctaaagaagacaaaaaattttaaatttggacattttagataaattttcatgaaaataatttaaatctaaataaccttatatatactccacaattttcagtaaaaaagttgactttttgtaaaatttttttaaaatattattccaaaatcttaaaaagatattttaataaaaaactaataaaaactcattggtttatgggatttcgcaaaaaagtatggtttttaagcccgcataagtgtgtagcgattttttaaaatcaacctgttgccatatggcaacaaatttatcttacgtatataattgatcaataaacgttacttttttgtat

SEQ ID NO: 291 - DNA, 591 bp, synthetic construct
  source 1..591: mol_type = other DNA
ttaacccatttaagcccaagcggtcgaaaacgaccagtgtatcattttatcccaagcggtcgaaaacgaccttgggggattttttttgttctacgcgccggcaatccaccgatgtacctacaatgtaacttaaatgtaacatttgacttctcggaactatgtggggttgagatctcgcaatttagtcagtgtggtagagagatcggcctcgtgaacttcgggaagtctattctacattggcatgtcaggaaaaatgtctcggtaagttttccattttttatttaagcactaaaaaaacatgtatttcataaaatagatagcctgttttattgtataccagcgaattacatgtttatctagagtcctattgagcctagagcctatttatctatgagtggtcgttttcgaccacttgggataatgcatgctctgcttgcttattttccgtagcgaatcgtgagatgaaggattcgttgtcccgatgtttttgcctatgggtttaagactgcccgccactctgaaagattaaaattctgtaaatgcttaattgaatatttttattttcgtacaacctattagttttatgttcct

SEQ ID NO: 292 - DNA, 333 bp, synthetic construct
  source 1..333: mol_type = other DNA
ttaaccctgcagtggtgaggttgggtctggtagacccaagggatattttatttatgttttattttaggttggcatcactgcagacggctggtgaattttgacagcttcaaggtcatccccccctacccactccacttgtcgtccattgctgccagttccgagttgattcccaataaatacacaagtgcggactgggtctgttagacccaacgttattactcgtgttattgtgttttgggtctattagaccctaggttaccattggcgtgatagtgtttttgagtgtcggatttgagtgttttcttcgttatagtgaacttttattgtttaatt

SEQ ID NO: 293 - DNA, 212 bp, synthetic construct
  source 1..212: mol_type = other DNA
tcgcgttgccttagtattatatttagtattattttatcaatttctttcttttttcataaaaaaaattaattttcaaattttcaaaaaaaaattcaaactttttctaaactttttttaatacctttttcttatcaaattcgttatacttattcattttcgacaatatttaaccattaataaataaaattagaccccgggactcaaaaggttaa

SEQ ID NO: 294 - DNA, 337 bp, synthetic construct
  source 1..337: mol_type = other DNA
taaccatatttattgataaaaataaaatagtttatttttcgattgaattttttgtcttttcttatttttacatattgattcgtagtttttagaaaaaaaatttcttgaaaaaaatcattcgttttgtttagttttataaaaatacacacagaaatgtgattttcattatagtttgatttattttagtttttagctaatattatgtaaacagtgtgttttatgaaatttttttatttgaaaaaaaattttcttttttaattttatataagaaaaaccacatttgatatttttagaactagtttgccactatagtggcggccggccctcaaagggttaa

SEQ ID NO: 295 - DNA, 322 bp, synthetic construct
  source 1..322: mol_type = other DNA
ttttattttattatttttcaaataaataaatattattattatattttgttacaaatttaggttcaattaaatattttcacagttaaaaatttgtttttttcttcaaatagtaactgaaaaataaaattacaatagtcgatccgtgatgcaattgaaaatccactacaattatttatccgctatttctattaaattttgaacaattttgcatcttttttttgggttttagtgaaaataaggttcaaatatgtattttttcaaataaaaaaaattgtttttttttgccggtttttttcaaaaaaagtgtgcgttaaggggttaa

SEQ ID NO: 296 - DNA, 242 bp, synthetic construct
  source 1..242: mol_type = other DNA
gaaagacaatatatggactgattctagaagaaaatccctcactgtaactttgatttctcatcaactgaatattattattttattgccgggtggcaagtatacttgccggtctgtatttcggtaacaaatacaatatttcattttttttcttcataattacttaaaggagactcgaataaatcagtttagttgaaaattgcgttttattaagctttgtagaattatcggcattaatgggttaa

SEQ ID NO: 297 - DNA, 165 bp, synthetic construct
  source 1..165: mol_type = other DNA
aatgtagtttatgtactcttgaataaaagaaaatatatttttatactggtaagctggctattggtctgcattcaatattgaaaaatataagttgtgaaagaccggtcatttgaccggccgcggtaggaataggtatacgtgaagtgtcggtaggaatagtgttaa

SEQ ID NO: 298 - DNA, 202 bp, synthetic construct
  source 1..202: mol_type = other DNA
attaaaaattcttattttttacgtttgtaatagaaatggtcaatatactatttatttttaattataataaattttatttttatgttaatttccttcctaacattcatatttcatacattcaagcaggaaacgtgaagtcaggtcatttgacccgctatggtagaaataggtatctaaaaagtgttggtatgattagtgttaa

SEQ ID NO: 299 - DNA, 191 bp, synthetic construct
  source 1..191: mol_type = other DNA
gtgttttgttatttggaaatatatataaatccatttatctattataattaatattgttcaactctatttttgtcatttttaagagtaaatatagactagaaaccacaagaagcctgttttttatgcaccagtcattttgactggtttggtagaaataggtatattcaaatcgctggtatatctagtgttaa

SEQ ID NO: 300 - DNA, 249 bp, synthetic construct
  source 1..249: mol_type = other DNA
aaatcaaaatcaaactaaattttaaaaaaagaatatcgttcctgaattattattattattttttctttaattgtttctgttgtaaaatactataatatgcttgttataaatgttttattgtatttttattatttaataaacaattatacatattgcctctcaaattgtgtaaagtatttcgtaacccgtcattttgaccgctcatggtagaactaggtatatcaaaaagtccggtagatctagtgttaa

SEQ ID NO: 301 - DNA, 190 bp, synthetic construct
  source 1..190: mol_type = other DNA
cttatcctggaatttcttgatgcaatgtcactgaaaggtagtggcaatcatattgccaccccttctccgtaaaagcctcttgtttttttctgtttttatttttttatgatcatttgtaactatataattgttatcaataaactttgagttttacatcacttttagtttcctttgccttagaaagggttaa

SEQ ID NO: 302 - DNA, 140 bp, synthetic construct
  source 1..140: mol_type = other DNA
attttttgataataaacatttcatttaaattgatttggaactattttctttgcaaaaaacttcaattttcttgtatgagtcaaattggctcacttcagtaaaaataggtatataatcaacatccgtaaatctagtgttaa

SEQ ID NO: 303 - DNA, 187 bp, synthetic construct
  source 1..187: mol_type = other DNA
atagcctttgcgtaatatgtctaaatgcatattgtagccatttggcaacaaaccctttttggatgaaatataaactttttggagaattataattttttttattatttttctgtagttgtagttactttattacaaagcgtcaaattaaaaaaaacgaaaaaaattaagtcatgcatttaagggttaa

SEQ ID NO: 304 - DNA, 206 bp, synthetic construct
  source 1..206: mol_type = other DNA
ttttttttctgtaatataatataatttaaattatcttaatcttaatcttatatattttgaataaaaaagtttttttcattaattaataaaaataacatgactattttttaacaaaaagatcataaaaagcattaaaatatatatggggtcgggttgaccccatgcacagactatggaaaatttactatgcacagactgaaggttaa

SEQ ID NO: 305 - DNA, 372 bp, synthetic construct
  source 1..372: mol_type = other DNA
aaactcaaaaaccgtatttatataaagcacttatattatttctaacatatgtataaatcaattgtcttttatctatcaaaaaatatcatatatgtatataagatatttccctttcttgtcctttctatataaaatgtgtatatgggtacccgggtaccccggctggtcaacaacgggctagtcaactttgacgatcaaaaattcaaaattttctcctaacatgtttttaagtaaatggttaaatttacgtaaaattgtccaaataacgaggatccaaagccttttaacgctattttaagtgaaacatatcgaaattgaaacaaaaagcacgaaatgggtacctggatacccgattggtcaatttagggttaa

SEQ ID NO: 306 - DNA, 275 bp, synthetic construct
  source 1..275: mol_type = other DNA
atcgacgagaggcgagaagagcacaacttacatataaaaagtatgattttgcgtaaaaatgaaaatataaacagaaacaaaattgatttctcttttttactctagttgcagttatcttcccaatggatcacaggtgatccatcataaaaattatctattttagtttcattcgaatatttgagtaaaactttacatcttttgttctggtaatttatccagatatcaaattttctattttttaactacaaaaaacgtcgtgggaaagaaagggttaa

SEQ ID NO: 307 - DNA, 205 bp, synthetic construct
  source 1..205: mol_type = other DNA
actgtaaaaattaagtcttttgttatttgacaatttccataaatccatttttgtataaaaattaatattgttgaattctttttaccattttttaagagtgaataaagactggaaactcagttaagaattttttttatacaccagtcatattgactggttatggtagaaatacgtatatttaaatggctggtaaatctagtgttaa

SEQ ID NO: 308 - DNA, 329 bp, synthetic construct
  source 1..329: mol_type = other DNA
atgagtgcacattcaagccattccattcctgaatgaaggacaattcaatgtaaatattgtgaacttcaattttcatgttgtgaacttcgctttacattctgtaaataaatttctttgctggcattatgtctattggtacatatatgtaccaacatttttagtttaaaaagccttatggtacatatatgtaccaggccgatttacccccccaaaccccattttttcagacttcaccttacacagttataaaaatgatccccttggagaaaaaaatcatctgttgtttggttgttttagcaacaaaaaaaaattcgacttgaaagggttaa

SEQ ID NO: 309 - DNA, 1576 bp, synthetic construct
  source 1..1576: mol_type = other DNA
tttcaattgtataaactatacatgtaagaggaaagccagagttctttttattgaaggttaattgagcttttgttattgtggtacatgtcttttccgcttttctctctgtgtgaaataagtatgatataaaatagaaatgctttcatgattataatgattatataaatacatcagagtattatttgaattcagaattattttgattcttggaatttagagtttttaagaaaattgcccttagttagactatccataagaaaaattcatgttacctgtctaagttagtctaaccaagggcaattttcttaaaagccctgaattccaagaaccaaaataattccaaattcaaataatactctgttttaccgttatacacaattgccttaaatggttattttaaaaaacaactggtattaaataactttgctttttaaatagcaatttcatgtacatgcagaaatatgttagatatatttttgttggttttgaaggctctcaacttgtccaaaaaatgtcaaccacttttcaaatttctgaaaagtttagtgacatgtatgttcatataaatgcatattagctgaagaatgcagtgctatgcaatgttatgcccgatttatattttgttgtatgaagttttgtttgaagctcaagctaacttagagtacagtgcctctttaaagttttgatgctattattaaagtaactatcttctctttttctatcaaatgtactgatgcatcaacagataagttggatggaaaactgtgtcagcgctcaaggttttctgacattattttttgctctatgcacagaataaaaacctgacccgatcattttataacatatttttcctactatgcaagtaattcaaatcatttctgaaattgtatgccaataatattaacataaatttcaacaaactggaagcactgtgcatgaaaaaatgtaattgatgaagtagtcagcataaataacgcattataaatgtttaaaatgtggtattgagttttaacttgctttggtgaccttgacctctttgtatgtgaccttgggaaatatcttcttaagaaacctagaaaaaatgccacctgtaaaatgattggtcttaactttttacagtgatatgataaaaattcaattaatgataagtgtgaaaatgttcttgaaattgtgtcaaatcacaaggtgatattagttcattgaaatgaaaattttatgactatgaattaattttgtattattttgttatttttgtatcatatctaaatcaattgtcctttggttcactgccatgaataaaattgatgctaatttaaaaggaattttaacgttttttctgcaattagtaaataaaatggttaccatggcaacaattctttatgaaattaatgcaaactttttcaaaagtacaaaacaaccatgctctacaactttggaaaatttcatttatatatctttggaaataaagattttatgcattgatgaagaattgatgtcatcaaaatgtgattttcccaaaacaattcagcacaggtgctctttaaatttgataacaatatagcagagcaagggttaa

SEQ ID NO: 310 - DNA, 1929 bp, synthetic construct
  source 1..1929: mol_type = other DNA
tatgatatcacattactcctcagtgttggagttttcacttagtgaagcaaacaactttgtttttatcttttgcattgaagagttacatgatacttctacatgaacatgcatgtataagtatttcaataagcagcagactttgaggagaaaacttgttcataagactgcacaatttaataattcttcattgtgtgtggatttttctttattgtatacagtccatgaaaaatataagaatacactaagattctttgtaaactttttgcatattagaatttgatattttttcaatattgtttaaaatggaaagtacgtaaactgtaaacatgaatttttttatgagtgaaataatacatttgaatcaaaaggatgttctgcttaagaaaacaataaaagtatgttcataaaaatggacaagaaatatcaacaactctaaacaagaagattattaaaatgaaaattaatgtatgcttctacagtggtgctgtctcctgaattttagtgtctgcacaaacataatgtttatagataggattttatcattaacattttagtattgtttgaatttagcaaaaaaaattctgcatctatatctatttaagcagcaattattttgcatcagttgaaacaacaataattgtgttttcagtgatacagtttgtgccattatccctaacagattttgtatcaataacagaaatattatatataatggtatgctgaacattgtattgtatgaaatttaaaatgactttttaccttacatataatggtatgctgaacattgtattgtacgaaatttaaaatgactttttaccttactgttattcattgttgtgaaaattctaaatacattttatgaaaaaaagtcttcccagtttttaaaaattttcattcaaaacaattgaaattaatgtttgtttggaatagccagtaacccctgttgttaagaattattgtcgaaatgatacttgtttcatcaatttaaatgtttgtgaattagtttgctcctcaaaatgctccactttttatcatttggattgattaaattcaattgggggaaaattatctcccagctatctcttcctttgatatgactttctccatgcggcagatccaaataaacatttttttaaaatgacagatatatacatattgatttgaaacatttggcttcaaataaaaaaaaaagtaaaaattgcaaacagagaacaatctctaaattcttgaatcaaaataaagtagagagcagggcaaacagataccatgaaaacacgaaaggtttcatttgggtaagtgtcacgcaggatggtctaatgctggtttgaatttataaacatgtactgcaatgaacatcatgtattgcattttgcaaaccaagttacatgttgaactgtattaaaaagacttttagattcccattaccctgttagatcagctacttataaatatcctgcctttattttgaaactgatcataggtggaacatgctattacgtgacaagttttatggtaaatataaagcttacaatatacgcaaaaacatacaatttactagactacacagtttttattcaagagcttcaatgctaggggatgaaaataattaaaaaccaatttttttgatttagaaattcatatgatgaaggtcaattttttctgtaaaattataaatttttcaattttccattttattgaccactaaaactgttgccatggcaaccgaatgttacaaaaaaggacaaaaatcttggaaaagataaatgaaacaattatcaaaatgtttggaaagttgcaggtttgtaactctatggatcatgattttatctgcatttaagtgtaaatcccctgattttgtggtaaattagccaaacaattcagcactgacactacttctatgacgtaacaatacagcagagtaggggttaa

SEQ ID NO: 311 - DNA, 349 bp, synthetic construct
  source 1..349: mol_type = other DNA
aaggtcattttagcgcttaatggcaaatattataaaaaatgtacgttatattgatgtaaactgactaatgaaactattaaatatattatgaatttaaatattttgtttactgtacttaaaacattgcaaataaatgcatttatgttaaatgaatttttgaaatatataaactttaattttgaacccaagcgtatttgatgtatttatgcaacataccgtttatccttaataaaagggtaaaaaatcaaaaatttgaaaaacgaaaaaaaagtgtattttttatttttacgtatctatctaataacaaaaaccgcatcaaaatcggagttgatgccatataaagggttaa

SEQ ID NO: 312 - DNA, 218 bp, synthetic construct
  source 1..218: mol_type = other DNA
aaattgaaaataaacaaaaataaccatcaactaaaaatgtattctttaatatacactatggcccaacgttgcccacaagcaacatccggtagcgcccaaactagtgggggtggggagttcaaactttactcaaatactgcttggcccaatacaaacagaatacaaaaaaaaaaatatttttttttcgacagggaaaaagttggggtcgaaagggttaa

SEQ ID NO: 313 - DNA, 280 bp, synthetic construct
  source 1..280: mol_type = other DNA
atagtgttaatctagtaaattacatataacaatgggtaatataatcaatagttatctaataaactacactttttgattacattctgttttgtcataaatggtcttttatttcctcttattatcctactgtcctacatataggacggctgtttccctgaaagccttacacttaatttttttttcataattttttttatgttgcataagatctaataaactaaataatgtaacttttttcaaaattttcagaaaattttagtttcaggcttatctgggttaa

SEQ ID NO: 314 - DNA, 343 bp, synthetic construct
  source 1..343: mol_type = other DNA
ttttattgattatgttgattttatttttttgttataacggttttttttaattctaatgttattttctgttttatttttttcaaataaagttatcttttaccacagtgttttttataatttgaacgaagttttttttcttaaaacgctatctatttaaacgccattaaattttaagaattgataaatataaagaaacaattggaaacaagtaaaaattagaatacttaaaattaacaaaaataaatttactatcaaaatcgtgtatccatatggatacacgacttgaagaaacgagagtttacgcgtgtatacatatggatacacgatgcggtcaacgtgttaa

SEQ ID NO: 315 - DNA, 254 bp, synthetic construct
  source 1..254: mol_type = other DNA
tataaaattttttgtaattattcaataataaataataaaaaagaaaatatttataattatttgatccatcataaaactgaacttttagtattaacttcccaggtccgcctcaaggcggagatcatataatcgggactggaggtcatattttttactttagaaaaaaattagcttgaaattgagttttaatagcctcaatttagcctagcataattttttggtgaagaaaaaaaattggggcactagagggttaa

SEQ ID NO: 316 - DNA, 320 bp, synthetic construct
  source 1..320: mol_type = other DNA
agaaaaaactttgttttcattgccattagcgagacaaagtatacattttacgtttaaagctaaaatatttgttatatttgtatgaacttttctgttactttattcaaactgtgaaattaaaaatcctgttccaccacgcctagatgatttactttacctgtccccgacgcccaagacctgtaaatcgcacgtatttgctacgaatgttctcgtatctgactttatgtaggaactcgtttcctacgagttaactcgtattgggcgccagtggccagttcggcacatacgagataactcgtagttggcagtcaacgtgttaa

SEQ ID NO: 317 - DNA, 348 bp, synthetic construct
  source 1..348: mol_type = other DNA
cagattccagtaacttgagagaaaagtgatacaaaggtgtgtgtagaccacgtacgagagtaatatggaccaagtatactaaatgtgtctacaagtttatacaaataggaaacttgttagcagtagcagcaataaatttgtaagatattttgctgttgaaataaataattattgattattattaattatggtgtttaataattattgtccctgctagagaaaaggtgtggattaacaaaaaaaaaaaacacttatttaagtctaatatgaaaatattttatgtgggggtcaaaatgacccctttccgccgttttaggaatgtcagaaactccgccgttctagtgttaa

SEQ ID NO: 318 - DNA, 424 bp, synthetic construct
  source 1..424: mol_type = other DNA
gcatagtgtccttttttgaaatctaccttgcaacattctttagaggaacatcttgtgttattcatgtaatcttttgtttctgatttcattgcttttgattattttaattttatttcgattagttaccagtttataaaaaatacttcttagttttgatattattcatacttatattgctgatgatatagaaataagcatatgttttatgaaataccttgtatgtactgtttatattgttcatttgttgttttcacaaaaatacaaatgcaaataaaaaaccatataaaaaaactgaaaactaaaaccaaaacattcagtggctcaaaactatgaaaagatactgtcaaaatacgatcacgcgccagagcgtgaagtggcacctagcatcatcgcgccagagcgtgaacggcagcgaacgtgttaa

SEQ ID NO: 319 - DNA, 389 bp, synthetic construct
  source 1..389: mol_type = other DNA
cgattttagaggcctatacctcacaaactattcatatgttcacaatttccatttatgtagtgataattcaacatttttgcttcttagttattgtacttattctcatttatattcatatttcaatgaaattttcttgtatattctgtttactaacatttggtactacaaagttgtatgtttttcagttttattttattataattattgtacaattcagacaggtaaaataatgtcactgtatcatttagtactatctgaataaaataaaaaatagtcattttatgaaaatatacatttcgtcacttgttaaaagtgttttatatattgggtctctcagacccagcccgaccgttcatgaaacttttttcgcccgaccgttctagggttaa

SEQ ID NO: 320 - DNA, 517 bp, synthetic construct
  source 1..517: mol_type = other DNA
agatatgatccttgacctctctgaaattactgagttgggctgtctgtaagctgttacaacaacatttcagtagacattcagtacgaacatatttgttttcaaagctgatagtaaaacataatcttttgtcagttaaattgtatgatttaaattcttggattttcatctacgtttataataagaagtgccattaactgtcacattaaataatgaaccttttttttgcaatgaaagtgataaatattatatacatatttcttgattagacatgtagtatttggaattgtggtgtcgaaataaattcgtattaattagccaaaaatgtgtaatatttcgatactattaatacctgagtgagtgatagtacatctaccttttagaaaattaactataaacattagtactgtcatgagttcatgtgaaaagtgtaatagaactgagtcaacgtacgtggcgcactcagtgcgccgcgcgactgctcctgcaccatgaaaatgcgcgactgctctagggttaa

SEQ ID NO: 321 - DNA, 483 bp, synthetic construct
  source 1..483: mol_type = other DNA
aagagtatttagtggcacacagaaggatatctttagtttgtactttttgttttataaggaaaatgtggcaacctttctgggagaatcgtaaatggaaaggagatttcgcatacaatttactataattcaaatgggatttttgtatctaatcacgctgtgaatttgtaatataatctctataatgataatctgtgggtgtgtagagaaataattacttgctgttcttttcgtagcacaaaaaataaaaatgtactgcaaattgcgctgtgttttacttgtcgtcatgttactcatactacattgttaaaaaaaagtcaaatttctactaatgccacctctaagaagtgtaatgaacgacaggaagtcatgaaaatatatatttagcaaaacactagtttaatatatggaaaataagagagcggtctgagagacccctccatagtagttgcgttcctgtcaacgtctatagtagttaagggttaa

SEQ ID NO: 322 - DNA, 350 bp, synthetic construct
  source 1..350: mol_type = other DNA
tctacacctttttatttcatgaatttattatgtataaaaggcacttcaattaggaaattgttaaaaaatgttcaaaacaagtttgttgcttattcaaaagcatctttattgcagatgtatactgaaaccgatttgcgaataattccttctgtttgtttttggtttacaaaataaaaaagtattctcaagttgtcttttgttttcattttttattcacaacactttcaagaagcgttgaagcatttgtacgcatagaaatgatgtttaaagtagcaataaattagtgcggtcaccatgaccgcacatggcctgttaagttacaaataatgacccggcctgtagagggttaa

SEQ ID NO: 323 - DNA, 349 bp, synthetic construct
  source 1..349: mol_type = other DNA
tgtgatgttttttgtgaaatgatttatgctttgtcagtggcaaaatcacctatttgaacactgtgaaaaataatattggtggtgttgtatcagttttcataatataatctattaaaaaattattcagggttaaaattatactaattatacattttaaaagccagtaaaatcattaaaatagccacagctttttatatacactcatgaacctcaaataaaaatattcgtgcatgataatatccacaaaacacttattttcatttaaattctcatattagacgtaacgggtccaatggacccagggtatgtaaatgtgcaaaaatatataggtcatgcatagaagggttaa

SEQ ID NO: 324 - DNA, 383 bp, synthetic construct
  source 1..383: mol_type = other DNA
tttgaacgtctctgatttttcatatggtacttcttcaagttaaaaacttaacattttttgtttcattagttctttaagaagaaatacaaaatataccaaagtatgaatgtcattattagctaatggaaaacgtataaatgtattagtgtacaatatgtgcagtaaccacttctgaatgcctgttcatcagtaactatttgatgttaatgttctgttttttcatgtttaattactttgtattaaagactctttcatatacaaaacagagaatttcagagttgttcatattgttaacattatacccacctataagtaagggggtcagattgaccgcaccacggtgttagtgtcccatttttgccaccacgttagttaaaggttaa

SEQ ID NO: 325 - DNA, 278 bp, synthetic construct
  source 1..278: mol_type = other DNA
aactctattctaattttgccttgtgattgtgcttgtgattatacgccaagtaaaactgttgtgcaacagatcattgagggcgagaacttacttcattgttgatattgttggcatttaatagtgtaaaatttatgaatattgtaacaaataaacctacattcataattataataattttaacggtttattttcattatacccttcaaataccacaccgggtcattatgacccgaaagaccatatgtgtataccttttttgaggaacacagcagggttaa

SEQ ID NO: 326 - DNA, 392 bp, synthetic construct
  source 1..392: mol_type = other DNA
gtaaatgtgtttgtaaaactgtaaaaaataattattactctgtgtgttctaatacgcagcttggctcaaaaagaaaggacgtgcgatatcaaagtacagagtgcactgtcagtttgtgtattattaattcttttcaaatgtatcattcgttgtagaaattcctagtaaatgtttttgtaaaaaaatacatattactttgtcatatctagccgctcgtcagtgtaatgtctttcttttgacgtgaaaaaataaaagaaaaataatcgaccgcgcgtaacaccgctttcccgacgccgcagcggaaaaaaaaaaatcgactgcgagaaactccgtttctggcgccgcatacgggtgttgcacacgctttcccgacgccgcagtgaaagggttaa

SEQ ID NO: 327 - DNA, 314 bp, synthetic construct
  source 1..314: mol_type = other DNA
actatgtttttaggtgtttttgcattgacattttctagtttttatgaaattatatgttatgtgtctaaagataaaatattgatttttagtatgatattttttgataaattcttattttgtaaattcaatttccatcaaaccaatgtactgagtactgaaatatctaatttaaagtaaagaacaacatttaagctttaatttcatatataacataaagtacatttcaaagtctgggttgtcccaataaatattctttcgttatcacacatttgctcttgttttgggcctcaggagcctgggtatgaaagggttaa

SEQ ID NO: 328 - DNA, 312 bp, synthetic construct
  source 1..312: mol_type = other DNA
aaagagttgtaaaaattcaatgaaataattttgtacatttattttataataaattctgatttcttgcaatttcaaactttttttctacctctgaatcatctaattttcaaaatataataattctgtaaatatatgatatcttacaaaaaactttaaacaaatgtttagatataggatgataacacacacatcagttaaaaggttttgctttccttcattaccttttggtcaggagccatgtaaaaaaggcgcaaaagtctacgacttctgagggattgcagtagtatctgtggcccgactgcaatgtgttaa

SEQ ID NO: 329 - DNA, 317 bp, synthetic construct
  source 1..317: mol_type = other DNA
cagtaacgttcattctcagcggtcgagccgcaaaaacctcatctctgtgagatttgtaagttgaagtggcttactagtgacttataaaattcgtgatcccggcctcagaatttccgcctgtggtgtcggacttcaaatccacgtaagtactctattttttgtgcacgagatacagcaaatcttctgcgatggcgtgaaaaaatttcgtattattacaacaaaaaatgccgtacttttaaattttttttcaatacccttaaaatcagccggcacgggggatttaaaagtttaaatggcagccggagggaaagtgttaa

SEQ ID NO: 330 - DNA, 195 bp, synthetic construct
  source 1..195: mol_type = other DNA
aaaggtatggaaagacatttattgcataaaatattgtatttttaatagttcctgaaaatttcatttttaaaaaccaatttttgttgaagttgtttagaaaaaagtaagcacatgcatcttagcgacataatgtttaaaaggcctgccaggacaggggtgggatttaaatacccttcgcgccagtcaaagagttaa

SEQ ID NO: 331 - DNA, 268 bp, synthetic construct
  source 1..268: mol_type = other DNA
aatattattttatttcttgaatttgaactaatttttataattatattttttgttcattttttaataaattttctgttcatgcaagccagcattgattttttgttcttttcccagaagccatctatcgagattagcactgatatatttcgagtttaatcgtagctgcacaaaaatggcaagtaaaaacaggacgagataactcgtagttggcgtccgagaaaagaacgcaatctacgagttatctcgtagttggcagtcaacgtgttaa

SEQ ID NO: 332 - DNA, 354 bp, synthetic construct
  source 1..354: mol_type = other DNA
agtttttttaattattttatgatgattataacctacgttaaaattagtagaatgtatgcgaaactttatagttcctttaaaaaattgtaattataagactgcatttgaattaccagaaaaatgagactgattgtaataatttctttgatatttttttctaataaataatcttactttgtattcatattttgttttacttaaggtgccaatgcacacattcctgccgcccgtgtggtgccagaataaaccctttacttagaaatggcaatagaaatcctacgaaacatctcgtacccggcacctgagaataacttaggctacgagatatatcgtagctggcattcaatgtgttaa

SEQ ID NO: 333 - DNA, 537 bp, synthetic construct
  source 1..537: mol_type = other DNA
aggcagatagcactttcgttgctcatttatttttcgacagttatgtgtcggtactattcgtaaactttgtatttattttatttgtttgagagtgtaatggataaagctctagattgtatttttcttagatttgacatacattttatttcagaagtttcttcgagatcttgtgtttgtatttgaatgtttctactatattttttgtccaataaatatgtcgtttgtcagtaggatgtttcgtttcatttatgaatataaaatgaaattgtagtaacctaaactgtcacactaaccaggaacaatacactagacgcattggcgcgtccacggccacccgctacagaatatggcctgatatgccactgacgccttagtgcgccccaaaacaactttgattcacgagagtaaatttgattgtgtacttataaaataaatacatgttatattacttttagcaatatattttcagattctattaagaaaaatattggttacgtggcaaagcaaggccaattacagtggcagtgaacgtgttaa

SEQ ID NO: 334 - DNA, 290 bp, synthetic construct
  source 1..290: mol_type = other DNA
tttagaaataaatgttttgagttcatgagttcgatttattatttctttacttatttaattggacactccgatcggagtgttcggcatctagtaaaaattttcacttgcaatgccgattactccgatcggagtgaaattttttttttctaatattttgaatggcacataaaaaaaacacatccaagactcattttaattcatatttaccacaataaaataaaaatatttggaaaaatcttaccggcgtcaaccacaaactcaaaaatatagatcggcagtcaacctgttaa

SEQ ID NO: 335 - DNA, 430 bp, synthetic construct
  source 1..430: mol_type = other DNA
gtctttttttcatttgttctttaaaaaatgttaattttgttattattttttgtgatttactcctaagaaaaattaattatttatgttttttttcgctacgttatgtgtttgcaaagttttagtgataagaattacacgtgcaatacataatataaaatatacctgttaaaagttctttgttttttattttaattctgactttaaaatttgactcaaatgttgagtcaccggcatattctaggagaaatatacctcaggggccggtgactcagctattgagtcaaagtagatttaggcccaaaaagcaaaaaaaaacacaatattaatcatacaaagtattaaaaatcctattttcgaactactttttgcaaatagtcccgacggcttctgaacaagggcacaaaatacttataggcagtcaacgtgttaa

SEQ ID NO: 336 - DNA, 445 bp, synthetic construct
  source 1..445: mol_type = other DNA
ctgtttatattttgttgatttatatgtttacatattttgagatacacatattttcctatcgttcacaaaaaatgttatgtttcaatacaaataatattttattttacattatgtttttgtttatattttattgaatgctgtgtttgaataaatatatattatattccatcgatagcttcattgtcttttcgaaaattgtcaacaagaaatttgaggctctgagaatcaacctatgattcacacaggcgcaggccaattatggtttctattagttacagccgtacattttaggctccataagtattacattgatgtgagaagcgttagttgctttagccggccactacaacgtgtagtgtctgagaatcatttcttgatactcgcggctgttatggccaaatctctatgcatcacattacgcttctcacggcagccaacgtgttaa

SEQ ID NO: 337 - DNA, 351 bp, synthetic construct
  source 1..351: mol_type = other DNA
tttttaagaactttgaaggacaacatagacaataagttttaagactgaaataattatgtttaagaagaagaattttagtttttttgtatgctcctatagaaaataaaagtcatcgttaaaaaaaaatcataactttttcatttttatacataatcaaaccccaatacactggtacattctccagggccaacgctttcaaacgccatactaaaatgtgtggaacggtgtattctgagctacatactggcccgcgaaattggcatagtactgtgtatactcaagtatacaccggcctgtgacaactcaccccttggtgtatacttaagtatacactggcgctgaccgtgttaa

SEQ ID NO: 338 - DNA, 387 bp, synthetic construct
  source 1..387: mol_type = other DNA
taatgtaataaaacatttttctttcatttaaaatagcttttttatttctgtaataaacccccagaaatcctataaattttaagatagagcaatttttttttttttaaattgtaggtataaaagtaaattttgtgtctggttgcttcacttttctgtaatagcaggccacgcactacaaaattaatatgaaatcacgaggtcgtagcggaccgcacggcctgctagtaaaaatacattgtgagcccgccagccgcccgtacggcctggtaaactcgttaaacatttttcaccgcggcctgcactggtgggcaaagatgcggccgccggtagaccgcatggcctgtaacgctgaaaaaatctatatatggatggcggtgagagtgttaa

SEQ ID NO: 339 - DNA, 353 bp, synthetic construct
  source 1..353: mol_type = other DNA
aaatgtttatttttataatagtttatatgaatatttgttaagttgcaaattatttacttttttataaaagtatttttatttttatcattattgaaataaaatggcaaaactgttccgccagcccaagccgttttaattaccttatttctaccaggccagaacactattccatgcacttaattcaaatacaacttttgccatatgtgactgacatgcctttaaacatataaatgactgtcagtcggatttgtctgacaaagcctcaaatgtgagtataggtgtcagaccaatacgactgacaaaacctgctgaagaacagcatgaaaaacactctggcagccagagtgttaaat

SEQ ID NO: 340 - DNA, 368 bp, synthetic construct
  source 1..368: mol_type = other DNA
gttattttataactaaataataaaaagtataatgaaattatctttatttttattaatatttttattgttttcttttgtatttttcatttcttcaacatgaataaagtttacctttttgttaaaaaaagtcatttttatcgttttatatcttggaaattttaacttttttcttatacgtatgttcaggtcttatttaaaaaaaaacattctctacacgctcagagcattagattactttattttccatcagttcatacaaaaatttgttaaaagcaataagacaaattgtgtatcacatatgatgcgcaagcgtattaatcataacgtctgtgcatcatatatgatacatgggacagtcaaagggttaa

SEQ ID NO: 341 - DNA, 287 bp, synthetic construct
  source 1..287: mol_type = other DNA
aaaaaaagttttacaacatttgcaatatggattaaaaataaatacattatgagttattttgtgaacttgtgacaaccctgtttataaattttgttcaaacaattttagtgattttagtgaagaaaacattgttattttttcgaaaaacatcgtgatctggtgagttttcacatttttaaatcaagactgtttataattaattcatttttattaaaaattattaatggtaaaatgcccggcgtacgggtactatctaagttttatatggccggcagggaaaaggttaa

SEQ ID NO: 342 - DNA, 433 bp, synthetic construct
  source 1..433: mol_type = other DNA
cttttgtaaaatatcataggaatttataaagtttttttattttgttttatttgatggtttttctaacctcccagaatgtatattttttgtttagttgtttattttgtttataaatttgagtttttgttattataaattttttgcatggaatatgttttttttattccctagcaaaaaaaaattaaaaagatgttttacaaatttttaacttataccagggtgttttcaaagttgagtatttcagtgagagtaactcaacttcttttggcgagttctacaagctggtaaaataaatataatatataaataaataaaaaaaaatactaatttaaattagccatacgctgctccggacgtagatatgcgtttcgggctagaaagtgccgcacggccggcacggcgtaaaaagctaacttagcagtgaaagggttaa

SEQ ID NO: 343 - DNA, 443 bp, synthetic construct
  source 1..443: mol_type = other DNA
aaagtaagttctacataacaaatgactatttggttttattttttgttttggataattttgtttttcttttgttatggataattttgttgtttttttgttatggataatttttttgtttttatttgttttgttaacatttgtttacattgaatttgaatgcaaaaatatgtgcaataattttttaatttgttttatctatctttctatcctaagtttttctaaacaatagcgtttttgtatttttttgcaaaatttattttgtttcaaaaaaaataaaaaaaattataaataattacccaattattttgtttatataaaacaacgttataaagaatcatttagtcgcattatttagcgcgaaagcggccgaccgttttgacgtcagaaatagccgcactggggcatgagtatcgccggagagagagccgcatcgaaagggttaa

SEQ ID NO: 344 - DNA, 240 bp, synthetic construct
  source 1..240: mol_type = other DNA
ttccagagagcatgagtgtgatgagtgaatgaaaaagttgagtttcgaccgctaaattttaaaggtaagcgaaagcaacatttatatacaacatataaaatttttaaaaatttatttgatgtactttatgagttattgcattttaatgaagacagtgtccaaaccgtgcatttgagacaaatttaaataccgccggagggtttaaatacccaaacataaaaagccggcttgaggggttaa

SEQ ID NO: 345 - DNA, 331 bp, synthetic construct
  source 1..331: mol_type = other DNA
actagtgggcaagttaggaaacagtttttttaaattcaaacttccctttattttataaacttaagtgtctttaaactttttttatagataagttatagaatttagagagttgtggtttataaattagtgaatgttataaagtgttcacggtacttctgttcaatgtaccgaaaattgaaatttttgaaaaaaaggccaaaataatgggtaattattattttttgtatattttttgtattttgtaatgggttttacattataatttaaaaattaaaaaatagtcatttttgaaaaaaaaaatcatcaataaattggtcagttaatgggttaa

SEQ ID NO: 346 - DNA, 235 bp, synthetic construct
  source 1..235: mol_type = other DNA
attttgtttgaaatagcaataacattatttgtttaaattgggtatgcttatattaatttacatcatggggcacagtatgatcatactttaaatacctgtaatgtgcttaatactatattaaatatatttttgtaagacgtatttactttttcaatccatttttaagctaaacattataaaaacccgccctgggggtacaacttggggaaaatttaaacaccactgaaggggttaa

SEQ ID NO: 347 - DNA, 265 bp, synthetic construct
  source 1..265: mol_type = other DNA
gacgataaggtaagatgaaattgaaaaatatatatgcagtatcgtatttttacgaaaaaagctctttgtacaaaagaaatattaaaaaggaatacattagataaaataacattggaataggaaaatgatttgcacattgtgtcacgacgcatgaatctaggtcaaaaattgggtccatgacggggatttagggtatttaaatccggacctcaaagggataactcaagtcgggcggtttcgggcgcttgggtcgggaaagggttaa

SEQ ID NO: 348 - DNA, 233 bp, synthetic construct
  source 1..233: mol_type = other DNA
tatactacatttgatttttttttcaataaagttgcatttcgacgactaatgggttatatttttcccactaaaaaagtctgactgggatatatatgtcccatacaaaatggcggacttttttttcgaataacattttaaggtgtttatttcacttaccttcatcttgttgttgtttaaaaaaattacaatctaatcaggatttaataataaaaaatccggctcgaaagggttaa

SEQ ID NO: 349 - DNA, 265 bp, synthetic construct
  source 1..265: mol_type = other DNA
atcttttttctaccattgtaaaattttagaataaatatgtatgctgattttggatatatttttttatttcccacacatctagagggacatatatatcccatcaataaaacctggagggacatatttgtcccaacgtcatatctaaaaaattaaaagtacaaaaattcaaatatcggttttaaccaatttctgacaatacatagagtaaagatttaaaaaaaaattataattttcaaaaataaaaaagtcgacgcgaaagggttaa

SEQ ID NO: 350 - DNA, 208 bp, synthetic construct
  source 1..208: mol_type = other DNA
gaacggggcagtggtacatatatagcaccagttgttttttcacatttatgtacaaaataataagtaaaaataaaaaataaacaaagttactagtattatttatgttcttaagttatagtttacagaaaaataaacataataagaaaaaaaaaatctgtgcccaaataaagaataaaaattttaaatttgttgcccctgaaagagttaa

SEQ ID NO: 351 - DNA, 255 bp, synthetic construct
  source 1..255: mol_type = other DNA
tttcttttacttttaagaatatattgtttattcatattttatttttaaatttgtgttaaagtatggtggcgcaaaaaataaaccacccaatctaagttcttttgcaatgtatttatatttttttatatataaatttgtgttcaagcttatttacttaaaataaatagacaacaatttgtgcatttttcaaatgttttttttctcgtaccgctggggttttttttaaagccaaattcccgtacttcaaagagttaa

SEQ ID NO: 352 - DNA, 249 bp, synthetic construct
  source 1..249: mol_type = other DNA
tgttaattatttacctgtttgttgaaaacaatatttgtaggtattgttgctagttgcacaatgctgcaaaatatagaccaccttgtaagtgacactttggcaatatttttttatttttttttatctgaaatactttgttttgttgttatttcttggaataaacttaaaaaaatatatattttttattttatttttttcgtgcaactaggccctttaaaatgtgtgaaatctcgtgcagcaaagagttaa

SEQ ID NO: 353 - DNA, 301 bp, synthetic construct
  source 1..301: mol_type = other DNA
gtcgttacattttctaaataaattcaatattgtaggataaatgcagttttatttctccaaataaataaaaaaaatcacaataacataacaaacaatacaacatttatatctgtaataatattgttaaaatggctccgtaacggaccatagggacacatatgtcccaaaaatttcctgaaaacctgagtttttttttcccaatttctctttcatttatgattatttgatcaataataagccaaaataatccgcttttatcttaaaataacttttaaacaccttatagtcctgaaagggttaa

SEQ ID NO: 354 - DNA, 203 bp, synthetic construct
  source 1..203: mol_type = other DNA
aaattaaattaagaactgttatttgtatttgtttttcctatctgcccaatgacgccatatggagtcattgttcaaaatcaatatttccgctccttttatagaaatacaacattttacggtaaaaatgaacataccggagcacaaaaagtaatacaaatatttttttctttcaaattctgaaaaattgggcatgaaagggttaa

SEQ ID NO: 355 - DNA, 229 bp, synthetic construct
  source 1..229: mol_type = other DNA
aatattatacttacataagaattaaaattatttttacttacctttatataaaattaatccataatgttccctttcgggaacatttctttttttggctaaaaataaaaaaataaaacaaaaaaaatgtttttaatacgtatacgtcgaaaaataagatatacagcataaaaatatattttttccagaaaaaaattttttttttggaaaattcatgcagtagagggttaat

SEQ ID NO: 356 - DNA, 228 bp, synthetic construct
  source 1..228: mol_type = other DNA
aattactacattttctgaactttagtatttttttaatcctttgttttgttgtatgggttcataatgttaccatttggcaaccaaaaataaaatgttgaaaaacttcaattttgccgaccaaaattattccaaacccaatataagttatttcaaagatcattatccgtttgttcaaattttttagaaacataattacaaatttcgataattcatgcattagagggttaa

SEQ ID NO: 357 - DNA, 202 bp, synthetic construct
  source 1..202: mol_type = other DNA
atcaaagtctatttggtaaaattgggtattttgtatgcttataggcctactgtaccacatacggtacgccccctcgtgccccccccttttatttaatttttttttttctatttttttttgagtctttaggtatactttaataaacttttgttaaaaaaactaaacttttatacaaaaattgttaccggtctaaatgggttaa

SEQ ID NO: 358 - DNA, 180 bp, synthetic construct
  source 1..180: mol_type = other DNA
attcgcaccccgtatgtgcccattgtactcatatgagtacagctcatatttcttagaattagaatatttttgaaattttattttattttatcctattcatatatcaataaagaactcagaaaatataacaaaaatttaaaatactaattttttagcaccttgggcggtaatgggttaaaa

SEQ ID NO: 359 - DNA, 161 bp, synthetic construct
  source 1..161: mol_type = other DNA
gcccagtgtccccatttgagcacggcacttttttttctagtaaataaaaaaaaattaaatatttttttttgatttttgttaaaaaactctattccctactacataaaatggatttattcttttttctacgattactttagtcttgggcgtaaatgggttaa

SEQ ID NO: 360 - DNA, 304 bp, synthetic construct
  source 1..304: mol_type = other DNA
aaatattttatttatgacctaagttttttatttgtataggtataacaaaaataactagtatattttcacttattttgttaaaaatactttaaatagatatagttttgtagtattaaaggagtagtatttttatacacccattagcccagtgtacttatttgagtacggcgccatattgcattcctaaaagcccagattgccatttttttatatttctaactattacttacacccaactgagttactttttctaataaaattgggtattttattattaatttgcatttgggccttaatgggttaa

SEQ ID NO: 361 - DNA, 191 bp, synthetic construct
  source 1..191: mol_type = other DNA
cttttcaccgttgttttattcaatgttctaacttaattggtgatttttctgtttgtaataaataattttttgaaagcaattgtttacgtatataaaaacttaccgcacaaacaatatacagatacgacgggcctaacaagcccgaactgcccggtaacgttagaaaattatcttgcccggataagggttaa

SEQ ID NO: 362 - DNA, 233 bp, synthetic construct
  source 1..233: mol_type = other DNA
agatattctagaaaaattcacaatgaataagtgtaatattatgtgttatattaacaaatattgcaataaataagcatttgttgaatggtatgggtttaattttatttaaatgtatgctctaaagagatgaattttaaaaatatttgcattttttgagagaaaatgtcgcagggcctcggaggccctgcgtatgcattcgtgtagtttcataggcccatgcatcctagggttaa

SEQ ID NO: 363 - DNA, 269 bp, synthetic construct
  source 1..269: mol_type = other DNA
agatactgaatattgaaaatctcagaaaatgtgacaagttaaattacaaaaaaaaagtgtttgtgaagaaggaaaaaaatattaaatatagtgttggaataaaaaaaagtattgtttgtctctttcctaaatgttgaaatattctaaaataaagttgatatcagtttaacctgttttttttattgttttgagtggatttacacagtatgggtcaaaatgacccgcaacataatcaaggtaattttttttcaacataatacgagggttaa

SEQ ID NO: 364 - DNA, 222 bp, synthetic construct
  source 1..222: mol_type = other DNA
ttcattaattgcataaaaaaaagttttttgtaaaatgaaatgtttttttggttagtttttcgcttttgttattgtttttttaataaatattataatttcacacaaagcaactaattctatagcttctactgcacactattatgaaataaaaacatggggtccagaggttaccccacttaggcatttacgtgacaaaaatatactcaggcctctaaaggttaa

SEQ ID NO: 365 - DNA, 198 bp, synthetic construct
  source 1..198: mol_type = other DNA
tttatagcaattgtttaattattgtacaataaacaaattactaaagttggtttgttctattttaatataagtttctaacacttaaaatagtcaaattatataaacgggcttaaacaaatagctgaaaaacactggggtgcatttgcaccccagttgaccttctgacggtgttctgtgagtcgaccttacgaaggttaa

SEQ ID NO: 366 - DNA, 338 bp, synthetic construct
  source 1..338: mol_type = other DNA
caaatcagtttattacttttcttactatttacgctactgtattacaagatttcacgaacatttttgtttaccctataatatagtatatctgtgaactttataaacacaaagtaagatgtatgtgattcgaaccctaaggggaatattgagttaaacaaaacaattaggtactaaagtacaattaaacatttctttttccatattactacgctctttattaaaatcctatccatttccatcaattcctggccaataaagtcataggaaacaattctgggtccggcagacccagagtagcaacgaagttgttcttatgcgcagtagcaacggagggttaa

SEQ ID NO: 367 - DNA, 237 bp, synthetic construct
  source 1..237: mol_type = other DNA
tttttaaatctgaaatctgggattgatatattatttcatattatccgatattttctgcatttttttattatattttgttcagttcagtttaataataaaatagaattacagaaaacttgcatttactattatttgtgcctaatagattgattctgaaacaattctagatatgcggactgtgactccgcgcgcgaccgttctatgccaaaatttaggcgcgactactgcagggttaat

SEQ ID NO: 368 - DNA, 310 bp, synthetic construct
  source 1..310: mol_type = other DNA
tgttctacttctgagaactcattttatacttacgtagtatagtgctaccactagaaaaaagtttatttgatacactgttttgtatttttagaaagaatgtaatgatttgttttatactcaatatttgtgttgttcatataattaataaataaataaaatcgtgttcaatagcgtgtctcaaaatatttcacaaaatttacatagttttaccgagtattcttttctaatttgaagagaatgcaagtgcggagtatcactccgcacgttatccttcttgtcagggctccacacgttatctctcgaaggttaa

SEQ ID NO: 369 - DNA, 389 bp, synthetic construct
  source 1..389: mol_type = other DNA
ttttaatattttattttgttattattttgtagaagtattaccatttatgtttctttattttatgtttccaatgtcataagacagactaaggctgatttcatagtatttatcccaaatttgtttctaggtttgttagtttaaaataatgttgtcgtaatatcttatagttataatgcatccaaagatattgtgctaatgctaataaaagagatttgatttatatttttgcctatattttattgataatgaatgtcctattttatatccttttcaatttaaaattgcataaacaatattaaaaaagaaatacaaggagcccaaaaccccggtgcaggagaccggggtcaccgacgatgtaagctaaatacacgtcaccagtggcgggttaa

SEQ ID NO: 370 - DNA, 347 bp, synthetic construct
  source 1..347: mol_type = other DNA
gtcaaacatagataaatatgtttattgatatgtttgttgttattaataagaagtttaatttgtttttaaaaagaagttcagattttattttaattttttagtacttaagaaaataatctcaaattttaagactgatttgtgataaaactataagttggtaaaataattaataaaataacttttagttatactgttttgtttcattacatcctaaactttaaaataatagtaatactttatgtgtttcttgttaaaaaaaatatgttgtaggggtacaaactatcggtctgtgacaccgtgcgcgagtataggtgttacaaaaattggcgcgagtataggaaggttaa

SEQ ID NO: 371 - DNA, 389 bp, synthetic construct
  source 1..389: mol_type = other DNA
ggcattttgttagtacacaaaaattcaacttatttgtttaaaaaaaaatgttagtagattttttttaagtgtatgctacttttttaatgtataattgttgttgttttaatattctggcagttagttagtaatgtttttattttatttaaaaaaatgtatacaacttttttataatttgtcaataaatttcttaaaactaaaatactattttattacgtacctaacctaactttatagccattcaatagagcctagattacctataaattgaatttattaaactctagtcttatcttctgtccttgaaaataataaaaactttaatgggtctattaggcttggcgccccgactgacgttacaatctggggcgccccggctccagggttaa

SEQ ID NO: 372 - DNA, 320 bp, synthetic construct
  source 1..320: mol_type = other DNA
gcaattttatatataagctgacgaattcatcagaaattatgattatatttttgttaaaatatatatttttaacgaatctagtatttttagtcacgaaattaggtagtcacagtatcatcgtcaatttttcaaatatataacaagttcctgatcaaaatcaagttttttgtgaacagtatatagagatgttgaagactacataccaaattacatctgaaaaaatttgcaactgcagtcaatattttttttaaattgagggtctatcagacccggcaccaccagttgtgatcgaatatagggcgccactagtgtaaggttaa

SEQ ID NO: 373 - DNA, 381 bp, synthetic construct
  source 1..381: mol_type = other DNA
atttgagaaacatttattgtaggaatatttacaaatgccatgttgctattttttcatatttgattttcttattccaagtaatttttaatgtaaaataaaacaattttgtgacactatcggcgtatatttttttttcctattgtggcctaactttttataacaataagtgtagaaaaaattatattagggtacatacaaaatttaaaatcatttatagttcaatatttctttaatttaaatgtttaactttatgtaaatactatattcaaatcatatactcatccctttgtagctttgaaagctcttaaaacaaagcgtgagcagtttgccttcgcgcgaccacttgtgttctaaaagtgggtgcgacctctgacgtgttaa

SEQ ID NO: 374 - DNA, 396 bp, synthetic construct
  source 1..396: mol_type = other DNA
ggccaattgtatgaatgaaattttgtatttttctttgctaatttttgttacttgcttttttaaattaattttaagtttgtgttgatctcaaatagttgtgcaatagaagaaaactgactgattttggcagaaaaaaagaatttaatcataagacaaacatgtgactacgataaaaattgtattctgttgttttaaaatttatttagcacaaataaataattaattttcaaatttctctttttttttctttaagacacttttaagtaattgaaaacgttaaaataatgtataaaatatatgtgcaaatataaacggaatgcgtttatttttggggtctctgaagaccccacgcgacgcctcacgtaagaaaaaaaggcgcgacgggaggagggttaa

SEQ ID NO: 375 - DNA, 342 bp, synthetic construct
  source 1..342: mol_type = other DNA
accaaagttaattattattattcttattttgaatttttattattagactaggtatatatactagtcttagatataaaaaagggttttgtgttcctaatttttgttttgatttatagaatgtttgagttatgtttattaatgtaaaaattgtttctttttcataattttaattactttttttgacaaattacactttaaacacaatcataatgtatctgagtacaaataaataagagaatatcgaataattagtgctttatttgtcttattcatcccataggtcttttcggttacgcgcgcccgctcccgttacaaatgaccgcgcgcccgcttgaaggttaa

SEQ ID NO: 376 - DNA, 467 bp, synthetic construct
  source 1..467: mol_type = other DNA
tgaaagactacaaagataccaaaaatttgtctaattctaagataacataatttttgcagagcatttttcgtgataagttttgaggctgattgttatagaactaagattttctaataacttataagatttttttaatgcaaagatactcatttgttgttataaaaaacatagttataattgtttattataaataaagatatatattacctaagtttcctacattgaagtattaaaaatcaagacattctagactatttttcaaattaaaattaaagttgattgtttcagtaaaataaataaacatgttaaataatattacgtgttttattgttaaataaatgataaagtcgcaattttattaaaaagatttgctttttaattcaaattatctatatactgtctgggtcctatcgacccgcgtgcgactgttggtgtcacaaaattaagcgcgagtactggcgggttaa

SEQ ID NO: 377 - DNA, 502 bp, synthetic construct
  source 1..502: mol_type = other DNA
tcaacttacaactagatgataatatatacataagcataaattgtaaataattttttttgtgaggatttcacaactgatttcagttttaaaaaaaaccttgttcttgttttttgtacttttttactataaaaaatgatttttaaatgaaaatctttcctgtaatatgttcctaataaatggttttacaaattataaaatttggtcctattgttgtcctgaaatttttttcgactattccaacttaaactttgcttattttttgtacagtcaaaaacaaaaaaaacgggtctattaaaaatatttttttactttcatttaaaaatgttttcaatataaaaacaaatataatagtacataaaaaggtgcaaagaagtatattagaatttatggtatctgtgtaccttgtagaaaatacaaaaaaaaaaaaattactcgctgggctacaactgtagcccacgcgagcattggtgtaaaaaaaatagcgcgagtaccgaagggttaa

SEQ ID NO: 378 - DNA, 267 bp, synthetic construct
  source 1..267: mol_type = other DNA
aactctgagtttaaatcagtcagtttgaactttgacatgcaaaaatgtttgaatttatgaattgagtcatttatatattacagtatttatgtaacaataaagtcagtgtattttgttgtaaaaatagttttatttcatagataaccctaattgaagttataaaaatgttatatagcccctaaaaaaaaaaaaaaaccgtgtgggatacgactgtaccccgcgcgactaattatgtctttattataggcgcgaccgctggagtgttaa

SEQ ID NO: 379 - DNA, 276 bp, synthetic construct
  source 1..276: mol_type = other DNA
tcggattatgtcaaaaatcttaattataagtataagtgccatttttctttagttttatcagtatgaagccaaatactttttattttttgttttagtacagaagtgtctatttttttgttgttttttgataagtcaataaaggctgattatatataaaaactataatattttatttctattcctatgttactaaaaaaatcacaatattttgggccacaattgtgccccgcgcgtggaatcgtgtcactattttaagcgcgtgtactggagtgttaa

SEQ ID NO: 380 - DNA, 419 bp, synthetic construct
  source 1..419: mol_type = other DNA
aattttaactgttgaaaaaaatatagctataataaaactttaaaaaaagctattaagatttttgtttttaaatgtttgctttgctattatgtttttagggatgttaaacaatataaaacaattaattagaatcattataacctttatcagtttccattattttgcttaaaagaaaataattgtagttttagaataaaaattaaatttttcatacaatgtcaaataaatatagttaattaatatttgtggattattttacccctacataataaaagaaacatatttagattgttatggttggtagagcaattttttacaactaaaacatgatacttattaacggccatgaatatggggcaactatttaccccacgcgagtactggtgtcataaaatatggtgcgagtactggagtgttaa

SEQ ID NO: 381 - DNA, 238 bp, synthetic construct
  source 1..238: mol_type = other DNA
taggtcgataaaatattttcttttgaaatattaacatatttagtttcgttttttggttaagaaagtcaaatatatttttttaaaagtactgttatttatcatttttcataaccttacagcaattccttaacatttatataaagaaaacccctcttacatggagaaaacttgatgacgtaaaatttacgacaagcgcccgctgaagggaaattatacagcgcgcccgctggaaggttaa

SEQ ID NO: 382 - DNA, 342 bp, synthetic construct
  source 1..342: mol_type = other DNA
aaggaagtaaactgttttggcttttgtttgtttgttccctttgttttttcaaaagtatttattgcaatctaaaattatctgcaaatctcctcaaaataactcagaaaataccaagtatttgagtgagaatcagtttaaaaatatatcttatgtacaataacatgtcaataaaaatgtaggttttttatttatatgtaattataaattatatatactatttaccccataattctcccaaaaaacaaatatagggcacacttttattgaatcagaatagtcgggtccatatgacccaacgccagtgtaagtgtaatagatctcacgccagtagttaagggttaa

SEQ ID NO: 383 - DNA, 242 bp, synthetic construct
  source 1..242: mol_type = other DNA
aagttaggtagatattgacgaggttacttatgtttcagtttaagaattgatgttgtgttctttataataaaagtattttcaatattatctttatttgatttcaagttaaaacatttatttcaagtaaaaaattactttataaaaaaaaacagctgataaaaacgtacaatagacaaggagtctgccagactccgcgtgtcggtgtatgcactcaaaacgagacgtgtcgtgttaagggttaa

SEQ ID NO: 384 - DNA, 389 bp, synthetic construct
  source 1..389: mol_type = other DNA
cttttttaacatgtgcaattttttttagataatgttgtttatgtaatgttaagtttttttttgtaactccatacctttatctttaagaaaaaataagccatattattttagaattattactttttgtttaatgtttattttaaaaaaaactgtaaaattttgaaaaaaaagccttatgaaataaaaacatttaaaaaaatatatatatttactggttttgtttgtttaccttcgattttcattaaaaaccttttaatatttattataaataataaaacaatatgaaaaacacaaaaatatattttttggcggtaattatatttgcggtcaccatgaccgcactacatgcgtaacgctattaaatcgacactacagtagttaagggttaa

SEQ ID NO: 385 - DNA, 223 bp, synthetic construct
  source 1..223: mol_type = other DNA
atattttgtttactagttctaagattagtagctatttttgtacccatttgtttattaaatacaaggtatattaataaaagcagtttttttattgttaaaaaaacaaaataaaataaaaaactaaaatataatgtagatatttacagcgcgcgtcgagccgggtctctgtgaccggatgtttctcattatgtactatcgaccaatgtttcggtttaagggttaa

SEQ ID NO: 386 - DNA, 166 bp, synthetic construct
  source 1..166: mol_type = other DNA
attttgcactaattgttaaaatatctatatgaataaactattttgacattaaaaacatttttttttatttaaaaagtggtttcatttttaagttaaaaaaatgggtcactgggacccacgcatagtaaatatgtgttttatttggggcatagtacttaagggttaa

SEQ ID NO: 387 - DNA, 420 bp, synthetic construct
  source 1..420: mol_type = other DNA
aattttaggagtagattttagacttttgatttgcgttgtcagttttttattttttttttattttaaaagaagttcctaatttttgttattaagattatataagaaaactatgagaaaaaagaaatttaagttatgtttacgtttttatataataaatagttcctaaagtaacaagtgcctaactaagtacaaaaaaattaaaatattttaaataagtaaaaaaaaatgtgatttatgattgtaatataccttaaaaactgattgatctctagattcaaagttgtatttctataaataaaacaaaaaattataaaaaaaatgttttttcattttatttttttatatttttttagtggggtcatgtttcaccccccgataccaataacgttactaattttagcgaaaccagctaagggttaa

SEQ ID NO: 388 - DNA, 402 bp, synthetic construct
  source 1..402: mol_type = other DNA
ttttgtaggcacataatgaagtgtcattttttttgtattgttagttttttctcttttataattttaatttgattttatgctccacttctgtacataataaaaaagaagtttaattagaagtcaatatttgttgatttttatgtcattttccctgacctatacaaacagaaaaccaaaatggcatgtttttgtgaatggctttaagtttcggataagactgattaatataagcacaataaacgactgaaataataaatacgttatcttttattatgtaaatataaaaatatgcaagttatttccagtaattacttgtaagcacacctaaacacggttggggtacttctagaccccacacgtgactgtacgttaccagaaacaacgcgtgtacttgaaggttaa

SEQ ID NO: 389 - DNA, 290 bp, synthetic construct
  source 1..290: mol_type = other DNA
taaagaaaaatattttaactagtcttactagtcctgaactagttttcaatgtacctagtcttaaaaaaaaaattatgtacatacttattttatttttaattttttaaatagtttatattgtttgctaaattcaataaatacgtatttaattacaagaagtatttattttacctaatattgacaaatattaccaaaattcaaagattaaaccagattttttgataccgggtcggaagtgacccgctagtgcttgaaacgttaaatattgcctagtgttgactgagggttaa

SEQ ID NO: 390 - DNA, 275 bp, synthetic construct
  source 1..275: mol_type = other DNA
aaacaacaatgaaacaacaaacaagagagtgattcagaattcagaaaaaaatgttgtagttcaacaatgttcaaatttaagtggcttattgttacctatggtttaattatccaggtataattgattgttcacatcctaatttacctttttcatctgtttttaataaaatttgaaatatttctaacaattaaattaatgtttctaaataggggtaaaataataccccacgttacacattgtgttcctagaattgtatgttactcatccagggttaa

SEQ ID NO: 391 - DNA, 179 bp, synthetic construct
  source 1..179: mol_type = other DNA
ttttttttaaccttttgttttattatgtggtaatttaaaagacattccatattttaagtagactttttctaaataaatactctagttttgataaatattgcggttttattttgttagtttgtaggactaatgtttagtgacagagtttgaaaaaaaaaagtttagtgatctagggttaa

SEQ ID NO: 392 - DNA, 389 bp, synthetic construct
  source 1..389: mol_type = other DNA
aagtctgagttcgacttagtctgcagtaagctatgtgatgattagactagttttttgttatgtttttcatactttatatattatattgtagtttttataccaaaacagtggtccaatgtcaacatttttttttttaatataatatcccttaaggctgtgttagaaagttaagtattatgtgtaaggctgattttaagacaaataagagtttctcagatatatatttttttgcatttcataaaggcagaggtatgcctgttatttgttataaattttgttacattttaataaaagtttagtactaaaattttgctttatttataaagtagcctcccaggcgataattagtattaagcagtaataaatttatattagtactctagggttaa

SEQ ID NO: 393 - DNA, 351 bp, synthetic construct
  source 1..351: mol_type = other DNA
gtaatttgatactcacggataattttaatcttttttcttcattatactgtaagtaaaagtagaaatttctaaataatagttcctaatttagtcgttgtaagaagttgtgccttattgatctcaatgaaacatgtgccatttaagatttgtgatttagtacttaaatattaaataaatattcatttttgttgtaattatctttaatttacctcccatatttcctataaatataataaccctaattaaaataacatatttttaaaataaaaaactcttttgaaaaatgctcgtcaaaaagtcgaatggttagcttatcatagaagtccaggaacgttagctatctagggttaa

SEQ ID NO: 394 - DNA, 359 bp, synthetic construct
  source 1..359: mol_type = other DNA
ttttttttttaatcgttatatagaagtttatttttatgttttttagtgttgatttagtaagagatattactgcttttgttataataattacttagaagtatattgatctcaagagtagactgtgtaattcatgataccaaagtgtattagatttagacctttagaaacaataaagttactatttttgtatattgtgttttatttgaggtgccaagtagtataaaaacgaacgttgcaggaggccaaacagtatagatcaccctacaaccaacaaagaacatctacgtactaacacgtagttggcctctcctgttactttaccttacgtaatattacgtagtcggcactcaatgggttaa

SEQ ID NO: 395 - DNA, 502 bp, synthetic construct
  source 1..502: mol_type = other DNA
attttgttcgtgtttgtctctatttgactatgttcgtttatttgatagagtgattttgtattttgtttttgagttttcatatttttcaacatagaattttattatgttggatttgaataaagtttagtttcagtttttttgttgccatatttgttttatttttcttttattttctgtgttttattgtttaactttaatatttatcataaattaaaatatgtatgtgtatcgtcttattatttacttcgactttttcgaaaaaggatatattatatgtcaattgaacgccgcagaggcggcatcgggcacaagctaagtagttggcttgccctgtgccttgcccgcccctagggcggccttgttttttttacaaaaaaatggccaatatcagctaagggcactaacctaaacctaacactagccttaaattaaaaaattattttaaaaaaataggcggcacacatgtgaccgctaggatttttttgggcagtgaatgtgttaa

SEQ ID NO: 396 - DNA, 482 bp, synthetic construct
  source 1..482: mol_type = other DNA
gcccaaacatccaatttatctcaagtacacttttgtttgaatttatttgtaatctttgtaatgtaaaagtttgatttatatgtcttataaaatatgtaaaataaacttgttcatggaataggtgtgtttttttgttaaaaattatccttgaatctaaagtatttcaacaaataaatagtggctcatatttgggacactcggtctatgataccattttgatgctatagaccgaccggctcatatatgcgccgtaataaaataatgctaaaaaccgcaaaaacagaaatatacatcaaatatatatataattagggttattacaaagtataataacctatttaaacaaaatcttaccatcggtctgtgatgaagggcacaccaatattaacttggtctgtgtgtaaagatcgtatttctgacgtcggtctctcgtaataacccggccggcgcatatatgcgccgctcggtagcgaacgtgttaa

SEQ ID NO: 397 - DNA, 493 bp, synthetic construct
  source 1..493: mol_type = other DNA
tatgttgatgctataaaaggtttcttacctcatcttttacagcatgttttgtcaaatattatggctaattatttatattcctaatggttgtttgtaattatggtcactaatgagaactttattttatttattttatttttcataaaaactgattgtatagtttgtgtttcgttaaaaaataaagactgctaattttcgtttaaataataaagactgatttgaattatatatcctgaatttttattttggttacttttttcgctatctttaaattttttcaattccttacaaggtatgtttgtcccactgcgcagtatagttctacagcgccgtgccggtcacggagtacgaataaagtaacagtcagtatggccggcggcgagaaaaagtagtttgaatatgactatacgttgaggtcaccggtgacccccatggcgctatgatagaggaagggtgaggtcaccggtgacccccacggcgtttaacgtgttaa

SEQ ID NO: 398 - DNA, 316 bp, synthetic construct
  source 1..316: mol_type = other DNA
caattaatcttattttgtgtctttctttgtagttgtttttttaatataattatgtagaaactaattcatgactttttttttcaccttcaattttccagtaccaatattttttggcatatgtctcataacccaatcccaaatatagcaaaccaaacgtccaggccagaggatattatttctaatacaaaatctatgaaaagcgtgtgcattattggtacacaacgctttgaacgcaataggtcgtgtgtatcatttttgatacactatgttttaacaagcgtgtgcaaaaatgatacactggcgttgagggtgttaa

SEQ ID NO: 399 - DNA, 351 bp, synthetic construct
  source 1..351: mol_type = other DNA
catttttactatgtacctaagtatttgtatgtaacaataaactatttaatctcctgtttacttgtatttatattttgttacaacttcaaagtattcaaaaacttatttggatttgagtcagagtaacgcggttgtaaacataggtacttgttgatatctagtaacaaaacctccattcagattaattaaatgtcgaaaaatgtacacgtcaagccaaaaaaaccaatgacgtaattaccggtcctcgaggaccggtaccgcatacaggaatagacatgttgccggtccccagggaccggtaacgcagacaacgcaacgctaaaattttcaatgaagcagtcaacctgttaa

SEQ ID NO: 400 - DNA, 398 bp, synthetic construct
  source 1..398: mol_type = other DNA
atatttcctttatactttcctaatagaagttatttttacatttttttttctttctttttccaaaggagtttgttatttactttaaaaataatccttaagaatttgactatgctctacttttatttcgtattcgttttatttcttgtttatgatgtataacatttttttaaaaagtcattgaataaaagttacatacaaattaccaaatgtttttacattttttaagacactacggcttcattaatgtagccaaggtaaaacttctattctactttctcaaattcgaattctataaagtttcaaacttaaggtatattgtgaatcatatttgattcatgagcgtgccgcgacagaggaccatgaatcatatatgcttcattggacagtgaacctgttaa

SEQ ID NO: 401 - DNA, 363 bp, synthetic construct
  source 1..363: mol_type = other DNA
acgtaatgataaaaatacgtataaattgttttagtacaatcataatatgcaagtacatatttgagtttctatatttttctttatttccagatggatggtacgagtaaacagagtgagacaccaaaatttttttctaaaaatgtgtaagataacaatgctaattgttaatgaccttggctaattgctaatttgtaaaagatattcatcaaaaaactatattctactcgtttctacagataagtaaccaaaaaaacattatttaagggcaataaaactttaataagaaaaaaattattttttgaaaaaaaaactcactcccgggggtccggtagcatgaaaaaaactcactactcaaagggttaa

SEQ ID NO: 402 - DNA, 285 bp, synthetic construct
  source 1..285: mol_type = other DNA
atgccggatatggagtgcaagaaaaaataaatgacacggaaaaatgtaagtatccatttataataatgtagagaaaacaattttgtattgactgtaatttttttaaatttttttgaagagcaactactgtgttaaaaatatttatgctaacgtctgcacaaatggtcggcactgcccagaaaaaagcaccctgagcgaattaaatacctccgattaaagtcacgctagaggcatgggatttagcggacgcaactgctgcgtcgcacacgctggaggggttaatca

SEQ ID NO: 403 - DNA, 626 bp, synthetic construct
  source 1..626: mol_type = other DNA
tgaacacaccagaaaaacattttttgtaacaataagatcatcataaaagcaattgatgaaagctaaaatataactattcatgttcttttgttataatttgtaacttagcaagtgacaatacttaattttatctgtacatacatttttcatgtgtttttcgtaatgttgtgcctgtggaactaaatgtgcattatttagttgtaaacctgtatctcagttatttgtgaatataactaagtcaaaatttagaatacaactcatgtttatatgtacaaggacataatgctttcatttttgagcagaaatttcattttatgccttctatatcaaatggaaacagaaaattttgaaaaaacattttttggtaagtatcatttttttttcaaaaaattgtgtgtgcatttttattataattctaggtgggcttatgttgtagtagagtcttagagaagtatattaaaaaaagaatcaataaaatccattgattaataaaaagtttgtgtaacttttgccgaagcactacattttcaataaatcttggccgggactggggcagatacccgagcgccagacgccgggacgtgggcagacgcccatgcgaaaactcccgggatcaaaagggttaa

SEQ ID NO: 404 - DNA, 651 bp, synthetic construct
  source 1..651: mol_type = other DNA
aggaatgtgaactgattgtatatatttgtaaatatgcttctttactaatatttgataatgttatttgtgtattttcctgtgttctttgaagaaacacttttaaattgacaattatcagtacctataaaacttccattactataacacgtaagtatttttaagcttatttaatataagtagctcttgaaatattagattattttcaaatgttaacatttcaaatttacttttgtaaacacactaattttctagatagagttctaaaaatttgtaaatagttagatatgtaacaagtgcacaaaatacaaatgtttgcgatgtataaaaatattttttttaccactgattttttagtctaaagttaattttttaaataaaaatatttttactttaggcgttatcacattatagaaaacttttttttatatgtagtgttatttaatcaacactattcgatagcgtatacaatatgctagaaaacaaaaaaaaaatcgttccatttgcataaaaaataaggatagcacagagcggaacgtaaaaggttgttaaaacgtcaattatgccttggcggtattgggtagtacagcggctgagacgcctggcggcgacgggtaaggccgcggtctcgggcgctggcgttcaaagggttaa

SEQ ID NO: 405 - DNA, 328 bp, synthetic construct
  source 1..328: mol_type = other DNA
ctgtaaatatgtggcaacaaaaaactttttacaaaaaacatttttggtgattcactaaacattgtgttacagtatgctcgagtgcttagtggggctgtggttagttgactaaccacaatttatgcttattaattatggttgatttgatgatatatgaatatctgacagctactgtatatataaaaagaaataaatatttgattaaaagtcaatttaaatgaattggagaaacttttatgttctgcccctctcagggttttttctaagtaatttttctgcccctcaaaggttttttcctgctcactttctctgcccttcaaagagttaa

SEQ ID NO: 406 - DNA, 321 bp, synthetic construct
  source 1..321: mol_type = other DNA
atcaaattttattcataaataaaattaagttcattttatgacatttgagaaaacaggtgtattcttgtttgaacagaaattctatactgtagaatataagtgctctaataaagttagtagttacataaagctgtatattattttgtatgagtaaccttagttcctaatggtacttttgagtccactttttgatttttttttaaaaattaaaataaaattctgaaaaaaaatacatagcctttttgtatcattagtttgtagaaaataacataagctctgcaatttttgatttttttctatatctgggcactaaagggttaa

SEQ ID NO: 407 - DNA, 326 bp, synthetic construct
  source 1..326: mol_type = other DNA
tttttcataataataatttcataattttgtataatgtagtcctaagaaaagtttgtaagtttgtaataaattttcaggaaataaacccattccaatttttttcttttccactcagttacactcaaaggtacttataagtaccagtcaaaaaaatatatgaaattgtaatttttttattttttttttcaattggattattaaaatttacctaaataaacaaaaataagtcacgaaataatacaaattaaaactacttataggggtggcattttaagaattactaattttccggtacttacgagtacctttggaaggcaaagggttaa

SEQ ID NO: 408 - DNA, 350 bp, synthetic construct
  source 1..350: mol_type = other DNA
tatatttgtcccctgtatttcatgtaaaaggtaatatgtaatctacatgaagttcaaaaatttgttttattaaataaattgataatttaacaatgtaatttgtacattatttacaaattttatgttgttttaaacctgtatactatttaaatgaaacttcagttagaacggaaatcattgctttcaaaaaggccactgattccatttggaatcaggtctgaaattttgtattaatatatatttgaacgcactaaagattctctgcacatatataacactttcaaagtcattttagtgcagtaaataaatttttttgctgcatattcattatttggactggaaagggttaa

SEQ ID NO: 409 - DNA, 331 bp, synthetic construct
  source 1..331: mol_type = other DNA
ataaaaaaattggtataataaagtcgtgttaaatgtaagattcaacatgtgttttatagcctaactaaaaaaattgttgtgttaatactttacaagaaaataattaagaaattacaacatttatatgtactcattattttgaaactaaataaaatttgaagtgtctttaagcggcaatgatgccatttggaatcgttagtttattttttaacgaacgccgttataaacattttttcagtgaaaatgcaatatttatgatctaaataatgaatattgatggcaattcattttttttaaaaaatatttctaaatgcgacgcagaaagggttaa

SEQ ID NO: 410 - DNA, 181 bp, synthetic construct
  source 1..181: mol_type = other DNA
gcatgagtttgctatttttgcacaatgttgcatttttgcaacaaacactaatttttcaataaaattttaggtttcaaaaaaaaatttttttttttatttttccatcatgttctgagtcatttccgatctaaaactaatttttaaataaaaaaataaaaaatcatgcatttaagggttaata

SEQ ID NO: 411 - DNA, 238 bp, synthetic construct
  source 1..238: mol_type = other DNA
atttttacttttttattactttttattcaataaaatatttttctttccctaaatttgacataatttttttactgctgatgacccaggtccgcctacaggcggggtgcagtttattgaatgcaccggcttaaatatatacctaatcgaaattttgagacgacagatctttttttattagtgttcttgacctagtattaatttttgaacaaaatcaaaaaatggggagttagagggttaa

SEQ ID NO: 412 - DNA, 188 bp, synthetic construct
  source 1..188: mol_type = other DNA
gtcatttaagatttctttatggccataatgcctgaagtatcaaaattgatacctgttttttttttaaataaatatgtaaaataaatattgtgatttatttagctatttttcccttcatctagaccctcaagcaaatacagtaatatttaaaaaagaagactcgcgtaaatcaggctactaagggttaa

SEQ ID NO: 413 - DNA, 207 bp, synthetic construct
  source 1..207: mol_type = other DNA
aattattaacccagtgctcttttgttcctgataggttactgctaataacataatgatgtaaatttccaccaacccattttttttaaatatagtattgtcattaatgaatatattttttttattatttttcccttgttatagagctaatcctacatccaaaaaaaaaatgtttgctcagaaagcaaaaatcatgcatttaagggttaa

SEQ ID NO: 414 - DNA, 400 bp, synthetic construct
  source 1..400: mol_type = other DNA
ttatcacatgcttgggtacaaacctgttggaactagataccttattgttcatatataaaagtgtataaaatatacaataaatgctcaatatttacaacattaatgtcctatttattattttaggatttttcccttcctatacaaagtatttaatcataatcaacaattaaattacgagagatttggtcaaaattgagggagcaaaataagcacatttattgtggctcgtaaggtgttaactcaaaatgtagctgatcagctacaaagcgattttcaacaatagtgctttgttaatttaatttaaaaaaattttcaatatttttttcgaaatgtaggcactataaactaaacatggtaatttttacagcttgaaataaaaaatcatgcatttaagggttaa

SEQ ID NO: 415 - DNA, 239 bp, synthetic construct
  source 1..239: mol_type = other DNA
taatgttatttataaaattactgacatttgtatttatgcataggaaaccatatatgtcttagtgaatttttgtgttaatgcatacgaacccatatatggtttcataaaataaaaactaaactacagtactttttttattttttttttacaaaattgttttttttatataattatagcaaataacatcaacaaatattttttgtttcacttctgtcatatatcatgcagtagagggttaa

SEQ ID NO: 416 - DNA, 218 bp, synthetic construct
  source 1..218: mol_type = other DNA
tgataactgtatagatttttcaaataaataacttttaaaattagcttaagcacattattacccagcgtatgaaatatcatacgttaaaaaaatcagagaaaacattaaaacaaaaattagaaacccatagaatcatgatgtagagcaaaaaagtaattaacaactaaaatttcaccaacattgggaaaaaaaagttactcgggcattattgggttaat

SEQ ID NO: 417 - DNA, 313 bp, synthetic construct
  source 1..313: mol_type = other DNA
aaagttttgacaagcatcaaaaaaacttgaaaaaaacactattcagtttattttgcactgtactgctacttataatgttcaaattttacaaatatttaataataaatatgtttgaagtaaaaagaattgttgaaggtctttgtacgttatatctgccatggtcctcatttggggaccattgatatataagctcctaaacaagttaatttgcaaaaaaatagtgtattattattacattattcattaattatgggaaaggaaacaaattttatcagaaatgagattttaaaaaaattggcagttaatgggttaa

SEQ ID NO: 418 - DNA, 251 bp, synthetic construct
  source 1..251: mol_type = other DNA
acgatacaaactgtattatttaaaagttcatacgtaatattatttttttttgtttatttagcagtttaaataaaataaatatcttttgttacaaacaaaaattattttttaataaactaaatacaatgttctaaattctgtgataaatgtttgatttattgagtaaaaattcccctataacttgttggggtctccaaagaccccaagtgctcttccgcgataaaattttaacgtgctcagtcgaaggttaa

SEQ ID NO: 419 - DNA, 236 bp, synthetic construct
  source 1..236: mol_type = other DNA
ttaggataatttaaaaaacaatgtaacaaacaatataatattttgtatgtgttttaattttaaaatggtttaagctttaattctaaacattcaatttgtcaaaataaatatctatgttcattaaaatacgcagaaaagctatttatttcaagaatatccagaaaatacacaccgggtcttttggacccgggtcatggaaaagtggtaaaatagtaggtcatgcatccaagggttaa

SEQ ID NO: 420 - DNA, 353 bp, synthetic construct
  source 1..353: mol_type = other DNA
gaaaaatgcaattttatgtgaacaatgaataataactttttgtcaaaatattgcctatatacataatattgtgaataatgagtatgttttaattgaagaaattggttgtaataaatgttataaaatgtaaactgcaacttataagtgaattaattaataaaatgttaatcaggcccaccagatcctgttactgtatcttaggaatctttcaatatgacagtaaatttgacagaaataaatttaactccaaaaaatgattatatgaaaagacgaaaatttaaaattaggcagggccttggaggccctgcatatgcattcatgtgtgttttcggcaccatgcatcctagggttaa

SEQ ID NO: 421 - DNA, 575 bp, synthetic construct
  source 1..575: mol_type = other DNA
gaaaagaactgtagtattttataagatgattgtattgaaaattctgttgtttcaaatttatgtgaatacttgtgcctaaactacaatcagtaagaagagaggattctaaagttgtagtgctttctatgttggaatgtaataaaaaaactgtagtgtgttctgtactgtagtgtgctctaagatgaatatctgtaatgacaactgtctgttgttagaaaatgtcaatacttacgtctaaactgtcaacaataagaatagaagtatggaaaaactgtagtgctttctaagttgaatgcctgtaatggaaactgtctgttgtttcaaatttatgtgcatatttaaatgaaaaatatccctcaataaagttgtatgcattgttattattattattattaccattattattattattattattattattattattactaacaaggtactggaatagaacaacaatgtcgatagctaaaactgtaccaaaatttatgtttctaaagcattttgatatgtcgggtcatattgacccgaacagtatatatgtcaagtaaaagtgaacagaacagcagggttaa

SEQ ID NO: 422 - DNA, 258 bp, synthetic construct
  source 1..258: mol_type = other DNA
ggaaaaaattcgtttcactcacaaaataggaacagtctagttttacaaaaaaaggatttctttaacattttgtattggtttcttacaataaattgcttttattttcatacgcttttaactagtgattaatcacaataaacataactgtagtttccatgaatttgttttgcagaaaactgcattttttcgtaggggtgcagatgcacccccccttgggtctttacgtaactattcggaccttggttcgttgaaggttaa

SEQ ID NO: 423 - DNA, 606 bp, synthetic construct
  source 1..606: mol_type = other DNA
gaacattccaccatcatggaacatgtgaaatttttgtaacttcattgcactgagtattttaaatgtcaatcacttcacccacatactgcactgatttatttgtacactttcttgcatctataagaaaagattccttgttcaaatcttgtacatcagaaataaaagtcatcaaggaatgaaaaagacagactgtgttgatctcttgctaaaaaaagagggtaaaataattttctgatcataatgtgaaaatatcatttgtcgttgcagcttgctagacaggttgtgcatacactggtcttagaaatattgtgctgagcatctccactgtgcaggggaagctggtggacttcctggtggagcttgccagtgagcttgcacattcttatttgggagccaagaaggtgaataggagagtgagagtgagcaaccccccacaacttggccaacgtgtggggggttgctcacagaacatctcatttacagaacaatagtgtcgttcacacctgagttaggatattagctaaaactaatcccgccatttggctgccctctgggcatggaaggtagaaacgccaaatggcgcagctttggtttttctagtgttaa

SEQ ID NO: 424 - DNA, 584 bp, synthetic construct
  source 1..584: mol_type = other DNA
atctgtatttgtaaatttacaataatttaactttataataaatgatagtttgtattgaaatttgttattattcaatttattctaactttcaaaatacaaaataacagtacgatatttgaaattaatttctaaaaaaagtgtttgaaaaaagcaaattttctgagattacgatatagaaaattaaaaagtttagatacaaaaataaattaataactttggaaaattatttaaaattttaaattttatgtcattttgctattatcaaagaaacctaaataattgacaagtaaaacgtctacggtatctgacagataccgtgcgacctttcatgtccttggattttcataattttataacggaaaatgtagcaatcatttgagattttttttgttcagtcagaccgatccaagtttacattaccagatattgtgtattcagttaaggagttaaactttcaataatgatatatgacatcgtgttaaatataattttaagtcatcgtaaaccactttaaagtaaaagtttcttatagatacggtgcgaccgatcacgtaatattttaggtgcgaccgacggagggttaa

SEQ ID NO: 425 - DNA, 339 bp, synthetic construct
  source 1..339: mol_type = other DNA
gaacagttcaaaagacaatgtgagttgcaattgaaatcttaaaagctatatttaaagttcagtttcatattttaaaattatttagttttgagttagcatagcaatataattttaaaacctttctcaaagtttaaaaccattttcaaagtttaaaaccattttcaaagtttaaaaccataagtttttgtactgttgtattagtaataaataatatttgagacttaaaaacacgattttttacttaatgacctttgttttactacatatagatagtgcggtctttctgacgccgcgcgaccactttaaggactaactaccgcgcgcccactctagggttaa

SEQ ID NO: 426 - DNA, 398 bp, synthetic construct
  source 1..398: mol_type = other DNA
aaccagcacacagttaaggtcggaataagtataattttcatcagatttattatattaacttctttttagtatgcactttttgaaaattattaactaaaaaattataaagtattgtatgtaaatttatgagttagtataaactaaaaaattgtagtaaaacttgacaattttcaaaattactaataatttactaagagtacatgtgtgttcaaaaatgtaataaacgtataaaattaattattgtatatcagtttattatttaacgccttgaaataattccaaaacaccgtaatttataacaataaatgcaattataatcaattttaccaaatgttaggtttgccctaacggcgcgagtgatcacgtaactatcaatagcgcgagtcctcgcgggttaa

SEQ ID NO: 427 - DNA, 623 bp, synthetic construct
  source 1..623: mol_type = other DNA
actcacgtgtgtaaagtgtgaaatgcaaacataaacatgaaagtgcatcaatatccttcttacaatacacaaaaacatctgtaaaatattacaagtgaagtgagaatgattgttacatccctgaattttttcattgctacacccctgagtgaggtattcggttatcaaatatgacgtaagtaaggtcagcagtagtgaacttgcgtttcccgtgggagagattaagagagatcaagagttcagatgaccttcaaacatcgagtgagcaacaatgctagataacctacataattatgatgatagtgaactaactcataactatgatgcaacaataaattatgactcaaagacacgaccagtgttgaaatattatgcaaacatgtcacttgaatgcaatttgtgtactatccatttcattattaatatttatatttaatacgttccgttatgcgtaccatatctattcttattactgtaataactgaaaacaatatatttcttaacatttaaatctaattccaatgtttaaaccttgtctgtaaacaaaaacaaatgtggattaccattgtaatccaggtgagtactggcgttacggaatttaacgcgagtaacgaagggttaat

SEQ ID NO: 428 - DNA, 340 bp, synthetic construct
  source 1..340: mol_type = other DNA
attttttctttgcattacattttagtattcattttctttgtgttgtaactataataatgtaaaaaaaaagtatttctaaaggagtaatgtgtaaacaacaaaacactgcaaattaaagtttttcctagtaataaacttgaatttactggtattttaacaaatattatggttcaaaaacctattattatgtttaattacaccttaaagagagagagaagcaacgtagaagtgcggaaaagtcataaaaatagagaaaaatacactttaaatgtcctagcggtctagcagaccgatgttccatcttaccgccgagtaaaagatgttccggttagagggttaa

SEQ ID NO: 429 - DNA, 293 bp, synthetic construct
  source 1..293: mol_type = other DNA
ttctttttaatagttttgctagaaaatgtttcaattttggttcctattttatatttagatttgtcataatgttacaagtgcttttaagataatttagttattattttcaaaacaaattttaatacttaatttgtacctagcaataaaagatattctttcgcaaataaagtaagtttaaatgactcctttttttcatgtttctataataaaaaaatgaaaatgtgagctgtgagtctgtgagactcacctgtgggattgccgggaaaatcagcacatgtggcggtacgggttaa

SEQ ID NO: 430 - DNA, 372 bp, synthetic construct
  source 1..372: mol_type = other DNA
aaatgatgtgttcatcattcatccaggaatattaagttttcgtgctgattactcttatttaagctactttttattacgtataccatgttcaataaaccaaacttaatgtgaaaaagtgtcatttttcattattcctttgttcaaatccaacaaaaatacccttgatgataataagtataacattctttaattatctaagctttgcattttaatgataagatttaactttaaaggcttttccaatgtaaaaaaaaaaacaaatactagcggtctgacagaccggtatttctctggcgaaaacatcgggcggtctgacataccgctatttccctggcgagtcaaaagtgtcctatttcggtttaagggttaaat

SEQ ID NO: 431 - DNA, 595 bp, synthetic construct
  source 1..595: mol_type = other DNA
gcatcgagtgcttttttttctctctctccctctctttctttatattgtatttatatgttgtatttatcatttgtctttctttacatgtctatttcttctatattgtaggctttctctttttaattcaatttttgtcatttttacaccagtatatattaaaaaaaagaaaattgtgaggaacctcaatttgcctttcgtaaaaatttattgcttggatgtcattgagcatatttcataaaatattaatctaatctattttattcataatttcctttttcatgggtatagtaagctgacacaaaattcatttattattttataattcatgttttggcaattgttactaaataaattgtagaagaaagattaaatataaagtaaatttattagtgtcaattatttattattaattttatattattttaaatttaaccagtttcttatggtattgggttgcaagaacacgtattaaaaggttaatgacccataaaaaaaatggggacgtatataataaaggaaataagtctggggtcccctagaccccacctcaccacttgtgattgtaaacttcacctcactagttaaaggttaaatt

SEQ ID NO: 432 - DNA, 368 bp, synthetic construct
  source 1..368: mol_type = other DNA
atctttgcaagtatgtcgtatatcgagattgactatttttttctacataattatatccttaaagagttggtttcaaatgtaattgtaatgatttgatattttttaatttcaatatttaactaaatgctatatatctttttttaattttatgctaataaataacactaagtatgagtaataagtatgatttgtaaattattttttatacatgtaatatgtcttaaatataataaataaaatgttccaacacatttgttttagtttttacaacagatagttacaaaaaaattcataaacaaaaaagtagtaaaatttacgaatgcttaccattaacgcgatatctagggagcttaccattccagggttaa

SEQ ID NO: 433 - DNA, 459 bp, synthetic construct
  source 1..459: mol_type = other DNA
taaaacaaaaacttttattgatgtactatttactatttcaaaagtttgtgagtttctatattacttttttacttatctacctaaatatgtttttattatattatctcgtttataacgttatatttaggaacctcatgtattctagtttacaaaaatttcattgtataataaaaaaaaaatatttaatattttttttatttattcccccaagacaattaattggtaattttttaagatacactgttgccaaatattattttctacattcttaataacgagttttactacaaaaatttatattatttttaataaccatagttaggtaaatcttaaactgattaaaaaatttttttaatctataaaatagttacaataaaaaaaaaataaattggcgtcgtcaaaattacgaaggttttgtgacaatgtaacaatgaaataggctttgtgctccagggttaa

SEQ ID NO: 434 - DNA, 494 bp, synthetic construct
  source 1..494: mol_type = other DNA
aaatcttgaactactctaagccaattcttgtgttacgcggttgatacgccgcactgtattttttctaattctagagatacttttgaaacttttttgtacaagtatttttataagaactttttcgtccaaaggaccaaagtacaagctctgttgagcgccatatttctctgtgttcttaggtagtctccggtgagcgccatttttctctgtgttcttaggttgtaagtttgattttcattcaaaagacactgtacagtacgaaaaattcctatttgcactagtctcaagttgatcttatgtaaagggtccaccctttactctttgtttttttttacgtgatttgaataaagactcttttaaaaatgattatagctcgtttttatcgcaactcaccctatgctcacaccgttaacaattaaaactatcaaaggagtcaaattgacgcatcgctagtaaaatgcgcctataagatatcgctagcgttccagggttaa

SEQ ID NO: 435 - DNA, 172 bp, synthetic construct
  source 1..172: mol_type = other DNA
gttttcaattgaaaaaaaaactattatatttgatgtaagtaagctttaatattaattttttctcattaaaaccctttttatgtaaaataaaaatgtttttattaatggtagtctccctgacgaatgtttagctgtaacgtgttttttacgaaccttagctttctagggttaa

SEQ ID NO: 436 - DNA, 433 bp, synthetic construct
  source 1..433: mol_type = other DNA
ataaatgaattcatttgcttatgttttttgttattttattaatttttgtagaaaactttctcgttgtatgaattatttgatttaagttttattatataatttaactttattttttattaatataaattttttatcgtaaatgaattattaaaatattattttctataagtttgaataaaaaattcaataaaattgttaaaaaataactttgactttttatatgaaaaaatttctttaaaggagatgaaagcgtgaattttcttatactttgagaaaaattgttgccatttggcaacagtcttaaaaacccacttcgaaaaattttttaatttttttttcttttgatttgaatttttgttattaaaactattaaaaaaatataaaaataaaatttttattgtccttgagtaaaaatatgtagcgaaagggttaa

SEQ ID NO: 437 - DNA, 353 bp, synthetic construct
  source 1..353: mol_type = other DNA
atgattattatgaatgcctgttaatatttaaacattgtttgaaataaataaagtcagaggcttgaaaatttaatttaaaaatgagttatgttatttgtgagctaaagaaaaagattagggatcagtgggagctgacaatgagtaaatttatgtcccattatcccaagtggtcgttttcgacctcaccagataaacaattttaacggttaaaattgtaaatgttgtttgtttatttgccacttgtgtgacattttaatgcaaatttaacatccaaaacactaaaaaaacatttttggaattgtgggcatttggaggtattatcgagtccagaattgtgggcttaaatgggttaa

SEQ ID NO: 438 - DNA, 527 bp, synthetic construct
  source 1..527: mol_type = other DNA
acattcaggtcagacacagagtatttgtttgatagaattgttatgtttcaaccaatttttgtagcccaacaactttttataattcacacatagagctatttttcatgtcaaacctataaatctcattagtgtttatttgatttgttatgtaatacacatgcgatataacataaatgtatatatatatatatatatatatatagatgcatacatgtatttacatataatgtctaaagactctaacctatataatcacctatataagccctatagccttttccatacatacatatagtaatgtttcatataaagctaacaaatattacaatattatataatttttttttataaatatacaaatattatcaatgtaaatatattaaatgatgtatattaaagtattctgagcttacttgtgaacagtcttttatttacagccattacaattgcattattacaaaatgggtctgtcagacccaacctcaccactcccgtcctctggtttcacctcaccactgtagggttaa

SEQ ID NO: 439 - protein, 927 aa, synthetic construct
MVCLSGEAELMAFGRASGRLQLPLPSVRVGPAHRPIGKQASYEAVGEEPPHGHPPLTTSAPPPSVTATESAQTPEVSAPQVSARVQPPATAPLTPEVNPAGLQPSTEVQAQLAITDGTEQDEALEGHDLDNTEHGDTEFTVPEDTEQERRNEGSDTEWNPREKSTEADEAKSESDDPADYRVDFDVFDSDNFMEGLRKERLFGPTDGDDVNVVAGQDASDSDSDADNEDVMNDNEVVLEVEGVEDLEDVSLNDDIPEEGLFDLTSGDLRDIAENGWITYAEAESGSRMIQISVWCNCTYSGLCVYYRESPDGRCNGTTDRADPHGLRSHTHSPLAMFFYFLPKELWIRIADETNRYREQNIGAVALSRRAKMLARQAEDPRVSVPNLEDIEAKLRKFKRIQAHEILHVVGLLFARAVAPIRDGLSNHWSTEEDGAIPRGMFSRFMKRDRFEAIMKFLHFNNNDDGGAHADRAWKIRPVLQAVEKTFRRGYRLGKVISFDEGMMPNRSKFNPMRIFMPDKPSKYGTKFYMTCCADTAYCSRYCILFCTSLNTQFRRLQNTLTLSHDMLYLSDLEWKSIVGQTAKRRRRQVHGPKAVVRNISKALVGQPRKRLIVTDSFYSTVSLSLKLLDMGLYHVGTTRIDRLGWCPIQFTQAKRPKRMPRGTYRIAQARDHPQLVALSWMDSKPVNMLAAGCSTHPASVMRLEKDGTRSTVPCPQLVVDYNRGMGGVDVHDQLRLQRYSIQKCISLRKYYKQLFLCIVDMAVVNGYIVHCVTLKKKGEKQPTHAEYLRRLHIQLLALQTINFETHPNAEDLVAVPIPRQQHALVNTDDFYSSGKQHKRRQYLCKVCSAFADSKTKSFETSFFCPRCSDAFGGRVPLCRHVRRVESGNTLTCSQIWHETWGDGKSVPPSLKKKIRFRKRKRESDEEA

SEQ ID NO: 440 - protein, 984 aa, synthetic construct
MSSAQKLNSGKEVRRMRHVGYIGRPFACHSRNCRMARIRSTKRGKTAAVAAAARDTDFRYLWRQLRRVGWTAKRPTGIETEWRYVAPDSSEVFVGEDAVVVHALESGLLAEDEGDEESELLAADQSETESGLLAGGHSEEDEGNASSTTTVNAGDGIVGASQIDTSTKLSQRTLDDLFGSESDSDVALSQAAVPRAFDLSPGDLEDAGSEHNAAASLQLLSEASAVESEADVGDDRAATPGPVLRPTISVKKDINFVPEDENLSSYESFSSGESDIGGIEDSNDGAAVVDTPSDDDVLSDDDAPQMDSAFVQSLLIGEDVLNKKAADARSNALRATKWTPVSSAFEEASTAYPGLNTTEEAKPVQELQNVCHSPLLTLFYFMPKSLWVAINVETNRYCMQQIARRAQAIHAKQTDRTRETPAQIRRRLQAKPAYQTHEILQVLGLLIARMLCPQNRRFAAHWSMTEDGAVPAGTFGRFMGRNRCQDILRDLHIVDNERERTRDKLWKIRPIVNKLQELFLAAWSLPPVFSFDEGVLPCTSRRNTTRMFMPDKPHRYGSKKFMVCDARTAYCHRYCIYNLCHNLRRVSSILISYVVTCRFEMYAGKRNAGDTGDSSFDHKTGAAAVVRNLKVVLNADRRHAWHLVVIDRFYSSVLLAIELLGMSVYVLGTIMTDRLGFDKNVKAVRKTRPESTPRGAFTFSRCVDVPTMVAYHWWDRKPVHYLCTGSVMSASNINRNVKRVGPVTVPCPAAVTDYQRWMGGVDVHDQLRLQKFSLQTSTKFQKYYMSLFLGLVDLALVNAYLSHKETARLSGTTAMARGEWFCVLQNQLLQLKAEDFAGILATPPSHCQKRKRTPVRLAHEVEQSDDWVTVSGVQKRRQRSCKVCALLRTNKKKKSFATTFFCERCSVDNAKCWLCNKIRHTYRGEAKTCFAIWHEEFDCGQSIPAALGAKVVLRRPGQAVGQRKKTRRELQLHNGDADDEGEGN

SEQ ID NO: 441 - protein, 830 aa, synthetic construct
MDEVGDVAGQAAGAEAARTTAPTAATSTAADVGTAAETGSAVATASVASTETVVNTGTVGDTGMADTDTAGPATDVGAARAVMNTGAVRAVVDTGAARPVVVDTRASSPVVGASQIDTTVGLSQGTLDHLFGSPNDSEDELSQAAVPHALGLSPHQFEADTSQREAAMNLHQLSEASGLESGADDDQERQRPATPPRTLRPRVVKKDVNFIPEDEDEKEYESMESGNESDGEGSRDYDFEEEPARDVVSDEDVLSEADAERMDEAFIRSLMIGNTSVTSQEKKQREDALRATQWTPVVSSDFEVDTPEYPGLGEHMALPVAELRAVASSPLQTFLYFMPKSLWVMIADETNRYSLQQLDQRAKGIQTKQHRRRRESLKQIRRRLKLKHEYKPHEILNVIGLLVARMLCPQKRRFSDHWTTVEDGAIPAGSFGRFMARDRCQDIPRNLHFADNSDAEATRDRAWKLRPVVETLQQRFLDGWSLPPVIAFDEAVLPSRSRFNAMRVFMKDKPHRWGTKLFMTCCSGTAYCLRYFVFFCVFSRVVSYDVHAHFLVMLYLHRFEVYAGKRQAGTAGDAAFDHKSGPAAVVRNLKVLFAPKARYQWHAVVADRFYTSVPLAMQLLSMGVYAVGTIMTNRLGYRQRIVDERQNRPPDIPRGTFTFRAVAVPAMAAFRWWDRKPVHLLCTGVGNERVERRRNIKLCRADQVPCPSAVTDYQRWMGGVDVHDQLRLQRFSLQTAVEFRKYYKTLFLGLVDMAIVNAYIVHKEAQQASRGRHASSTRSGLDSSRPSCCNSRLMTSSDSVSYSDGGYAETHTNAHPPYARAGAERRLGHC

SEQ ID NO: 442 - protein, 939 aa, synthetic construct
MDEVGDVAGQAAGAEAARTTAPTAATSTAADVGTAAETGSAVATASVASTETVVNTGTVGDTGMADTDTAGPATDVGAARAVMNTGAVRAVVDTGAARPVVVDTRASSPVVGASQIDTTVGLSQGTLDHLFGSPNDSEDELSQAAVPHALGLSPHQFEADTSQREAAMNLHQLSEASGLESGADDDQERQRPATPPRTLRPRVVKKDVNFIPEDEDEKEYESMESGNESDGEGSRDYDFEEEPARDVVSDEDVLSEADAERMDEAFIRSLMIGNTSVTSQEKKQREDALRATQWTPVVSSDFEVDTPEYPGLGEHMALPVAELRAVASSPLQTFLYFMPKSLWVMIADETNRYSLQQLDQRAKGIQTKQHRRRRESLKQIRRRLKLKHEYKPHEILNVIGLLVARMLCPQKRRFSDHWSMVEDGAIPAGNFGRFMGRNRCQDILRDLHFVDNEAPRTRDKLWKLRPIIDKLQQRFLEGWSLPAVISFDEGVLPSTSRRNTTRMFMPDKPHRYGSKMFMTCDSRTSYCHRYFGFFVCFLKSPDMSYLHRFELYAGKRQAGTAGDAAFDHKTGAAAVVRNLKVLFAAKARYQWHTVVVDRYYSSVLLAIELLSMGVYVIGTIMTNRLGYDQRIVDKRQSRPSGIPRGTFTFSRSVAVPAMVAFHWWDRKPVHYLCTGSAMRESSVGRNIKCVGQISVPCPSAVSDYQRWMGGVDVHDQLRLQRFSLQTSTKFKKYYRSLFLGFVDMALVNSFLTHKETARIEGTPCLKRGEWLGQLQNQLLQLKAHDFVGVVATPTVDTQRRTRTRTRHTHAPEQSDDWVTVSGVQKRRQRSCRVCALLRSSTSKKSFQTTWFCERCSVDDAKCWLCNKIRHEYRGVMKTCYEIWHEDFDAGEAIPTHLGKRVVLRRPAQKAGARKKTRRELQLRARDESKSGNGEESEDE

SEQ ID NO: 443 - protein, 564 aa, synthetic construct
MCGSLPQSISMAKIISAQRALEIIFAEREASVEGASSTLEDEEFSEYEDHVSVNSESDSELEEEDEIDPQPAPGPARQQPAHQQPAGGEIWMSKNCEIEWSSCPRNEPPRMAANVIRMQPGPTRMAVTHVQDIKSSFELFIPDTIQKIILDCTNLEGRRVFGERWKEMDQTHLHAYFGVLILAGVFRSNGESTESLWDAETGRELFRATMSLENFHIISRIIRFDNRDTRPARRQRDKLAAIRSVWDKWVDRLPLFYNPGPNVTVDEQLMPFRGRCPFRQYIPSKPAKYGIKIWAACDAASSYAWNLQVYTGKPDGGAPEKNQGMRVVLDVTQGLRGHNITCDNFFTSHKLGQELLKRKLTMVGTVRKNKPELPPQLLNTRNRPINSSKFVFTADTSLVSYVPKKGKNVVLMSTLHRDGRICGQEHQKPEIIMDYNATKGGVDNLDKLVTGYSCKRRTLRWPLVIFFNILDISAYNAFVIWMALNPDWNRGKLQRRRLFLEELGKALVRPQIQRRQHIPRTPASAAIVRRIQEEDAGAPSARPTEPTTPIPEVSVSDVVACVCV

SEQ ID NO: 444 - protein, 465 aa, synthetic construct
MFISLKMSKKWDLTNPLHLEKAMEYLFSLPEEDELSDVEIDDGAREELDELNTTTTSRFLESVNEEMVETEAEMDLEMDIPAPIEPEMNEDVQDWNYDCSFFQCDAETNFRQNASFDDNFSNYKAIDFFTQLFDQCMIENVVSETNRYARQKQSSMWVDTSQEEIKAFVGSLIIMGIHRLPHLEDYWSSDPFLSVPAVATVMTKNRFKKLIENIHFNDNDKNVDRSDPNRDKLHKMRPLIDKLNENCNKIITPSSFLSIDESMIPFKGRSSLKQYMPMKPTKRGYKVWCMADSTLGYIVKFDIYTGKSVAPLCKETTLGERTVLNLTKDISSRGTHVVVFDNFFTSSKLLLKLMSKGIFSCGTTRITRKDVPPIFKENKKILRGEYQYQTRGQLAAVRWMDNKPVHFLSTIYNPRDTTTVLRKNKNGQRETVSCPLVVSKYNKIMGGVDLFDQYRERSIVLVGDL

SEQ ID NO: 445 - protein, 715 aa, synthetic construct
MFLKLPPIFISLCDSTLTMAPKDMRLVPNATVHAKANRVVEGILCRQFYGSMWKTKEVLGTVISTETKKPNLKTQTFVKGSWELPSGNVIKTVLLRHVRAGTPAPRGTDGQILHHARHETADVIDFAPAPPEAPHTQPSSCDVFPPTAAQGHVMEQTACHGQIWSSYEEDGHVNGMVRAKRWEMKSPDGSTVSEGSEWRERNVLDYFFMTFPMEYLPKIVQLTSESLASAGEKATTASELLQFFGVLILMTRFEVCGSRRFLWGTKPVSKYLPTPNFSSIMPRRRFDALFANFKFSKGPIRSAVTEDAPDRWCMISDFVEAINNHRVKFFVPSETICIDESISRWYGLGGHWIDIGLPHYVALDRKPEAGCEIQNAACGSSGIMLRLELVMCADDTARKTFEGTTNHGTAVVRRLTEPWTGSRRMVVADSYFASVETAEQLMKTGLRFTGVVKTATKRFPMSELGRTELTGRGDTVCLQTTIEPAGTVLLGLCWVDRARRYFVSTAGSMRPGAPYERVRWRQESDGPRRVHIEVRQPQLVEHYYSASSAIDRHNRCRQDDLGMERKVEVKDWHKRVNLTLLSICVVDSWLLYKGGRGAKGHMPQHVFYAKLAEGLVDNVFGTTAFLRVSSSNSIGNEGFIPSSGVREHLSPTKRRRLCFGKVSNAVYQGRCSVCQQKSKFVCSGCRDDKNRDIFLCHSQTGRHCFNKHTVAEHYY

SEQ ID NO: 446 - protein, 813 aa, synthetic construct
MKKEKDRIYVQAISPSRVSLVHTTPIFIVRHNRQHLVFIMAIDPRLIAGIMVNAKATTVTSASHCQRLFGAGWKDGWFQGKVIRAVQKSEQRKTRTIKRWYVTVRWAVGIKLKDKEMLLCHVKAGPHPIPKEGIPDREIEAPFPYHILSETDAESIAPPATNNAGKPQTQGGLSDSSPTTPSADGAGPSTHSSNVEDTAQPIFGLQNDVLAPQVEAAYAESREKSQAPPCQENDVAAVAHGQEWKLVNGKGIINKRPPKRAWSVTGPSGVLITRGTAPAHLAPIDYLFMMFPVTHLFAIVTMTNKKLRMENSADTTAGEVLKFFGAMILLTRMKFSSRREVWSTTSDCEFIPPMSFGQVMPLHRFETLLRCVRFSYQTEEQGEWSDPEYRWQLCEGFLDAINAHREATVTPSELLCVDESISRWYGIGGSWIAKGLPHYVAIDRKPESGCEIQNIADANGGFMLRLMLVKGADVNFGDHVQGDLNHGTNVLMKLCAPWENTWRTVCADSYFASVQAAKALYEKGVHFIGVVKNATRMYPMSYLGNLPMPGRFVQKALTTSMGSSRGPFDILAAMWVDRNRRYFVASTGSMDDAEAYTRARWTQTRHGPIRKNIEVKMPAVCATYFSAAGAIDQHNRLRQDDLDLEKTIETNDWSVRVNSSLLGICVSDAYMLYKGGRGTRSCMKPHKFFRLLGSELVKNTYDVTGLRLRRPGTAANKPHFAGPPTSGVYEHLTPTRDHRKDKDGRIQKAKRQRKCRTCGMKSKWVCSGCRAKTFGEKHFCHSDTGRSCYREHLGENHNSGEVRASLQDSELEF

SEQ ID NO: 447 - protein, 1112 aa, synthetic construct
MAEVLPSNDSTEAPGELISANQPPASTELPGTTTPLQSAGNNETPADGAGANGIPTPTTPHVYVVRSTARGRGRGRGRRRGRGRGSIRRATRNSQVNALTPCDGWRIKAVMGERIQMDNVFLSNALTERARLFCRSALDHMHTEMNVRRLDHSRASHIVLLCDEVLHKCLDWTNESIVLKHLSMSPLRMSDMYSFLGVFVFSQCSGFSLAKSLFFLSKLDCPVPTLQLVRFISSNILAFSATGRGDQGESVWNSQRDQTVHLSEFEQAAYRMSCKVFLSPGHTFATLDDDLYGTRAIDNQVKTLSARKADKEGHTVDAIADALSRTTLMVRFRRRGEPQTVNVEKLFDAMLETRGEQTLTGFVVTADRGYGKFEMLEGLGMRGIGFMFIMPEHLLKCHPFVGRSYFIATRHDLEEDNAASTVDGDDSIAGNGVHFGADVYDRDAALVIDDSPDKGPLSKYAEKAFRSENNTSRQTVTAVAVRERGTNKFSKVLRFAYCLPGRLSSLLKCWVAVPRALSQRNLLFSKRSSDGRLVIPQQGSMETKDLIELHCISACKVLTIDQRCADWFILRQFRITGTIAGETLLADEHVCSLLGLQTNPTAEHRTSSVKFRNLASSWFSYARSSEAMIRGTVNEKAVLKALLNKEFILGLQECGMLADKENPWMACSPDGIALIDIQRTAFADSTRTEQSDKISCIEIKTSVCPSSLARSLMNASVDLVCAKIGDENFKQRITREHTGQLMQQVVITRTNYCLYVACSESALLSCTLVYVPDSAVQSFRTSLKPNAEPLVAWAHEDSPSVPSFVDAETKKFVRDRLPFWKLANDHVTQNDAFIPLKLFKHGMQAIYSKTKGGVDGSAQARAILRSPTSTMKWEQKVVSQTLKTLVVNAFIGWRFAEKKDLVDYGERFQNLTSFRSALNSIGSLADFCADASVELLIYADSIARREDEHPGGANQNDTTIQGGEAARLRGLAKKRRRNRIQFFNSDDGIKLRLNVPGHCQKQTKNERWCSLCGMNRVSDGGFGGHRSCFRCSHCDLHLCIRTYARFRRSCWDVWHSNKRLEPRQESRSSRSRSPSSQGGTSCLTTPTEVRTRRRRRSVDSEDRSARLRARTS

SEQ ID NO: 448 - protein, 1244 aa, synthetic construct
MDSPLTLYSRGCKTQVNLIPRESSYRRHLDFQYSKSDKFSKRYKQPPLHYLATSAPLNSLPQMASLDLIDVFIEDESTQLNVQQLDADAQGAVGVAGPGTSTPVPIAEGVPNVGPLSRRRGRGTGATATTGQERALTGGITRRGRGTGRGRARGRGRGRGRGRARGRGRGRGRGGSAQNNSHVRDPSIGWTEVRQPCSSVMMEREFTGSMIDERARLFCKSAIVNMKMECTAKCLSLTRAGLVVLFMNDVLKQCVQWVNEGLALRGESETKVTLADGYRYVSILLFSHCTGFSFEKAISILRLSGSTVPSLERARFFATNILAYSPTGRGNDGNFVWQSQRDQTRQLSEFETLAFKETKRVFFSPLHLFATLDDDLFGTRAGDNQVKMISSRKADKEGHSADAIADALFRVVLGIRFRRRGEKQLDSVRKLINTLVDGRGEESLNGCVVTADRGYGKEAFMKILTEHKLGSILVMPEHLTKCHPFVARSFLKPFRDDDIEEFESDSPPTAEAVHGPSTPRDTPSSEAVSPVPNVHNEEGVSSTVDGGTSSTVAGQGTVGADQHLANDRRNNFIIDDSPELGPASFFARKRVSECARLGIQKVSAIAVREHGTEKYGKVLRFLYTVPEHVQDRLNTWLAVPKAGMDTIRTLFGSEGTESNVLKAEIETHLSGTCEILTVGQRCADWFVLRQFRVTGTNAGIVLLKSSAFRTIIGLETTNAHNEMTCREWFEVFFKGWFSSKVSTEAMMRGSANEGAVLSALTSLPFIVTVFECGMLAMKNTRYLACSPDGIALVDAQILQKFNTEEGHLVNENGIPCVLATVEIKTRVASKSLNTALELATGDAVFCEVGDETFRKYIPELHTAQVLHQMMILDVNVAIYLAAGETGLLYAVYVYCSDVHLVTCQSILTAQTSELVQWAYIEAGTVPEFINDEYKECVMSRHQFWKLVDDRVRLSGPFPPLKLFKHASQSFYSKTKAGVDGATQQRAILRSSTSHFQWEQKLVSQILKTIAINSFIGWRMSERVDLLESTEAFKSLEKYRNALNKVQATADFMFDVSQELLSHADTLQENSNTEEARMEITWGSSEGSALITKAKARKRNRLTFYNSTDGRKLRLNVKNHTPIQKTGVNRCSLCGQNRTGYRGHQTSYVCSICPVRLCVRTYAGLRKSCFSIWHSSKVVETRQLSHPVSRNIVAAEPTETDLTAARAGTGRQGDNGATCSVVGEDDEIQPATPTPRIGSRVRARS

SEQ ID NO: 449 - protein, 314 aa, synthetic construct
MYNFFSPDGKIRPLVDHFNRVAGYFYTPDKNICVDESLVGTRGRTAMLQYIPSKAAKFGVKFWVLAESASGYVHNIKCYLGKKFSPVTQLQQGTDVVLDLMRRSNLLNKGYHIICDSFFCSLDLAKRLLNLNTYITGTLRANRQMPLTIKNANIDVNETVYVRQGNILATAYKGRNGRKPVRMLSTYAHAASLNGKPAIIELYNKNMGGVDGADQLIHFYNDNRKSIKVWKKLAINLIHRMCNNAYILYKKNTCHMPIKSRLRFVQDVVEGLATEHLAAREDREGHVGRAIQPRRVVAIVRQIAGKTIVCQVAL

SEQ ID NO: 450 - protein, 487 aa, synthetic construct
MQKKADAKKVAQHKAVNRNWKECKVWGDVGRQSRADDDSPRPQFKERAIQFAPLEEPGPVLSQFSRFSSPLGVFKTMLGGEDTLYLLREATNDYIAEFWNSRKTPPSRKVGGKPEGRLIGPRLPAGGFVTDHELIAFFGIQFLIGYHRLPELSMFWEQQPDTGLGLGIIQQAMTRERFKFISKHIACASPWDDVDPNSDDPDQEPERPDPIRKIRPLVKRLNESYHVCRKPPRGQSIDESMVKYKGRSMLRQTMKNKPIKSGFKIWSRCCLRGYTYKFEIYQGARFGEKQKRSRNNEAVERVVVDLCQPLTDQGFVVAFDRFFTSIALLDKLRENGVNAVGTILPSRVNQPIMTKNESNLRPDEFAAKFGGEPGTCRKGIFVWRDTKAFRVASNYHGSDIVKVRRKQRDGSFRSKSCPKAIDDYVNNMGGVDTANQLRSYYERDRKAKKWWHRLLYSLLETCLVNSWICFNDMVRSQMAEIKTLNTQ

SEQ ID NO: 451 - protein, 556 aa, synthetic construct
MGDQVEFIYNTRLRSRRVSADFYSRLTNALLNDMQQTNRDEDEEGEEIDPFDTGDHDDAEYVPNDDASDIENDVITIEPNEIFDSDEDIEDETDQVAGQCDDGFFYGKDGTKWSKNEPSAVIRTRQHNIMRFRFGPRQQNAIPIEVFKLFFSPNISFIIVTQTNRHGKEATRKWNAENPDSKPRVWNDLTSTELDAFVGILLAAGLSHNNMQTPEVLWLSDALPIFRAAMSYRRFKQLSRYIRFDDSRTREFRQQSDKAAPIRDIWNFLNENLANNYDAHDVVTIDEQLFPYRGRTKFTQYIPSKPAKYGIKVWWACDAKTKYPLQGKLYTGKEGTNREVNQGENVLMQLSNRYSMSGRTIVADNFFVTLDAAKRLAVIGLALVGTIRSNKRCLPSEMKKHDSRPVLSSLFGFHENVSICSYVPKKNRAVNLLSTVHYSKFCEGEAKKPEAIMFYNANKAGVDSMDQMVSHFTTKRPTRRWTYAFFCNMLDIMALAAYCICKEVDGYEKNSARRTFLNNLSNTLDPCKYRKPDGRYSRHKQERVSLSFTRKRKSAP

SEQ ID NO: 452 - protein, 740 aa, synthetic construct
MKRKAKQRPAPDPRSGLVGRYLGVPCSFFNVNVEGARYLARVKSLHKSQKSMLWIKFNQGGYGEDAFAPLAVVKQWLISDDEALDPKSDWYEDPDESDDDSDVEDGDEAEGGANARRAPAARRRVDEPRGSVKGSRWQPAEARKTPHAVDNIVWRKATGAVPSHKPKVFARKLPPGVSEGLQQPLPNHGPINVELFNRQWDIRCWELVSKYSKQRVRDLKIGTVDAVSRKPKMKNRADGSKGKQRKAPSMSVPFLIRLHVIITAMSICRLPARKYYWRKGKFVGCPGISVIMSENDFAQGIRCLYDCDPSKLVPRGDMTQPPPVGYDKMAKTSELNELVMENTLNAVCPKKTLSDDEASAEWRGKNGEGVVVSFNKDKPAKWAQRNQCFNGLDGSRVVWHVLNCPRRVAAEHGETYKSTLGLAAKLHAKYGEGFVIGQDSLYNSPVVVVDGAKKFKFNFFGTAGWNRRGMVEDADEPADTRGSCVSYYATVDGIPLTATYMRDNKIVRFLSSAHGVPTAVNGSKKPRWNKSERKYEEVYMPPVKSNYDAGKVGTDLFGQYMELLDTTFKSFHPWMVHHLWLWFGCFVNVHCWRNRFIDEGVRRLAGAKKTTLIEDTLQTMEELLQMADRLEREGAARSKKRKSRPDPQAPVRKSPRRDPVVSRLGSPTRHVPVTFTEKGRCRWCRKLIYSGCGHPGCGHMHEGTCWMKAHSNDPKIRAKLKALSFQPEIADGTQARRAPR

SEQ ID NO: 453 - protein, 528 aa, synthetic construct
MNRNKKITVSDVLSYVFDGESDFNDTDDSDNEFYNENDTANTAIEEDSDNEPLANIASRLSKNLKETTNNPNKNKPSTYKWKSKNFEPLDDVTFKEPEMKPTPNVETTPFEYFKLFVSDNMLNSITNESNIYILQSKGSEKNISKKDIEKFIGAYLRMGLVKLPSQRSYWETFMTYNGVSSIMGRNKFETILRNIHFVNNLEISKEEKADDRVWKLRTWITELRNNFQKVSPEEFHAVDEIMVPFKGKSLLRQYLPKKPHKWGFKLWGRSGISGFLYDFDIYQGKSKNNSDTSLGVSADVVIYMTSSLPDKHNFKVFADNYFTSLPLIVELRKRNIYYVGTIRDKRMKKCPLLLEKDLKKQGRGAYDFRVEQESNVVAVRWYDNKPVNLVSSYVSIEPLHTVRRYDRSLKKKIDVKQPNIVHVYNQYMGGIDKLDMMCALYKPRLRTRRWYIYIWFHTIQIAVVNAWFLYRRDLKICRPDTKFMQLQCFIAEVAESLIKVTSSRGRPSLDNITQPPKKLCSGSRKSFQ

SEQ ID NO: 454 - protein, 686 aa, synthetic construct
MCYAIRILYCNXSLHNVFGXAEYVLERCSXFAPINLLNFLKYAIAMXFIDDWQIFYTXLTPQYFXFRXLLNIFXSDXDILLLLGILMQKSFQRMNGLKNSPSEIMKTVTQXRRRRKKCFLQNLHPQLKVKRNPRSRGIPNTXSLSTVXNSTVRRPLVSRELGFHRDRNYRQVFQYSPDEVAGSSEPLSPTDYSNGYISENILESXTVXTQXXYFAKTGNPLQVNKDIMKKFIGINFIMGVLKFPRQNMYWKERTRISIIADAMARNLFQVLRAHLKLVDDITVTPAKKKESKLWKLEPLLLSIRQRCMELERTPSVAVDEMIIPFTGRTVLKQFCPRKPNPHGLKVFVLASPDGLILDFEFYQGKEQLMSSVARTNLRVGGFNNLTIGEAGVLRFVNSVPQGTSFYFDRFFTTPRLLEIMTEFGMGATGTLXKNLVPKSCKHKTEKQLKSLGRGTSDCLVRDDRGFSVTVWYDKKAISIASNQHGIEPTSQCQRWCKKTKTFLNIQRPRVIEAYNESMGGVDIADQVLSYYRNKSRTAKYTVRAMLHMFDMACSNSWLEYRRDCTKANKKPMDSMEFKLQLADSLIGGNCSGSSEGAVSSGEEEGEEXLKRRPRPIPNVETRTTGACHMPQVLPAYAIRNRCRYPGCSEKTDFLAGPAKSSFVLQITEIVIIYFISLRGSFTCFME

SEQ ID NO: 455 - protein, 569 aa, synthetic construct
MKKSWRFLREVTGRMNYLILTKVIALPQNLKTVVINRPRQEGVSMQSHVSATDVSCSESEESESDSEMPTKKSRLSDKFDWKENDFRPVNHAFDDALSGHVRQIGNEPSILSVFMLFFTEELMKYIADETNRFYLFTKENTPESVHSRLSKWKDTGYEEMYCFIAVCLLMPRVKNCKLLYTGPEMFHVFSELMSRDRFLLLLRMLHFSDNSANTGGDRLFKIRNIVDKVRQAFRRAFNPRQKLCIDESLLLFKGRLSFRQFIPSKRSRFGIKTFVLCDCKTGYILDFIVYTGTSTETEFHNLGKSGDVVATLMKPYLERGHTLYVDNWYTSPDLFSWLHSRGTNACGTVRKNRRNMPKLHKKLKRGDIQFMCTNTMLAIKWCDKREVWMLTTCNTTEMVETRKTDRHAGEKIKKPQSIVDYNLNMGAVDRSDMMLSSVGCIRKTMKWYEFFFHVLDLCVLNSHVLHRSLTGRNMSIAEFHLSLIRELTETYASERRKAGRGRCCDDNPLRFVGRHFPNIITSDQSKKSTLTRRCIVAGNRKCVEKAGTNVKLARYHCASYPVLKITTQK

SEQ ID NO: 456 - protein, 620 aa, synthetic construct
MDDKLELSKSEYSRLSYEAKEEYIRKLLTIDTEEDVASDFSDEDSTDEWIPSKEDAAASDIEDAIGACSDIEVEIETSDTSSDEHDRADENSEATVFSAGILRGKDGTKWHKEHFRTRKLLKHNILRQRAGPTRTTKLPSIKDTFKSIFTEEMCDIIIRETNRKANQVFTNYNINSEGPAKVWKPLTTEEFDAYLGILITAGVRFSNTEHTKEMWKTNAFPLYRASLSLNRFWQISRFIRFDNVNTRSERLKSASAAISDIFEMLNSNLRKNYVPSDCLTVDEQLFPYKGKTRFTQYMPSKPAKYGLKVWWVCDSRNSYPITGQIYTGKSASGRESNQGERVVKNLCYHFKGTGRNIVTDNFFTSLPLARLLMTWDLSIVGTVKRNKPYIPPEMQPSSSRQELSSIFGFIEDNVSLCSYVPKKKKAVLMLSTMHYDASTSGPLSKPDVIHYYNSTKGGVDNMDKLCSHYSVKRRTRGGRYHFLHILDIACLAAYIIYTENNPQAKNVTHRRRLFLLDLGKELAMPSIISRSSNHNCLRKFSVKSGIECMIGGPLILVPEQVEAQPKRDASGRLKITGDCFVCKSHQTKGRGKQEKVARHVRKQYVMSIALQQRCVDFVSK

SEQ ID NO: 457 - protein, 650 aa, synthetic construct
MPGFRHAAALSPVCSGHLNAQSGISAERLAPARRSASSVGVSCEFAAVDLSCAVRLSFFSFFYWILMTLPFLRRKCTDEEIFEMLTRSDSENELFDCDDSDDSSTESRSNSPVPHSSGAITEPSVSRRDENPSESDESETEGEMPVKRGRFSNTFDWKKNDYRPFVHAFDDSASGHKCVLGPDSSILLFLTECLMKFVADETNRFYLYTKQNTPESEHSRLSRWKDTEFEELYSFVAVCLLMARVKKLKISNYWSRDILLNTPIFSEIMSRDRFLLILRMLHFSDNSASNEGDRLFKIRKIVYTGRTTFHSAFHPYQKLCIDESLLLFKGRLSFKQFIPSKRSRFGIKMFVLCDCRTGYILDFIVYTGTTTEIGFHNLGKSGDIVATLLKPYLQRGHTLYVDNWYSSPDLFFWLHNHGTNACGTVRKNRRNLLKLQDNLKRGEVQFMSTDTMLAMKWCDKREVWMLTTCNTAEMCDMEKTDRKTGERRMKPQCVADYNVSMGAVDRTDMLLSSVESVRKMVKWYKKFFFHVLDLCVLNAHALHREVTGRKMPIAEFHLSLVRELVETYATERRKAGRGRRSDDENPLRFTGRHFPNITENEHSKKSRPMRRRAVCSKQKVRRETRYECKTCNVPLCVVPCFEAYHTKKYI

SEQ ID NO: 458 - protein, 576 aa, synthetic construct
MSFWFFFRLTVNEIVSCIDESDDDDFYSEIPILPPLLEGDVTDEDSGEEDVVCVDNLPRNQLVAEVVLHPESFDEEDDIPISCFVPKKKARTMNFSWKKKDLDETVTDFCYEACGLNPSSAPLDLFLLFFDETLFEHITKYSNLYAQQHNLLADIQINELKTFIGVLILSGYNSVSRRRMYWENEEDSQNILISKSITRNRFDHIFRVLHCCDNTQLDPNDRYSKVRPLFHHLLQKFLLFAPVVENYSVDEGMIPYFGRHPTKQFIRGKPIRWGYKFWVGTTPTGYIVWFEPYLGSRVSSTPKNKEHSLGTQVVLNFCHALIGRGLNLSFHLFFDNFFSSLELFQEINALKIRGTGTIRDNRLKNCSLRGSDKALKQKGRGAYSYELCSELNAVVCKWNDNNIVNMISNSVPVHPTNLVKRYSRAQKKEISVSQPKLIRTYNKNMGGVDRSDQNIGLYRIAIRGKKWYYPLFAHCIDMAISNAWQLHKHNKGTLDNLEFRRRIARSLLGTHGKHNRPRNVRPASAENVRYDQINHLIVEQDKRTRCRLCHTQAANRCEKCGVALHVRCFKTYHTMP

SEQ ID NO: 459 - DNA, 494 bp, synthetic construct
  source 1..494: mol_type = other DNA
ttaacccttctagcccggtattctgtatcctacagaataagatgtttaacaggatttagtacccagcaaagttggctgctgcccccaagtccgcatatgagaatttgtatacgtggtaatcagcagatccttcccgttggattggtgaagggtagatagacggggattcttctgtgtgagtagaaagaccaatataaaatcagaacaggtttgaaaattagagtgaaagcccaaaactgccgcgtggcaatcagtccctcccattcgaaaatcatctctgaacaatgaacggccccggagagaagacattcaagcagctgtggcgggagttatgcaaggacggatggaagccccgcaaacccacgcgcatggccaagcatcaccgctacgtgaagccaggaatcaagggacgtcttgatgaatcgagacgcggcattgactttttttagggtacgttgcaatgctaactgacctaataatgctcaatacttaca

SEQ ID NO: 460 - DNA, 147 bp, synthetic construct
  source 1..147: mol_type = other DNA
ttaaccccctggttcccgacgtcataacttacgccatacgaaatccgctatgtcgtaacttacgacatagcgggggtgacattagaaaactctattgacaatagtttatccgagatatattcaactatataagatgcgagagaccaa

SEQ ID NO: 461 - DNA, 530 bp, synthetic construct
  source 1..530: mol_type = other DNA
ttaaccccttggttcccgacgtcgtaacttcagacatagcgtttttgttatgtcataacttacgacattgaagaggaaacaatagaaattggctcggtcaatactttattcaagtcatttcaaaagtataagaccaaagaggcatttgagaaaaaccaactacgttacatagcctgcaaaagatcaaagttgggatctgcgtgccgctcgtccgcagcctttcatttcagaatctgccggatggcgcgcatccggtctagccagcgcaggaagagtgctaccgccgctgcagttgcccgcgatctcgacttcccacacttctggcggcaactgcgggcggcggggtggacgtacaaaaagccgaccggcctcgctacggaagggaggtacgtttatctaatgtacgaatgtgctatgtcgctctagactaatgtaacttgtggggatgctatagatatgctacccccgatggcaatggggtattcgtgggtgaaaccgcggttgttgcgtatgcgctggagtacgggctg

SEQ ID NO: 462 - DNA, 530 bp, synthetic construct; identical to SEQ ID NO: 461
  source 1..530: mol_type = other DNA

SEQ ID NO: 463 - DNA, 559 bp, synthetic construct
  source 1..559: mol_type = other DNA
ttaacccttgtgtggtgttcgggtctgtgggacccattttcaatgtttactaaaaaaaattaaatattttttcaacctgagactcattggccttggctcattttctgtgaagaacatgtaaaataacacattttcattgagtgcacactgtgcaccccctacacatttatattacatatgtggtgttcgggtccactggacccgagggtaataaaagtgtggaaaagtgtgtgtgtaggtgaaaacaagtaaaaatgaaacaaaaaggtttgctgtgtgccttcactctgtcttcctcctccctggcctgctgtttcaacatagcaccaagaacctgtctgtctccctgcctgtctgcctgtctcccgcttttctcgcactctttaccctttgtagtgtgtgaaactacacaatgtcttgcgggaacctgcacaatgttattacaatacattatttcgatacattatttcgatacattgtaaaaaatgcagtattttcccacactctaccccagccaggtgcaaattgggggagggacacaacaaataaatagctttgc

SEQ ID NO: 464 - DNA, 391 bp, synthetic construct
  source 1..391: mol_type = other DNA
ttaaccctttcgtgcccagtgggaatacgtattcccatttttgttttgccctgataagaccgatggaataccttttcccgatatttactactactgccatctagtatcgatcattagctaatcggtaaaacaggaatctgttttatcagacacactttgatttggcggttagttatcacacgtggcttgttgttgtggtcgtgtaaaagtgggcgaaattagtgtttttttgacaaaattcttcattgtatgtctcaataaggattttaacggttgattttagttccagagacattcttttacacattgatacataaatacgtgcttatagtgtttaattttgaaagtaagtcatattttatatatcatttcataaatcaatttataaacc

SEQ ID NO: 465 - DNA, 192 bp, synthetic construct
  source 1..192: mol_type = other DNA
ctagtgtctattcgacaagtgtcctcatatgaggactggcgcctcatatgagtacagtatttaaacagttcccgccacttgctggttggcgggaaaccccaaccgatactctctcctattccaaacacccctcgttggtgctggacaggcaagattctccttataaaactcccatcttgttccgaacacaag

SEQ ID NO: 466 - DNA, 150 bp, synthetic construct
  source 1..150: mol_type = other DNA
ctagtgtctaagcgtacaggggcctcatatgaggccacagtcctcatatgaggactcgccttccatcttccgtagcgccctaacacttacgtggaaccacaatacgacctcaaatatagaattcccgccctttttctgagcctgacagcc

SEQ ID NO: 467 - DNA, 372 bp, synthetic construct
  source 1..372: mol_type = other DNA
atatccctagtattttttatgtacgatgcatcgtacataatagaaatcttgattttgacgaaaacaccttatgtacgatgtatcgtacataacgtccctatttgaagcacattcattctaccattcctccgcaacaaatgtcccaatcccattggtaagtcattcgccgttacaagtgggctcttatgagcaatccacgtgttcatttctgggaaagacccagaaatgatcgtgttaaggtgtgaattgagtgaatccagactacagtacgactttcgtaagcaaacctgctctagcgcttaagctgaagtttctcgaaaccctcttccatcttatcccatcttcacgtatccttcagcatcttctttccca

SEQ ID NO: 468 - DNA, 112 bp, synthetic construct
  source 1..112: mol_type = other DNA
atatccctaatgctccatgtggccagtatactggacataacataaatattaaaaaagtcagatttgctcttatgtccagtatactggccacaagagcgttttttaaccgtct

SEQ ID NO: 469 - DNA, 2299 bp, synthetic construct
  source 1..2299: mol_type = other DNA
ttaaccacttctgtgccatggggccgattttgggccgatcacacaatcgcctagagtgccacggggccgattttgggccgattttgattaatattttagttgttttgtaaaaggtctttaataggtctttaagttgtcaaacttattatttttatcttggagatttgatagtttacttaatttaccaattttttataattttttcgcaaaaaaatactaataaagtatacgttgaacataactctgaaacgtcaattatgttaagttttcatttataatgacacagtttcgatcagttttaaaggtacttttacaattttaatatcatacagaataatgtacatgtatatcggtaaaagccaaaatcggggggggggggggggggagacactatataaaacagatatggtgatatgctgtctgaataataatttggaaataaactgaaaaagtgtgcttgatttaaaaaaaagtacaagtactttacaactgtttaactgaattattactaagccttgtagtagctatatagtatacatgtttttaaagcaagtagtaaaaaaacaaattaaaaaaaataattgctaatagctttatacaatattgattcgcaataacgtcatttttacgtaaatgacgtcataacaacacgtgcgccccagcatacgcgattaagatggctggtgtttggtttcgcgtcatgccacctgaaccagaggaggacaaagagtttaccggtaatccaggaccgaagaatatgccgccacggaattcatctcctctgcagtattttcttttgttttttaacttgaacctcatcagagaagttgttcgacagactaacaggtatttcaaaacatatgaaatctacccgttccaaactttgttccaaaattgagaaatgatcatgattaaaccatttttaaatagtccgatcaacaaaatattacttacatttatttgaaatatggatggacgatgcattcggacgagtagcccaattatttggaaaaatcatagttattagttcggaggatttaattgtctgtttataaaacacgaaaatgcagagagagagagagagaggtgcatgtaaaattgttatgctttatttttcgaatttcaattaaaatttacaatagtacattttgcatttatcctcaagcagtaaattcgcattttacagtcttgacactcaccgggtttcattgtattttttgtttttataaaacacactccgtatgataatcttcagtaacatcactatttaattatttttatgttaataatattaagttatatcagctggagaatttatctaatttgtattgagaactacagcatcaatgttaaactcattgccatcgtatctttccaatacatttaaacaaatcttacaaatcactttttaatttccctctcacatttataaattcatttaattaaacaactgttcttccattgaataactcaaatgacaattgagaaactctctctctctctctctctctctctctctctctctctctctctctctctcgtgttctctcatattatgactaactcgtccgtacatttttcgattctgtgcaaacaaaatgttaatttacggaacaatatttcacttacttagttttaacattaataattgaagtatttatcccgtgttttaatgacggtaatttttctttagatacgctcgcactcatattcggaactctgtgctctctcgtcattcgcgagcgaggaaatgggtaccagttacgattaaggagatgaaaggttttttggctgttatcctaaacatgggccttgtaaggaaaccaactattcccgaatactggaacaagagtcacgctagtcaactaagccgatggttcatgaaaatgttttcgagaaatcgatttcaactaattttgaagttttttcacatcgtagacaataaaagaattccacatcgcaatagccctaattacaggtgatcaaaaaccatataacagaaactttcgcactttatttattgtaataatctacatttgagtaggtttaagaagacacataatatatatatatatatatatatatatgtatatatcacacatgcagtttactgtaatttggaatacaattctatcatttgcataatgcatgttctctgactcaaaaaaatgacgcatgctatgtatcggtatcgtctttgagtatttttgataaaaactagttcggcgaaggatattctatttttgcacaatgataaataaattgaagtttactggattaattaaactataataaaggt

SEQ ID NO: 470 - DNA, 3956 bp, synthetic construct
  source 1..3956: mol_type = other DNA
ttaaccctttttcgactgacgggacgtttctttttcgacggctttcagtagggtctggtgggacctcgcccacagacacgagagatttcaaatcggctaaattcattcctatcaaagcttatatctcgaaatccatcctacagaattgaattgccttacaacgaaaaattaattgacctaccgtctttcaatgataaaaaggaggagaaattaaaacgagtatttctcgtgttttaccgagttttcggtgacgggacatttctggcccgtcaggcggctctgatcgaagtctacacgagaaccttcgatctggactgtcgggacatataagtctccgccagatatgtccctccaggcggctgcaattttatcaaaagcccggccagatttgaaccaccaggtggttgggcactttttcaagtattgatcagtggtgagctcctagtgtaacgtgcactgggatgatctgaaattcggttcatttcgatagtaggatcatttcaggagtcagaattagtgatgccgcgctcagtagatatttgataaaagggaaccaaccaccctggttagagatgtcaggaattgtgaggttttatttgtgttcctcattaaaaaaaacaaagaagtagagctgaccagccaggcaggcgcggtcgaaagaaatgggatcgagcgatggttggatagatatcgaagggaaggcgaaagaggcgaaaaggaaagcgcgaaatttctttagtagctttgacgcaaaaactttggtatcgacggggagctttgggaaatggaacgaagacgcagaatgggaagttgtccgattcggcgttagttgtagtttcatttccatcgtagctgagctcctttccgaccatcaattggttttattttgattccgcacgggcatatatgagttcgagagcatggcgggtgaaagaaatgaattcgaagttgtgtttggatgggaagctctgttggaatgggtcaaaaacgaagaatcagaagtgtccgattcaaagttactagtttcattccttgtggccagagctcctgaacctacccgccgcttcaaatgaattcagtttcgattccgttgcgtcgcttctcggtcttatagtacccacctgtctacggccggaagatataaatccgaaccgaaagattatgagagagacgagggtatcgacccgcgaaagcaattttaccctaggaagtctcgaaagtcggttgcttaatcaggaagtgaatacctgatggtgatggtattattaattcttccttgattaccgatggaacagcaaagcgaaagattggagacgaaaagaggagagttcatccccaattcgaatcttagtagttttcatctttcgtgttctactcaccggtccaagatcgtggaattccacttctgtacacagttcgcgtaatggttttaaatgatggatatggtcgaagcggtcggtgaatggttcgaatcagagacgaaagcgttttggcagataactcctaacacgtttatttgggctggtccccgtgcccattgcagtaccgggcgaatgcgccaggcgccgagtcttgaaaaatcgaaccttgccgagtcccgacgacggggtagtgacgtcgttgccgggacttggatgaatgcgagcttgtggaagacaaaataatagacgaacggtgcaattcatcctgctgaactttttgttcgaatactgatgaaacttaaaaacctagtaatctttccaatacaagagtaactccgattcatttgcccgattgccgggttttgggccgcctagaaatgatcctgattcaaccagaaatctttccggatttatagtgcccgttgggtggtcagcaatgcctcttgtcttgtcttttctgtgcgttgatcacaaatgtgtggcaatctgtggcagatgtgaaaagcgctggataagatgagaaacaaaccccgtattattgaatgtgcatgcatgtcccagcatgaggagaccgtcgaaccaaataaaattggatccttttggccaatatagaagccttgaataaacatggtaataaaaaataaagtcctaagagttttttggaagtggcgtagagacgaatgtctcgtgaattggctcctaatttaacctagagccaggtgcaaattcgattaagccagagcagactggccggttagagatgtcagaaattcaaagttcagatatgtcagaaatatattggacaacacggatgaaagaaaaatgaaggggtggttgtaatgaattcccctgggaattagcgaaaacagaacgggaagtgtcagttctaaatagtttttttccaggcggtatttttattccgttccggtccgtgtaatcgaatgaaatgaatttgaagccggtggttggatagatatcgaagggaaggcgaaagaggcgaaaaggaaagcgcgaaatttctttagtagcttcgacgcaaaaactttggtatcgatggggagctttgggaaatggaacgaagacgcagaatgggaagttgtccggttcggcgttagttgtagtttcatttccaccgtagctgagctcctttccgcccgacgctccttatttatttttattcctatctgggtgccgcgcgacttaagtgtccgcgatgcctcaaatacaaataaaaaaagggttcaagtgaatttgaagccggtggttggatagatatcgaagggaaggcgaaagaggcgaaaaggaaagcgcgaaatttctttagtagcttcgacgcaaaaactttggtatcgatggggagctttgggaaatggaacgaagacgcagaatgggaagttgtccggttcggcgttagttgtagtttcatttccatcgtagctgagctcctttccgtccaacggttcttttgaattgatcttgaagtcttcgaggcgctattagtgcccgtgtaattaaattttacacaaagcctcttgtactttcttatccgtgcgttgattactgctgtctcgtgaggaaactccgaaaggcggtgaaaggcgctggataagaatcagccccggtaactcctgcatgtgcatgcttttcccctgcatgataaaataagaccgtcgtataaggcttaggacgtaaggcaattcgttatccttttgccgaattcacaaagactaatcgtgccttttttttcgattctgtattttcagattgattaaaaatggcgaaaaataaaaaaagagagacccaagatatactaagtcgagagccgtgaaaaattcagccaagaatgccgccgccgcagctgtattaaaaaggaggcagctgaaagaagaaagagaacgtttagctggagatcaagagccggtaatcaattttgatactgtaccggaggctcaagatcaggaggaggaggcggttgccgtcgaggagccggtcgaggagccggtcgaggagccggtcgaggagccggtcgaggagtcggttgagaagccggatgaggagcctggtcaggagttggttgcttcgcagcggccggaacttgatcaggatccggaaatagaggatgaacaatctgcctcagaggatgatgaacaaaaagctgggacagaagaagaggagccggtggtagagccattaaccccgaaagacgttacagttcaacttgaacgattggaactatcaccggcgacgcttcaaagactcaaaagagttcaaaaagcgaaaaaaagaaaggaacgtgaagacagttcaaagagtggctcctccccaccaaaacgaaccagaagtcagacccgaagaccaactagtgcagggtcttcttctgaaacggccgatcccgatggtgaagtaacacggcactacacagggaaaaaggtcggtctcacgaacgcgactttgaggtgtcgtgggcctcgcgttcttacagcgcatccgttccggctggccaaaaaaacgaactcagattcggccaagctgaac

SEQ ID NO: 471 - DNA, 339 bp, synthetic construct
  source 1..339: mol_type = other DNA
ttaaccttcggacggacacatgggtctggccagacccctatacattttgtatggaaatgtatggaatatcacacattttgaccgttctacttgctttttttgttttcaaattgaagcatgcatgaacatgaacatatagaatacatatacacatacacatgcgtgaacaagtggaattcatatacaacacataagcacacttagcggcaatcttacacaaaaagtgtgtatgtacagcatttacttacttcctcttgcatcagtctacaaaaaaacgttgagcaattagcatttgtgccacgaatttttttatttgtttacttatttcaataaactata

SEQ ID NO: 472 - DNA, 234 bp, synthetic construct
  source 1..234: mol_type = other DNA
ttaacccctaaacggctgtggttcccatatgggaacttcgcccaattctgtgaaagagaaaaccggtcggctaaggagcgaagtcggccgatccggcgacccgctttcgcttggcagacggcccgggtgctgtgccgcctaccaccagccgcttgactccggagtcgcatctcggagagagaatctaagattccttcagattccctagattcccgcagcacccttcggtagcga

SEQ ID NO: 473 - DNA, 472 bp, synthetic construct
  source 1..472: mol_type = other DNA
ttaacccttaatgaccccaggtatcagatctgatacacacattttaaaccacagtattttttagacaaataactatttttatctgaaaacacccgttgttgctaatatgagaaactagttttcggttccgtataaacacttcttttcatccgggtcaattttcatataataaacattttaaaatagcacatgttaaaagtgttttacgtggtgttcaccaagtgttgtttctgtatttatttcgcgttggttatatcaattttaatgattttttttttctttaatttttatattaaaggtaaaagaaaactttattaactgtttcatgtttttttttttatcaaatatgcataaatattgattactgaatttgtatgtatcaaatttgatacaacgggtttttccagtataaagccttgtgaaaagttctttgtttatgtatatttttaattttttatttatgtttttctag

SEQ ID NO: 474 - DNA, 659 bp, synthetic construct
  source 1..659: mol_type = other DNA
ttaacccattcgtacccattatagtaaatttgtgacaaatttttggtaccctttttccacgagttttgtagaacgaggatacatgatagtgtccctttaactattgcttcgttttctgtgattactgacgtatcgaaaacgaccatttggagctgcatttaaaccctctgagactgtcatggaagccgagaggtgagcagggacctaggatcctcagattttcatcaccatgggtaagttaaattgaattcttgccttccaaagcaatgttactgatatatagaaaatatagcaccattgagtaatgccaagagatgttgttaaccattagttattgatataatcaatgattttaacaggaggaaaaaaatgatgaagtagcttgtgtctatgttagcacatcgggagtttcacaatgtaataattctgcatagttgtcattatatatcaatattattcctatagagacatttaggataccaggatgtatggcatctgtgaaagaattttgtcaacggtttttccctgtaacatgtagcataaaatattactcctatagtgaccttgtgtgatggcaggctgaggtgtgggccatttatggcacactgggaataccacacaggatttggtctctatgtagtttacattgtactaacatc

SEQ ID NO: 475 - DNA, 354 bp, synthetic construct
  source 1..354: mol_type = other DNA
ttaaccctttgactgctgttgacgagtttgtcatgctgcctgccgctccctgggtgctgatgcgagtttagtcgcggctgccggctttcccctgagcgctgttgacgagttaagtcgcagggttttccggccctgagcactggaatagccgcaccgctacctgagtgctgttgatgagttgcactgctttccccccatgtctcgtagcctttcacagctgtggccactagcttgtcgtccgattcttcatctgttgtgaaccaggcttcaccaacacagttcatcagaatttatatcagtgttcttccatcctgtattttacaaagaaaatggcaagacgttgtgggtgtactg

SEQ ID NO: 476 - DNA, 240 bp, synthetic construct
  source 1..240: mol_type = other DNA
ttaacccttgtgtccacaataaaaaaattatactttgttctataaccaggttacctgggtaacccaatgaatttattgccgtacagtcacggttggcaacacttagccctgggtatatacgcaattgttgcaacaaggcattctaattgttgctggctacagttgctactgtttcacagcgttgtgaactgttcattattgtgtaagctttgttactgtgaacatcaactctagattctc

SEQ ID NO: 477 - DNA, 137 bp, synthetic construct
  source 1..137: mol_type = other DNA
ttaaccctttaactgctctggacgtgttaacacgcgcctttatacctgtcccactgtgctctagacgtgtttacgtgcaccgcgctccttcctggtgctctggacgtgttaatgcgcgccgccctaatggctcctgt

SEQ ID NO: 478 - DNA, 391 bp, synthetic construct
  source 1..391: mol_type = other DNA
ttaacccgaatcatcccaatgtaccgcatatggtacacctaatttaaaaaatgtaagctcagaaaaaaagcattttatcgactacttttttgaataatccagtttaaatataagtaggcgaagatatttatgtgtcaatttaaacttattttggttcaattagatgtaaagttgaagtgaagtgtgtgtattttttcgtagcaaaacaatgtcgaggaaaaggtaagtaatttcttaataatgtactcaaataagttaaataaacattattactatagcttaaacaataaaaaatagtaattatgaacaattttaaaaattccaatatgcgatgttactgttacatggacgtaaaatttgatgtaccgcatatggtacagtgggataatac

SEQ ID NO: 479 - DNA, 479 bp, synthetic construct
  source 1..479: mol_type = other DNA
gaaagatagtaggaagggtaagtccaaatacctggtaatgcgcggcgcgcgcgcagagaagagtttgccctgttcctcgtcgctaaaccgttagttaggaactgggcaaattctttctctgcgcgcgcgccgcgcattttggcgccacgaacggatggctgtcgtcaaggagttgggctgctcgatcggcttcatgagcctaaacgaaaatctcgggtattggtagaatgcaggtattgactgtgctctaaccgaaaatgaaccaatagaaaatcgcaatttgcgcagctgttgggtacattgtggacgctatgtccactgtcgtaatgtttctgtgaaaaattcgcctagttcttggccatggagagcgaagggggttgcagcaggataattatgggaggtttctgaagtatacttttatcaagtataattccttattctgtaggatacagaataaaagctcgggctagaagggttaa

SEQ ID NO: 480 - DNA, 380 bp, synthetic construct
  source 1..380: mol_type = other DNA
gcaggcgatcaggagttagttccgtccgtgcacaagagttaggtggcagctttgatgagtggcagcttttgttgaatcgaaatgcactttttgctgatttgtgtttcgtctttttcctctgactggatactttgtgtcactgtgggcgtcttttttttctggttaaggtgtcagtgacgacattgagtctctggatggcgctgacaaagtgtataagttacgacattgaaaacaaaacctgttgagactttatagcgcttcccgcagacgtctatgcacaacatacaataattaagtgaatacgtacgtcgtaagttatgacatagacgtaccgtaactaacgtgtttggggtccaaaattccgggaaccaaggggttaa

SEQ ID NO: 481 - DNA, 702 bp, synthetic construct
  source 1..702: mol_type = other DNA
tggagtccagaagcgcagacagcgctcttgcaaggtgtgcgcactgctgcggtcgagcacgtccaagaagtcgttccagacgacatggttctgcgagcggtgctccgtggacgatgccaagtgctggctctgcaacaagattcgccatgagtacaggggtgtgatgaagacctgctatgagatatggcatgaagactttgatgccggtgaggccatccctacgcaccttgggaagcgagtggtcttgcgccgccccgcccagaaagctggcgcgcgtaagaagacacgccgcgagcttcagcttcgtgcgcgtgacgaaagtaagagcggcaatggcgaagagagcgaggatgagtagtgagcgtgtcacttgatgagtgatgctgctggaggttcataaaatgcatacttcttcattcaatgcaccttctttctttgcttgtctggctgggctggattatctgtgcacagtgtttatcatgagatttgagctgcagtagtaagttacgacattcgtcggtgcccttggcagctaaaagatgtataacttacgacattgagctgaaaacctctcgaaactttatgcacctagccccacgcggtttttacaccacatacaaatgcactggtacatgaattcgtcgtaagatatgacattcgcttacgtgttccggggccttttttcgggaaccaaggggttaa

SEQ ID NO: 482 - DNA, 345 bp, synthetic construct
  source 1..345: mol_type = other DNA
tgagcgtgtcacttgatgagtgatgctgctggaggttcataaaatgcatacttcttcattcaatgcaccttctttctttgcttgtctggctgggctggattatctgtgcacagtgtttatcatgagatttgagctgcagtagtaagttacgacattcgtcggtgcccttggcagctaaaagatgtataacttacgacattgagctgaaaacctctcgaaactttatgcacctagccccacgcggtttttacaccacatacaaatgcactggtacatgaattcgtcgtaagatatgacattcgcttacgtgttccggggcctttttttcgggaaccaaggggttaa

SEQ ID NO: 483 - DNA, 679 bp, synthetic construct
  source 1..679: mol_type = other DNA
ctctcctaccttggcctgctgtaactatatatgtgagtgagtgagatgttagtaaaattcctgaactaagtgttttcatcattctagattgcagccggtagcaacaagaagaagcgctgcgatgtgtgtggacccaagaaggacaggaagacacagtacacatgcatcaagtgcaagaaatacatttgcaacacacacacagtaaaactctgtccctcatgtggtgtgtagaccggccttaatttgtgttcaatggggctcatttatcatttccataaaatactgtatgtaaaatgtgtccttccaatttgttcagttcaaagcaataaacatcaatagtgatgaaaaccttgtttcatttatatttgttcaagataaacatgatttattccacccatgtcttgattataatgatttttgtttacaaaaatcacattttattaaaaaaacgaatagcgcttttattgataaatgtgatctagcagaggtaaatggcaaatattaaccatgtatgctgtctatattgcttgtaattgatataagtcaacatctaagtattaagtatttttacgtaaattgttatggctgtattgttttaaaaacccaataatgttgtgggtccatcagacccgcgaacattgggtgaataacaaaaacatgaacaccacacaagggttaa

SEQ ID NO: 484 - DNA, 627 bp, synthetic construct
  source 1..627: mol_type = other DNA
agtggtggcatcgcattatgtactacttgatagatatttccattgtaaacggatttttaatgtataaaaataaaaagcaaaataaagaagatcaactgtcatttaggatctctttagcaagacaattgatcggaaaattttcttcaagaaaacgtcgtggccgaccaacacaattcctagctaataaaggtcgtgtaccagatgatgtacgtttagaaggtgtaggtaaacacatgcctttgaagggtgacacatatagaagatgtagactatgcagtacaaaaggtcaagagaaacgaactaagtgtatgtgtaagggctgtaatgtgccattatgcatttctccatgttttgatatatttcataagaaataatttttattgtatttttttaatttatttcaattgtaattaactgtcgtaatttcaatatttttttattttttattttaagtcatgtatggccgtaaagaccagtgggaagtatagatcccagtttttttacataaaaaaataaattaaacaaaaaatattttgtgcatataataaaatatatattaatacataagaatttaaaataaaaaggttaaaaatcccaatataaataatttgggcacgaaagggttaa

SEQ ID NO: 485 - DNA, 269 bp, synthetic construct
  source 1..269: mol_type = other DNA
acgattctacgacaaagagttacaatgcggtaaagattgggactactgtaccccaaaaacacctgctggacccagggattaagtccttaaagttatccaggagtcgggggctttagggagcgagcacacagtgctttagagctgcaagaatgggatgcagtctttgttaaggagaccagtacacggaataactacatatactttcgactgcctcaaatgaggcaaacgaagcctcatatgaggaaaaaattgtgtcgaatagacactag

SEQ ID NO: 486 - DNA, 237 bp, synthetic construct
  source 1..237: mol_type = other DNA
gaccgtgggacaaacctcgaatgttgtctcttttcttgcatcttgattagaagctatgtagtctcgcagttgtaagaatactaaatgttctttgatgccctgtagcgttaagtgcggtatttgaccgttacaaccgatttgtctcttttgtattttcaaaaaagtagaatttcagggcctctggtgaggaccacccgggtctcatatgaggacaaatgttgtacgcttggacactag

SEQ ID NO: 487 - DNA, 282 bp, synthetic construct
  source 1..282: mol_type = other DNA
ttgtgggaaagaacaggcatttgtttgtgccttcttatgtacgatacatcgtacatagcgaaaaaagcaatacatttgcgtgcctctagacccgatttttaccagtagaattgatcgacatgaagatgtccttcggtaatgaagtcttgcataccgtttcttcacacgccttggcgttgctccgtgaccccagaactgggaacaaatgatgccgggctgaactctgcgagtgtgaactttaatgtacgatacatcgtacataaaataaaatattagggatat

SEQ ID NO: 488 - DNA, 349 bp, synthetic construct
  source 1..349: mol_type = other DNA
tatttcggtaatacggacctgttgcatgtcgaacgtttgaggtggtcttgaatgtgtagaataaaatgtggaaccttacctttatgcaagctatgttttcccatgtggccagtatactggccataaaggattttgtcactctgaaacttggtcagatagcttcaactttgggttacagttgaagacccataattaaggcaaagtctctcatgtttcgtccaatgaatgtagcaagtctgctgcgccgtgttacctggttcgtttcagggattgaccagattggagttacttctgtacttttaaagtttgtggccagtatactggccacaagggaaagcattagggatat

SEQ ID NO: 489 - DNA, 1113 bp, synthetic construct
  source 1..1113: mol_type = other DNA
tccgtatgtttgtgtgtattcaagtttaccctcgctataatctcttacatattaaatgtacagttgtttattctgttgttggtcatttattatttcatatagcactcttgtattatcaatttcaaagcacatatttgcatttaaggaaaaaaagaaaaggattgcatagtttgctctgatcgcgcgaatggcgtgaggagaaggtccaggatgcagtgtacgaagtgcaacaaagggcttcacttccagtgtaggaaaaggcacgcttgctgtgaggagtgaatggacaatgagacactataaactgacatttttgagtaaaacgagttttgttatcctgaagattggtatacatgtagaaaagtaaaatgttgatatttcactaactgtctctgacatttaacttaacgggtaagagcaaaatgtcgtatgtacgttaagactgaatcgacaagggtctgctgtatatactactattgatgatgtgatacgtattacaaatggcgattagctgcagaactgtacatagctctccggtctaaaaatgttgggactggagagctacatgtacagtttggcagttaaaacagtgatgtgcggtcatttatctcggacgatgaataccggtcactgcaaaacagggagatcaaactaagtttacacttctgagtggttcaatcctagatgtgttcgcaagcgcaaataatatatcaatgtagcttctagtctgttcttgatatcgtataaaattgttgcttactcatgagtcaagtaaattttatttttaactgttttgaagatactgggctaaaatatattcaacataatctttctaagatataatggttttgtatatttgggtacacgtgtacacggtactaaccttcccaatctgataaaaattcaaatctctcatataatgcgcagagagagagagagagagagagagagagagagagagagagagcatgtgcctgtacgtcataaacataaacataaaaaataacgttgagcaaggcaacgtcatacaatttcttacgtcaaattaatttgacgttgcaattttggcaatttgaaaatttacgtcaaaaacgctggcagcgaaagggttaa

SEQ ID NO: 490 - DNA, 1239 bp, synthetic construct
  source 1..1239: mol_type = other DNA
tttccgttttttttcttgacttaggtcgaggaaaattatctggaggattacgaagttcagatgccgtttctcgagttcaaacggaacgtcacgatgggtcttttgtcccacgccctgaacgaaaataaaaccaaagctggtcgagccgggcgaatgatgccaaccattcacccttccgctgaaccaggagcaaagcggcggaaaagtcgactgagtgtaagggacgacattcggttaacttgtgtgggcaaccatctacccatctttggggaggcgagaggcaggtgcgagtggtgccaggcaacgacacccaaaaaattggaatcaagaccattctcaaagtgcaaacaatgtaatgtgtttttgtgtctcgggaagaagagaaattgctttgtcgagtttcacgatgataattacactcaatctgaggaggaggctccgctggacgaggtggaagctgtctattccgcttcggaggaatcagacggccaagcttctgaccatgagccaaccaacaaggagtgttcttcttcatccgctgattccgacgagctagaaaattctaaccgacgtgaaggctatatgattgatgattatgatgatttccaagaccgtgatggatttgtaattgatgaatatgatgaatttcagcaataaatttgtgttttgtttcgtacgtccttgtgcatttttactcgaaagtgcggctacttgccagagtggtgaccgacgaaggatctgactgacttttctaattagagatcctattccttttcataaggagccgtcgactggaaagacccctggtcgatggtaaagacctcaacgtcaggcggggcgcctttcattctgccaggtgaatgagagtcaagcccgcctgacgggacacaagaggcacattaaacggcaaaagtctctcgggacaaatatgacgaaaaaaactcgagccgtctgtcgggacaaaaatttgaacaagtggtagttgcatgcaactgtcgggacaaattttttgtcccgctagacggctcgagtcgaaaaaagtgtctctttttagagaccatgtccgcctgaagggacattcctgtcgggacaacggtggccgaaacgcggttaggccggaaaaaatcggatattccgaatttttttttaatgagtggtcttaggaccactcaatgatatttcgatcgagcattcaatcgaatccgaccatcggttttgtggcggttgataaagggttaa

SEQ ID NO: 491 - DNA, 426 bp, synthetic construct
  source 1..426: mol_type = other DNA
aactcgttttttttgtactgcttgcaatcagccagtatgccaacaacactcaaaaacagcttatacatgctttgtttgtgtttctcagaatccgtaagaaaattggaattatattttccatcatacaattacatttcattccatttctattgttaaaacaacaacaaattaattaaaaataaaaaacaaaagtgaacgaaagaaaaatgtcttttttccgtaatccgattttttttcgggtatatattttcttccacatatagctcaatgcatggcaaatcgattaagaatggtaagaaattaaaatatctacctactcggtacatatatgagccagaatatatggttaatttctatttaaatgcatgcaaatgcatgtaaatacataggggtctggccagacccaagtgtccgtccgaaggttaa

SEQ ID NO: 492 - DNA, 798 bp, synthetic construct
  source 1..798: mol_type = other DNA
agagaccgtcacaatcccgctctgcgccttcggtggcaggcggtggcaagggaatggggagacgatacgccgtccaatccggcagagagttgtctttcggcagcacggggggtgagagcgtttggcgtggtcgatggtgcaccgccgtcacggtcacacgcacacacacgcgcgcgccgcgcgcgcgcacaccacacacacacacacccatcctgatcgcgccgtgcgcttggggggccctcaggtgtgcatccgcaaggtgaagcggacgcgcaagcccaagcggaagttgcgctacaacgaggaggagggcggcgccggggccgacgccgacgccgaggaggaggacgacgacggtgatggcgacgatgacgctccggctccgcccgccaagcgccgcagcacccgcgcgcagcagcagcgccagggcgcgcggtgagacctctcgccgacgcttgaaccggctttgctgcactctgcgacgctgcggtacgtgtgtacctgtctagtagtgaccggtggagtgagcggcgggcgtgatgtcaccggttgagtgccagtgtgcgtgcagaatcggcgatcgctgcccccgtctggcctgtcctggcggcgacggacgtgtttgagggagatttcgaggtgacgtctttgccgtccctagcctccaagtgtcactaagtaatgtgcaacaccctccctacgcatgctggggtgctgggcgggcgaatagacgcgcgagaagccctctcaaaaatcgacccctcggaccccgtttttagctagccgtttaggggttaa

SEQ ID NO: 493 - DNA, 398 bp, synthetic construct
  source 1..398: mol_type = other DNA
tgtaagaaaggatggttttgatcatatgccaagttacaatgaaaaaagacagaggtgtttgtcctgtaagtcaggactttcatatataagttgtaaaaagtgtaatgcatggctttgttttaagaaagaaagaaactgttttgaagtttttcattcataatcttattatgaatgtgctaatatgtttagcttatatgtactatgtagtatgtttaatataaatattttgataaataattacccgttgtatcatatctgatacaactcaaaaaagtgcctagtcatcatttttttgtctcaaatttttgtcagcaatcaacttatgccataagggttacttgtatttgaaataagatttttttgagtcttttaaactgttttgggtatttaagggttaa

SEQ ID NO: 494 - DNA, 588 bp, synthetic construct
  source 1..588: mol_type = other DNA
ttagttgattcaagatctagttttttttcttttctaaacaggactgtataatttcttagtgatttctttaaattttgaaactagcttaatgtttccaaaattgtttcatcttcattttgattttatatacttatagaatgatcagtttatttaagaattttctagccataagtgatgcttaaacacttcacttcttgtttaaagtattctttgttttccaatgaatgctattattttttcacatgctgtgatatttttgtcctcaaatacctcaaatgctcatatcagagtttgcaaagagagttttttttgtattgatatatttttcttacaagaataaaggttattttcttcaaaatgcaatatcaattataatacctttgtttaaatagaaaatgttcatttacataccaaatccgaaatccattgtgacaaatttggaacaagcttaatccagctaaagctttaaaagtatttgagatatcagattttttgttgttgcatttacatgtacgattttttatccataagtcctgtgattttcatgcaaatcagaaacaaaaaagttcttgggtacgaatgggttaa

SEQ ID NO: 495 - DNA, 977 bp, synthetic construct
  source 1..977: mol_type = other DNA
attactaatcgcaacaaagtacgaatacaccagtgcaaaagaaaaaaaaataattaaaaaaatcgccagtgccggtccaaagcccggatcaaaaaggatggaaaatatattcagaaggtgtgaacattagtaatgtttaacgatcgtcaaaataatactgactgatatagtcgcttgaatggcttgatgcgcaaatttgaactaaatttactcggtatgggaaagaggtacataaatgaaactttatatttgagaaccttgtgttgttaggttactacataccaagtttcactttcgtcatactttcagtccctgagatataagaagccgaacttgagatttttttctggtgccgattttttaggatttatttatttatttctttattattcttaagataacgaatatagttgtacaagatagccaattttaatatattttaacaaaatattaaccgagatataacagttcaaattccccagaaattcacacgcactctgcgcaatgtcgtgcatgaagtcaaacaagtgactatatcacatccagtataacatcgacgattgttaaacttcaccaatgttcatagtggatatatgagaatgttcatataaaattacttcgaaatccgttttagtcgattgggaaaatgttccaaatttatttatttgacgtggaatgacccataagcagtttcaatgcacgtactgtctgttcagtaattaatctgcagtaattcttctgccacggaggaagtatgcagatagcgtacattgtgtgtcgtgatacactcagaaggtgacacagccgcgaccgcctggctacagaaatattcagcacgggcaaggtcggccgagtgacgggctggcggttgcggcagccgtcgggaagcacagtagcgcaggtgcggggattaggcggcctaagggcgcgcgcgacctgaccgactggatgctcggcggcagcagctaaagggttaa

SEQ ID NO: 496 - DNA, 453 bp, synthetic construct
  source 1..453: mol_type = other DNA
taattccaagtgtatctttccattgtaacgagcagcaaaatttagtagctaataattttatgtaatttcgttacatgaagtaaaaaactgatatatttttcacttatttatgtctatatctgttgtactattacatggttgtctaataaataataaaacagttaatatattatataaaataaagtattacatttatgaacttcacacctgttataatggagatatatggttacctgggtaacccaaatatagattagagggtgtgttctgttgcttcccatttaaatgctcttctatgaaagtatttaaatttttcctgttattatgacgtttataaagatgcaaatgaggaataacctgaggcttgtgtgcactagtttcaatacacctgaaaaatgggctgttacagttagaaaggttacccgggtaacccaatgtggacacaagggttaa

SEQ ID NO: 497 - DNA, 1067 bp, synthetic construct
  source 1..1067: mol_type = other DNA
ctgtcgattatcgcactgcaaaatgaaattttttaagtaaatgaaagcaaaaataaataaaaattaaaaaaggaaaaaaatgacaaaaaatatatttttaacttcgctagtgccggtccaaagcccggatcaaaaaggaggatggaaaatatattcagaaggtgtgaacattagtaatgtttaacgatcgtcaaaacaatactgactgagatatagtcacttgtatggcttgatgcgcaaatttgaactaaatttactcggtatgggaaagaggtacatgaatgaaactttatatttgagaaccttgtgttgttaggttactacataccaagtttcactttcgtcatactttcagtccctgagatataagaagccgaacttcgagatttttttctggcgccgattttttatggcgaatttgcttaatattttcggctgaatatggctcgtaaaattcttttctttattattcttaagataacgaatatagttgtacaaggtagccaattttaatatattttaacaaaatattaaccgagatataacagttcaaattccccagaaattcacgcgcactctgcgcaacgtcgcgcatgaagtcaaacaagtgactatatcacatccagtataacatcgacgattgttaaacttcaccaatgttcatagtggacatatgagaatgttcatataaaattacttcgaaatccgttttagtcgatcgggaaaatgttccgaaatttatttatttgacgtggaatgacccataagcagtttcaatgcacgtactgtctgttcagtaattcatctgcagtaattcttctgccacagaggaagtatgcagatggcgtacattgtgtgtcggtgatcacactcagaaggcgacacagccgcgaccgccctggctccaaaaatattcagcacagggcaaggtcggccaagtgatgggctgagcagcaggccggcaggtcgatcgggaagcacaatagcgcaggtgcggggattaggcggcctcaagggcggcgtgacccaacgcgaccgaatgctcggtggcagcagctaagggttaa

SEQ ID NO: 498 - DNA, 264 bp, synthetic construct
  source 1..264: mol_type = other DNA
gcatttatgtaaccacttttcaataatattcaaataaaaacttactgcctaatgaaagaatactttaaataacgttatgtattaatattgatttatgacagtgcattatcccactgtaccatatagggtacacatcatccccaggaaatggtaagtaatttttttttttggctttctttgtgtattacaattacaaaatgtggactaatttccaaaatcatagaaaaattacaaaaaaaaaatttctgggataattcgggttaa

SEQ ID NO: 499 - DNA, 584 bp, synthetic construct
  source 1..584: mol_type = other DNA
gacattgattattgactagttattaatagtaatcaattacggggtcattagttcatagcccatatatggagttccgcgttacataacttacggtaaatggcccgcctggctgaccgcccaacgacccccgcccattgacgtcaataatgacgtatgttcccatagtaacgccaatagggactttccattgacgtcaatgggtggagtatttacggtaaactgcccacttggcagtacatcaagtgtatcatatgccaagtacgccccctattgacgtcaatgacggtaaatggcccgcctggcattatgcccagtacatgaccttatgggactttcctacttggcagtacatctacgtattagtcatcgctattaccatggtgatgcggttttggcagtacatcaatgggcgtggatagcggtttgactcacggggatttccaagtctccaccccattgacgtcaatgggagtttgttttggcaccaaaatcaacgggactttccaaaatgtcgtaacaactccgccccattgacgcaaatgggcggtaggcgtgtacggtgggaggtctatataagcagagct

SEQ ID NO: 500 - DNA, 225 bp, synthetic construct
  source 1..225: mol_type = other DNA
ctgtgccttctagttgccagccatctgttgtttgcccctcccccgtgccttccttgaccctggaaggtgccactcccactgtcctttcctaataaaatgaggaaattgcatcgcattgtctgagtaggtgtcattctattctggggggtggggtggggcaggacagcaagggggaggattgggaagacaatagcaggcatgctggggatgcggtgggctctatgg

SEQ ID NO: 501 - DNA, 720 bp, synthetic construct
  source 1..720: mol_type = other DNA
atggtgagcaagggcgaggagctgttcaccggggtggtgcccatcctggtcgagctggacggcgacgtaaacggccacaagttcagcgtgtccggcgagggcgagggcgatgccacctacggcaagctgaccctgaagttcatctgcaccaccggcaagctgcccgtgccctggcccaccctcgtgaccaccctgacctacggcgtgcagtgcttcagccgctaccccgaccacatgaagcagcacgacttcttcaagtccgccatgcccgaaggctacgtccaggagcgcaccatcttcttcaaggacgacggcaactacaagacccgcgccgaggtgaagttcgagggcgacaccctggtgaaccgcatcgagctgaagggcatcgacttcaaggaggacggcaacatcctggggcacaagctggagtacaactacaacagccacaacgtctatatcatggccgacaagcagaagaacggcatcaaggtgaacttcaagatccgccacaacatcgaggacggcagcgtgcagctcgccgaccactaccagcagaacacccccatcggcgacggccccgtgctgctgcccgacaaccactacctgagcacccagtccgccctgagcaaagaccccaacgagaagcgcgatcacatggtcctgctggagttcgtgaccgccgccgggatcactctcggcatggacgagctgtacaagtaa

SEQ ID NO: 502 - DNA, 600 bp, synthetic construct
  source 1..600: mol_type = other DNA
atggggaccgagtacaagcccacggtgcgcctcgccacccgcgacgacgtcccccgggccgtacgcaccctcgccgccgcgttcgccgactaccccgccacgcgccacaccgtcgacccggaccgccacatcgagcgggtcaccgagctgcaagaactcttcctcacgcgcgtcgggctcgacatcggcaaggtgtgggtcgcggacgacggcgccgcggtggcggtctggaccacgccggagagcgtcgaagcgggggcggtgttcgccgagatcggcccgcgcatggccgagttgagcggttcccggctggccgcgcagcaacagatggaaggcctcctggcgccgcaccggcccaaggagcccgcgtggttcctggccaccgtcggcgtctcgcccgaccaccagggcaagggtctgggcagcgccgtcgtgctccccggagtggaggcggccgagcgcgccggggtgcccgccttcctggagacctccgcgccccgcaacctccccttctacgagcggctcggcttcaccgtcaccgccgacgtcgaggtgcccgaaggaccgcgcacctggtgcatgacccgcaagcccggtgcc

SEQ ID NO: 503 - DNA, 500 bp, synthetic construct
  source 1..500: mol_type = other DNA
gggtaggggaggcgcttttcccaaggcagtctggagcatgcgctttagcagccccgctgggcacttggcgctacacaagtggcctctggcctcgcacacattccacatccaccggtaggcgccaaccggctccgttctttggtggccccttcgcgccaccttctactcctcccctagtcaggaagttcccccccgccccgcagctcgcgtcgtgcaggacgtgacaaatggaagtagcacgtctcactagtctcgtgcagatggacagcaccgctgagcaatggaagcgggtaggcctttggggcagcggccaatagcagctttgctccttcgctttctgggctcagaggctgggaaggggtgggtccgggggcgggctcaggggcgggctcaggggcggggcgggcgcccgaaggtcctccggaggcccggcattctgcacgcttcaaaagcgcacgtctgccgcgctgttctcctcttcctcatctccgggcctttcg

SEQ ID NO: 504 - DNA, 57 bp, synthetic construct
  source 1..57: mol_type = other DNA
gccactaacttctccctgttgaaacaagcaggggatgtcgaagagaatcccgggcca

(19) United States
(12) Patent Application Publication
(10) Pub. No.: US 2025/0270521 A1
(43) Pub. Date: Aug. 28, 2025

(54) ISOLATED TRANSPOSASE AND USE THEREOF

(71) Applicant: Beijing AstraGenomics Technology Co., Ltd., Beijing (CN)

(72) Inventors: Daqi YU, Beijing (CN); Chen ZHAO, Beijing (CN); Ting WEI, Beijing (CN); Chengxi SHI, Beijing (CN)

(21) Appl. No.: 18/866,304

(22) PCT Filed: Mar. 26, 2024
(86) PCT No.: PCT/CN2024/083808

§ 371 Date: Nov. 15, 2024

(30) Foreign Application Priority Data
Mar. 27, 2023 (CN) 202310304620.3

(51) Int. Cl. C12N 9/12; C12N 15/52; C12N 15/85; C12N 15/90

(52) U.S. Cl. CPC C12N 9/1241; C12N 15/52; C12N 15/85; C12N 15/90; C12N 2830/50

(57) ABSTRACT

Provided are a nucleic acid and a nucleic acid construct encoding the transposase, a nucleic acid set and a nucleic acid set construct, and a composition, a recombinant vector, a recombinant host cell and a kit comprising the transposase. Further specifically provided are a method for introducing an exogenous nucleic acid fragment into the genome of a host cell, a method for editing the genome of a host cell, and a method for obtaining a host cell containing an exogenous nucleic acid fragment in the genome.

### CROSS-REFERENCE TO RELATED APPLICATIONS

This application claims priority to Chinese Patent Application No. 2023103046203, filed with the China National Intellectual Property Administration on Mar. 27, 2023, the entire contents of which are hereby incorporated by reference in their entirety for all purpose.

### TECHNICAL FIELD

The present application relates to the field of molecular biology, and specifically to an isolated transposase and the use thereof. The present application further specifically relates to: a nucleic acid and a nucleic acid construct encoding the transposase, a nucleic acid set and a nucleic acid set construct, and a composition, a recombinant vector, a recombinant host cell and a kit comprising the transposase. The present application further specifically relates to: a method for introducing an exogenous nucleic acid fragment into the genome of a host cell, a method for editing the genome of a host cell, and a method for obtaining a host cell containing an exogenous nucleic acid fragment in the genome. The present application further specifically relates to the use of the transposase, the nucleic acid and the nucleic acid construct, the nucleic acid set and the nucleic acid set construct, the composition, the recombinant vector, or the recombinant host cell for introducing an exogenous nucleic acid fragment gene into the genome of a host cell or preparing a drug or a preparation for gene therapy, cell therapy, genome research, or stem cell induction and post-induction differentiation.

### BACKGROUND

A transposon is a DNA sequence that can be inserted into or excised from the genome to transfer its own sequence or a complete copy of its own sequence within or between genomes. Transposons fall into two main categories, which are referred to herein primarily as type II transposons (DNA transposons), consisting of a terminal inverted repeat (TIR) at both ends and a gene encoding a transposase. Transposons have a “cut-and-paste” transposition mechanism, where DNA is cleaved from chromosomes and directly inserted into other parts of the genome.

Transposases are sequence-specific DNA-binding proteins expressed by DNA transposon sequences, comprising catalytic domains that mediate DNA breakage and ligation. Transposases can recognize and bind to TIRs at both ends of transposons, forming a bulge complex, and then remove the DNA transposon from the original site and integrate it into a new site. The transposition activity of a transposon is mainly dependent on the expression level and activity of transposases. Therefore, DNA transposons having a high transposase activity are a major requirement for the development of transposon function-based gene editing tools.

Gene insertion and integration of large fragments have important application value in fields such as gene therapy, molecular breeding of animals and plants, and engineering of industrial microorganisms. Currently, there is a lack of effective tools and systems for insertion and integration of a large fragment gene in the industry. In recent years, the scientific community has developed some tools and methods capable of inserting and integrating a large fragment gene, but these methods still have some problems. For example, in cellular immunotherapy and gene therapy for genetic diseases, lentivirus or retrovirus are most commonly used to integrate gene sequences, and based on this, there are several therapeutic products for the treatment of tumors and genetic diseases (Aiuti, A., Roncarolo, M. G. and Naldini, L. (2017) Gene therapy for ADA-SCID, the first marketing approval of an ex vivo gene therapy in Europe: paving the road for the next generation of advanced therapy medicinal products. EMBO Mol. Med. 9, 737-740; Aiuti, A. et al. (2009) Gene therapy for immunodeficiency due to adenosine deaminase deficiency. N. Engl. J. Med. 360, 447-458). However, using viruses to integrate a large fragment gene has some potential application limitations: first, the randomness of virus integration in the genome creates the risk of cancer; second, the size of an exogenous gene the virus can carry is also limited, which is not conducive to the transfer of a therapeutic large fragment gene; third, the immunogenicity of the virus may affect the long-term expression of an exogenous therapeutic gene and re-administration; fourth, the production of viruses needs to be completed with the help of living cells, which makes the quality control and downstream processing of such products more complex and more expensive, and has certain disadvantages in terms of industrialization. Therefore, non-viral large fragment integration can avoid various disadvantages caused by viral integration and become a valuable tool in gene therapy.

As a non-viral gene integration tool, DNA transposons not only can achieve the integration in a host genome and stable expression of a large fragment of an exogenous gene, but also can circumvent negative effects such as immunogenicity, and thus some transposons have been used in gene therapy. Although transposons have been proved to be widely present in various fields from prokaryotes to eukaryotes, during evolution, in order to maintain genomic stability, a large number of transposon fragments become silently inactive. At present, a few highly active and valuable transposon tools, such as Sleeping Beauty (SB), PiggyBac (PB) and Tol2, are used in gene therapy studies. Therefore, the excavation of more highly active transposon tools and the verification and detection of their functions can provide more, better and flexible choices for the development of gene therapy strategies.

It should be noted that methods described in this section are not necessarily methods that have been previously conceived or employed. It should not be assumed that any of the methods described in this section is considered to be the prior art just because they are included in this section, unless otherwise indicated expressly. Similarly, the problem mentioned in this section should not be considered to be universally recognized in any prior art, unless otherwise indicated expressly.

### SUMMARY

Based on this, in order to seek more advanced and more effective non-viral gene integration tools, the present application provides an isolated transposase, wherein the transposase has a transposase sequence selected from the following (i) or a variant sequence of the aforementioned transposase having a transposase activity in (ii)-(iv): (i) at least one amino acid sequence as shown in any one of SEQ ID NOs: 1-146; (ii) at least one of sequences obtained by performing deletion, substitution, insertion, or mutation of 1, 2, 3, 4, 5, 6, 7, 8, 9 or 10 amino acids on the amino acid sequence as shown in any one of SEQ ID NOs: 1-146; (iii) at least one of amino acid sequences having at least 70%, 80%, 90%, 95% or 99% identity to the amino acid sequence as shown in any one of SEQ ID NOs: 1-146; and (iv) at least one of sequences obtained by further fusing the amino acid sequence as shown in any one of SEQ ID NOs: 1-146 with other sequences. The transposase provided in the present application has equal or even higher transposition activity compared to Sleeping Beauty (SB) and PiggyBac (PB), which are widely used now, providing more or better choices for the development of gene integration tools.

According to an embodiment of the present application, an isolated transposase can be provided, wherein the transposase comprises an amino acid sequence as shown in the following formula:

DE (X₁)ₐ K (X₂)_{b} G (X₃)_{c} K (X₄)_{d} G

wherein a, b, c and d are the numbers of amino acids; D is aspartic acid; E is glutamic acid; K is lysine; G is glycine; (X₁) is any amino acid, and a is 17, 18 or 19; (X₂) is any amino acid, and b is 3, 4 or 5; (X₃) is any amino acid, and c is 1; and (X₄) is any amino acid, and d is 17, 18 or 19.

According to an embodiment of the present application, an isolated transposase can be provided, wherein the transposase comprises an amino acid sequence as shown in the following formula:

P (X₅)ₑ Y (X₆)_{f} D

wherein e and f are the numbers of amino acids; P is proline; Y is tyrosine; D is aspartic acid; (X₅) is any amino acid, and e is 5; and (X₆) is any amino acid, and f is 7.

According to an embodiment of the present application, an isolated transposase can be provided, wherein the transposase comprises an amino acid sequence as shown in the following formula:

C (X₇)_{g} C (X₈)ₕ C (X₉)ᵢ C

wherein g, h and i are the numbers of amino acids; C is cysteine; (X₇) is any amino acid, and g is 2, 3 or 4; (X₈) is any amino acid, and h is 7, 8, 9, 10, 11, 12, 13, 14, 15, 16, 17, 18, 19, 20, 21, 22, 23, 24, 25, 26, 27 or 28; and (X₉) is any amino acid, and i is 2.

According to an embodiment of the present application, an isolated transposase can be provided, wherein the transposase comprises at least two of the following amino acid sequences (1)-(3):
(1) D E (X₁)ₐ K (X₂)_{b} G (X₃)_{c} K (X₄)_{d} G;
(2) P (X₅)_{c} Y (X₆)_{f} D; or
(3) C (X₇)_{g} C (X₈)ₕ C (X₉)ᵢ C,

wherein a, b, c, d, e, f, g, h and i are the numbers of amino acids; D is aspartic acid; E is glutamic acid; K is lysine; G is glycine; P is proline; Y is tyrosine; D is aspartic acid; C is cysteine; (X₁) is any amino acid, and a is 17, 18 or 19; (X₂) is any amino acid, and b is 3, 4 or 5; (X₃) is any amino acid, and c is 1; (X₄) is any amino acid, and d is 17, 18 or 19; (X₅) is any amino acid, and e is 5; (X₆) is any amino acid, and f is 7; (X₇) is any amino acid, and g is 2, 3 or 4; (X₈) is any amino acid, and h is 7, 8, 9, 10, 11, 12, 13, 14, 15, 16, 17, 18, 19, 20, 21, 22, 23, 24, 25, 26, 27 or 28; and (X₉) is any amino acid, and i is 2.

According to an embodiment of the present application, an isolated transposase can be provided, wherein the transposase comprises the following amino acid sequences (1)-(3):
(1) D E (X₁)ₐ K (X₂)_{b} G (X₃)_{c} K (X₄)_{d} G;
(2) P (X₅)ₑ Y (X₆)_{f} D; and
(3) C (X₇)_{g} C (X₈)ₕ C (X₉)ᵢ C,

wherein a, b, c, d, e, f, g, h and i are the numbers of amino acids; D is aspartic acid; E is glutamic acid; K is lysine; G is glycine; P is proline; Y is tyrosine; D is aspartic acid; C is cysteine; (X₁) is any amino acid, and a is 17, 18 or 19; (X₂) is any amino acid, and b is 3, 4 or 5; (X₃) is any amino acid, and c is 1; (X₄) is any amino acid, and d is 17, 18 or 19; (X₅) is any amino acid, and e is 5; (X₆) is any amino acid, and f is 7; (X₇) is any amino acid, and g is 2, 3 or 4; (X₈) is any amino acid, and h is 7, 8, 9, 10, 11, 12, 13, 14, 15, 16, 17, 18, 19, 20, 21, 22, 23, 24, 25, 26, 27 or 28; and (X₉) is any amino acid, and i is 2.

According to an embodiment of the present application, a nucleic acid can be provided, wherein, the nucleic acid encodes the transposase described in the present application.

According to an embodiment of the present application, a nucleic acid construct can be provided, comprising the nucleic acid according to the present application, and further comprising a promoter.

According to an embodiment of the present application, a nucleic acid set can be provided, comprising a 5′ recognition sequence, wherein the 5′ recognition sequence comprises at least one of the nucleotide sequences as shown in SEQ ID NOs: 147-292.

According to an embodiment of the present application, a nucleic acid set can be provided, comprising a 3′ recognition sequence, wherein the 3′ recognition sequence comprises at least one of the nucleotide sequences as shown in SEQ ID NOs: 293-438.

According to an embodiment of the present application, a nucleic acid set can be provided, comprising a 5′ recognition sequence and a 3′ recognition sequence, wherein the 5′ recognition sequence comprises the nucleotide sequence as shown in any one of SEQ ID NOs: 147-292 or a variant thereof, the 3′ recognition sequence comprises the nucleotide sequence as shown in any one of SEQ ID NOs: 293-438 or a variant thereof, and the nucleic acid set can be recognized by a specific transposase.

According to an embodiment of the present application, a nucleic acid set construct can be provided, the nucleic acid set construct includes the nucleic acid set described in the present application and further includes an exogenous nucleic acid fragment.

According to an embodiment of the present application, a composition may be provided, wherein, the composition includes: a PiggyBac family transposase or a functional fragment thereof, or a nucleic acid encoding the PiggyBac family transposase or the functional fragment thereof, wherein the transposase or the functional fragment thereof has a function of catalyzing the insertion of an exogenous nucleic acid fragment into the genome of a cell; and a nucleic acid set, wherein the nucleic acid set can be recognized by a specific transposase or a functional fragment thereof.

According to an embodiment of the present application, a recombinant vector can be provided, wherein, the recombinant vector comprises the nucleic acid encoding the transposase described in the present application, the nucleic acid described in the present application, the nucleic acid construct described in the present application, the nucleic acid set described in the present application, the nucleic acid set construct described in the present application, or the composition described in the present application.

According to an embodiment of the present application, a recombinant host cell can be provided, wherein, the recombinant host cell comprises the transposase described in the present application, the nucleic acid encoding the transposase described in the present application, the nucleic acid described in the present application, the nucleic acid construct described in the present application, the nucleic acid set described in the present application, the nucleic acid set construct described in the present application, the composition described in the present application, or the recombinant vector described in the present application.

According to an embodiment of the present application, a method for introducing an exogenous nucleic acid fragment into the genome of a host cell can be provided, wherein, the method comprises: delivering the transposase described in the present application, the nucleic acid encoding the transposase described in the present application, the nucleic acid described in the present application, the nucleic acid construct described in the present application, the nucleic acid set described in the present application, the nucleic acid set construct described in the present application, the composition described in the present application, or the recombinant vector described in the present application into a host cell.

According to an embodiment of the present application, a method for editing the genome of a host cell can be provided, wherein, the method comprises: delivering the transposase described in the present application, the nucleic acid encoding the transposase described in the present application, the nucleic acid described in the present application, the nucleic acid construct described in the present application, the nucleic acid set described in the present application, the nucleic acid set construct described in the present application, the composition described in the present application, or the recombinant vector described in the present application into a host cell.

According to an embodiment of the present application, a method for obtaining a host cell containing an exogenous nucleic acid fragment in the genome can be provided, wherein, the method comprises: delivering the transposase described in the present application, the nucleic acid encoding the transposase described in the present application, the nucleic acid described in the present application, the nucleic acid construct described in the present application, the nucleic acid set described in the present application, the nucleic acid set construct described in the present application, the composition described in the present application, or the recombinant vector described in the present application into a host cell.

According to an embodiment of the present application, the use of the transposase described in the present application, the nucleic acid encoding the transposase described in the present application, the nucleic acid described in the present application, the nucleic acid construct described in the present application, the nucleic acid set described in the present application, the nucleic acid set construct described in the present application, the composition described in the present application, the recombinant vector described in the present application, or the recombinant host cell described in the present application for introducing an exogenous nucleic acid fragment into the genome of a host cell can be provided.

According to an embodiment of the present application, the use of the transposase described in the present application, the nucleic acid encoding the transposase described in the present application, the nucleic acid described in the present application, the nucleic acid construct described in the present application, the nucleic acid set described in the present application, the nucleic acid set construct described in the present application, the composition described in the present application, the recombinant vector described in the present application, or the recombinant host cell described in the present application for preparing a drug or a preparation for gene therapy, cell therapy, genome research, or stem cell induction and post-induction differentiation can be provided.

According to an embodiment of the present application, a kit can be provided, wherein, the kit comprises the transposase described in the present application, the nucleic acid encoding the transposase described in the present application, the nucleic acid described in the present application, the nucleic acid construct described in the present application, the nucleic acid set described in the present application, the nucleic acid set construct described in the present application, the composition described in the present application, the recombinant vector described in the present application, or the recombinant host cell described in the present application.

It should be understood that the content described in this section is not intended to identify critical or important features of the examples of the present application, and is not used to limit the scope of the present application. Other features of the present application will be easily understood through the following description.

### BRIEF DESCRIPTION OF THE DRAWINGS

The accompanying drawings exemplarily show embodiments and form a part of the specification, and are used to explain exemplary implementations of the embodiments together with a written description of the specification. The embodiments shown are merely for illustrative purposes and do not limit the scope of the claims. Throughout the accompanying drawings, the same reference numerals denote similar but not necessarily same elements.

FIG. 1 shows a schematic diagram of two plasmid vectors in the transposon activity detection system in example 1. Plasmid 1 is a plasmid expressing a transposase (Tn), and plasmid 2 is a transposon donor plasmid.

FIG. 2 shows the relative transposition efficiency results of PB01_B9, PB01_B10, PB01_B11, PB01_B12, PB01_C3, PB01_C4, PB01_C7, PB01_C8, PB01_C9, PB01_C12, PB01_D1, PB01_D3, PB01_D4, PB02_A2, PB02_A4, PB02_A9, PB02_A12, PB02_B2, PB02_B8, PB02_B9, PB02_C6, PB02_C11, PB02_D3, PB02_D12, PB02_E1, PB02_E4, PB02_E5, PB02_E6, PB02_E7, PB02_E8, PB02_E9, PB02_E10, PB02_E12, PB02_F5, PB02_F11, PB03_A1, PB03_A2, PB03_A3, PB03_A4, PB03_A5, PB03_A6, PB03_A8, PB03_A9, PB03_A10, PB03_A11, PB03_A12, PB03_B2, PB03_B3, PB03_B4, PB03_B5, PB03_B6, PB03_B8, PB03_B10, PB03_B11, PB03_B12, PB03_C1, PB03_C2, PB03_C3, PB03_C4, PB03_C5, PB03_C7, PB03_C8, PB03_C9, PB03_C10, PB03_C11, PB03_D3, PB03_D4, PB03_D5, PB03_D7, PB03_D8, PB03_D9, PB03_D10, PB03_E1, PB03_E6, PB03_E7, PB03_E8, PB03_E9, PB03_E10, PB03_E11, PB03_F1, PB03_F2, PB03_F3, PB03_F4, PB03_F5, PB03_F6, PB03_F7, PB03_F8, PB03_F9, PB03_F11, PB03_F12, PB03_G1, PB03_G2, PB03_G3, PB03_G4, PB03_G6, PB03_G7, PB03_G8, PB03_G9, PB03_G10, PB03_G11, PB03_G12, PB04_A1, PB04_A3, PB04_A5, PB04_A7, PB04_A10, PB04_A11, PB04_A12, PB04_B5, PB04_B7, PB04_B9, PB04_B10, PB04_B12, PB04_C3, PB04_C5, PB04_C8, PB04_C9, PB04_C11, PB04_D1, PB04_D2, PB04_D3, PB04_D4, PB04_D6, PB04_D9, PB04_E3, PB04_E7, PB04_E8, PB04_E9, PB04_E10, PB04_E12, PB04_F1, PB04_F3, PB04_F7, PB04_F8, PB04_F10, PB04_F11, PB04_F12, PB04_G1, PB04_G4, PB04_G7, PB04_G8, PB04_G10, PB04_G12, PB04_D7, PB04_D11, PB04_F2, SB100X, and PiggyBac in 293T cells in example 2. Tn+ represents co-transfection of a transposase plasmid and a donor plasmid, and Tn-represents transfection of a donor plasmid only.

FIG. 3, FIG. 4, and FIG. 5 are the partial enlarged pictures of FIG. 2.

FIG. 6 shows the cloning screening results of PB01_B9, PB01_B10, PB01_B11, PB01_B12, PB01_C3, PB01_C4, PB01_C7, PB01_C8, PB01_C9, PB01_C12, PB01_D1, PB01_D3, PB01_D4, PB02_A2, PB02_A4, PB02_A9, PB02_A12, PB02_B2, PB02_B8, PB02_B9, PB02_C6, PB02_C11, PB02_D3, PB02_D12, PB02_E1, PB02_E4, PB02_E5, PB02_E6, PB02_E7, PB02_E8, PB02_E9, PB02_E10, PB02_E12, PB02_F5, PB02_F11, PB03_A1, PB03_A4, PB03_A5, PB03_A10, PB03_B3, PB03_B12, PB03_C1, PB03_C7, PB03_C11, PB03_D10, PB03_E6, PB03_F1, PB03_F5, PB03_F6, PB03_F7, PB03_F12, PB03_G3, PB03_G4, PB03_G8, PB04_A1, PB04_A3, PB04_A5, PB04_A7, PB04_A10, PB04_A11, PB04_A12, PB04_B5, PB04_B7, PB04_B9, PB04_B10, PB04_B12, PB04_C3, PB04_C5, PB04_C8, PB04_C9, PB04_C11, PB04_D1, PB04_D2, PB04_D3, PB04_D4, PB04_D7, PB04_D9, PB04_D11, PB04_E3, PB04_E7, PB04_E8, PB04_E9, PB04_E10, PB04_E12, PB04_F1, PB04_F2, PB04_F3, PB04_F7, PB04_F10, PB04_F11, PB04_F12, PB04_G1, PB04_G4, PB04_G7, PB04_G8, PB04_G10, and PB04_G12 in example 3. Tn+ represents co-transfection of a transposase plasmid and a donor plasmid, and Tn-represents transfection of a donor plasmid only.

FIG. 7 shows the transposition activity detection results of PB01_B9, PB01_B10, PB01_B11, PB01_B12, PB01_C3, PB01_C4, PB01_C7, PB01_C8, PB01_C9, PB01_C12, PB01_D1, PB01_D3, PB01_D4, PB02_A2, PB02_A4, PB02_A9, PB02_A12, PB02_B2, PB02_B8, PB02_B9, PB02_C6, PB02_C11, PB02_D3, PB02_D12, PB02_E1, PB02_E4, PB02_E5, PB02_E6, PB02_E7, PB02_E8, PB02_E9, PB02_E10, PB02_E12, PB02_F5, PB02_F11, PB03_A1, PB03_A4, PB03_A5, PB03_A10, PB03_B3, PB03_B12, PB03_C1, PB03_C7, PB03_C11, PB03_D10, PB03_E6, PB03_F1, PB03_F5, PB03_F6, PB03_F7, PB03_F12, PB03_G3, PB03_G4, PB03_G8, PB04_A1, PB04_A3, PB04_A5, PB04_A7, PB04_A10, PB04_A11, PB04_A12, PB04_B5, PB04_B7, PB04_B9, PB04_B10, PB04_B12, PB04_C3, PB04_C5, PB04_C8, PB04_C9, PB04_C11, PB04_D1, PB04_D2, PB04_D3, PB04_D4, PB04_D7, PB04_D9, PB04_D11, PB04_E3, PB04_E7, PB04_E8, PB04_E9, PB04_E10, PB04_E12, PB04_F1, PB04_F2, PB04_F3, PB04_F7, PB04_F10, PB04_F11, PB04_F12, PB04_G1, PB04_G4, PB04_G7, PB04_G8, PB04_G10, and PB04_G12 in example 3.

FIG. 8 and FIG. 9 are the partial enlarged pictures of FIG. 7.

FIG. 10 shows an evolutionary branching diagram of PB01_B9, PB01_B10, PB01_B11, PB01_B12, PB01_C3, PB01_C4, PB01_C7, PB01_C8, PB01_C9, PB01_C12, PB01_D1, PB01_D3, PB01_D4, PB02_A2, PB02_A4, PB02_A9, PB02_A12, PB02_B2, PB02_B8, PB02_B9, PB02_C6, PB02_C11, PB02_D3, PB02_D12, PB02_E1, PB02_E4, PB02_E5, PB02_E6, PB02_E7, PB02_E8, PB02_E9, PB02_E10, PB02_E12, PB02_F5, PB02_F11, PB03_A1, PB03_A2, PB03_A3, PB03_A4, PB03_A5, PB03_A6, PB03_A8, PB03_A9, PB03_A10, PB03_A11, PB03_A12, PB03_B2, PB03_B3, PB03_B4, PB03_B5, PB03_B6, PB03_B8, PB03_B10, PB03_B11, PB03_B12, PB03_C1, PB03_C2, PB03_C3, PB03_C4, PB03_C5, PB03_C7, PB03_C8, PB03_C9, PB03_C10, PB03_C11, PB03_D3, PB03_D4, PB03_D5, PB03_D7, PB03_D8, PB03_D9, PB03_D10, PB03_E1, PB03_E6, PB03_E7, PB03_E8, PB03_E9, PB03_E10, PB03_E11, PB03_F1, PB03_F2, PB03_F3, PB03_F4, PB03_F5, PB03_F6, PB03_F7, PB03_F8, PB03_F9, PB03_F11, PB03_F12, PB03_G1, PB03_G2, PB03_G3, PB03_G4, PB03_G6, PB03_G7, PB03_G8, PB03_G9, PB03_G10, PB03_G11, PB03_G12, PB04_A1, PB04_A3, PB04_A5, PB04_A7, PB04_A10, PB04_A11, PB04_A12, PB04_B5, PB04_B7, PB04_B9, PB04_B10, PB04_B12, PB04_C3, PB04_C5, PB04_C8, PB04_C9, PB04_C11, PB04_D1, PB04_D2, PB04_D3, PB04_D4, PB04_D6, PB04_D9, PB04_E3, PB04_E7, PB04_E8, PB04_E9, PB04_E10, PB04_E12, PB04_F1, PB04_F3, PB04_F7, PB04_F8, PB04_F10, PB04_F11, PB04_F12, PB04_G1, PB04_G4, PB04_G7, PB04_G8, PB04_G10, PB04_G12, PB04_D7, PB04_D11, PB04_F2, and PiggyBac based on protein sequences in example 2.

FIG. 11 shows the results of protein sequence similarity among PB01_B9, PB01_B10, PB01_B11, PB01_B12, PB01_C3, PB01_C4, PB01_C7, PB01_C8, PB01_C9, PB01_C12, PB01_D1, PB01_D3, PB01_D4, PB02_A2, PB02_A4, PB02_A9, PB02_A12, PB02_B2, PB02_B8, PB02_B9, PB02_C6, PB02_C11, PB02_D3, PB02_D12, PB02_E1, PB02_E4, PB02_E5, PB02_E6, PB02_E7, PB02_E8, PB02_E9, PB02_E10, PB02_E12, PB02_F5, PB02_F11, PB03_A1, PB03_A2, PB03_A3, PB03_A4, PB03_A5, PB03_A6, PB03_A8, PB03_A9, PB03_A10, PB03_A11, PB03_A12, PB03_B2, PB03_B3, PB03_B4, PB03_B5, PB03_B6, PB03_B8, PB03_B10, PB03_B11, PB03_B12, PB03_C1, PB03_C2, PB03_C3, PB03_C4, PB03_C5, PB03_C7, PB03_C8, PB03_C9, PB03_C10, PB03_C11, PB03_D3, PB03_D4, PB03_D5, PB03_D7, PB03_D8, PB03_D9, PB03_D10, PB03_E1, PB03_E6, PB03_E7, PB03_E8, PB03_E9, PB03_E10, PB03_E11, PB03_F1, PB03_F2, PB03_F3, PB03_F4, PB03_F5, PB03_F6, PB03_F7, PB03_F8, PB03_F9, PB03_F11, PB03_F12, PB03_G1, PB03_G2, PB03_G3, PB03_G4, PB03_G6, PB03_G7, PB03_G8, PB03_G9, PB03_G10, PB03_G11, PB03_G12, PB04_A1, PB04_A3, PB04_A5, PB04_A7, PB04_A10, PB04_A11, PB04_A12, PB04_B5, PB04_B7, PB04_B9, PB04_B10, PB04_B12, PB04_C3, PB04_C5, PB04_C8, PB04_C9, PB04_C11, PB04_D1, PB04_D2, PB04_D3, PB04_D4, PB04_D6, PB04_D9, PB04_E3, PB04_E7, PB04_E8, PB04_E9, PB04_E10, PB04_E12, PB04_F1, PB04_F3, PB04_F7, PB04_F8, PB04_F10, PB04_F11, PB04_F12, PB04_G1, PB04_G4, PB04_G7, PB04_G8, PB04_G10, PB04_G12, PB04_D7, PB04_D11, PB04_F2, and PiggyBac in example 2.

FIG. 12, FIG. 13, and FIG. 14 are the partial enlarged pictures of FIG. 11.

### DETAILED DESCRIPTION OF EMBODIMENTS

Unless otherwise indicated or contradicts the context, the terms or expressions used herein should be read in conjunction with the entire content of the present disclosure and as understood by those of ordinary skill in the art. All technical and scientific terms used herein have the same meanings as commonly understood by those of ordinary skill in the art, unless otherwise defined.

In the present application, the terms “nucleic acid” and “polynucleotide” are used interchangeably, and refer to polymerization forms of nucleotides of any length, including deoxyribonucleotides, ribonucleotides, combinations thereof, and analogs thereof.

In the present application, the terms “polypeptide” and “peptide” are used interchangeably, and refer to polymers of amino acids of any length. Therefore, polypeptides, oligopeptides, proteins, antibodies and enzymes are all included in the definition of polypeptide.

As described in the present application, the “fragment” of a sequence refers to a portion of a sequence. For example, the fragment of a nucleic acid sequence refers to a portion of the nucleic acid sequence, and the fragment of an amino acid sequence refers to a portion of the amino acid sequence.

As described in the present application, a “variant” of a sequence is a polynucleotide or polypeptide that differs from a reference polynucleotide or polypeptide, respectively, but retains essential properties. A typical variant of a polynucleotide differs in nucleic acid sequence from another reference polynucleotide, and the differences in nucleic acid sequence may or may not alter the amino acid sequence of the polypeptide encoded by the reference polynucleotide. A typical variant of a polypeptide differs in amino acid sequence from another reference polypeptide. Generally, the differences are limited so that the sequences of the reference polypeptide and the variant are generally very similar, and are identical in many regions. A variant polypeptide and a reference polypeptide may differ in amino acid sequence by one or more substitutions, additions, deletions in any combination. The substituted or inserted amino acid residue may or may not be a residue encoded by the genetic code. Variants of polynucleotides or polypeptides may be naturally occurring, such as allelic variations, or they may be unknown naturally occurring variants. Non-naturally occurring polynucleotide and polypeptide variants can be produced by mutagenesis techniques, direct synthesis, and other recombinant methods known to the skilled artisan.

Amino acids are usually classified by the properties of their side chains. For example, side chains may render amino acids weak acids (e.g., amino acids D and E) or weak bases (e.g., amino acids K, R and H); and if the side chains are polar, the amino acids become hydrophilic (e.g., amino acids L and I), or if the side chains are nonpolar, the amino acids become hydrophobic (e.g., amino acids S and C).

The term “family” as used in the present application refers to a group of nucleic acids or proteins having high structural similarity produced by the same ancestor by means of replication and variation, which usually have related or even the same functions. The “superfamily” refers to a group of nucleic acids or proteins having roughly the same structure produced by the same ancestor by means of replication and variation, which belong to different families and usually have different functions.

The term “transposase” as used in the present application refers to a polypeptide that catalyzes the excision of a transposon (comprising an exogenous nucleic acid and transposase recognition sequences at both sides thereof) from a first nucleic acid (a vector comprising a transposase recognition sequence and an exogenous nucleic acid) and the integration into a second nucleic acid, i.e., a target site (for example, a genomic or extrachromosomal DNA comprising a target site duplication (TSD) sequence in a cell). In some embodiments, the transposase binds to at least one terminal inverted repeat (TIR).

The term “recognition sequence” as used in the present application refer to the nucleic acid sequence located at both ends of a transposable element and one flanking a transposable first nucleic acid sequence, wherein the recognition sequence located at the 5′ end of the first nucleic acid sequence is called the 5′ recognition sequence, and the recognition sequence located at the 3′ end of the first nucleic acid sequence is called the 3′ recognition sequence. In some embodiments, the recognition sequence comprises at least one terminal inverted repeat that can bind to a transposase.

The term “nucleic acid construct” as used in the present application is defined as a single-stranded or double-stranded nucleic acid molecule herein, and preferably refers to an artificially constructed nucleic acid molecule. Optionally, the nucleic acid construct further includes one or more operably linked regulatory sequences, which can direct the expression of a coding sequence in a suitable host cell under compatible conditions. The term “expression” is understood to include any step involved in the production of a protein or polypeptide, including, but not limited to, transcription, post-transcriptional modification, translation, post-translational modification and secretion. The term “regulatory sequence” includes all components necessary or advantageous for expression of the polypeptide/protein of the present application. Each regulatory sequence may be naturally present or exogenous to the nucleic acid sequence encoding the protein or polypeptide. These regulatory sequences include, but are not limited to, leader sequences, polyadenylation [poly(A)] signal sequences, propeptide sequences, promoters, signal sequences, and transcription terminators. At a minimum, the regulatory sequences should include promoters and initiation and termination signals for transcription and translation. Regulatory sequences with linkers can be provided for the purpose of introduction into specific restriction sites for linking the regulatory sequences to the coding region of a nucleic acid sequence encoding a protein or polypeptide.

The term “promoter” as used in the present application refers to a polynucleotide sequence that can control the transcription of a coding sequence. Promoter sequences include specific sequences sufficient to enable RNA polymerase to recognize, bind, and initiate transcription. In addition, promoter sequences may include sequences that optionally modulate the recognition, binding and transcription initiation activities of RNA polymerase in the nucleic acid construct or the nucleic acid set construct provided in the present application. A promoter can affect the transcription of a gene located on the same nucleic acid molecule as the promoter or a gene located on a different nucleic acid molecule from the promoter.

The term “exogenous nucleic acid fragment” used in the present application includes any gene of interest or any gene or fragment thereof that is transposable. In some non-limiting embodiments, the exogenous nucleic acid fragment is of a different origin than the terminal repeat, for example, a nucleic acid sequence isolated from an organism different from that of the terminal inverted repeat, that is, the exogenous nucleic acid fragment is exogenous to the terminal inverted repeat. In some non-limiting embodiments, the exogenous nucleic acid fragment is of a different origin than the host cell, for example, a nucleic acid sequence isolated from an organism different from the host cell, i.e., the exogenous nucleic acid fragment is exogenous to the host cell.

The term “host cell” as used in the present application include, but are not limited to, an animal cell, a plant cell, an algal cell, a fungal cell, a yeast cell, or a bacterial cell. This term includes a progeny of an original cell into which an exogenous nucleic acid fragment has been introduced. Exemplary host cell includes human embryonic kidney cell HEK293T. It is understood that, due to natural, accidental or intentional mutations, the progeny of a single parent cell may not necessarily be identical to the original parent morphologically or in terms of genome or total DNA complement.

The term “vector” as used in the present application refers to a nucleic acid molecule capable of transporting another nucleic acid molecule connected to it. Examples of vectors include, but are not limited to, plasmids, viruses, bacteria, phages, and insertable DNA fragments. The term “plasmid” refers to a circular double-stranded DNA capable of accepting an exogenous nucleic acid fragment and replicating in prokaryotic or eukaryotic cells.

### Transposase

The present application provides an isolated transposase, wherein the transposase has a transposase sequence selected from the following (i) or a variant sequence of the aforementioned transposase having a transposase activity in (ii)-(iv): (i) at least one amino acid sequence as shown in any one of SEQ ID NOs: 1-146; (ii) at least one of sequences obtained by performing deletion, substitution, insertion, or mutation of 1, 2, 3, 4, 5, 6, 7, 8, 9 or 10 amino acids on the amino acid sequence as shown in any one of SEQ ID NOs: 1-146; (iii) at least one of amino acid sequences having at least 70%, 80%, 90%, 95% or 99% identity to the amino acid sequence as shown in any one of SEQ ID NOs: 1-146; and (iv) at least one of sequences obtained by further fusing the amino acid sequence as shown in any one of SEQ ID NOs: 1-146 with other sequences.

According to an embodiment of the present application, an isolated transposase can be provided, wherein the transposase comprises an amino acid sequence as shown in the following formula:

D E (X₁)ₐ K (X₂)_{b} G (X₃)_{c} K (X₄)_{d} G

wherein a, b, c and d are the numbers of amino acids; D is aspartic acid; E is glutamic acid; K is lysine; G is glycine; (X₁) is any amino acid, and a is 17, 18 or 19; (X₂) is any amino acid, and b is 3, 4 or 5; (X₃) is any amino acid, and c is 1; and (X₄) is any amino acid, and d is 17, 18 or 19.

According to an embodiment of the present application, an isolated transposase can be provided, wherein the transposase comprises an amino acid sequence as shown in the following formula:

P (X₅)ₑ Y (X₆)_{f} D

wherein e and f are the numbers of amino acids; P is proline; Y is tyrosine; D is aspartic acid; (X₅) is any amino acid, and e is 5; and (X₆) is any amino acid, and f is 7.

According to an embodiment of the present application, an isolated transposase can be provided, wherein the transposase comprises an amino acid sequence as shown in the following formula:

C (X₇)_{g} C (X₈)ₕ C (X₉)ᵢ C

wherein g, h and i are the numbers of amino acids; C is cysteine; (X₇) is any amino acid, and g is 2, 3 or 4; (X₈) is any amino acid, and h is 7, 8, 9, 10, 11, 12, 13, 14, 15, 16, 17, 18, 19, 20, 21, 22, 23, 24, 25, 26, 27 or 28; and (X₉) is any amino acid, and i is 2.

According to an embodiment of the present application, an isolated transposase can be provided, wherein the transposase comprises at least two of the following amino acid sequences (1)-(3):
(1) D E (X₁)ₐ K (X₂)_{b} G (X₃)_{c} K (X₄) d G;
(2) P (X₅)ₑ Y (X₆)_{f} D; or
(3) C (X₇)_{g} C (X₈)ₕ C (X₉)ᵢ C,

wherein a, b, c, d, e, f, g, h and i are the numbers of amino acids; D is aspartic acid; E is glutamic acid; K is lysine; G is glycine; P is proline; Y is tyrosine; D is aspartic acid; C is cysteine; (X₁) is any amino acid, and a is 17, 18 or 19; (X₂) is any amino acid, and b is 3, 4 or 5; (X₃) is any amino acid, and c is 1; (X₄) is any amino acid, and d is 17, 18 or 19; (X₅) is any amino acid, and e is 5; (X₆) is any amino acid, and f is 7; (X₇) is any amino acid, and g is 2, 3 or 4; (X₈) is any amino acid, and h is 7, 8, 9, 10, 11, 12, 13, 14, 15, 16, 17, 18, 19, 20, 21, 22, 23, 24, 25, 26, 27 or 28; and (X₉) is any amino acid, and i is 2.

According to an embodiment of the present application, an isolated transposase can be provided, wherein the transposase comprises the following amino acid sequences (1)-(3):
(1) D E (X₁)ₐ K (X₂)_{b} G (X₃)_{c} K (X₄)_{d} G;
(2) P (X₅)ₑ Y (X₆)_{f} D; and
(3) C (X₇)_{g} C (X₈)ₕ C (X₉)ᵢ C,

wherein a, b, c, d, e, f, g, h and i are the numbers of amino acids; D is aspartic acid; E is glutamic acid; K is lysine; G is glycine; P is proline; Y is tyrosine; D is aspartic acid; C is cysteine; (X₁) is any amino acid, and a is 17, 18 or 19; (X₂) is any amino acid, and b is 3, 4 or 5; (X₃) is any amino acid, and c is 1; (X₄) is any amino acid, and d is 17, 18 or 19; (X₅) is any amino acid, and e is 5; (X₆) is any amino acid, and f is 7; (X₇) is any amino acid, and g is 2, 3 or 4; (X₈) is any amino acid, and h is 7, 8, 9, 10, 11, 12, 13, 14, 15, 16, 17, 18, 19, 20, 21, 22, 23, 24, 25, 26, 27 or 28; and (X₉) is any amino acid, and i is 2.

In some embodiments, the transposase belongs to the PiggyBac family.

In some embodiments, the species sources of the transposase include Arthropoda, Platyhelminthes, Cnidaria, Mollusca, Annelida, or Chordata. In some embodiments, the species sources of the transposase include Insecta, Actinopteri, Amphibia, Rhabditophora, Bivalvia, Hydrozoa, Ascidiacea, Anthozoa, or Clitellata. In some embodiments, the species sources of the transposase include Aedes aegypti, Aelia acuminata, Agrypnus murinus, Anthonomus grandis, Apoderus coryli, Aporophyla lueneburgensis, Atethmia centrago, Blastobasis adustella, Bombyx mori, Calamotropha paludella, Catocala fraxini, Chrysoteuchia culmella, Ciona savignyi, Coptotermes formosanus, Coremacera marginata, Crassostrea gigas, Crassostrea virginica, Cryptotermes secundus, Diabrotica virgifera virgifera, Drosophila bipectinata, Drosophila elegans, Eubasilissa regina, Euschistus heros, Gonioctena quinquepunctata, Gymnosoma rotundatum, Heliconius melpomene, Hermetia illucens, Hesperophylax magnus, Homalodisca vitripennis, Hydra vulgaris, Hyles vespertilio, Ips nitidus, Ips typographus, Ischnura elegans, Lamprigera yunnana, Lasiommata megera, Limonius californicus, Locusta migratoria, Macaria notata, Malachius bipustulatus, Mamestra brassicae, Marasmarcha lunaedactyla, Marronus borbonicus, Melanotaenia boesemani, Mythimna impura, Nematostella vectensis, Ochropleura plecta, Ocypus olens, Orius insidiosus, Oryzias sinensis, Pachyrhynchus sulphureomaculatus, Parnassius apollo, Periplaneta americana, Philaenus spumarius, Philonthus cognatus, Pieris napi, Pissodes strobi, Platycnemis pennipes, Schistocerca americana, Schistocerca piceifrons, Schmidtea mediterranea, Sesamia nonagrioides, Sesia apiformis, Sitophilus oryzae, Solenopsis invicta, Teleogryllus occipitalis, Timema shepardi, Timema tahoe, Vandiemenella viatica, Ypsolopha sequella, or Zophobas atratus.

According to an embodiment of the present application, a nucleic acid can be provided, wherein, the nucleic acid encodes the transposase described in the present application.

According to an embodiment of the present application, a nucleic acid construct can be provided, comprising a nucleic acid encoding the transposase described in the present application. In some embodiments, the nucleic acid construct further comprises a promoter. The promoter can be any suitable promoter sequence, that is, a nucleic acid sequence that can be recognized by a host cell expressing the nucleic acid sequence. The promoter sequence contains a transcriptional regulatory sequence that mediates the expression of the protein or polypeptide. The promoter can be any nucleic acid sequence having transcriptional activity in a selected host cell, including mutant, truncated and heterozygous promoters, and can be derived from genes encoding extracellular or intracellular proteins or polypeptides homologous or heterologous to the host cell. In some embodiments, the promoter includes CMV, EF1a, SV40, PGK, UbC, human beta actin, CAG, TRE, UAS, Ac5, GFAP, Polyhedrin promotor, TBG, ALB, ApoEHCR-hAAT, CaMKIIa, GAL1, TEF1, GDS, ADH1, CaMV35S, Ubi, H1, U6, T7, T7lac, Sp6, araBAD, trp, lac, Ptac, or pL.

In some embodiments, the nucleic acid construct further comprises a poly(A) sequence. Poly(A) tailing signal sequences well known in the art, as well as various truncated forms of poly(A) tailing signals, can be used in the present application.

In some embodiments, the nucleic acid construct further includes any transcription termination sequence, i.e., a sequence that is recognized by the host cell to terminate transcription. The termination sequence is operably linked to the 3′-terminus of the nucleic acid sequence encoding the protein or polypeptide. Any terminator that is functional in the host cell of choice can be used in the present invention.

Optionally, the nucleic acid construct may further include a suitable leader sequence, that is, an untranslated region in the mRNA that is important for translation in the host cell. The leader sequence is operably linked to the 5′-terminus of the nucleic acid sequence encoding the polypeptide. Any leader sequence that is functional in the host cell of choice can be used in the present invention.

Optionally, the nucleic acid construct may further include a propeptide coding region, which encodes an amino acid sequence located at the amino terminus of the polypeptide. The resulting polypeptide is called a zymogen or a propolypeptide. The propolypeptide is usually inactive and can be converted into a mature active polypeptide by catalytic or autocatalytic cleavage of the propeptide from the propolypeptide.

Optionally, the nucleic acid construct may further include a regulatory sequence that can regulate the expression of the polypeptide according to the growth conditions of the host cell. Examples of the regulatory sequence are systems that turn gene expression on or off in response to chemical or physical stimuli, including in the presence of regulatory compounds. Other examples of the regulatory sequence are those that enable gene amplification. In these instances, the nucleic acid sequence encoding the protein or polypeptide should be operably linked to the regulatory sequence.

### Nucleic Acid Construct

According to an embodiment of the present application, a nucleic acid set can be provided, comprising a 5′ recognition sequence, wherein the 5′ recognition sequence comprises at least one of the nucleotide sequences as shown in SEQ ID NOs: 147-292.

According to an embodiment of the present application, a nucleic acid set can be provided, comprising a 3′ recognition sequence, wherein the 3′ recognition sequence comprises at least one of the nucleotide sequences as shown in SEQ ID NOs: 293-438.

According to an embodiment of the present application, a nucleic acid set can be provided, comprising a 5′ recognition sequence and a 3′ recognition sequence, wherein the 5′ recognition sequence comprises the nucleotide sequence as shown in any one of SEQ ID NOs: 147-292 or a variant thereof, the 3′ recognition sequence comprises the nucleotide sequence as shown in any one of SEQ ID NOs: 293-438 or a variant thereof, and the nucleic acid set can be recognized by a specific transposase.

In some embodiments, the 5′ recognition sequence or the 3′ recognition sequence comprises a terminal inverted repeat of at least one of 1-800 nt, 1-600 nt, 1-400 nt, 1-200 nt, 1-100 nt, 5-50 nt, 5-25 nt, or 10-20 nt in length.

According to an embodiment of the present application, a nucleic acid set construct can be provided, the nucleic acid set construct includes the nucleic acid set described in the present application and further includes an exogenous nucleic acid fragment. In some embodiments, the exogenous nucleic acid fragment is operably inserted into the nucleic acid set construct through a polyclonal insertion site, and there may be one or more exogenous nucleic acid fragments, which may be the same or different; and a promoter can also be inserted to control the expression of the exogenous nucleic acid fragment. In some embodiments, the exogenous nucleic acid fragment includes any gene of interest or any gene that is transposable, e.g., a gene of a natural functional protein, an artificial chimeric gene, or a gene of a non-coding RNA. In some embodiments, the gene of a non-coding RNA includes a variety of RNAs with known functions and RNAs with unknown functions, such as rRNA, tRNA, small interfering RNA (siRNA), small nuclear RNA (snRNA), small nucleolar RNA (snoRNA), and microRNA (miRNA). In some embodiments, the gene of a natural functional protein includes a fluorescence-based reporter gene, a luciferase gene, and a resistance gene. In some embodiments, the artificial chimeric gene includes a gene of a chimeric antigen receptor. In some embodiments, the fluorescence-based reporter gene is selected from at least one of genes encoding a green fluorescent protein, a red fluorescent protein, a blue fluorescent protein, or a yellow fluorescent protein. In some embodiments, the luciferase gene is selected from at least one of genes encoding firefly luciferase and sea kidney luciferase. In some embodiments, the resistance gene is selected from at least one of genes encoding puromycin resistance, G418 resistance, kanamycin resistance, tetracycline resistance, and bleomycin resistance.

In some embodiments, a promoter can also be inserted into the nucleic acid set construct to control the expression of the exogenous nucleic acid fragment. The promoter can be any suitable promoter sequence, that is, a nucleic acid sequence that can be recognized by a host cell expressing the exogenous nucleic acid fragment. The promoter sequence contains a transcriptional regulatory sequence that mediates the expression of the protein or polypeptide. The promoter can be any nucleic acid sequence having transcriptional activity in a selected host cell, including mutant, truncated and heterozygous promoters, and can be derived from genes encoding extracellular or intracellular proteins or polypeptides homologous or heterologous to the host cell. In some embodiments, the promoter includes CMV, EF1a, SV40, PGK, UbC, human beta actin, CAG, TRE, UAS, Ac5, GFAP, Polyhedrin promotor, TBG, ALB, ApoEHCR-hAAT, CaMKIIa, GAL1, TEF1, GDS, ADH1, CaMV35S, Ubi, H1, U6, T7, T7lac, Sp6, araBAD, trp, lac, Ptac, or pL.

In some embodiments, the nucleic acid set construct further includes any transcription termination sequence (i.e., a sequence that is recognized by the host cell to terminate transcription) to control the expression of the exogenous nucleic acid fragment. Any terminator that is functional in the host cell of choice can be used in the present invention.

Optionally, the nucleic acid set construct may further include a suitable leader sequence (i.e., an untranslated region in the mRNA that is important for translation in the host cell) to control the expression of the exogenous nucleic acid fragment. The leader sequence is operably linked to the 5′-terminus of the nucleic acid sequence encoding the polypeptide. Any leader sequence that is functional in the host cell of choice can be used in the present invention.

Optionally, the nucleic acid set construct may further include a propeptide coding region to control the expression of the exogenous nucleic acid fragment, the propeptide coding region encodes an amino acid sequence located at the amino terminus of the polypeptide. The resulting polypeptide is called a zymogen or a propolypeptide. The propolypeptide is usually inactive and can be converted into a mature active polypeptide by catalytic or autocatalytic cleavage of the propeptide from the propolypeptide.

Optionally, the nucleic acid set construct may further include a regulatory sequence that can regulate the expression of the exogenous nucleic acid fragment according to the growth conditions of the host cell. Examples of the regulatory sequence are systems that turn gene expression on or off in response to chemical or physical stimuli, including in the presence of regulatory compounds. Other examples of the regulatory sequence are those that enable gene amplification. In these instances, the exogenous nucleic acid fragment should be operably linked to the regulatory sequence.

### Transposition Composition

According to an embodiment of the present application, a composition may be provided, wherein, the composition includes: a PiggyBac family transposase or a functional fragment thereof, or a nucleic acid encoding the PiggyBac family transposase or the functional fragment thereof, wherein the transposase or the functional fragment thereof has a function of catalyzing the insertion of an exogenous nucleic acid fragment into the genome of a cell; and a nucleic acid set, wherein the nucleic acid set can be recognized by a specific transposase or a functional fragment thereof.

In some embodiments, the composition is selected from at least one of the following groups (1)-(147), and any one of the following groups (1)-(146) comprises: a transposase-related sequence and a nucleic acid set,
(1) the transposase-related sequence is an amino acid sequence comprising the sequence as shown in SEQ ID NO: 1 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 147, and the 3′ recognition sequence is a nucleotide sequence comprising the sequence as shown in SEQ ID NO: 293;
(2) the transposase-related sequence is an amino acid sequence comprising the sequence as shown in SEQ ID NO: 2 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 148, and the 3′ recognition sequence is a nucleotide sequence comprising the sequence as shown in SEQ ID NO: 294;
(3) the transposase-related sequence is an amino acid sequence comprising the sequence as shown in SEQ ID NO: 3 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 149, and the 3′ recognition sequence is a nucleotide sequence comprising the sequence as shown in SEQ ID NO: 295;
(4) the transposase-related sequence is an amino acid sequence comprising the sequence as shown in SEQ ID NO: 4 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 150, and the 3′ recognition sequence is a nucleotide sequence comprising the sequence as shown in SEQ ID NO: 296;
(5) the transposase-related sequence is an amino acid sequence comprising the sequence as shown in SEQ ID NO: 5 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 151, and the 3′ recognition sequence is a nucleotide sequence comprising the sequence as shown in SEQ ID NO: 297;
(6) the transposase-related sequence is an amino acid sequence comprising the sequence as shown in SEQ ID NO: 6 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 152, and the 3′ recognition sequence is a nucleotide sequence comprising the sequence as shown in SEQ ID NO: 298;
(7) the transposase-related sequence is an amino acid sequence comprising the sequence as shown in SEQ ID NO: 7 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 153, and the 3′ recognition sequence is a nucleotide sequence comprising the sequence as shown in SEQ ID NO: 299;
(8) the transposase-related sequence is an amino acid sequence comprising the sequence as shown in SEQ ID NO: 8 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 154, and the 3′ recognition sequence is a nucleotide sequence comprising the sequence as shown in SEQ ID NO: 300;
(9) the transposase-related sequence is an amino acid sequence comprising the sequence as shown in SEQ ID NO: 9 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 155, and the 3′ recognition sequence is a nucleotide sequence comprising the sequence as shown in SEQ ID NO: 301;
(10) the transposase-related sequence is an amino acid sequence comprising the sequence as shown in SEQ ID NO: 10 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 156, and the 3′ recognition sequence is a nucleotide sequence comprising the sequence as shown in SEQ ID NO: 302;
(11) the transposase-related sequence is an amino acid sequence comprising the sequence as shown in SEQ ID NO: 11 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 157, and the 3′ recognition sequence is a nucleotide sequence comprising the sequence as shown in SEQ ID NO: 303;
(12) the transposase-related sequence is an amino acid sequence comprising the sequence as shown in SEQ ID NO: 12 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 158, and the 3′ recognition sequence is a nucleotide sequence comprising the sequence as shown in SEQ ID NO: 304;
(13) the transposase-related sequence is an amino acid sequence comprising the sequence as shown in SEQ ID NO: 13 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 159, and the 3′ recognition sequence is a nucleotide sequence comprising the sequence as shown in SEQ ID NO: 305;
(14) the transposase-related sequence is an amino acid sequence comprising the sequence as shown in SEQ ID NO: 14 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 160, and the 3′ recognition sequence is a nucleotide sequence comprising the sequence as shown in SEQ ID NO: 306;
(15) the transposase-related sequence is an amino acid sequence comprising the sequence as shown in SEQ ID NO: 15 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 161, and the 3′ recognition sequence is a nucleotide sequence comprising the sequence as shown in SEQ ID NO: 307;
(16) the transposase-related sequence is an amino acid sequence comprising the sequence as shown in SEQ ID NO: 16 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 162, and the 3′ recognition sequence is a nucleotide sequence comprising the sequence as shown in SEQ ID NO: 308;
(17) the transposase-related sequence is an amino acid sequence comprising the sequence as shown in SEQ ID NO: 17 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 163, and the 3′ recognition sequence is a nucleotide sequence comprising the sequence as shown in SEQ ID NO: 309;
(18) the transposase-related sequence is an amino acid sequence comprising the sequence as shown in SEQ ID NO: 18 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 164, and the 3′ recognition sequence is a nucleotide sequence comprising the sequence as shown in SEQ ID NO: 310;
(19) the transposase-related sequence is an amino acid sequence comprising the sequence as shown in SEQ ID NO: 19 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 165, and the 3′ recognition sequence is a nucleotide sequence comprising the sequence as shown in SEQ ID NO: 311;
(20) the transposase-related sequence is an amino acid sequence comprising the sequence as shown in SEQ ID NO: 20 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 166, and the 3′ recognition sequence is a nucleotide sequence comprising the sequence as shown in SEQ ID NO: 312;
(21) the transposase-related sequence is an amino acid sequence comprising the sequence as shown in SEQ ID NO: 21 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 167, and the 3′ recognition sequence is a nucleotide sequence comprising the sequence as shown in SEQ ID NO: 313;
(22) the transposase-related sequence is an amino acid sequence comprising the sequence as shown in SEQ ID NO: 22 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 168, and the 3′ recognition sequence is a nucleotide sequence comprising the sequence as shown in SEQ ID NO: 314;
(23) the transposase-related sequence is an amino acid sequence comprising the sequence as shown in SEQ ID NO: 23 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 169, and the 3′ recognition sequence is a nucleotide sequence comprising the sequence as shown in SEQ ID NO: 315;
(24) the transposase-related sequence is an amino acid sequence comprising the sequence as shown in SEQ ID NO: 24 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 170, and the 3′ recognition sequence is a nucleotide sequence comprising the sequence as shown in SEQ ID NO: 316;
(25) the transposase-related sequence is an amino acid sequence comprising the sequence as shown in SEQ ID NO: 25 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 171, and the 3′ recognition sequence is a nucleotide sequence comprising the sequence as shown in SEQ ID NO: 317;
(26) the transposase-related sequence is an amino acid sequence comprising the sequence as shown in SEQ ID NO: 26 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 172, and the 3′ recognition sequence is a nucleotide sequence comprising the sequence as shown in SEQ ID NO: 318;
(27) the transposase-related sequence is an amino acid sequence comprising the sequence as shown in SEQ ID NO: 27 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 173, and the 3′ recognition sequence is a nucleotide sequence comprising the sequence as shown in SEQ ID NO: 319;
(28) the transposase-related sequence is an amino acid sequence comprising the sequence as shown in SEQ ID NO: 28 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 174, and the 3′ recognition sequence is a nucleotide sequence comprising the sequence as shown in SEQ ID NO: 320;
(29) the transposase-related sequence is an amino acid sequence comprising the sequence as shown in SEQ ID NO: 29 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 175, and the 3′ recognition sequence is a nucleotide sequence comprising the sequence as shown in SEQ ID NO: 321;
(30) the transposase-related sequence is an amino acid sequence comprising the sequence as shown in SEQ ID NO: 30 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 176, and the 3′ recognition sequence is a nucleotide sequence comprising the sequence as shown in SEQ ID NO: 322;
(31) the transposase-related sequence is an amino acid sequence comprising the sequence as shown in SEQ ID NO: 31 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 177, and the 3′ recognition sequence is a nucleotide sequence comprising the sequence as shown in SEQ ID NO: 323;
(32) the transposase-related sequence is an amino acid sequence comprising the sequence as shown in SEQ ID NO: 32 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 178, and the 3′ recognition sequence is a nucleotide sequence comprising the sequence as shown in SEQ ID NO: 324;
(33) the transposase-related sequence is an amino acid sequence comprising the sequence as shown in SEQ ID NO: 33 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 179, and the 3′ recognition sequence is a nucleotide sequence comprising the sequence as shown in SEQ ID NO: 325;
(34) the transposase-related sequence is an amino acid sequence comprising the sequence as shown in SEQ ID NO: 34 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 180, and the 3′ recognition sequence is a nucleotide sequence comprising the sequence as shown in SEQ ID NO: 326;
(35) the transposase-related sequence is an amino acid sequence comprising the sequence as shown in SEQ ID NO: 35 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 181, and the 3′ recognition sequence is a nucleotide sequence comprising the sequence as shown in SEQ ID NO: 327;
(36) the transposase-related sequence is an amino acid sequence comprising the sequence as shown in SEQ ID NO: 36 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 182, and the 3′ recognition sequence is a nucleotide sequence comprising the sequence as shown in SEQ ID NO: 328;
(37) the transposase-related sequence is an amino acid sequence comprising the sequence as shown in SEQ ID NO: 37 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 183, and the 3′ recognition sequence is a nucleotide sequence comprising the sequence as shown in SEQ ID NO: 329;
(38) the transposase-related sequence is an amino acid sequence comprising the sequence as shown in SEQ ID NO: 38 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 184, and the 3′ recognition sequence is a nucleotide sequence comprising the sequence as shown in SEQ ID NO: 330;
(39) the transposase-related sequence is an amino acid sequence comprising the sequence as shown in SEQ ID NO: 39 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 185, and the 3′ recognition sequence is a nucleotide sequence comprising the sequence as shown in SEQ ID NO: 331;
(40) the transposase-related sequence is an amino acid sequence comprising the sequence as shown in SEQ ID NO: 40 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 186, and the 3′ recognition sequence is a nucleotide sequence comprising the sequence as shown in SEQ ID NO: 332;
(41) the transposase-related sequence is an amino acid sequence comprising the sequence as shown in SEQ ID NO: 41 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 187, and the 3′ recognition sequence is a nucleotide sequence comprising the sequence as shown in SEQ ID NO: 333;
(42) the transposase-related sequence is an amino acid sequence comprising the sequence as shown in SEQ ID NO: 42 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 188, and the 3′ recognition sequence is a nucleotide sequence comprising the sequence as shown in SEQ ID NO: 334;
(43) the transposase-related sequence is an amino acid sequence comprising the sequence as shown in SEQ ID NO: 43 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 189, and the 3′ recognition sequence is a nucleotide sequence comprising the sequence as shown in SEQ ID NO: 335;
(44) the transposase-related sequence is an amino acid sequence comprising the sequence as shown in SEQ ID NO: 44 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 190, and the 3′ recognition sequence is a nucleotide sequence comprising the sequence as shown in SEQ ID NO: 336;
(45) the transposase-related sequence is an amino acid sequence comprising the sequence as shown in SEQ ID NO: 45 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 191, and the 3′ recognition sequence is a nucleotide sequence comprising the sequence as shown in SEQ ID NO: 337;
(46) the transposase-related sequence is an amino acid sequence comprising the sequence as shown in SEQ ID NO: 46 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 192, and the 3′ recognition sequence is a nucleotide sequence comprising the sequence as shown in SEQ ID NO: 338;
(47) the transposase-related sequence is an amino acid sequence comprising the sequence as shown in SEQ ID NO: 47 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 193, and the 3′ recognition sequence is a nucleotide sequence comprising the sequence as shown in SEQ ID NO: 339;
(48) the transposase-related sequence is an amino acid sequence comprising the sequence as shown in SEQ ID NO: 48 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 194, and the 3′ recognition sequence is a nucleotide sequence comprising the sequence as shown in SEQ ID NO: 340;
(49) the transposase-related sequence is an amino acid sequence comprising the sequence as shown in SEQ ID NO: 49 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 195, and the 3′ recognition sequence is a nucleotide sequence comprising the sequence as shown in SEQ ID NO: 341;
(50) the transposase-related sequence is an amino acid sequence comprising the sequence as shown in SEQ ID NO: 50 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 196, and the 3′ recognition sequence is a nucleotide sequence comprising the sequence as shown in SEQ ID NO: 342;
(51) the transposase-related sequence is an amino acid sequence comprising the sequence as shown in SEQ ID NO: 51 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 197, and the 3′ recognition sequence is a nucleotide sequence comprising the sequence as shown in SEQ ID NO: 343;
(52) the transposase-related sequence is an amino acid sequence comprising the sequence as shown in SEQ ID NO: 52 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 198, and the 3′ recognition sequence is a nucleotide sequence comprising the sequence as shown in SEQ ID NO: 344;
(53) the transposase-related sequence is an amino acid sequence comprising the sequence as shown in SEQ ID NO: 53 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 199, and the 3′ recognition sequence is a nucleotide sequence comprising the sequence as shown in SEQ ID NO: 345;
(54) the transposase-related sequence is an amino acid sequence comprising the sequence as shown in SEQ ID NO: 54 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 200, and the 3′ recognition sequence is a nucleotide sequence comprising the sequence as shown in SEQ ID NO: 346;
(55) the transposase-related sequence is an amino acid sequence comprising the sequence as shown in SEQ ID NO: 55 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 201, and the 3′ recognition sequence is a nucleotide sequence comprising the sequence as shown in SEQ ID NO: 347;
(56) the transposase-related sequence is an amino acid sequence comprising the sequence as shown in SEQ ID NO: 56 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 202, and the 3′ recognition sequence is a nucleotide sequence comprising the sequence as shown in SEQ ID NO: 348;
(57) the transposase-related sequence is an amino acid sequence comprising the sequence as shown in SEQ ID NO: 57 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 203, and the 3′ recognition sequence is a nucleotide sequence comprising the sequence as shown in SEQ ID NO: 349;
(58) the transposase-related sequence is an amino acid sequence comprising the sequence as shown in SEQ ID NO: 58 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 204, and the 3′ recognition sequence is a nucleotide sequence comprising the sequence as shown in SEQ ID NO: 350;
(59) the transposase-related sequence is an amino acid sequence comprising the sequence as shown in SEQ ID NO: 59 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 205, and the 3′ recognition sequence is a nucleotide sequence comprising the sequence as shown in SEQ ID NO: 351;
(60) the transposase-related sequence is an amino acid sequence comprising the sequence as shown in SEQ ID NO: 60 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 206, and the 3′ recognition sequence is a nucleotide sequence comprising the sequence as shown in SEQ ID NO: 352;
(61) the transposase-related sequence is an amino acid sequence comprising the sequence as shown in SEQ ID NO: 61 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 207, and the 3′ recognition sequence is a nucleotide sequence comprising the sequence as shown in SEQ ID NO: 353;
(62) the transposase-related sequence is an amino acid sequence comprising the sequence as shown in SEQ ID NO: 62 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 208, and the 3′ recognition sequence is a nucleotide sequence comprising the sequence as shown in SEQ ID NO: 354;
(63) the transposase-related sequence is an amino acid sequence comprising the sequence as shown in SEQ ID NO: 63 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 209, and the 3′ recognition sequence is a nucleotide sequence comprising the sequence as shown in SEQ ID NO: 355;
(64) the transposase-related sequence is an amino acid sequence comprising the sequence as shown in SEQ ID NO: 64 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 210, and the 3′ recognition sequence is a nucleotide sequence comprising the sequence as shown in SEQ ID NO: 356;
(65) the transposase-related sequence is an amino acid sequence comprising the sequence as shown in SEQ ID NO: 65 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 211, and the 3′ recognition sequence is a nucleotide sequence comprising the sequence as shown in SEQ ID NO: 357;
(66) the transposase-related sequence is an amino acid sequence comprising the sequence as shown in SEQ ID NO: 66 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 212, and the 3′ recognition sequence is a nucleotide sequence comprising the sequence as shown in SEQ ID NO: 358;
(67) the transposase-related sequence is an amino acid sequence comprising the sequence as shown in SEQ ID NO: 67 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 213, and the 3′ recognition sequence is a nucleotide sequence comprising the sequence as shown in SEQ ID NO: 359;
(68) the transposase-related sequence is an amino acid sequence comprising the sequence as shown in SEQ ID NO: 68 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 214, and the 3′ recognition sequence is a nucleotide sequence comprising the sequence as shown in SEQ ID NO: 360;
(69) the transposase-related sequence is an amino acid sequence comprising the sequence as shown in SEQ ID NO: 69 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 215, and the 3′ recognition sequence is a nucleotide sequence comprising the sequence as shown in SEQ ID NO: 361;
(70) the transposase-related sequence is an amino acid sequence comprising the sequence as shown in SEQ ID NO: 70 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 216, and the 3′ recognition sequence is a nucleotide sequence comprising the sequence as shown in SEQ ID NO: 362;
(71) the transposase-related sequence is an amino acid sequence comprising the sequence as shown in SEQ ID NO: 71 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 217, and the 3′ recognition sequence is a nucleotide sequence comprising the sequence as shown in SEQ ID NO: 363;
(72) the transposase-related sequence is an amino acid sequence comprising the sequence as shown in SEQ ID NO: 72 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 218, and the 3′ recognition sequence is a nucleotide sequence comprising the sequence as shown in SEQ ID NO: 364;
(73) the transposase-related sequence is an amino acid sequence comprising the sequence as shown in SEQ ID NO: 73 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 219, and the 3′ recognition sequence is a nucleotide sequence comprising the sequence as shown in SEQ ID NO: 365;
(74) the transposase-related sequence is an amino acid sequence comprising the sequence as shown in SEQ ID NO: 74 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 220, and the 3′ recognition sequence is a nucleotide sequence comprising the sequence as shown in SEQ ID NO: 366;
(75) the transposase-related sequence is an amino acid sequence comprising the sequence as shown in SEQ ID NO: 75 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 221, and the 3′ recognition sequence is a nucleotide sequence comprising the sequence as shown in SEQ ID NO: 367;
(76) the transposase-related sequence is an amino acid sequence comprising the sequence as shown in SEQ ID NO: 76 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 222, and the 3′ recognition sequence is a nucleotide sequence comprising the sequence as shown in SEQ ID NO: 368;
(77) the transposase-related sequence is an amino acid sequence comprising the sequence as shown in SEQ ID NO: 77 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 223, and the 3′ recognition sequence is a nucleotide sequence comprising the sequence as shown in SEQ ID NO: 369;
(78) the transposase-related sequence is an amino acid sequence comprising the sequence as shown in SEQ ID NO: 78 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 224, and the 3′ recognition sequence is a nucleotide sequence comprising the sequence as shown in SEQ ID NO: 370;
(79) the transposase-related sequence is an amino acid sequence comprising the sequence as shown in SEQ ID NO: 79 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 225, and the 3′ recognition sequence is a nucleotide sequence comprising the sequence as shown in SEQ ID NO: 371;
(80) the transposase-related sequence is an amino acid sequence comprising the sequence as shown in SEQ ID NO: 80 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 226, and the 3′ recognition sequence is a nucleotide sequence comprising the sequence as shown in SEQ ID NO: 372;
(81) the transposase-related sequence is an amino acid sequence comprising the sequence as shown in SEQ ID NO: 81 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 227, and the 3′ recognition sequence is a nucleotide sequence comprising the sequence as shown in SEQ ID NO: 373;
(82) the transposase-related sequence is an amino acid sequence comprising the sequence as shown in SEQ ID NO: 82 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 228, and the 3′ recognition sequence is a nucleotide sequence comprising the sequence as shown in SEQ ID NO: 374;
(83) the transposase-related sequence is an amino acid sequence comprising the sequence as shown in SEQ ID NO: 83 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 229, and the 3′ recognition sequence is a nucleotide sequence comprising the sequence as shown in SEQ ID NO: 375;
(84) the transposase-related sequence is an amino acid sequence comprising the sequence as shown in SEQ ID NO: 84 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 230, and the 3′ recognition sequence is a nucleotide sequence comprising the sequence as shown in SEQ ID NO: 376;
(85) the transposase-related sequence is an amino acid sequence comprising the sequence as shown in SEQ ID NO: 85 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 231, and the 3′ recognition sequence is a nucleotide sequence comprising the sequence as shown in SEQ ID NO: 377;
(86) the transposase-related sequence is an amino acid sequence comprising the sequence as shown in SEQ ID NO: 86 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 232, and the 3′ recognition sequence is a nucleotide sequence comprising the sequence as shown in SEQ ID NO: 378;
(87) the transposase-related sequence is an amino acid sequence comprising the sequence as shown in SEQ ID NO: 87 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 233, and the 3′ recognition sequence is a nucleotide sequence comprising the sequence as shown in SEQ ID NO: 379;
(88) the transposase-related sequence is an amino acid sequence comprising the sequence as shown in SEQ ID NO: 88 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 234, and the 3′ recognition sequence is a nucleotide sequence comprising the sequence as shown in SEQ ID NO: 380;
(89) the transposase-related sequence is an amino acid sequence comprising the sequence as shown in SEQ ID NO: 89 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 235, and the 3′ recognition sequence is a nucleotide sequence comprising the sequence as shown in SEQ ID NO: 381;
(90) the transposase-related sequence is an amino acid sequence comprising the sequence as shown in SEQ ID NO: 90 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 236, and the 3′ recognition sequence is a nucleotide sequence comprising the sequence as shown in SEQ ID NO: 382;
(91) the transposase-related sequence is an amino acid sequence comprising the sequence as shown in SEQ ID NO: 91 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 237, and the 3′ recognition sequence is a nucleotide sequence comprising the sequence as shown in SEQ ID NO: 383;
(92) the transposase-related sequence is an amino acid sequence comprising the sequence as shown in SEQ ID NO: 92 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 238, and the 3′ recognition sequence is a nucleotide sequence comprising the sequence as shown in SEQ ID NO: 384;
(93) the transposase-related sequence is an amino acid sequence comprising the sequence as shown in SEQ ID NO: 93 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 239, and the 3′ recognition sequence is a nucleotide sequence comprising the sequence as shown in SEQ ID NO: 385;
(94) the transposase-related sequence is an amino acid sequence comprising the sequence as shown in SEQ ID NO: 94 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 240, and the 3′ recognition sequence is a nucleotide sequence comprising the sequence as shown in SEQ ID NO: 386;
(95) the transposase-related sequence is an amino acid sequence comprising the sequence as shown in SEQ ID NO: 95 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 241, and the 3′ recognition sequence is a nucleotide sequence comprising the sequence as shown in SEQ ID NO: 387;
(96) the transposase-related sequence is an amino acid sequence comprising the sequence as shown in SEQ ID NO: 96 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 242, and the 3′ recognition sequence is a nucleotide sequence comprising the sequence as shown in SEQ ID NO: 388;
(97) the transposase-related sequence is an amino acid sequence comprising the sequence as shown in SEQ ID NO: 97 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 243, and the 3′ recognition sequence is a nucleotide sequence comprising the sequence as shown in SEQ ID NO: 389;
(98) the transposase-related sequence is an amino acid sequence comprising the sequence as shown in SEQ ID NO: 98 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 244, and the 3′ recognition sequence is a nucleotide sequence comprising the sequence as shown in SEQ ID NO: 390;
(99) the transposase-related sequence is an amino acid sequence comprising the sequence as shown in SEQ ID NO: 99 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 245, and the 3′ recognition sequence is a nucleotide sequence comprising the sequence as shown in SEQ ID NO: 391;
(100) the transposase-related sequence is an amino acid sequence comprising the sequence as shown in SEQ ID NO: 100 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 246, and the 3′ recognition sequence is a nucleotide sequence comprising the sequence as shown in SEQ ID NO: 392;
(101) the transposase-related sequence is an amino acid sequence comprising the sequence as shown in SEQ ID NO: 101 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 247, and the 3′ recognition sequence is a nucleotide sequence comprising the sequence as shown in SEQ ID NO: 393;
(102) the transposase-related sequence is an amino acid sequence comprising the sequence as shown in SEQ ID NO: 102 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 248, and the 3′ recognition sequence is a nucleotide sequence comprising the sequence as shown in SEQ ID NO: 394;
(103) the transposase-related sequence is an amino acid sequence comprising the sequence as shown in SEQ ID NO: 103 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 249, and the 3′ recognition sequence is a nucleotide sequence comprising the sequence as shown in SEQ ID NO: 395;
(104) the transposase-related sequence is an amino acid sequence comprising the sequence as shown in SEQ ID NO: 104 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 250, and the 3′ recognition sequence is a nucleotide sequence comprising the sequence as shown in SEQ ID NO: 396;
(105) the transposase-related sequence is an amino acid sequence comprising the sequence as shown in SEQ ID NO: 105 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 251, and the 3′ recognition sequence is a nucleotide sequence comprising the sequence as shown in SEQ ID NO: 397;
(106) the transposase-related sequence is an amino acid sequence comprising the sequence as shown in SEQ ID NO: 106 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 252, and the 3′ recognition sequence is a nucleotide sequence comprising the sequence as shown in SEQ ID NO: 398;
(107) the transposase-related sequence is an amino acid sequence comprising the sequence as shown in SEQ ID NO: 107 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 253, and the 3′ recognition sequence is a nucleotide sequence comprising the sequence as shown in SEQ ID NO: 399;
(108) the transposase-related sequence is an amino acid sequence comprising the sequence as shown in SEQ ID NO: 108 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 254, and the 3′ recognition sequence is a nucleotide sequence comprising the sequence as shown in SEQ ID NO: 400;
(109) the transposase-related sequence is an amino acid sequence comprising the sequence as shown in SEQ ID NO: 109 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 255, and the 3′ recognition sequence is a nucleotide sequence comprising the sequence as shown in SEQ ID NO: 401;
(110) the transposase-related sequence is an amino acid sequence comprising the sequence as shown in SEQ ID NO: 110 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 256, and the 3′ recognition sequence is a nucleotide sequence comprising the sequence as shown in SEQ ID NO: 402;
(111) the transposase-related sequence is an amino acid sequence comprising the sequence as shown in SEQ ID NO: 111 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 257, and the 3′ recognition sequence is a nucleotide sequence comprising the sequence as shown in SEQ ID NO: 403;
(112) the transposase-related sequence is an amino acid sequence comprising the sequence as shown in SEQ ID NO: 112 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 258, and the 3′ recognition sequence is a nucleotide sequence comprising the sequence as shown in SEQ ID NO: 404;
(113) the transposase-related sequence is an amino acid sequence comprising the sequence as shown in SEQ ID NO: 113 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 259, and the 3′ recognition sequence is a nucleotide sequence comprising the sequence as shown in SEQ ID NO: 405;
(114) the transposase-related sequence is an amino acid sequence comprising the sequence as shown in SEQ ID NO: 114 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 260, and the 3′ recognition sequence is a nucleotide sequence comprising the sequence as shown in SEQ ID NO: 406;
(115) the transposase-related sequence is an amino acid sequence comprising the sequence as shown in SEQ ID NO: 115 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 261, and the 3′ recognition sequence is a nucleotide sequence comprising the sequence as shown in SEQ ID NO: 407;
(116) the transposase-related sequence is an amino acid sequence comprising the sequence as shown in SEQ ID NO: 116 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 262, and the 3′ recognition sequence is a nucleotide sequence comprising the sequence as shown in SEQ ID NO: 408;
(117) the transposase-related sequence is an amino acid sequence comprising the sequence as shown in SEQ ID NO: 117 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 263, and the 3′ recognition sequence is a nucleotide sequence comprising the sequence as shown in SEQ ID NO: 409;
(118) the transposase-related sequence is an amino acid sequence comprising the sequence as shown in SEQ ID NO: 118 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 264, and the 3′ recognition sequence is a nucleotide sequence comprising the sequence as shown in SEQ ID NO: 410;
(119) the transposase-related sequence is an amino acid sequence comprising the sequence as shown in SEQ ID NO: 119 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 265, and the 3′ recognition sequence is a nucleotide sequence comprising the sequence as shown in SEQ ID NO: 411;
(120) the transposase-related sequence is an amino acid sequence comprising the sequence as shown in SEQ ID NO: 120 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 266, and the 3′ recognition sequence is a nucleotide sequence comprising the sequence as shown in SEQ ID NO: 412;
(121) the transposase-related sequence is an amino acid sequence comprising the sequence as shown in SEQ ID NO: 121 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 267, and the 3′ recognition sequence is a nucleotide sequence comprising the sequence as shown in SEQ ID NO: 413;
(122) the transposase-related sequence is an amino acid sequence comprising the sequence as shown in SEQ ID NO: 122 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 268, and the 3′ recognition sequence is a nucleotide sequence comprising the sequence as shown in SEQ ID NO: 414;
(123) the transposase-related sequence is an amino acid sequence comprising the sequence as shown in SEQ ID NO: 123 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 269, and the 3′ recognition sequence is a nucleotide sequence comprising the sequence as shown in SEQ ID NO: 415;
(124) the transposase-related sequence is an amino acid sequence comprising the sequence as shown in SEQ ID NO: 124 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 270, and the 3′ recognition sequence is a nucleotide sequence comprising the sequence as shown in SEQ ID NO: 416;
(125) the transposase-related sequence is an amino acid sequence comprising the sequence as shown in SEQ ID NO: 125 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 271, and the 3′ recognition sequence is a nucleotide sequence comprising the sequence as shown in SEQ ID NO: 417;
(126) the transposase-related sequence is an amino acid sequence comprising the sequence as shown in SEQ ID NO: 126 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 272, and the 3′ recognition sequence is a nucleotide sequence comprising the sequence as shown in SEQ ID NO: 418;
(127) the transposase-related sequence is an amino acid sequence comprising the sequence as shown in SEQ ID NO: 127 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 273, and the 3′ recognition sequence is a nucleotide sequence comprising the sequence as shown in SEQ ID NO: 419;
(128) the transposase-related sequence is an amino acid sequence comprising the sequence as shown in SEQ ID NO: 128 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 274, and the 3′ recognition sequence is a nucleotide sequence comprising the sequence as shown in SEQ ID NO: 420;
(129) the transposase-related sequence is an amino acid sequence comprising the sequence as shown in SEQ ID NO: 129 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 275, and the 3′ recognition sequence is a nucleotide sequence comprising the sequence as shown in SEQ ID NO: 421;
(130) the transposase-related sequence is an amino acid sequence comprising the sequence as shown in SEQ ID NO: 130 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 276, and the 3′ recognition sequence is a nucleotide sequence comprising the sequence as shown in SEQ ID NO: 422;
(131) the transposase-related sequence is an amino acid sequence comprising the sequence as shown in SEQ ID NO: 131 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 277, and the 3′ recognition sequence is a nucleotide sequence comprising the sequence as shown in SEQ ID NO: 423;
(132) the transposase-related sequence is an amino acid sequence comprising the sequence as shown in SEQ ID NO: 132 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 278, and the 3′ recognition sequence is a nucleotide sequence comprising the sequence as shown in SEQ ID NO: 424;
(133) the transposase-related sequence is an amino acid sequence comprising the sequence as shown in SEQ ID NO: 133 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 279, and the 3′ recognition sequence is a nucleotide sequence comprising the sequence as shown in SEQ ID NO: 425;
(134) the transposase-related sequence is an amino acid sequence comprising the sequence as shown in SEQ ID NO: 134 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 280, and the 3′ recognition sequence is a nucleotide sequence comprising the sequence as shown in SEQ ID NO: 426;
(135) the transposase-related sequence is an amino acid sequence comprising the sequence as shown in SEQ ID NO: 135 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 281, and the 3′ recognition sequence is a nucleotide sequence comprising the sequence as shown in SEQ ID NO: 427;
(136) the transposase-related sequence is an amino acid sequence comprising the sequence as shown in SEQ ID NO: 136 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 282, and the 3′ recognition sequence is a nucleotide sequence comprising the sequence as shown in SEQ ID NO: 428;
(137) the transposase-related sequence is an amino acid sequence comprising the sequence as shown in SEQ ID NO: 137 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 283, and the 3′ recognition sequence is a nucleotide sequence comprising the sequence as shown in SEQ ID NO: 429;
(138) the transposase-related sequence is an amino acid sequence comprising the sequence as shown in SEQ ID NO: 138 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 284, and the 3′ recognition sequence is a nucleotide sequence comprising the sequence as shown in SEQ ID NO: 430;
(139) the transposase-related sequence is an amino acid sequence comprising the sequence as shown in SEQ ID NO: 139 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 285, and the 3′ recognition sequence is a nucleotide sequence comprising the sequence as shown in SEQ ID NO: 431;
(140) the transposase-related sequence is an amino acid sequence comprising the sequence as shown in SEQ ID NO: 140 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 286, and the 3′ recognition sequence is a nucleotide sequence comprising the sequence as shown in SEQ ID NO: 432;
(141) the transposase-related sequence is an amino acid sequence comprising the sequence as shown in SEQ ID NO: 141 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 287, and the 3′ recognition sequence is a nucleotide sequence comprising the sequence as shown in SEQ ID NO: 433;
(142) the transposase-related sequence is an amino acid sequence comprising the sequence as shown in SEQ ID NO: 142 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 288, and the 3′ recognition sequence is a nucleotide sequence comprising the sequence as shown in SEQ ID NO: 434;
(143) the transposase-related sequence is an amino acid sequence comprising the sequence as shown in SEQ ID NO: 143 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 289, and the 3′ recognition sequence is a nucleotide sequence comprising the sequence as shown in SEQ ID NO: 435;
(144) the transposase-related sequence is an amino acid sequence comprising the sequence as shown in SEQ ID NO: 144 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 290, and the 3′ recognition sequence is a nucleotide sequence comprising the sequence as shown in SEQ ID NO: 436;
(145) the transposase-related sequence is an amino acid sequence comprising the sequence as shown in SEQ ID NO: 145 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 291, and the 3′ recognition sequence is a nucleotide sequence comprising the sequence as shown in SEQ ID NO: 437;
(146) the transposase-related sequence is an amino acid sequence comprising the sequence as shown in SEQ ID NO: 146 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 292, and the 3′ recognition sequence is a nucleotide sequence comprising the sequence as shown in SEQ ID NO: 438; or
(147) a variant of any one of the aforementioned groups (1)-(146),

wherein the transposase-related sequence is the amino acid sequence of the variant of the transposase in each group or a nucleic acid sequence encoding the variant, and the variant has a variant sequence of the aforementioned transposase having a transposase activity selected from the following (i)-(iii):
(i) at least one of sequences obtained by performing deletion, substitution, insertion, or mutation of 1, 2, 3, 4, 5, 6, 7, 8, 9 or 10 amino acids on the amino acid sequence of the transposase in each group;
(ii) at least one of amino acid sequences having at least 70%, 80%, 90%, 95% or 99% identity to the amino acid sequence as shown in any one of SEQ ID Nos: 1-146; and
(iii) at least one of sequences obtained by further fusing the amino acid sequence as shown in any one of SEQ ID Nos: 1-146 with other sequences.

In some embodiments, the nucleic acid encoding the amino acid sequence further comprises a promoter. The promoter can be any suitable promoter sequence, that is, a nucleic acid sequence that can be recognized by a host cell expressing the nucleic acid sequence. The promoter sequence contains a transcriptional regulatory sequence that mediates the expression of the protein or polypeptide. The promoter can be any nucleic acid sequence having transcriptional activity in a selected host cell, including mutant, truncated and heterozygous promoters, and can be derived from genes encoding extracellular or intracellular proteins or polypeptides homologous or heterologous to the host cell. In some embodiments, the promoter includes CMV, EF1a, SV40, PGK, UbC, human beta actin, CAG, TRE, UAS, Ac5, GFAP, Polyhedrin promotor, TBG, ALB, ApoEHCR-hAAT, CaMKIIa, GAL1, TEF1, GDS, ADH1, CaMV35S, Ubi, H1, U6, T7, T7lac, Sp6, araBAD, trp, lac, Ptac, or pL. In some embodiments, the nucleic acid encoding the amino acid sequence further comprises a poly(A) sequence. Poly(A) tailing signal sequences well known in the art, as well as various truncated forms of poly(A) tailing signals, can be used in the present application.

In some embodiments, the nucleic acid set further includes an exogenous nucleic acid fragment. In some embodiments, the exogenous nucleic acid fragment is operably inserted into the nucleic acid set through a polyclonal insertion site, and there may be one or more exogenous nucleic acid fragments, which may be the same or different; and a promoter can also be inserted to control the expression of the exogenous nucleic acid fragment. In some embodiments, the exogenous nucleic acid fragment includes any gene of interest or any gene that is transposable, e.g., a gene of a natural functional protein, an artificial chimeric gene, or a gene of a non-coding RNA. In some embodiments, the gene of a non-coding RNA includes a variety of RNAs with known functions and RNAs with unknown functions, such as rRNA, tRNA, small interfering RNA (siRNA), small nuclear RNA (snRNA), small nucleolar RNA (snoRNA), and microRNA (miRNA). In some embodiments, the gene of a natural functional protein includes a fluorescence-based reporter gene, a luciferase gene, and a resistance gene. In some embodiments, the artificial chimeric gene includes a gene of a chimeric antigen receptor. In some embodiments, the fluorescence-based reporter gene includes a gene encoding a green fluorescent protein, a red fluorescent protein, a blue fluorescent protein, or a yellow fluorescent protein. In some embodiments, the luciferase gene includes a gene encoding firefly luciferase or sea kidney luciferase. In some embodiments, the resistance gene includes a gene encoding puromycin resistance, G418 resistance, kanamycin resistance, tetracycline resistance, or bleomycin resistance. In some embodiments, a promoter can also be inserted into the nucleic acid set to control the expression of the exogenous nucleic acid fragment. The promoter can be any suitable promoter sequence, that is, a nucleic acid sequence that can be recognized by a host cell expressing the exogenous nucleic acid fragment. The promoter sequence contains a transcriptional regulatory sequence that mediates the expression of the protein or polypeptide. The promoter can be any nucleic acid sequence having transcriptional activity in a selected host cell, including mutant, truncated and heterozygous promoters, and can be derived from genes encoding extracellular or intracellular proteins or polypeptides homologous or heterologous to the host cell. In some embodiments, the promoter includes CMV, EF1a, SV40, PGK, UbC, human beta actin, CAG, TRE, UAS, Ac5, GFAP, Polyhedrin promotor, TBG, ALB, ApoEHCR-hAAT, CaMKIIa, GAL1, TEF1, GDS, ADH1, CaMV35S, Ubi, H1, U6, T7, T7lac, Sp6, araBAD, trp, lac, Ptac, or pL.

In some embodiments, the nucleic acid encoding the amino acid sequence and/or the nucleic acid set further comprises any transcription termination sequence that controls the expression of the exogenous nucleic acid fragment, i.e., a sequence that is recognized by a host cell to terminate transcription. Any terminator that is functional in the host cell of choice can be used in the present invention.

In some embodiments, the nucleic acid encoding the amino acid sequence and/or the nucleic acid set further includes any transcription termination sequence, i.e., a sequence that is recognized by the host cell to terminate transcription. The termination sequence is operably linked to the 3′-terminus of the nucleic acid sequence encoding the protein or polypeptide. Any terminator that is functional in the host cell of choice can be used in the present invention.

Optionally, the nucleic acid encoding the amino acid sequence and/or the nucleic acid set may further comprise a suitable leader sequence, i.e., an untranslated region in the mRNA that is important for translation in the host cell. The leader sequence is operably linked to the 5′-terminus of the nucleic acid sequence encoding the polypeptide. Any leader sequence that is functional in the host cell of choice can be used in the present invention.

Optionally, the nucleic acid encoding the amino acid sequence and/or the nucleic acid set may further comprise a propeptide coding region, which encodes an amino acid sequence located at the amino terminus of the polypeptide. The resulting polypeptide is called a zymogen or propolypeptide. The propolypeptide is usually inactive and can be converted into a mature active polypeptide by catalytic or autocatalytic cleavage of the propeptide from the propolypeptide.

Optionally, the nucleic acid encoding the amino acid sequence and/or the nucleic acid set may further comprise a regulatory sequence that can regulate the expression of the polypeptide according to the growth conditions of the host cell. Examples of the regulatory sequence are systems that turn gene expression on or off in response to chemical or physical stimuli, including in the presence of regulatory compounds. Other examples of the regulatory sequence are those that enable gene amplification. In these instances, the nucleic acid sequence encoding the protein or polypeptide should be operably linked to the regulatory sequence.

### Recombinant Vector, Recombinant Host Cell and Kit

According to an embodiment of the present application, a recombinant vector can be provided, wherein, the recombinant vector comprises the nucleic acid encoding the transposase described in the present application, the nucleic acid described in the present application, the nucleic acid construct described in the present application, the nucleic acid set described in the present application, the nucleic acid set construct described in the present application, or the composition described in the present application. The recombinant vector can be any suitable vector. In some embodiments, the recombinant vector includes, but is not limited to, a recombinant cloning vector, a recombinant eukaryotic expression plasmid, or a recombinant viral vector. In some embodiments, the recombinant eukaryotic expression plasmid includes pcDNA3.1, pCMV, pUC18, pUC19, pUC57, pBAD, pET, pENTR, pGenlenti, or pAAV. In some embodiments, the recombinant virus vector includes a recombinant adenovirus vector, a recombinant adeno-associated virus vector, a recombinant retrovirus vector, a recombinant herpes simplex virus vector, or a recombinant vaccinia virus vector. The recombinant vector of the present invention can be constructed using methods well known in the art. For example, depending on the restriction sites contained in the backbone vector used, appropriate restriction sites can be added to both ends of the nucleic acid construct of the present invention, and then loaded into the backbone vector.

According to an embodiment of the present application, a recombinant host cell can be provided, wherein, the recombinant host cell comprises the transposase described in the present application, the nucleic acid encoding the transposase described in the present application, the nucleic acid described in the present application, the nucleic acid construct described in the present application, the nucleic acid set described in the present application, the nucleic acid set construct described in the present application, the composition described in the present application, or the recombinant vector described in the present application. The recombinant host cell can be any host cell in which transposases can be used. In some embodiments, the recombinant host cell includes, but is not limited to, an animal cell, a plant cell, an algal cell, a fungal cell, a yeast cell, or a bacterial cell. In some embodiments, the animal cell includes a mammalian cell. In some embodiments, the mammalian cell includes a primary cell (e.g., a mesenchymal stem cell, an endothelial cell, an epithelial cell, a fibroblast, a keratinocyte, a melanocyte, a smooth muscle cell, and an immune cell), an immortalized cell line (e.g., HEK293, NIH-3T3, RAW-264.7, STO, VERO, CT26, hTERT immortalized human endothelial/epithelial/fibroblast/keratinocyte/ductal/cell lines), a cancer cell line (e.g., Hela, HepG2/3, HL-60, HT-1080, HT-29, A549, SW620, HCT-15, HCT116, MDA-MB-231, MCF7, SK-OV-3, PANC-1, AsPc-1, THP-1, Huh7, KG-1, RAJI, HB-CB, Jurkat, K562, CRL5826, CHO, MDCK, and Renca), an embryonic stem cell line (e.g., H1, H9, WIBR2, WIBR3, G-Olig2, ESF158, RW.4, R1, and D3) and differentiated cells thereof, or an induced pluripotent stem cell line and differentiated cells thereof.

According to an embodiment of the present application, a kit can be provided, wherein, the kit comprises the transposase described in the present application, the nucleic acid encoding the transposase described in the present application, the nucleic acid described in the present application, the nucleic acid construct described in the present application, the nucleic acid set described in the present application, the nucleic acid set construct described in the present application, the composition described in the present application, the recombinant vector described in the present application, or the recombinant host cell described in the present application.

### Method and Use

The transposase-based tools and methods for large fragment gene insertion and integration provided in the present application can be applied to many fields such as gene and cell therapy, molecular breeding in animals and plants, and industrial microorganism engineering. Particularly in the field of cell therapy, the transposition system provided by the present application can be applied to the integration of CAR sequences in cell immunotherapy (CAR-T, CAR-NK, CAR-M, etc.); in the field of gene therapy, the transposition system provided by the present application can be used to insert or integrate a healthy gene into the genome of a cell, thereby facilitating the treatment of diseases caused by gene mutations or gene defects; in terms of molecular breeding, the transposition system provided by the present application can be used as a tool for breeding many crops such as rice, corn and wheat, and can also accelerate the breeding process of animals and plants in a targeted manner; and in terms of industrial microorganism engineering, due to the defects such as instability and easy loss of plasmids in gene expression, the transposition system provided by the present application can stably integrate a gene into the chromosome of a microorganism.

According to an embodiment of the present application, a method for introducing an exogenous nucleic acid fragment into the genome of a host cell can be provided, wherein, the method comprises: delivering the transposase described in the present application, the nucleic acid encoding the transposase described in the present application, the nucleic acid described in the present application, the nucleic acid construct described in the present application, the nucleic acid set described in the present application, the nucleic acid set construct described in the present application, the composition described in the present application, or the recombinant vector described in the present application into a host cell.

According to an embodiment of the present application, a method for editing the genome of a host cell can be provided, wherein, the method comprises: delivering the transposase described in the present application, the nucleic acid encoding the transposase described in the present application, the nucleic acid described in the present application, the nucleic acid construct described in the present application, the nucleic acid set described in the present application, the nucleic acid set construct described in the present application, the composition described in the present application, or the recombinant vector described in the present application into a host cell.

According to an embodiment of the present application, a method for obtaining a host cell containing an exogenous nucleic acid fragment in the genome can be provided, wherein, the method comprises: delivering the transposase described in the present application, the nucleic acid encoding the transposase described in the present application, the nucleic acid described in the present application, the nucleic acid construct described in the present application, the nucleic acid set described in the present application, the nucleic acid set construct described in the present application, the composition described in the present application, or the recombinant vector described in the present application into a host cell.

The method of delivery into the host cell can be any suitable method. In some embodiments, the delivery method includes but is not limited to cationic liposome delivery, lipoid nanoparticulate delivery, cationic polymer delivery, vesicle-exosome delivery, gold nanoparticulate delivery, polypeptide and protein delivery, retrovirus delivery, lentivirus delivery, adenovirus delivery, adeno-associated virus delivery, electroporation, agrobacterium infection, or gene gun. The methods of cell transfection and culture are routine methods in the art, and appropriate transfection and culture methods can be selected according to different cell types.

The host cell can be any host cell in which transposases can be used. In some embodiments, the host cell includes, but is not limited to, an animal cell, a plant cell, an algal cell, a fungal cell, a yeast cell, or a bacterial cell. In some embodiments, the animal cell includes a mammalian cell. In some embodiments, the mammalian cell includes a primary cell (e.g., a mesenchymal stem cell, an endothelial cell, an epithelial cell, a fibroblast, a keratinocyte, a melanocyte, a smooth muscle cell, and an immune cell), an immortalized cell line (e.g., HEK293, NIH-3T3, RAW-264.7, STO, VERO, CT26, hTERT immortalized human endothelial/epithelial/fibroblast/keratinocyte/ductal/cell lines), a cancer cell line (e.g., Hela, HepG2/3, HL-60, HT-1080, HT-29, A549, SW620, HCT-15, HCT116, MDA-MB-231, MCF7, SK-OV-3, PANC-1, AsPc-1, THP-1, Huh7, KG-1, RAJI, HB-CB, Jurkat, K562, CRL5826, CHO, MDCK, and Renca), an embryonic stem cell line (e.g., H1, H9, WIBR2, WIBR3, G-Olig2, ESF158, RW.4, R1, and D3) and differentiated cells thereof, or an induced pluripotent stem cell line and differentiated cells thereof.

According to an embodiment of the present application, the use of the transposase described in the present application, the nucleic acid encoding the transposase described in the present application, the nucleic acid described in the present application, the nucleic acid construct described in the present application, the nucleic acid set described in the present application, the nucleic acid set construct described in the present application, the composition described in the present application, the recombinant vector described in the present application, or the recombinant host cell described in the present application for introducing an exogenous nucleic acid fragment into the genome of a host cell can be provided. The host cell can be any host cell in which transposases can be used. In some embodiments, the host cell includes, but is not limited to, an animal cell, a plant cell, an algal cell, a fungal cell, a yeast cell, or a bacterial cell. In some embodiments, the animal cell includes a mammalian cell. In some embodiments, the mammalian cell includes a primary cell (e.g., a mesenchymal stem cell, an endothelial cell, an epithelial cell, a fibroblast, a keratinocyte, a melanocyte, a smooth muscle cell, and an immune cell), an immortalized cell line (e.g., HEK293, NIH-3T3, RAW-264.7, STO, VERO, CT26, hTERT immortalized human endothelial/epithelial/fibroblast/keratinocyte/ductal/cell lines), a cancer cell line (e.g., Hela, HepG2/3, HL-60, HT-1080, HT-29, A549, SW620, HCT-15, HCT116, MDA-MB-231, MCF7, SK-OV-3, PANC-1, AsPc-1, THP-1, Huh7, KG-1, RAJI, HB-CB, Jurkat, K562, CRL5826, CHO, MDCK, and Renca), an embryonic stem cell line (e.g., H1, H9, WIBR2, WIBR3, G-Olig2, ESF158, RW.4, R1, and D3) and differentiated cells thereof, or an induced pluripotent stem cell line and differentiated cells thereof.

According to an embodiment of the present application, the use of the transposase described in the present application, the nucleic acid encoding the transposase described in the present application, the nucleic acid described in the present application, the nucleic acid construct described in the present application, the nucleic acid set described in the present application, the nucleic acid set construct described in the present application, the composition described in the present application, the recombinant vector described in the present application, or the recombinant host cell described in the present application for preparing a drug or a preparation for gene therapy, cell therapy, genome research, or stem cell induction and post-induction differentiation can be provided.

The above various embodiments and preferences for the present application can be combined with each other (as long as they are not inherently contradictory to each other) and are suitable for the use of the present application, and the various embodiments formed by such combinations are considered as a part of the present application.

### EXAMPLES

Exemplary embodiments of the present application are described below in conjunction with the accompanying drawings, where various details of the examples of the present application are included to facilitate understanding. It should be understood that they are considered to be exemplary only and not intended to limit the protection scope of the present application. The protection scope of the present application is only defined by the claims. Therefore, those of ordinary skill in the art should be aware that various changes and modifications can be made to the examples described herein, without departing from the scope of the present application. Likewise, for clarity and conciseness, the description of well-known functions and structures is omitted in the following description.

Unless otherwise stated, the reagents and instruments used in the following examples are conventional products that are commercially available. Unless otherwise stated, experiments are performed under conventional conditions or conditions recommended by the manufacturer.

### Example 1: Construction of Transposon Activity Detection System

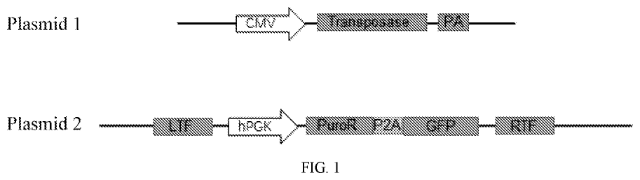

A set of detection system of a fluorescence-based reporter gene combined with an antibiotic screening marker was established to verify the activity of candidate transposons. The system performed verification using two plasmid vectors, as shown in FIG. 1: plasmid 1 was a plasmid expressing a transposase (Tn), comprising a constitutive promoter CMV (sequence as shown in SEQ ID NO: 499) that can initiate transcription in an eukaryotic cell, a sequence of a candidate transposase (as shown in Table 1), and a poly(A) sequence (PA, sequence as shown in SEQ ID NO: 500) that terminates transcription; and plasmid 2 was a transposon donor plasmid, comprising a GFP gene (sequence as shown in SEQ ID NO: 501), a puromycin resistance screening gene (PuroR, sequence as shown in SEQ ID NO: 502), promoter PGK (sequence as shown in SEQ ID NO: 503), P2A (sequence as shown in SEQ ID NO: 504) and a Poly(A) element (sequence as shown in SEQ ID NO: 500), wherein transposon sequences (LTF and RTF in FIG. 1, sequences as shown in Table 1) that can be specifically recognized by the transposase were inserted at both ends of these sequences.

TABLE 1
Plasmid construction related sequences

| Name | Plasmid 1 | Plasmid 2 |
|---|---|---|
| PB01_B9 | SEQ ID NO: 1 | LTF (SEQ ID NO: 147), RTF |
| | | (SEQ ID NO: 293) |
| PB01_B10 | SEQ ID NO: 2 | LTF (SEQ ID NO: 148), RTF |
| | | (SEQ ID NO: 294) |
| PB01_B11 | SEQ ID NO: 3 | LTF (SEQ ID NO: 149), RTF |
| | | (SEQ ID NO: 295) |
| PB01_B12 | SEQ ID NO: 4 | LTF (SEQ ID NO: 150), RTF |
| | | (SEQ ID NO: 296) |
| PB01_C3 | SEQ ID NO: 5 | LTF (SEQ ID NO: 151), RTF |
| | | (SEQ ID NO: 297) |
| PB01_C4 | SEQ ID NO: 6 | LTF (SEQ ID NO: 152), RTF |
| | | (SEQ ID NO: 298) |
| PB01_C7 | SEQ ID NO: 7 | LTF (SEQ ID NO: 153), RTF |
| | | (SEQ ID NO: 299) |
| PB01_C8 | SEQ ID NO: 8 | LTF (SEQ ID NO: 154), RTF |
| | | (SEQ ID NO: 300) |
| PB01_C9 | SEQ ID NO: 9 | LTF (SEQ ID NO: 155), RTF |
| | | (SEQ ID NO: 301) |
| PB01_C12 | SEQ ID NO: 10 | LTF (SEQ ID NO: 156), RTF |
| | | (SEQ ID NO: 302) |
| PB01_D1 | SEQ ID NO: 11 | LTF (SEQ ID NO: 157), RTF |
| | | (SEQ ID NO: 303) |
| PB01_D3 | SEQ ID NO: 12 | LTF (SEQ ID NO: 158), RTF |
| | | (SEQ ID NO: 304) |
| PB01_D4 | SEQ ID NO: 13 | LTF (SEQ ID NO: 159), RTF |
| | | (SEQ ID NO: 305) |
| PB02_A2 | SEQ ID NO: 14 | LTF (SEQ ID NO: 160), RTF |
| | | (SEQ ID NO: 306) |
| PB02_A4 | SEQ ID NO: 15 | LTF (SEQ ID NO: 161), RTF |
| | | (SEQ ID NO: 307) |
| PB02_A9 | SEQ ID NO: 16 | LTF (SEQ ID NO: 162), RTF |
| | | (SEQ ID NO: 308) |
| PB02_A12 | SEQ ID NO: 17 | LTF (SEQ ID NO: 163), RTF |
| | | (SEQ ID NO: 309) |
| PB02_B2 | SEQ ID NO: 18 | LTF (SEQ ID NO: 164), RTF |
| | | (SEQ ID NO: 310) |
| PB02_B8 | SEQ ID NO: 19 | LTF (SEQ ID NO: 165), RTF |
| | | (SEQ ID NO: 311) |
| PB02_B9 | SEQ ID NO: 20 | LTF (SEQ ID NO: 166), RTF |
| | | (SEQ ID NO: 312) |
| PB02_C6 | SEQ ID NO: 21 | LTF (SEQ ID NO: 167), RTF |
| | | (SEQ ID NO: 313) |
| PB02_C11 | SEQ ID NO: 22 | LTF (SEQ ID NO: 168), RTF |
| | | (SEQ ID NO: 314) |
| PB02_D3 | SEQ ID NO: 23 | LTF (SEQ ID NO: 169), RTF |
| | | (SEQ ID NO: 315) |
| PB02_D12 | SEQ ID NO: 24 | LTF (SEQ ID NO: 170), RTF |
| | | (SEQ ID NO: 316) |
| PB02_E1 | SEQ ID NO: 25 | LTF (SEQ ID NO: 171), RTF |
| | | (SEQ ID NO: 317) |
| PB02_E4 | SEQ ID NO: 26 | LTF (SEQ ID NO: 172), RTF |
| | | (SEQ ID NO: 318) |
| PB02_E5 | SEQ ID NO: 27 | LTF (SEQ ID NO: 173), RTF |
| | | (SEQ ID NO: 319) |
| PB02_E6 | SEQ ID NO: 28 | LTF (SEQ ID NO: 174), RTF |
| | | (SEQ ID NO: 320) |
| PB02_E7 | SEQ ID NO: 29 | LTF (SEQ ID NO: 175), RTF |
| | | (SEQ ID NO: 321) |
| PB02_E8 | SEQ ID NO: 30 | LTF (SEQ ID NO: 176), RTF |
| | | (SEQ ID NO: 322) |
| PB02_E9 | SEQ ID NO: 31 | LTF (SEQ ID NO: 177), RTF |
| | | (SEQ ID NO: 323) |
| PB02_E10 | SEQ ID NO: 32 | LTF (SEQ ID NO: 178), RTF |
| | | (SEQ ID NO: 324) |
| PB02_E12 | SEQ ID NO: 33 | LTF (SEQ ID NO: 179), RTF |
| | | (SEQ ID NO: 325) |
| PB02_F5 | SEQ ID NO: 34 | LTF (SEQ ID NO: 180), RTF |
| | | (SEQ ID NO: 326) |
| PB02_F11 | SEQ ID NO: 35 | LTF (SEQ ID NO: 181), RTF |
| | | (SEQ ID NO: 327) |
| PB03_A1 | SEQ ID NO: 36 | LTF (SEQ ID NO: 182), RTF |
| | | (SEQ ID NO: 328) |
| PB03_A2 | SEQ ID NO: 37 | LTF (SEQ ID NO: 183), RTF |
| | | (SEQ ID NO: 329) |
| PB03_A3 | SEQ ID NO: 38 | LTF (SEQ ID NO: 184), RTF |
| | | (SEQ ID NO: 330) |
| PB03_A4 | SEQ ID NO: 39 | LTF (SEQ ID NO: 185), RTF |
| | | (SEQ ID NO: 331) |
| PB03_A5 | SEQ ID NO: 40 | LTF (SEQ ID NO: 186), RTF |
| | | (SEQ ID NO: 332) |
| PB03_A6 | SEQ ID NO: 41 | LTF (SEQ ID NO: 187), RTF |
| | | (SEQ ID NO: 333) |
| PB03_A8 | SEQ ID NO: 42 | LTF (SEQ ID NO: 188), RTF |
| | | (SEQ ID NO: 334) |
| PB03_A9 | SEQ ID NO: 43 | LTF (SEQ ID NO: 189), RTF |
| | | (SEQ ID NO: 335) |
| PB03_A10 | SEQ ID NO: 44 | LTF (SEQ ID NO: 190), RTF |
| | | (SEQ ID NO: 336) |
| PB03_A11 | SEQ ID NO: 45 | LTF (SEQ ID NO: 191), RTF |
| | | (SEQ ID NO: 337) |
| PB03_A12 | SEQ ID NO: 46 | LTF (SEQ ID NO: 192), RTF |
| | | (SEQ ID NO: 338) |
| PB03_B2 | SEQ ID NO: 47 | LTF (SEQ ID NO: 193), RTF |
| | | (SEQ ID NO: 339) |
| PB03_B3 | SEQ ID NO: 48 | LTF (SEQ ID NO: 194), RTF |
| | | (SEQ ID NO: 340) |
| PB03_B4 | SEQ ID NO: 49 | LTF (SEQ ID NO: 195), RTF |
| | | (SEQ ID NO: 341) |
| PB03_B5 | SEQ ID NO: 50 | LTF (SEQ ID NO: 196), RTF |
| | | (SEQ ID NO: 342) |
| PB03_B6 | SEQ ID NO: 51 | LTF (SEQ ID NO: 197), RTF |
| | | (SEQ ID NO: 343) |
| PB03_B8 | SEQ ID NO: 52 | LTF (SEQ ID NO: 198), RTF |
| | | (SEQ ID NO: 344) |
| PB03_B10 | SEQ ID NO: 53 | LTF (SEQ ID NO: 199), RTF |
| | | (SEQ ID NO: 345) |
| PB03_B11 | SEQ ID NO: 54 | LTF (SEQ ID NO: 200), RTF |
| | | (SEQ ID NO: 346) |
| PB03_B12 | SEQ ID NO: 55 | LTF (SEQ ID NO: 201), RTF |
| | | (SEQ ID NO: 347) |
| PB03_C1 | SEQ ID NO: 56 | LTF (SEQ ID NO: 202), RTF |
| | | (SEQ ID NO: 348) |
| PB03_C2 | SEQ ID NO: 57 | LTF (SEQ ID NO: 203), RTF |
| | | (SEQ ID NO: 349) |
| PB03_C3 | SEQ ID NO: 58 | LTF (SEQ ID NO: 204), RTF |
| | | (SEQ ID NO: 350) |
| PB03_C4 | SEQ ID NO: 59 | LTF (SEQ ID NO: 205), RTF |
| | | (SEQ ID NO: 351) |
| PB03_C5 | SEQ ID NO: 60 | LTF (SEQ ID NO: 206), RTF |
| | | (SEQ ID NO: 352) |
| PB03_C7 | SEQ ID NO: 61 | LTF (SEQ ID NO: 207), RTF |
| | | (SEQ ID NO: 353) |
| PB03_C8 | SEQ ID NO: 62 | LTF (SEQ ID NO: 208), RTF |
| | | (SEQ ID NO: 354) |
| PB03_C9 | SEQ ID NO: 63 | LTF (SEQ ID NO: 209), RTF |
| | | (SEQ ID NO: 355) |
| PB03_C10 | SEQ ID NO: 64 | LTF (SEQ ID NO: 210), RTF |
| | | (SEQ ID NO: 356) |
| PB03_C11 | SEQ ID NO: 65 | LTF (SEQ ID NO: 211), RTF |
| | | (SEQ ID NO: 357) |
| PB03_D3 | SEQ ID NO: 66 | LTF (SEQ ID NO: 212), RTF |
| | | (SEQ ID NO: 358) |
| PB03_D4 | SEQ ID NO: 67 | LTF (SEQ ID NO: 213), RTF |
| | | (SEQ ID NO: 359) |
| PB03_D5 | SEQ ID NO: 68 | LTF (SEQ ID NO: 214), RTF |
| | | (SEQ ID NO: 360) |
| PB03_D7 | SEQ ID NO: 69 | LTF (SEQ ID NO: 215), RTF |
| | | (SEQ ID NO: 361) |
| PB03_D8 | SEQ ID NO: 70 | LTF (SEQ ID NO: 216), RTF |
| | | (SEQ ID NO: 362) |
| PB03_D9 | SEQ ID NO: 71 | LTF (SEQ ID NO: 217), RTF |
| | | (SEQ ID NO: 363) |
| PB03_D10 | SEQ ID NO: 72 | LTF (SEQ ID NO: 218), RTF |
| | | (SEQ ID NO: 364) |
| PB03_E1 | SEQ ID NO: 73 | LTF (SEQ ID NO: 219), RTF |
| | | (SEQ ID NO: 365) |
| PB03_E6 | SEQ ID NO: 74 | LTF (SEQ ID NO: 220), RTF |
| | | (SEQ ID NO: 366) |
| PB03_E7 | SEQ ID NO: 75 | LTF (SEQ ID NO: 221), RTF |
| | | (SEQ ID NO: 367) |
| PB03_E8 | SEQ ID NO: 76 | LTF (SEQ ID NO: 222), RTF |
| | | (SEQ ID NO: 368) |
| PB03_E9 | SEQ ID NO: 77 | LTF (SEQ ID NO: 223), RTF |
| | | (SEQ ID NO: 369) |
| PB03_E10 | SEQ ID NO: 78 | LTF (SEQ ID NO: 224), RTF |
| | | (SEQ ID NO: 370) |
| PB03_E11 | SEQ ID NO: 79 | LTF (SEQ ID NO: 225), RTF |
| | | (SEQ ID NO: 371) |
| PB03_F1 | SEQ ID NO: 80 | LTF (SEQ ID NO: 226), RTF |
| | | (SEQ ID NO: 372) |
| PB03_F2 | SEQ ID NO: 81 | LTF (SEQ ID NO: 227), RTF |
| | | (SEQ ID NO: 373) |
| PB03_F3 | SEQ ID NO: 82 | LTF (SEQ ID NO: 228), RTF |
| | | (SEQ ID NO: 374) |
| PB03_F4 | SEQ ID NO: 83 | LTF (SEQ ID NO: 229), RTF |
| | | (SEQ ID NO: 375) |
| PB03_F5 | SEQ ID NO: 84 | LTF (SEQ ID NO: 230), RTF |
| | | (SEQ ID NO: 376) |
| PB03_F6 | SEQ ID NO: 85 | LTF (SEQ ID NO: 231), RTF |
| | | (SEQ ID NO: 377) |
| PB03_F7 | SEQ ID NO: 86 | LTF (SEQ ID NO: 232), RTF |
| | | (SEQ ID NO: 378) |
| PB03_F8 | SEQ ID NO: 87 | LTF (SEQ ID NO: 233), RTF |
| | | (SEQ ID NO: 379) |
| PB03_F9 | SEQ ID NO: 88 | LTF (SEQ ID NO: 234), RTF |
| | | (SEQ ID NO: 380) |
| PB03_F11 | SEQ ID NO: 89 | LTF (SEQ ID NO: 235), RTF |
| | | (SEQ ID NO: 381) |
| PB03_F12 | SEQ ID NO: 90 | LTF (SEQ ID NO: 236), RTF |
| | | (SEQ ID NO: 382) |
| PB03_G1 | SEQ ID NO: 91 | LTF (SEQ ID NO: 237), RTF |
| | | (SEQ ID NO: 383) |
| PB03_G2 | SEQ ID NO: 92 | LTF (SEQ ID NO: 238), RTF |
| | | (SEQ ID NO: 384) |
| PB03_G3 | SEQ ID NO: 93 | LTF (SEQ ID NO: 239), RTF |
| | | (SEQ ID NO: 385) |
| PB03_G4 | SEQ ID NO: 94 | LTF (SEQ ID NO: 240), RTF |
| | | (SEQ ID NO: 386) |
| PB03_G6 | SEQ ID NO: 95 | LTF (SEQ ID NO: 241), RTF |
| | | (SEQ ID NO: 387) |
| PB03_G7 | SEQ ID NO: 96 | LTF (SEQ ID NO: 242), RTF |
| | | (SEQ ID NO: 388) |
| PB03_G8 | SEQ ID NO: 97 | LTF (SEQ ID NO: 243), RTF |
| | | (SEQ ID NO: 389) |
| PB03_G9 | SEQ ID NO: 98 | LTF (SEQ ID NO: 244), RTF |
| | | (SEQ ID NO: 390) |
| PB03_G10 | SEQ ID NO: 99 | LTF (SEQ ID NO: 245), RTF |
| | | (SEQ ID NO: 391) |
| PB03_G11 | SEQ ID NO: 100 | LTF (SEQ ID NO: 246), RTF |
| | | (SEQ ID NO: 392) |
| PB03_G12 | SEQ ID NO: 101 | LTF (SEQ ID NO: 247), RTF |
| | | (SEQ ID NO: 393) |
| PB04_A1 | SEQ ID NO: 102 | LTF (SEQ ID NO: 248), RTF |
| | | (SEQ ID NO: 394) |
| PB04_A3 | SEQ ID NO: 103 | LTF (SEQ ID NO: 249), RTF |
| | | (SEQ ID NO: 395) |
| PB04_A5 | SEQ ID NO: 104 | LTF (SEQ ID NO: 250), RTF |
| | | (SEQ ID NO: 396) |
| PB04_A7 | SEQ ID NO: 105 | LTF (SEQ ID NO: 251), RTF |
| | | (SEQ ID NO: 397) |
| PB04_A10 | SEQ ID NO: 106 | LTF (SEQ ID NO: 252), RTF |
| | | (SEQ ID NO: 398) |
| PB04_A11 | SEQ ID NO: 107 | LTF (SEQ ID NO: 253), RTF |
| | | (SEQ ID NO: 399) |
| PB04_A12 | SEQ ID NO: 108 | LTF (SEQ ID NO: 254), RTF |
| | | (SEQ ID NO: 400) |
| PB04_B5 | SEQ ID NO: 109 | LTF (SEQ ID NO: 255), RTF |
| | | (SEQ ID NO: 401) |
| PB04_B7 | SEQ ID NO: 110 | LTF (SEQ ID NO: 256), RTF |
| | | (SEQ ID NO: 402) |
| PB04_B9 | SEQ ID NO: 111 | LTF (SEQ ID NO: 257), RTF |
| | | (SEQ ID NO: 403) |
| PB04_B10 | SEQ ID NO: 112 | LTF (SEQ ID NO: 258), RTF |
| | | (SEQ ID NO: 404) |
| PB04_B12 | SEQ ID NO: 113 | LTF (SEQ ID NO: 259), RTF |
| | | (SEQ ID NO: 405) |
| PB04_C3 | SEQ ID NO: 114 | LTF (SEQ ID NO: 260), RTF |
| | | (SEQ ID NO: 406) |
| PB04_C5 | SEQ ID NO: 115 | LTF (SEQ ID NO: 261), RTF |
| | | (SEQ ID NO: 407) |
| PB04_C8 | SEQ ID NO: 116 | LTF (SEQ ID NO: 262), RTF |
| | | (SEQ ID NO: 408) |
| PB04_C9 | SEQ ID NO: 117 | LTF (SEQ ID NO: 263), RTF |
| | | (SEQ ID NO: 409) |
| PB04_C11 | SEQ ID NO: 118 | LTF (SEQ ID NO: 264), RTF |
| | | (SEQ ID NO: 410) |
| PB04_D1 | SEQ ID NO: 119 | LTF (SEQ ID NO: 265), RTF |
| | | (SEQ ID NO: 411) |
| PB04_D2 | SEQ ID NO: 120 | LTF (SEQ ID NO: 266), RTF |
| | | (SEQ ID NO: 412) |
| PB04_D3 | SEQ ID NO: 121 | LTF (SEQ ID NO: 267), RTF |
| | | (SEQ ID NO: 413) |
| PB04_D4 | SEQ ID NO: 122 | LTF (SEQ ID NO: 268), RTF |
| | | (SEQ ID NO: 414) |
| PB04_D6 | SEQ ID NO: 123 | LTF (SEQ ID NO: 269), RTF |
| | | (SEQ ID NO: 415) |
| PB04_D9 | SEQ ID NO: 124 | LTF (SEQ ID NO: 270), RTF |
| | | (SEQ ID NO: 416) |
| PB04_E3 | SEQ ID NO: 125 | LTF (SEQ ID NO: 271), RTF |
| | | (SEQ ID NO: 417) |
| PB04_E7 | SEQ ID NO: 126 | LTF (SEQ ID NO: 272), RTF |
| | | (SEQ ID NO: 418) |
| PB04_E8 | SEQ ID NO: 127 | LTF (SEQ ID NO: 273), RTF |
| | | (SEQ ID NO: 419) |
| PB04_E9 | SEQ ID NO: 128 | LTF (SEQ ID NO: 274), RTF |
| | | (SEQ ID NO: 420) |
| PB04_E10 | SEQ ID NO: 129 | LTF (SEQ ID NO: 275), RTF |
| | | (SEQ ID NO: 421) |
| PB04_E12 | SEQ ID NO: 130 | LTF (SEQ ID NO: 276), RTF |
| | | (SEQ ID NO: 422) |
| PB04_F1 | SEQ ID NO: 131 | LTF (SEQ ID NO: 277), RTF |
| | | (SEQ ID NO: 423) |
| PB04_F3 | SEQ ID NO: 132 | LTF (SEQ ID NO: 278), RTF |
| | | (SEQ ID NO: 424) |
| PB04_F7 | SEQ ID NO: 133 | LTF (SEQ ID NO: 279), RTF |
| | | (SEQ ID NO: 425) |
| PB04_F8 | SEQ ID NO: 134 | LTF (SEQ ID NO: 280), RTF |
| | | (SEQ ID NO: 426) |
| PB04_F10 | SEQ ID NO: 135 | LTF (SEQ ID NO: 281), RTF |
| | | (SEQ ID NO: 427) |
| PB04_F11 | SEQ ID NO: 136 | LTF (SEQ ID NO: 282), RTF |
| | | (SEQ ID NO: 428) |
| PB04_F12 | SEQ ID NO: 137 | LTF (SEQ ID NO: 283), RTF |
| | | (SEQ ID NO: 429) |
| PB04_G1 | SEQ ID NO: 138 | LTF (SEQ ID NO: 284), RTF |
| | | (SEQ ID NO: 430) |
| PB04_G4 | SEQ ID NO: 139 | LTF (SEQ ID NO: 285), RTF |
| | | (SEQ ID NO: 431) |
| PB04_G7 | SEQ ID NO: 140 | LTF (SEQ ID NO: 286), RTF |
| | | (SEQ ID NO: 432) |
| PB04_G8 | SEQ ID NO: 141 | LTF (SEQ ID NO: 287), RTF |
| | | (SEQ ID NO: 433) |
| PB04_G10 | SEQ ID NO: 142 | LTF (SEQ ID NO: 288), RTF |
| | | (SEQ ID NO: 434) |
| PB04_G12 | SEQ ID NO: 143 | LTF (SEQ ID NO: 289), RTF |
| | | (SEQ ID NO: 435) |
| PB04_D7 | SEQ ID NO: 144 | LTF (SEQ ID NO: 290), RTF |
| | | (SEQ ID NO: 436) |
| PB04_D11 | SEQ ID NO: 145 | LTF (SEQ ID NO: 291), RTF |
| | | (SEQ ID NO: 437) |
| PB04_F2 | SEQ ID NO: 146 | LTF (SEQ ID NO: 292), RTF |
| | | (SEQ ID NO: 438) |
| PB01_A5 | SEQ ID NO: 439 | LTF (SEQ ID NO: 459), RTF |
| | | (SEQ ID NO: 479) |
| PB01_A7 | SEQ ID NO: 440 | LTF (SEQ ID NO: 460), RTF |
| | | (SEQ ID NO: 480) |
| PB01_B1 | SEQ ID NO: 441 | LTF (SEQ ID NO: 461), RTF |
| | | (SEQ ID NO: 481) |
| PB01_B3 | SEQ ID NO: 442 | LTF (SEQ ID NO: 462), RTF |
| | | (SEQ ID NO: 482) |
| PB01_B6 | SEQ ID NO: 443 | LTF (SEQ ID NO: 463), RTF |
| | | (SEQ ID NO: 483) |
| PB01_B8 | SEQ ID NO: 444 | LTF (SEQ ID NO: 464), RTF |
| | | (SEQ ID NO: 484) |
| PB01_E3 | SEQ ID NO: 445 | LTF (SEQ ID NO: 465), RTF |
| | | (SEQ ID NO: 485) |
| PB01_E9 | SEQ ID NO: 446 | LTF (SEQ ID NO: 466), RTF |
| | | (SEQ ID NO: 486) |
| PB01_F3 | SEQ ID NO: 447 | LTF (SEQ ID NO: 467), RTF |
| | | (SEQ ID NO: 487) |
| PB01_F12 | SEQ ID NO: 448 | LTF (SEQ ID NO: 468), RTF |
| | | (SEQ ID NO: 488) |
| PB02_B1 | SEQ ID NO: 449 | LTF (SEQ ID NO: 469), RTF |
| | | (SEQ ID NO: 489) |
| PB02_B4 | SEQ ID NO: 450 | LTF (SEQ ID NO: 470), RTF |
| | | (SEQ ID NO: 490) |
| PB02_B11 | SEQ ID NO: 451 | LTF (SEQ ID NO: 471), RTF |
| | | (SEQ ID NO: 491) |
| PB02_B12 | SEQ ID NO: 452 | LTF (SEQ ID NO: 472), RTF |
| | | (SEQ ID NO: 492) |
| PB02_C12 | SEQ ID NO: 453 | LTF (SEQ ID NO: 473), RTF |
| | | (SEQ ID NO: 493) |
| PB02_D8 | SEQ ID NO: 454 | LTF (SEQ ID NO: 474), RTF |
| | | (SEQ ID NO: 494) |
| PB02_E2 | SEQ ID NO: 455 | LTF (SEQ ID NO: 475), RTF |
| | | (SEQ ID NO: 495) |
| PB02_F2 | SEQ ID NO: 456 | LTF (SEQ ID NO: 476), RTF |
| | | (SEQ ID NO: 496) |
| PB02_F4 | SEQ ID NO: 457 | LTF (SEQ ID NO: 477), RTF |
| | | (SEQ ID NO: 497) |
| PB03_D1 | SEQ ID NO: 458 | LTF (SEQ ID NO: 478), RTF |
| | | (SEQ ID NO: 498) |

When both plasmids were co-transfected into HEK293T cells, the transcription of the transposase gene from plasmid 1 was initiated to express a transposase protein, the transposase protein then recognized and bound to the transposon recognition sequences on plasmid 2, and cut all the sequences including the transposon recognition sequences, the GFP gene and the puromycin resistance gene from the plasmid vector and integrated them into the genome of a cell. When the cells were continuously cultured with a medium containing a certain concentration of puromycin, only the cells in which the transposition event occurred survived because they contained the puromycin resistance gene in their genome. The transposition activity level of the candidate transposase was reflected by the number of surviving cells or their ability to form monoclonal cells.

### Method for DNA Synthesis and Plasmid Construction:

Construction of plasmid 1: a DNA sequence corresponding to the amino acid sequence of the transposase was synthesized by Beijing Tsingke Biotech Co., Ltd. and GENERAL Biosystems (Anhui) Co., Ltd., and cloned into a plasmid vector pICOZ that contains a CMV promoter element via EcoRI site at the 5′ end and NotI site at the 3′ end, so that the transposase gene is transcribed and subsequently translated into a functional protein in eukaryotic cells under the control of the CMV promoter.

Construction of plasmid 2: the transposon sequences (including terminal inverted repeats) are located at both sides of the open reading frame of the transposase, the left transposon fragment (LTF) comprises all DNA sequences from the target site duplication (TSD) sequence at the 5′ end to the sequence before the transposase start codon, and the right transposon fragment (RTF) comprises all DNA sequences from the first base after the transposase stop codon to the TSD sequence at the 3′ end. In principle, the terminal repeats at both sides recognized by the transposase are comprised in the transposon sequences at both sides, respectively. LTF and RTF fragments were synthesized by BGI Tech Solutions (Beijing Liuhe) Co., Ltd., and were respectively cloned into a pMV plasmid vector that contains elements such as a PGK promoter, a puromycin resistance gene (PuroR), P2A, a green fluorescent protein gene (GFP) and poly(A), so that LTF was located upstream of the PGK promoter and RTF was located downstream of the poly(A).

Plasmid 1 corresponds to plasmid 2 one by one.

### Example 2: High Throughput Screening of Transposition Activity

### 2.1 Cell Treatment (Day 0):

A HEK293T cells (commercially purchased) stably expressing the firefly luciferase gene were established for high throughput screening assay. When cultured to the logarithmic growth phase, the cells were digested and dispersed into single cells with 0.25% Trypsin (Thermo), and added to a 96-well cell culture plate pre-coated with PDL (Sigma) at a cell concentration of 1.0×10⁴ cells/well, and cultured overnight at 37° C. in 5% CO2.

### 2.2 Cell Transfection (Day 1):

Two plasmids corresponding to each transposon system were mixed at a dose of 20 ng for plasmid 1 and 10 ng for plasmid 2, then mixed with a transfection reagent Lipofectamine 2000 (Thermo) at a ratio of the mass of the transfection plasmid (μg):the volume of the transfection reagent (μL) being 1:2, and left to stand at room temperature for 15 min to form a transfection complex. The transfection complex was transferred to the cell culture plate and incubated with the cells, and two parallel tests were performed for each sample to be screened.

### 2.3 Cell Screening (Day 3)

48 h after transfection, the culture medium was replaced to DMEM (Thermo) screening medium containing 2 μg/mL puromycin (Invivogen), 10% fetal bovine serum and 1% penicillin/streptomycin (Thermo), and cultured for 4 days at 37° C. in 5% CO2. Then, the cells were digested into single cells with 0.25% Trypsin, diluted at a ratio of 1:5, transferred to another 96-well culture plate pre-coated with PDL, and cultured for 4 days at 37° C. in a DMEM screening medium containing 2 μg/mL puromycin, 10% fetal bovine serum and 1% penicillin/streptomycin.

### 2.4 Detection of Cell Viability (Day 11)

2.4.1 preparation of detection reagents: The Steady-Glo® Luciferase Assay System (Promega) was mixed with PBS at volume ratio of 1:5. The detection reagents was prepared at a dose of 50 μL/well, and 5 mL of detection reagents was prepared for a 96-well plate.

2.4.2 The cells screened by puromycin for 8 days were removed from the incubator. After the culture medium were removed, the detection reagent was added at a dose of 50 μL/well. After incubated at room temperature for 5 minutes in the dark, a multifunctional microplate reader with luminescence detection function was used for detection. The more cells survived after puromycin screening, the stronger the luminescence signal detected, indicating the higher transposition activity of the sample.

### 2.5 Statistical Results

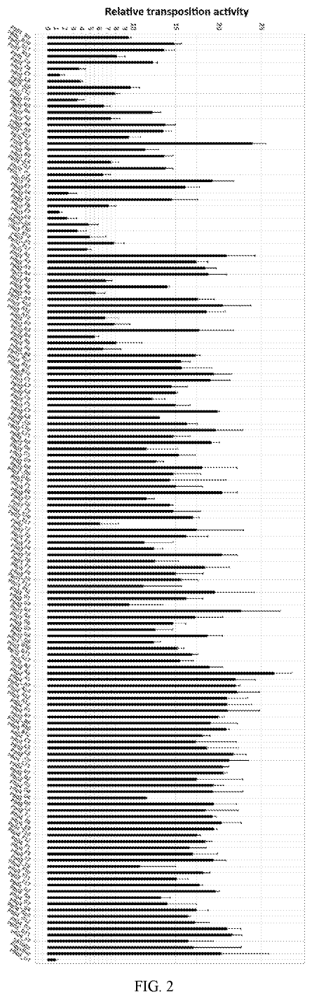

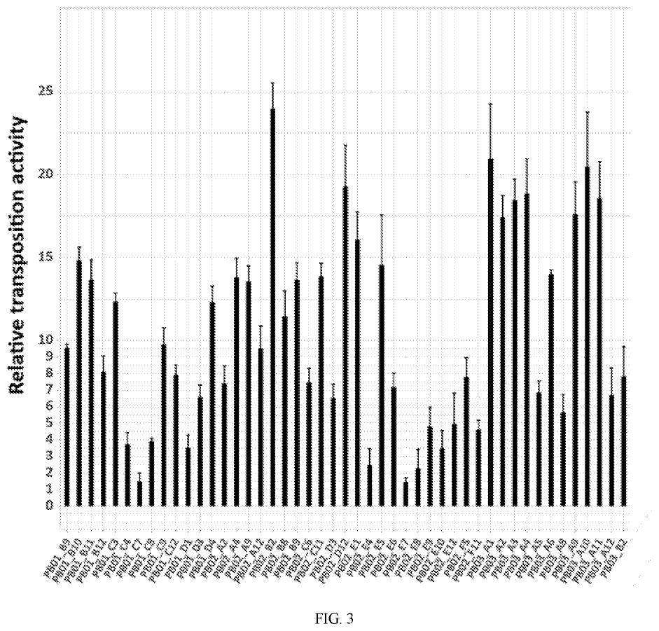

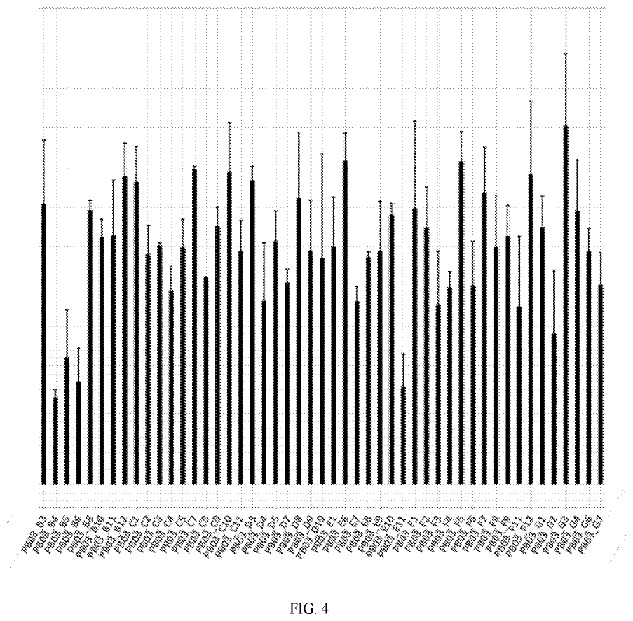

During high-throughput screening, positive and negative control were set up on each plate. According to the reading value of the luminescence signal detected by the microplate reader in each hole, the fold change of the reading value of each sample relative to the average reading value of positive control (SB100X) is calculated. Such calculated fold change of each sample was then divided by the calculated fold change of an inactive transposase (PB03_D1), thus obtaining the relative transposition activity of all transposases as shown in FIG. 2 and Table 2.

TABLE 2
The results of relative transposition activity in example 2

| | Relative transposition activity |
|---|---|
| Name | (mean ± standard deviation) |
| PB01_B9 | 9.55 ± 0.24 |
| PB01_B10 | 14.83 ± 0.82 |
| PB01_B11 | 13.66 ± 1.21 |
| PB01_B12 | 8.13 ± 0.94 |
| PB01_C3 | 12.34 ± 0.52 |
| PB01_C4 | 3.75 ± 0.70 |
| PB01_C7 | 1.50 ± 0.50 |
| PB01_C8 | 3.92 ± 0.20 |
| PB01_C9 | 9.75 ± 1.01 |
| PB01_C12 | 7.92 ± 0.58 |
| PB01_D1 | 3.54 ± 0.77 |
| PB01_D3 | 6.59 ± 0.73 |
| PB01_D4 | 12.32 ± 0.97 |
| PB02_A2 | 7.42 ± 1.05 |
| PB02_A4 | 13.80 ± 1.15 |
| PB02_A9 | 13.58 ± 0.93 |
| PB02_A12 | 9.52 ± 1.36 |
| PB02_B2 | 23.99 ± 1.56 |
| PB02_B8 | 11.46 ± 1.53 |
| PB02_B9 | 13.66 ± 1.03 |
| PB02_C6 | 7.47 ± 0.85 |
| PB02_C11 | 13.86 ± 0.81 |
| PB02_D3 | 6.53 ± 0.83 |
| PB02_D12 | 19.29 ± 2.49 |
| PB02_E1 | 16.12 ± 1.65 |
| PB02_E4 | 2.51 ± 0.97 |
| PB02_E5 | 14.56 ± 3.00 |
| PB02_E6 | 7.19 ± 0.82 |
| PB02_E7 | 1.46 ± 0.26 |
| PB02_E8 | 2.31 ± 1.14 |
| PB02_E9 | 4.82 ± 1.14 |
| PB02_E10 | 3.50 ± 1.06 |
| PB02_E12 | 4.95 ± 1.87 |
| PB02_F5 | 7.80 ± 1.16 |
| PB02_F11 | 4.63 ± 0.56 |
| PB03_A1 | 20.96 ± 3.30 |
| PB03_A2 | 17.43 ± 1.32 |
| PB03_A3 | 18.46 ± 1.27 |
| PB03_A4 | 18.86 ± 2.08 |
| PB03_A5 | 6.86 ± 0.69 |
| PB03_A6 | 14.00 ± 0.26 |
| PB03_A8 | 5.67 ± 1.07 |
| PB03_A9 | 17.63 ± 1.92 |
| PB03_A10 | 20.50 ± 3.28 |
| PB03_A11 | 18.60 ± 2.18 |
| PB03_A12 | 6.73 ± 1.61 |
| PB03_B2 | 7.86 ± 1.78 |
| PB03_B3 | 17.71 ± 4.03 |
| PB03_B4 | 5.53 ± 0.47 |
| PB03_B5 | 8.04 ± 2.99 |
| PB03_B6 | 6.52 ± 2.10 |
| PB03_B8 | 17.33 ± 0.61 |
| PB03_B10 | 15.62 ± 1.10 |
| PB03_B11 | 15.70 ± 3.49 |
| PB03_B12 | 19.45 ± 2.09 |
| PB03_C1 | 19.10 ± 2.22 |
| PB03_C2 | 14.54 ± 1.81 |
| PB03_C3 | 15.08 ± 0.18 |
| PB03_C4 | 12.26 ± 1.47 |
| PB03_C5 | 14.96 ± 1.76 |
| PB03_C7 | 19.90 ± 0.17 |
| PB03_C8 | 13.07 ± 0.03 |
| PB03_C9 | 16.31 ± 1.20 |
| PB03_C10 | 19.71 ± 3.14 |
| PB03_C11 | 14.72 ± 1.95 |
| PB03_D3 | 19.20 ± 0.88 |
| PB03_D4 | 11.58 ± 3.68 |
| PB03_D5 | 15.37 ± 1.90 |
| PB03_D7 | 12.73 ± 0.84 |
| PB03_D8 | 18.07 ± 4.10 |
| PB03_D9 | 14.75 ± 3.19 |
| PB03_D10 | 14.28 ± 6.55 |
| PB03_E1 | 15.01 ± 3.13 |
| PB03_E6 | 20.46 ± 1.73 |
| PB03_E7 | 11.59 ± 0.90 |
| PB03_E8 | 14.35 ± 0.33 |
| PB03_E9 | 14.74 ± 3.12 |
| PB03_E10 | 17.01 ± 0.70 |
| PB03_E11 | 6.18 ± 2.09 |
| PB03_F1 | 17.42 ± 5.49 |
| PB03_F2 | 16.22 ± 2.56 |
| PB03_F3 | 11.34 ± 3.37 |
| PB03_F4 | 12.44 ± 1.00 |
| PB03_F5 | 20.39 ± 1.86 |
| PB03_F6 | 12.58 ± 2.76 |
| PB03_F7 | 18.42 ± 2.86 |
| PB03_F8 | 14.99 ± 3.23 |
| PB03_F9 | 15.67 ± 1.94 |
| PB03_F11 | 11.24 ± 4.43 |
| PB03_F12 | 19.58 ± 4.58 |
| PB03_G1 | 16.22 ± 1.97 |
| PB03_G2 | 9.52 ± 3.97 |
| PB03_G3 | 22.64 ± 4.55 |
| PB03_G4 | 17.28 ± 3.21 |
| PB03_G6 | 14.71 ± 1.45 |
| PB03_G7 | 12.61 ± 2.03 |
| PB03_G8 | 18.70 ± 1.76 |
| PB03_G9 | 12.44 ± 0.75 |
| PB03_G10 | 15.23 ± 0.73 |
| PB03_G11 | 16.94 ± 0.67 |
| PB03_G12 | 15.52 ± 1.51 |
| PB04_A1 | 18.99 ± 1.52 |
| PB04_A3 | 26.54 ± 2.09 |
| PB04_A5 | 22.01 ± 2.29 |
| PB04_A7 | 21.98 ± 0.54 |
| PB04_A10 | 22.18 ± 2.60 |
| PB04_A11 | 20.99 ± 2.39 |
| PB04_A12 | 21.03 ± 2.84 |
| PB04_B5 | 21.08 ± 3.68 |
| PB04_B7 | 19.97 ± 0.70 |
| PB04_B9 | 19.08 ± 3.15 |
| PB04_B10 | 20.96 ± 0.31 |
| PB04_B12 | 18.16 ± 0.86 |
| PB04_C3 | 17.32 ± 4.77 |
| PB04_C5 | 18.63 ± 3.66 |
| PB04_C8 | 21.72 ± 1.50 |
| PB04_C9 | 21.27 ± 2.21 |
| PB04_C11 | 20.49 ± 0.73 |
| PB04_D1 | 20.59 ± 0.41 |
| PB04_D2 | 17.42 ± 5.41 |
| PB04_D3 | 19.41 ± 1.14 |
| PB04_D4 | 19.36 ± 3.50 |
| PB04_D6 | 11.52 ± 0.11 |
| PB04_D9 | 19.46 ± 2.65 |
| PB04_E3 | 18.50 ± 3.79 |
| PB04_E7 | 19.32 ± 0.44 |
| PB04_E8 | 20.35 ± 2.31 |
| PB04_E9 | 19.48 ± 0.37 |
| PB04_E10 | 17.52 ± 0.35 |
| PB04_E12 | 18.47 ± 0.75 |
| PB04_F1 | 16.60 ± 1.96 |
| PB04_F3 | 17.02 ± 2.88 |
| PB04_F7 | 19.40 ± 1.52 |
| PB04_F8 | 10.84 ± 4.18 |
| PB04_F10 | 18.19 ± 0.83 |
| PB04_F11 | 15.09 ± 1.32 |
| PB04_F12 | 17.76 ± 0.36 |
| PB04_G1 | 19.69 ± 0.40 |
| PB04_G4 | 13.26 ± 1.10 |
| PB04_G7 | 14.02 ± 3.43 |
| PB04_G8 | 17.39 ± 1.37 |
| PB04_G10 | 16.42 ± 0.33 |
| PB04_G12 | 17.22 ± 1.74 |
| PB04_D7 | 21.04 ± 1.60 |
| PB04_D11 | 21.59 ± 1.16 |
| PB04_F2 | 16.45 ± 2.93 |
| SB100X | 17.07 ± 5.59 |
| PiggyBac | 20.34 ± 5.51 |
| PB03_D1 | 1.00 ± 0.27 |

### Example 3: Transposition Activity Assay

### 3.1 Cell Treatment (Day 0):

After HEK293T cells (commercially purchased) were cultured to the logarithmic growth phase, they were digested and dispersed into single cells with 0.25% Trypsin (Thermo), and added to a 24-well cell culture plate pre-coated with PDL (Sigma) at a cell concentration of 1.2× 10⁵ cells/well, and cultured overnight at 37° C. in 5% CO2.

### 3.2 Cell Transfection (Day 1):

Two plasmids corresponding to each transposon system were mixed at a dose of 200 ng for plasmid 1 and 100 ng for plasmid 2, then mixed with a transfection reagent Lipofectamine 2000 (Thermo) at a ratio of the mass of the transfection plasmid (μg):the volume of the transfection reagent (μL) being 1:2, and left to stand at room temperature for 15 min to form a transfection complex. The transfection complex was transferred to the cell culture plate and incubated with the cells, and two parallel tests were performed for each sample to be screened.

### 3.3 Cell Screening (Day 3)

48 h after transfection, the cells were digested and dispersed into single cells with 0.25% Trypsin, the cells were added to a DMEM (Thermo) screening medium containing 2 μg/mL puromycin (Invivogen), 10% fetal bovine serum and 1% penicillin/streptomycin (Thermo), diluted at a ratio of 1:2000, and transferred to a 6-well culture plate for further culture. After 10 days of continuous screening culture with the puromycin resistance medium, the clones were counted, and the transposition activity of the transposase was calculated.

### 3.4 Cell Staining (Day 13)

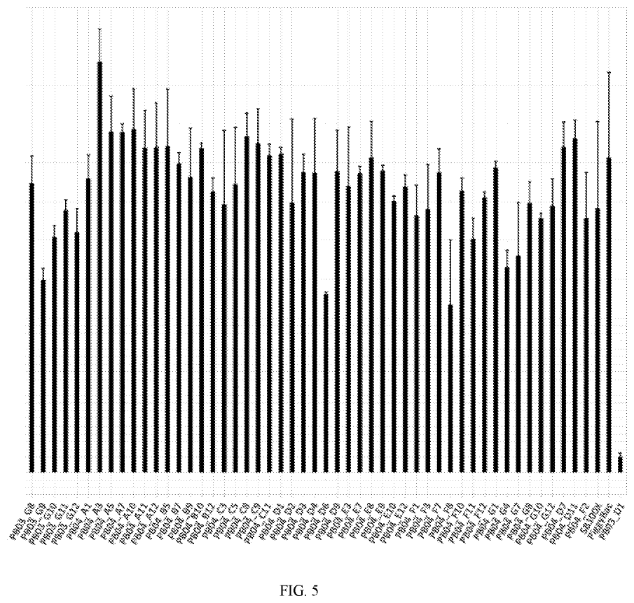

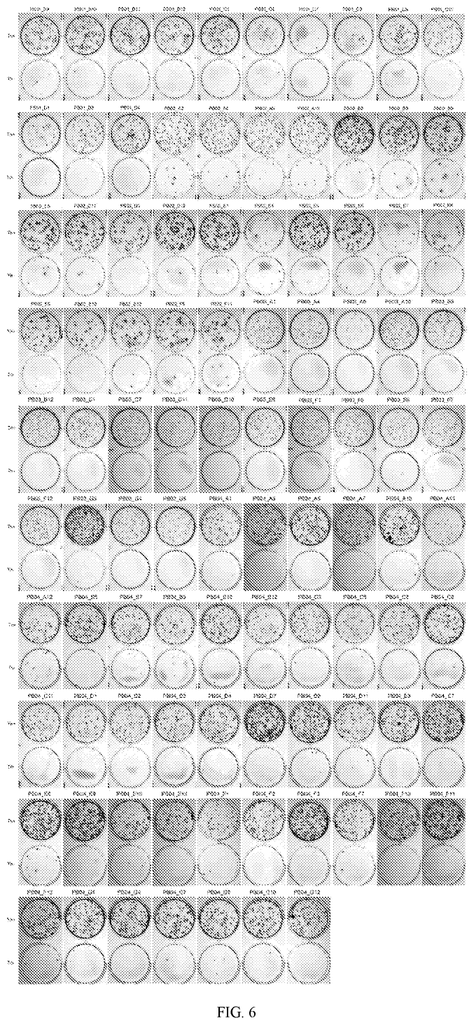

The cells that were screened by puromycin and cultured in the 6-well plate were washed with PBS, and then fixed at room temperature for 15 min with 4% paraformaldehyde. The waste liquid was discarded, and a 0.2% methylene blue staining solution was added to the cells. The cells were stained at room temperature for 1 h. The stained cell clones were washed with PBS, and photographed in an imaging system (BioRad). The number of cell clones in each well was counted. The cloning and screening results of the transposases of the present application in HEK293T cells were as shown in FIG. 6. The staining results of the surviving cell clones after puromycin resistance screening were as shown in the figures, showing that the transposition event occurred successfully. Tn+ represents co-transfection of a transposase plasmid and a donor plasmid, and Tn− represents transfection of a donor plasmid only, as a negative control for each transposase sample.

### 3.5 Statistical Results

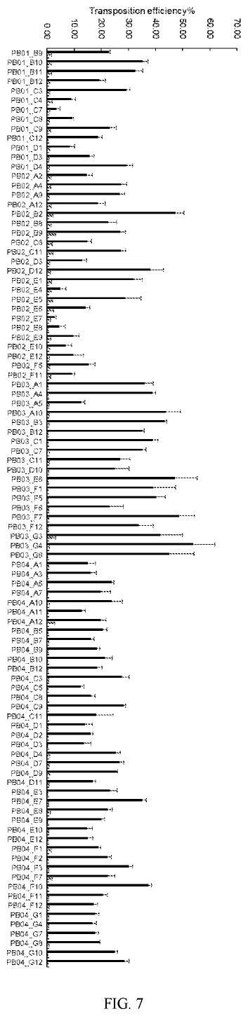

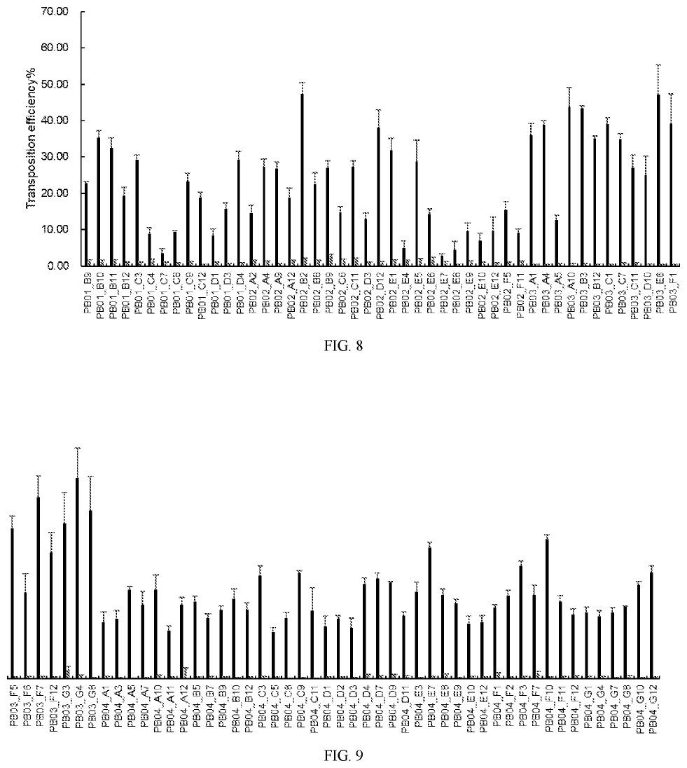

The statistical results of transposition activity were as shown in FIG. 7 and Table 3. Tn+ represents co-transfection of a transposase plasmid and a donor plasmid, and Tn-represents transfection of a donor plasmid only. The y-axis in the figure showed the percentage of transposition activity according to calculation, and the specific calculation formula was as follows: transposition efficiency (%)=the number of cell clones per well/(the number of cells plated per well x transfection efficiency (GFP positive cells %))×100%.

TABLE 3
The statistical results of transposition activity in example 3

| | Tn+ | Tn− |
|---|---|---|
| Name | (mean ± standard deviation) | (mean ± standard deviation) |
| PB01_B9 | 22.69 ± 0.56 | 1.20 ± 0.53 |
| PB01_B10 | 35.24 ± 1.95 | 1.10 ± 0.50 |
| PB01_B11 | 32.47 ± 2.87 | 1.30 ± 0.39 |
| PB01_B12 | 19.32 ± 2.23 | 0.99 ± 0.16 |
| PB01_C3 | 29.32 ± 1.23 | 0.74 ± 0.29 |
| PB01_C4 | 8.91 ± 1.65 | 1.27 ± 0.55 |
| PB01_C7 | 3.57 ± 1.20 | 0.61 ± 0.47 |
| PB01_C8 | 9.31 ± 0.47 | 0.67 ± 0.23 |
| PB01_C9 | 23.17 ± 2.40 | 1.03 ± 0.25 |
| PB01_C12 | 18.84 ± 1.39 | 0.35 ± 0.12 |
| PB01_D1 | 8.41 ± 1.82 | 0.93 ± 0.19 |
| PB01_D3 | 15.66 ± 1.73 | 0.65 ± 0.07 |
| PB01_D4 | 29.27 ± 2.30 | 0.59 ± 0.32 |
| PB02_A2 | 14.63 ± 2.08 | 1.30 ± 0.28 |
| PB02_A4 | 27.22 ± 2.27 | 1.22 ± 0.11 |
| PB02_A9 | 26.78 ± 1.84 | 0.63 ± 0.06 |
| PB02_A12 | 18.78 ± 2.67 | 1.48 ± 0.06 |
| PB02_B2 | 47.31 ± 3.08 | 1.98 ± 0.27 |
| PB02_B8 | 22.60 ± 3.01 | 1.17 ± 0.42 |
| PB02_B9 | 26.94 ± 2.02 | 2.84 ± 0.30 |
| PB02_C6 | 14.74 ± 1.68 | 1.45 ± 0.39 |
| PB02_C11 | 27.33 ± 1.59 | 1.91 ± 0.30 |
| PB02_D3 | 12.89 ± 1.63 | 0.85 ± 0.28 |
| PB02_D12 | 38.05 ± 4.90 | 0.71 ± 0.53 |
| PB02_E1 | 31.79 ± 3.25 | 1.56 ± 0.14 |
| PB02_E4 | 4.95 ± 1.92 | 1.27 ± 0.34 |
| PB02_E5 | 28.71 ± 5.92 | 1.92 ± 0.08 |
| PB02_E6 | 14.19 ± 1.62 | 1.70 ± 0.60 |
| PB02_E7 | 2.89 ± 0.51 | 0.79 ± 0.42 |
| PB02_E8 | 4.56 ± 2.24 | 0.33 ± 0.07 |
| PB02_E9 | 9.50 ± 2.25 | 0.95 ± 0.42 |
| PB02_E10 | 6.91 ± 2.10 | 0.60 ± 0.49 |
| PB02_E12 | 9.77 ± 3.69 | 0.80 ± 0.10 |
| PB02_F5 | 15.38 ± 2.29 | 0.87 ± 0.25 |
| PB02_F11 | 9.14 ± 1.10 | 1.19 ± 0.27 |
| PB03_A1 | 35.94 ± 3.18 | 0.32 ± 0.16 |
| PB03_A4 | 38.78 ± 1.15 | 0.27 ± 0.12 |
| PB03_A5 | 12.61 ± 1.26 | 0.53 ± 0.29 |
| PB03_A10 | 43.73 ± 5.41 | 0.45 ± 0.26 |
| PB03_B3 | 43.42 ± 0.64 | 0.41 ± 0.31 |
| PB03_B12 | 35.07 ± 0.78 | 0.22 ± 0.14 |
| PB03_C1 | 39.08 ± 1.79 | 0.22 ± 0.15 |
| PB03_C7 | 34.94 ± 1.40 | 0.51 ± 0.34 |
| PB03_C11 | 27.03 ± 3.58 | 0.73 ± 0.16 |
| PB03_D10 | 24.96 ± 5.26 | 0.38 ± 0.18 |
| PB03_E6 | 47.13 ± 8.20 | 0.31 ± 0.18 |
| PB03_F1 | 39.17 ± 8.13 | 0.22 ± 0.17 |
| PB03_F5 | 40.31 ± 3.34 | 0.30 ± 0.17 |
| PB03_F6 | 23.10 ± 5.08 | 0.45 ± 0.09 |
| PB03_F7 | 48.64 ± 5.76 | 0.43 ± 0.10 |
| PB03_F12 | 33.76 ± 5.35 | 0.39 ± 0.05 |
| PB03_G3 | 41.58 ± 8.35 | 2.16 ± 1.04 |
| PB03_G4 | 53.79 ± 7.97 | 0.55 ± 0.39 |
| PB03_G8 | 45.10 ± 9.11 | 0.16 ± 0.08 |
| PB04_A1 | 15.05 ± 2.83 | 0.40 ± 0.16 |
| PB04_A3 | 16.11 ± 2.05 | 0.21 ± 0.12 |
| PB04_A5 | 23.86 ± 0.88 | 0.09 ± 0.00 |
| PB04_A7 | 19.82 ± 3.57 | 0.18 ± 0.15 |
| PB04_A10 | 23.83 ± 4.02 | 0.57 ± 0.29 |
| PB04_A11 | 12.76 ± 1.41 | 0.24 ± 0.24 |
| PB04_A12 | 19.87 ± 1.92 | 2.76 ± 0.07 |
| PB04_B5 | 20.67 ± 1.41 | 0.33 ± 0.05 |
| PB04_B7 | 16.27 ± 1.17 | 0.39 ± 0.13 |
| PB04_B9 | 18.28 ± 1.15 | 0.31 ± 0.08 |
| PB04_B10 | 21.44 ± 2.63 | 0.17 ± 0.09 |
| PB04_B12 | 18.49 ± 1.85 | 0.33 ± 0.16 |
| PB04_C3 | 27.66 ± 2.64 | 0.29 ± 0.27 |
| PB04_C5 | 12.54 ± 1.09 | 0.23 ± 0.22 |
| PB04_C8 | 16.23 ± 1.49 | 0.03 ± 0.05 |
| PB04_C9 | 28.29 ± 0.57 | 0.17 ± 0.15 |
| PB04_C11 | 18.11 ± 6.31 | 0.05 ± 0.08 |
| PB04_D1 | 13.99 ± 2.73 | 0.26 ± 0.18 |
| PB04_D2 | 16.04 ± 0.83 | 0.33 ± 0.13 |
| PB04_D3 | 13.60 ± 2.66 | 0.17 ± 0.11 |
| PB04_D4 | 25.31 ± 1.60 | 0.76 ± 0.26 |
| PB04_D7 | 26.88 ± 1.47 | 0.51 ± 0.25 |
| PB04_D9 | 25.73 ± 0.27 | 0.87 ± 0.22 |
| PB04_D11 | 16.95 ± 0.94 | 0.49 ± 0.33 |
| PB04_E3 | 23.24 ± 2.54 | 0.12 ± 0.10 |
| PB04_E7 | 35.04 ± 1.60 | 0.35 ± 0.16 |
| PB04_E8 | 22.38 ± 1.65 | 0.68 ± 0.52 |
| PB04_E9 | 20.16 ± 1.18 | 0.26 ± 0.08 |
| PB04_E10 | 14.76 ± 1.95 | 0.45 ± 0.15 |
| PB04_E12 | 15.15 ± 1.81 | 0.34 ± 0.09 |
| PB04_F1 | 18.98 ± 0.75 | 1.02 ± 0.56 |
| PB04_F2 | 22.23 ± 1.50 | 0.35 ± 0.09 |
| PB04_F3 | 30.17 ± 1.31 | 0.54 ± 0.11 |
| PB04_F7 | 22.45 ± 2.57 | 1.32 ± 0.58 |
| PB04_F10 | 37.46 ± 1.09 | 0.25 ± 0.12 |
| PB04_F11 | 20.63 ± 1.65 | 0.36 ± 0.16 |
| PB04_F12 | 17.26 ± 1.47 | 0.71 ± 0.04 |
| PB04_G1 | 17.68 ± 1.48 | 0.27 ± 0.24 |
| PB04_G4 | 16.76 ± 1.37 | 0.24 ± 0.28 |
| PB04_G7 | 17.66 ± 1.30 | 0.20 ± 0.10 |
| PB04_G8 | 19.26 ± 0.26 | 0.44 ± 0.39 |
| PB04_G10 | 25.04 ± 1.00 | 0.18 ± 0.06 |
| PB04_G12 | 28.48 ± 1.74 | 0.28 ± 0.19 |

During the execution of all examples described above, two transposons, SB100X and PiggyBac, were used as positive controls for assessing the transposition activity of the transposases of the present application. These two transposons were commercially used DNA transposons that had been recently patented, and were synthesized and cloned into the corresponding plasmid vectors using the same method as that in example 1 with reference to sequences reported by Lajos Ma'te's et al. (Molecular evolution of a novel hyperactive Sleeping Beauty transposase enables robust stable gene transfer in vertebrates, Nature Genetics, 2009 41 (6): 753-761) and Cary, L. C. et al. (Transposon mutagenesis of baculoviruses: analysis of Trichoplusia ni transposon IFP2 insertions within the FP-locus of nuclear polyhedrosis viruses, Virology, 1989, 172 (1): 156-169).

The statistical results of the transposition activity of the transposases of the present application were as shown in FIG. 2 and FIG. 7. The above results showed that 146 transposases of the present application (PB01_B9, PB01_B10, PB01_B11, PB01_B12, PB01_C3, PB01_C4, PB01_C7, PB01_C8, PB01_C9, PB01_C12, PB01_D1, PB01_D3, PB01_D4, PB02_A2, PB02_A4, PB02_A9, PB02_A12, PB02_B2, PB02_B8, PB02_B9, PB02_C6, PB02_C11, PB02_D3, PB02_D12, PB02_E1, PB02_E4, PB02_E5, PB02_E6, PB02_E7, PB02_E8, PB02_E9, PB02_E10, PB02_E12, PB02_F5, PB02_F11, PB03_A1, PB03_A2, PB03_A3, PB03_A4, PB03_A5, PB03_A6, PB03_A8, PB03_A9, PB03_A10, PB03_A11, PB03_A12, PB03_B2, PB03_B3, PB03_B4, PB03_B5, PB03_B6, PB03_B8, PB03_B10, PB03_B11, PB03_B12, PB03_C1, PB03_C2, PB03_C3, PB03_C4, PB03_C5, PB03_C7, PB03_C8, PB03_C9, PB03_C10, PB03_C11, PB03_D3, PB03_D4, PB03_D5, PB03_D7, PB03_D8, PB03_D9, PB03_D10, PB03_E1, PB03_E6, PB03_E7, PB03_E8, PB03_E9, PB03_E10, PB03_E11, PB03_F1, PB03_F2, PB03_F3, PB03_F4, PB03_F5, PB03_F6, PB03_F7, PB03_F8, PB03_F9, PB03_F11, PB03_F12, PB03_G1, PB03_G2, PB03_G3, PB03_G4, PB03_G6, PB03_G7, PB03_G8, PB03_G9, PB03_G10, PB03_G11, PB03_G12, PB04_A1, PB04_A3, PB04_A5, PB04_A7. PB04_A10, PB04_A11, PB04_A12, PB04_B5, PB04_B7, PB04_B9, PB04_B10, PB04_B12, PB04_C3, PB04_C5, PB04_C8, PB04_C9, PB04_C11, PB04_D1, PB04_D2, PB04_D3, PB04_D4, PB04_D6, PB04_D9, PB04_E3, PB04_E7, PB04_E8, PB04_E9, PB04_E10, PB04_E12, PB04_F1, PB04_F3, PB04_F7, PB04_F8, PB04_F10, PB04_F11, PB04_F12, PB04_G1, PB04_G4, PB04_G7, PB04_G8, PB04_G10, PB04_G12, PB04_D7, PB04_D11, and PB04_F2) had good transposition activity.

Meanwhile, a large number of transposases with inactive or low transposition activity were also found during the screening process (e.g. PB01_A5, PB01_A7, PB01_B1, PB01_B3, PB01_B6, PB01_B8, PB01_E3, PB01_E9, PB01_F3, PB01_F12, PB02_B1, PB02_B4, PB02_B11, PB02_B12, PB02_C12, PB02_D8, PB02_E2, PB02_F2, PB02_F4, PB03_D1 in Table 1 of this application). Compared with these transposases with inactive or low transposition activity, the transposition activity of the 146 transposases of the present application were markedly higher (PB01_B9, PB01_B10, PB01_B11, PB01_B12, PB01_C3, PB01_C4, PB01_C7, PB01_C8, PB01_C9, PB01_C12, PB01_D1, PB01_D3, PB01_D4, PB02_A2, PB02_A4, PB02_A9, PB02_A12, PB02_B2, PB02_B8, PB02_B9, PB02_C6, PB02_C11, PB02_D3, PB02_D12, PB02_E1, PB02_E4, PB02_E5, PB02_E6, PB02_E7, PB02_E8, PB02_E9, PB02_E10, PB02_E12, PB02_F5, PB02_F11, PB03_A1, PB03_A2, PB03_A3, PB03_A4, PB03_A5, PB03_A6, PB03_A8, PB03_A9, PB03_A10, PB03_A11, PB03_A12, PB03_B2, PB03_B3, PB03_B4, PB03_B5, PB03_B6, PB03_B8, PB03_B10, PB03_B11, PB03_B12, PB03_C1, PB03_C2, PB03_C3, PB03_C4, PB03_C5, PB03_C7, PB03_C8, PB03_C9, PB03_C10, PB03_C11, PB03_D3, PB03_D4, PB03_D5, PB03_D7, PB03_D8, PB03_D9, PB03_D10, PB03_E1, PB03_E6, PB03_E7, PB03_E8, PB03_E9, PB03_E10, PB03_E11, PB03_F1, PB03_F2, PB03_F3, PB03_F4, PB03_F5, PB03_F6, PB03_F7, PB03_F8, PB03_F9, PB03_F11, PB03_F12, PB03_G1, PB03_G2, PB03_G3, PB03_G4, PB03_G6, PB03_G7, PB03_G8, PB03_G9, PB03_G10, PB03_G11, PB03_G12, PB04_A1, PB04_A3, PB04_A5, PB04_A7, PB04_A10, PB04_A11, PB04_A12, PB04_B5, PB04_B7, PB04_B9, PB04_B10, PB04_B12, PB04_C3, PB04_C5, PB04_C8, PB04_C9, PB04_C11, PB04_D1, PB04_D2, PB04_D3, PB04_D4, PB04_D6, PB04_D9, PB04_E3, PB04_E7, PB04_E8, PB04_E9, PB04_E10, PB04_E12, PB04_F1, PB04_F3, PB04_F7, PB04_F8, PB04_F10, PB04_F11, PB04_F12, PB04_G1, PB04_G4, PB04_G7, PB04_G8, PB04_G10, PB04_G12, PB04_D7, PB04_D11, and PB04_F2), and most of them were comparable to or better than that of SB100X and PiggyBac.

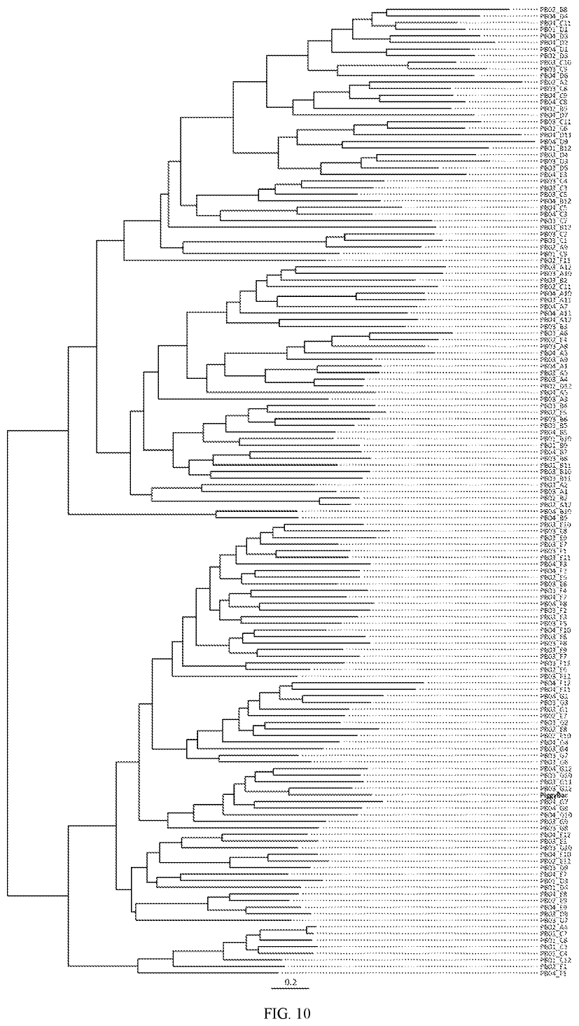

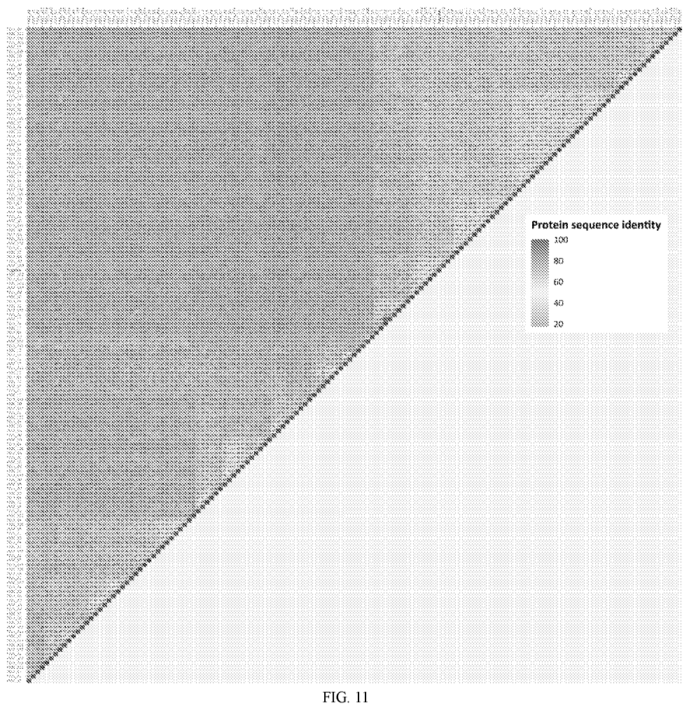

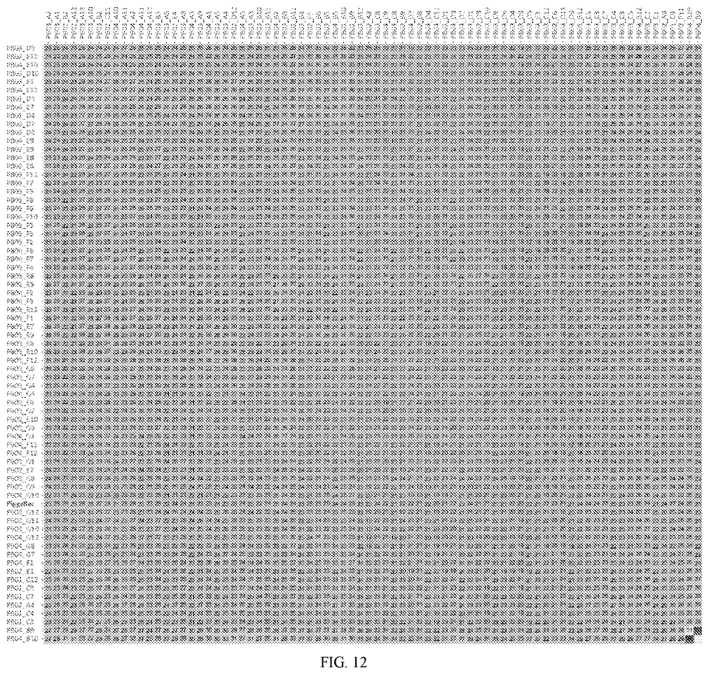

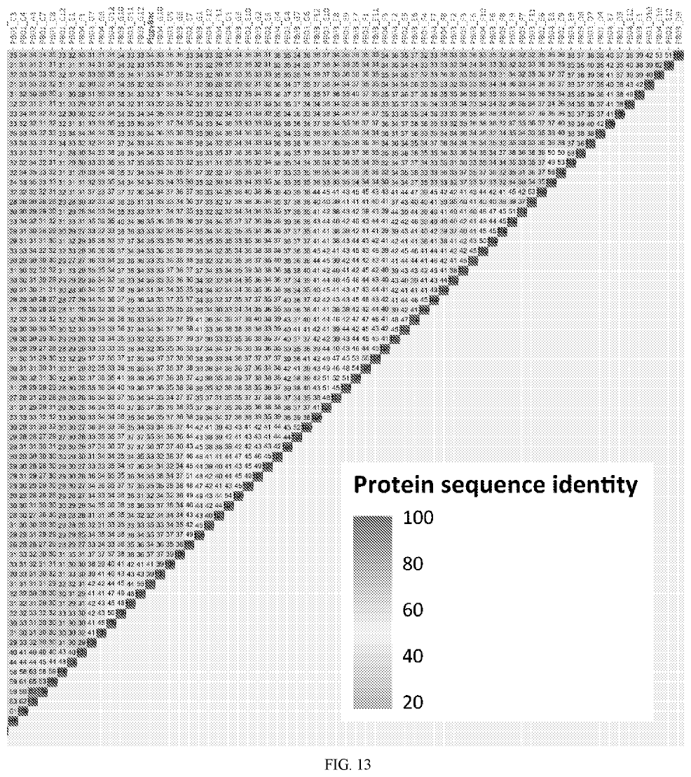

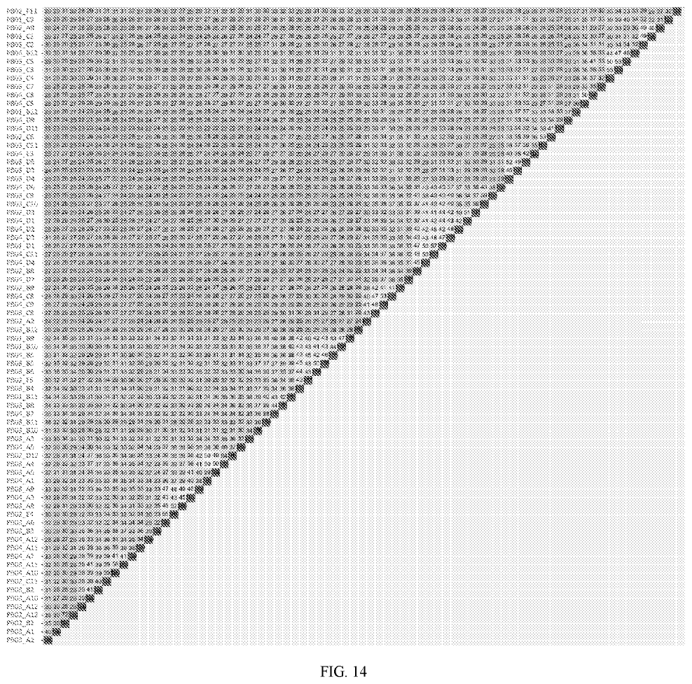

In addition, FIG. 10 showed an evolutionary branching diagram of the transposons of the PiggyBac superfamily in the present application based on protein sequences. FIG. 11 showed the protein sequence similarity (%) among the transposons of the PiggyBac superfamily in the present application. The results showed that these transposons covered different branches of the superfamily, and PiggyBac was also included.

It should be stated that the above are only the preferred examples of the present application and are not intended to limit the present application. For those of ordinary skill in the art, various modifications and changes can be made to the present application. Although the specific embodiments have been described, for the applicant or a person skilled in the art, the substitutions, modifications, changes, improvements, and substantial equivalents of the above embodiments may exist or cannot be foreseen currently. Therefore, the submitted appended claims and claims that may be modified are intended to cover all such substitutions, modifications, changes, improvements, and substantial equivalents. It is important that, as the technology evolves, many elements described herein may be replaced with equivalent elements that appear after the present application.

1. An isolated transposase, wherein the transposase has a transposase sequence selected from the following (i) or a variant sequence of the aforementioned transposase having a transposase activity in (ii)-(iv):
(i) at least one amino acid sequence as shown in any one of SEQ ID NOs: 1-146;
(ii) at least one of sequences obtained by performing deletion, substitution, insertion, or mutation of 1, 2, 3, 4, 5, 6, 7, 8, 9 or 10 amino acids on the amino acid sequence as shown in any one of SEQ ID NOs: 1-146;
(iii) at least one of amino acid sequences having at least 70%, 80%, 90%, 95% or 99% identity to the amino acid sequence as shown in any one of SEQ ID NOs: 1-146; and
(iv) at least one of sequences obtained by further fusing the amino acid sequence as shown in any one of SEQ ID NOs: 1-146 with other sequences.

2. An isolated transposase, wherein the transposase comprises an amino acid sequence as shown in the following formula:
D E (X₁)ₐ K (X₂)_{b} G (X₃)_{c} K (X₄)_{d} G
wherein
a, b, c and d are the numbers of amino acids;
D is aspartic acid;
E is glutamic acid;
K is lysine;
G is glycine;
(X₁) is any amino acid, and a is 17, 18 or 19;
(X₂) is any amino acid, and b is 3, 4 or 5;
(X₃) is any amino acid, and c is 1; and
(X₄) is any amino acid, and d is 17, 18 or 19.

3. An isolated transposase, wherein the transposase comprises an amino acid sequence as shown in the following formula:
P (X₅)ₑ Y (X₆)_{f} D
wherein
e and f are the numbers of amino acids;
P is proline;
Y is tyrosine;
D is aspartic acid;
(X₅) is any amino acid, and e is 5; and
(X₆) is any amino acid, and f is 7.

4. An isolated transposase, wherein the transposase comprises an amino acid sequence as shown in the following formula:
C (X₇)_{g} C (X₈)ₕ C (X₉)ᵢ C
wherein
g, h and i are the numbers of amino acids;
C is cysteine;
(X₇) is any amino acid, and g is 2, 3 or 4;
(X₈) is any amino acid, and h is 7, 8, 9, 10, 11, 12, 13, 14, 15, 16, 17, 18, 19, 20, 21, 22, 23, 24, 25, 26, 27 or 28; and
(X₉) is any amino acid, and i is 2.

5. An isolated transposase, wherein the transposase comprises at least two of the following amino acid sequences (1)-(3):
(1) D E (X₁)ₐ K (X₂)_{b} G (X₃)_{c} K (X₄)_{d} G;
(2) P (X₅)ₑ Y (X₆)_{f} D; or
(3) C (X₇)_{g} C (X₈) n C (X₉)ᵢ C;
wherein
a, b, c, d, e, f, g, h and i are the numbers of amino acids;
D is aspartic acid; E is glutamic acid; K is lysine; G is glycine; P is proline; Y is tyrosine; D is aspartic acid; C is cysteine;
(X₁) is any amino acid, and a is 17, 18 or 19;
(X₂) is any amino acid, and b is 3, 4 or 5;
(X₃) is any amino acid, and c is 1;
(X₄) is any amino acid, and d is 17, 18 or 19;
(X₅) is any amino acid, and e is 5;
(X₆) is any amino acid, and f is 7;
(X₇) is any amino acid, and g is 2, 3 or 4;
(X₈) is any amino acid, and h is 7, 8, 9, 10, 11, 12, 13, 14, 15, 16, 17, 18, 19, 20, 21, 22, 23, 24, 25, 26, 27 or 28; and
(X₉) is any amino acid, and i is 2.

6. An isolated transposase, wherein the transposase comprises the following amino acid sequences (1)-(3):
(1) D E (X₁)ₐ K (X₂)_{b} G (X₃)_{c} K (X₄)_{d} G;
(2) P (X₅)ₑ Y (X₆)_{f} D; and
(3) C (X₇)_{g} C (X₈)ₕ C (X₉)ᵢ C;
wherein
a, b, c, d, e, f, g, h and i are the numbers of amino acids;
D is aspartic acid; E is glutamic acid; K is lysine; G is glycine; P is proline; Y is tyrosine; D is aspartic acid;
C is cysteine;
(X₁) is any amino acid, and a is 17, 18 or 19;
(X₂) is any amino acid, and b is 3, 4 or 5;
(X₃) is any amino acid, and c is 1;
(X₄) is any amino acid, and d is 17, 18 or 19;
(X₅) is any amino acid, and e is 5;
(X₆) is any amino acid, and f is 7;
(X₇) is any amino acid, and g is 2, 3 or 4;
(X₈) is any amino acid, and h is 7, 8, 9, 10, 11, 12, 13, 14, 15, 16, 17, 18, 19, 20, 21, 22, 23, 24, 25, 26, 27 or 28; and
(X₉) is any amino acid, and i is 2.

7.-10. (canceled)

11. A nucleic acid, wherein the nucleic acid encodes the transposase according to claim 1.

12. A nucleic acid construct, comprising the nucleic acid according to claim 11, wherein the nucleic acid construct further comprises a promoter and a poly(A) sequence.

13.-15. (canceled)

16. A nucleic acid set, comprising a 5′ recognition sequence, wherein the 5′ recognition sequence comprises at least one of the nucleotide sequences as shown in SEQ ID NOs: 147-292.

17. A nucleic acid set, comprising a 3′ recognition sequence, wherein the 3′ recognition sequence comprises at least one of the nucleotide sequences as shown in SEQ ID NOs: 293-438.

18. A nucleic acid set, comprising a 5′ recognition sequence and a 3′ recognition sequence, wherein the 5′ recognition sequence comprises the nucleotide sequence as shown in any one of SEQ ID NOs: 147-292 or a variant thereof, the 3′ recognition sequence comprises the nucleotide sequence as shown in any one of SEQ ID NOs: 293-438 or a variant thereof, and the nucleic acid set can be recognized by a specific transposase.

19. The nucleic acid set according to claim 18, wherein the 5′ recognition sequence or the 3′ recognition sequence comprises a terminal inverted repeat of at least one of 1-800 nt, 1-600 nt, 1-400 nt, 1-200 nt, 1-100 nt, 5-50 nt, 5-25 nt, or 10-20 nt in length.

20. A nucleic acid set construct, comprising the nucleic acid set according to claim 18, and further comprising an exogenous nucleic acid fragment.

21. The nucleic acid set construct according to claim 20, wherein the exogenous nucleic acid fragment is operably inserted into the nucleic acid set construct through a polyclonal insertion site, and there may be one or more exogenous nucleic acid fragments, which may be the same or different; and a promoter can also be inserted to control the expression of the exogenous nucleic acid fragment.

22. The nucleic acid set construct according to claim 21, wherein the exogenous nucleic acid fragment includes any gene of interest or any gene that is transposable.

23. The nucleic acid set construct according to claim 64, wherein the gene of a natural functional protein includes a fluorescence-based reporter gene, a luciferase gene, and a resistance gene.

24.-26. (canceled)

27. The nucleic acid set construct according to claim 64, wherein the artificial chimeric gene includes a gene of a chimeric antigen receptor.

28. (canceled)

29. A composition, wherein the composition includes:
a PiggyBac family transposase or a functional fragment thereof, or a nucleic acid encoding the PiggyBac family transposase or the functional fragment thereof, wherein the transposase or the functional fragment thereof has a function of catalyzing the insertion of an exogenous nucleic acid fragment into the genome of a cell; and
a nucleic acid set, wherein the nucleic acid set can be recognized by a specific transposase or a functional fragment thereof.

30. The composition according to claim 29, wherein the composition is selected from at least one of the following groups (1)-(147), and any one of the following groups (1)-(146) comprises: a transposase-related sequence and a nucleic acid set,
(1) the transposase-related sequence is an amino acid sequence comprising the sequence as shown in SEQ ID NO: 1 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 147, and the 3′ recognition sequence is a nucleotide sequence comprising the sequence as shown in SEQ ID NO: 293;
(2) the transposase-related sequence is an amino acid sequence comprising the sequence as shown in SEQ ID NO: 2 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 148, and the 3′ recognition sequence is a nucleotide sequence comprising the sequence as shown in SEQ ID NO: 294;
(3) the transposase-related sequence is an amino acid sequence comprising the sequence as shown in SEQ ID NO: 3 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 149, and the 3′ recognition sequence is a nucleotide sequence comprising the sequence as shown in SEQ ID NO: 295;
(4) the transposase-related sequence is an amino acid sequence comprising the sequence as shown in SEQ ID NO: 4 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 150, and the 3′ recognition sequence is a nucleotide sequence comprising the sequence as shown in SEQ ID NO: 296;
(5) the transposase-related sequence is an amino acid sequence comprising the sequence as shown in SEQ ID NO: 5 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 151, and the 3′ recognition sequence is a nucleotide sequence comprising the sequence as shown in SEQ ID NO: 297;
(6) the transposase-related sequence is an amino acid sequence comprising the sequence as shown in SEQ ID NO: 6 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 152, and the 3′ recognition sequence is a nucleotide sequence comprising the sequence as shown in SEQ ID NO: 298;
(7) the transposase-related sequence is an amino acid sequence comprising the sequence as shown in SEQ ID NO: 7 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 153, and the 3′ recognition sequence is a nucleotide sequence comprising the sequence as shown in SEQ ID NO: 299;
(8) the transposase-related sequence is an amino acid sequence comprising the sequence as shown in SEQ ID NO: 8 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 154, and the 3′ recognition sequence is a nucleotide sequence comprising the sequence as shown in SEQ ID NO: 300;
(9) the transposase-related sequence is an amino acid sequence comprising the sequence as shown in SEQ ID NO: 9 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 155, and the 3′ recognition sequence is a nucleotide sequence comprising the sequence as shown in SEQ ID NO: 301;
(10) the transposase-related sequence is an amino acid sequence comprising the sequence as shown in SEQ ID NO: 10 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 156, and the 3′ recognition sequence is a nucleotide sequence comprising the sequence as shown in SEQ ID NO: 302;
(11) the transposase-related sequence is an amino acid sequence comprising the sequence as shown in SEQ ID NO: 11 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 157, and the 3′ recognition sequence is a nucleotide sequence comprising the sequence as shown in SEQ ID NO: 303;
(12) the transposase-related sequence is an amino acid sequence comprising the sequence as shown in SEQ ID NO: 12 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 158, and the 3′ recognition sequence is a nucleotide sequence comprising the sequence as shown in SEQ ID NO: 304;
(13) the transposase-related sequence is an amino acid sequence comprising the sequence as shown in SEQ ID NO: 13 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 159, and the 3′ recognition sequence is a nucleotide sequence comprising the sequence as shown in SEQ ID NO: 305;
(14) the transposase-related sequence is an amino acid sequence comprising the sequence as shown in SEQ ID NO: 14 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 160, and the 3′ recognition sequence is a nucleotide sequence comprising the sequence as shown in SEQ ID NO: 306;
(15) the transposase-related sequence is an amino acid sequence comprising the sequence as shown in SEQ ID NO: 15 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 161, and the 3′ recognition sequence is a nucleotide sequence comprising the sequence as shown in SEQ ID NO: 307;
(16) the transposase-related sequence is an amino acid sequence comprising the sequence as shown in SEQ ID NO: 16 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 162, and the 3′ recognition sequence is a nucleotide sequence comprising the sequence as shown in SEQ ID NO: 308;
(17) the transposase-related sequence is an amino acid sequence comprising the sequence as shown in SEQ ID NO: 17 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 163, and the 3′ recognition sequence is a nucleotide sequence comprising the sequence as shown in SEQ ID NO: 309;
(18) the transposase-related sequence is an amino acid sequence comprising the sequence as shown in SEQ ID NO: 18 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 164, and the 3′ recognition sequence is a nucleotide sequence comprising the sequence as shown in SEQ ID NO: 310;
(19) the transposase-related sequence is an amino acid sequence comprising the sequence as shown in SEQ ID NO: 19 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 165, and the 3′ recognition sequence is a nucleotide sequence comprising the sequence as shown in SEQ ID NO: 311;
(20) the transposase-related sequence is an amino acid sequence comprising the sequence as shown in SEQ ID NO: 20 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 166, and the 3′ recognition sequence is a nucleotide sequence comprising the sequence as shown in SEQ ID NO: 312;
(21) the transposase-related sequence is an amino acid sequence comprising the sequence as shown in SEQ ID NO: 21 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 167, and the 3′ recognition sequence is a nucleotide sequence comprising the sequence as shown in SEQ ID NO: 313;
(22) the transposase-related sequence is an amino acid sequence comprising the sequence as shown in SEQ ID NO: 22 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 168, and the 3′ recognition sequence is a nucleotide sequence comprising the sequence as shown in SEQ ID NO: 314;
(23) the transposase-related sequence is an amino acid sequence comprising the sequence as shown in SEQ ID NO: 23 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 169, and the 3′ recognition sequence is a nucleotide sequence comprising the sequence as shown in SEQ ID NO: 315;
(24) the transposase-related sequence is an amino acid sequence comprising the sequence as shown in SEQ ID NO: 24 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 170, and the 3′ recognition sequence is a nucleotide sequence comprising the sequence as shown in SEQ ID NO: 316;
(25) the transposase-related sequence is an amino acid sequence comprising the sequence as shown in SEQ ID NO: 25 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 171, and the 3′ recognition sequence is a nucleotide sequence comprising the sequence as shown in SEQ ID NO: 317;
(26) the transposase-related sequence is an amino acid sequence comprising the sequence as shown in SEQ ID NO: 26 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 172, and the 3′ recognition sequence is a nucleotide sequence comprising the sequence as shown in SEQ ID NO: 318;
(27) the transposase-related sequence is an amino acid sequence comprising the sequence as shown in SEQ ID NO: 27 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 173, and the 3′ recognition sequence is a nucleotide sequence comprising the sequence as shown in SEQ ID NO: 319;
(28) the transposase-related sequence is an amino acid sequence comprising the sequence as shown in SEQ ID NO: 28 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 174, and the 3′ recognition sequence is a nucleotide sequence comprising the sequence as shown in SEQ ID NO: 320;
(29) the transposase-related sequence is an amino acid sequence comprising the sequence as shown in SEQ ID NO: 29 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 175, and the 3′ recognition sequence is a nucleotide sequence comprising the sequence as shown in SEQ ID NO: 321;
(30) the transposase-related sequence is an amino acid sequence comprising the sequence as shown in SEQ ID NO: 30 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 176, and the 3′ recognition sequence is a nucleotide sequence comprising the sequence as shown in SEQ ID NO: 322;
(31) the transposase-related sequence is an amino acid sequence comprising the sequence as shown in SEQ ID NO: 31 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 177, and the 3′ recognition sequence is a nucleotide sequence comprising the sequence as shown in SEQ ID NO: 323;
(32) the transposase-related sequence is an amino acid sequence comprising the sequence as shown in SEQ ID NO: 32 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 178, and the 3′ recognition sequence is a nucleotide sequence comprising the sequence as shown in SEQ ID NO: 324;
(33) the transposase-related sequence is an amino acid sequence comprising the sequence as shown in SEQ ID NO: 33 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 179, and the 3′ recognition sequence is a nucleotide sequence comprising the sequence as shown in SEQ ID NO: 325;
(34) the transposase-related sequence is an amino acid sequence comprising the sequence as shown in SEQ ID NO: 34 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 180, and the 3′ recognition sequence is a nucleotide sequence comprising the sequence as shown in SEQ ID NO: 326;
(35) the transposase-related sequence is an amino acid sequence comprising the sequence as shown in SEQ ID NO: 35 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 181, and the 3′ recognition sequence is a nucleotide sequence comprising the sequence as shown in SEQ ID NO: 327;
(36) the transposase-related sequence is an amino acid sequence comprising the sequence as shown in SEQ ID NO: 36 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 182, and the 3′ recognition sequence is a nucleotide sequence comprising the sequence as shown in SEQ ID NO: 328;
(37) the transposase-related sequence is an amino acid sequence comprising the sequence as shown in SEQ ID NO: 37 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 183, and the 3′ recognition sequence is a nucleotide sequence comprising the sequence as shown in SEQ ID NO: 329;
(38) the transposase-related sequence is an amino acid sequence comprising the sequence as shown in SEQ ID NO: 38 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 184, and the 3′ recognition sequence is a nucleotide sequence comprising the sequence as shown in SEQ ID NO: 330;
(39) the transposase-related sequence is an amino acid sequence comprising the sequence as shown in SEQ ID NO: 39 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 185, and the 3′ recognition sequence is a nucleotide sequence comprising the sequence as shown in SEQ ID NO: 331;
(40) the transposase-related sequence is an amino acid sequence comprising the sequence as shown in SEQ ID NO: 40 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 186, and the 3′ recognition sequence is a nucleotide sequence comprising the sequence as shown in SEQ ID NO: 332;
(41) the transposase-related sequence is an amino acid sequence comprising the sequence as shown in SEQ ID NO: 41 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 187, and the 3′ recognition sequence is a nucleotide sequence comprising the sequence as shown in SEQ ID NO: 333;
(42) the transposase-related sequence is an amino acid sequence comprising the sequence as shown in SEQ ID NO: 42 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 188, and the 3′ recognition sequence is a nucleotide sequence comprising the sequence as shown in SEQ ID NO: 334;
(43) the transposase-related sequence is an amino acid sequence comprising the sequence as shown in SEQ ID NO: 43 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 189, and the 3′ recognition sequence is a nucleotide sequence comprising the sequence as shown in SEQ ID NO: 335;
(44) the transposase-related sequence is an amino acid sequence comprising the sequence as shown in SEQ ID NO: 44 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 190, and the 3′ recognition sequence is a nucleotide sequence comprising the sequence as shown in SEQ ID NO: 336;
(45) the transposase-related sequence is an amino acid sequence comprising the sequence as shown in SEQ ID NO: 45 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 191, and the 3′ recognition sequence is a nucleotide sequence comprising the sequence as shown in SEQ ID NO: 337;
(46) the transposase-related sequence is an amino acid sequence comprising the sequence as shown in SEQ ID NO: 46 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 192, and the 3′ recognition sequence is a nucleotide sequence comprising the sequence as shown in SEQ ID NO: 338;
(47) the transposase-related sequence is an amino acid sequence comprising the sequence as shown in SEQ ID NO: 47 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 193, and the 3′ recognition sequence is a nucleotide sequence comprising the sequence as shown in SEQ ID NO: 339;
(48) the transposase-related sequence is an amino acid sequence comprising the sequence as shown in SEQ ID NO: 48 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 194, and the 3′ recognition sequence is a nucleotide sequence comprising the sequence as shown in SEQ ID NO: 340;
(49) the transposase-related sequence is an amino acid sequence comprising the sequence as shown in SEQ ID NO: 49 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 195, and the 3′ recognition sequence is a nucleotide sequence comprising the sequence as shown in SEQ ID NO: 341;
(50) the transposase-related sequence is an amino acid sequence comprising the sequence as shown in SEQ ID NO: 50 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 196, and the 3′ recognition sequence is a nucleotide sequence comprising the sequence as shown in SEQ ID NO: 342;
(51) the transposase-related sequence is an amino acid sequence comprising the sequence as shown in SEQ ID NO: 51 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 197, and the 3′ recognition sequence is a nucleotide sequence comprising the sequence as shown in SEQ ID NO: 343;
(52) the transposase-related sequence is an amino acid sequence comprising the sequence as shown in SEQ ID NO: 52 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 198, and the 3′ recognition sequence is a nucleotide sequence comprising the sequence as shown in SEQ ID NO: 344;
(53) the transposase-related sequence is an amino acid sequence comprising the sequence as shown in SEQ ID NO: 53 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 199, and the 3′ recognition sequence is a nucleotide sequence comprising the sequence as shown in SEQ ID NO: 345;
(54) the transposase-related sequence is an amino acid sequence comprising the sequence as shown in SEQ ID NO: 54 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 200, and the 3′ recognition sequence is a nucleotide sequence comprising the sequence as shown in SEQ ID NO: 346;
(55) the transposase-related sequence is an amino acid sequence comprising the sequence as shown in SEQ ID NO: 55 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 201, and the 3′ recognition sequence is a nucleotide sequence comprising the sequence as shown in SEQ ID NO: 347;
(56) the transposase-related sequence is an amino acid sequence comprising the sequence as shown in SEQ ID NO: 56 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 202, and the 3′ recognition sequence is a nucleotide sequence comprising the sequence as shown in SEQ ID NO: 348;
(57) the transposase-related sequence is an amino acid sequence comprising the sequence as shown in SEQ ID NO: 57 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 203, and the 3′ recognition sequence is a nucleotide sequence comprising the sequence as shown in SEQ ID NO: 349;
(58) the transposase-related sequence is an amino acid sequence comprising the sequence as shown in SEQ ID NO: 58 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 204, and the 3′ recognition sequence is a nucleotide sequence comprising the sequence as shown in SEQ ID NO: 350;
(59) the transposase-related sequence is an amino acid sequence comprising the sequence as shown in SEQ ID NO: 59 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 205, and the 3′ recognition sequence is a nucleotide sequence comprising the sequence as shown in SEQ ID NO: 351;
(60) the transposase-related sequence is an amino acid sequence comprising the sequence as shown in SEQ ID NO: 60 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 206, and the 3′ recognition sequence is a nucleotide sequence comprising the sequence as shown in SEQ ID NO: 352;
(61) the transposase-related sequence is an amino acid sequence comprising the sequence as shown in SEQ ID NO: 61 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 207, and the 3′ recognition sequence is a nucleotide sequence comprising the sequence as shown in SEQ ID NO: 353;
(62) the transposase-related sequence is an amino acid sequence comprising the sequence as shown in SEQ ID NO: 62 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 208, and the 3′ recognition sequence is a nucleotide sequence comprising the sequence as shown in SEQ ID NO: 354;
(63) the transposase-related sequence is an amino acid sequence comprising the sequence as shown in SEQ ID NO: 63 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 209, and the 3′ recognition sequence is a nucleotide sequence comprising the sequence as shown in SEQ ID NO: 355;
(64) the transposase-related sequence is an amino acid sequence comprising the sequence as shown in SEQ ID NO: 64 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 210, and the 3′ recognition sequence is a nucleotide sequence comprising the sequence as shown in SEQ ID NO: 356;
(65) the transposase-related sequence is an amino acid sequence comprising the sequence as shown in SEQ ID NO: 65 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 211, and the 3′ recognition sequence is a nucleotide sequence comprising the sequence as shown in SEQ ID NO: 357;
(66) the transposase-related sequence is an amino acid sequence comprising the sequence as shown in SEQ ID NO: 66 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 212, and the 3′ recognition sequence is a nucleotide sequence comprising the sequence as shown in SEQ ID NO: 358;
(67) the transposase-related sequence is an amino acid sequence comprising the sequence as shown in SEQ ID NO: 67 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 213, and the 3′ recognition sequence is a nucleotide sequence comprising the sequence as shown in SEQ ID NO: 359;
(68) the transposase-related sequence is an amino acid sequence comprising the sequence as shown in SEQ ID NO: 68 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 214, and the 3′ recognition sequence is a nucleotide sequence comprising the sequence as shown in SEQ ID NO: 360;
(69) the transposase-related sequence is an amino acid sequence comprising the sequence as shown in SEQ ID NO: 69 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 215, and the 3′ recognition sequence is a nucleotide sequence comprising the sequence as shown in SEQ ID NO: 361;
(70) the transposase-related sequence is an amino acid sequence comprising the sequence as shown in SEQ ID NO: 70 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 216, and the 3′ recognition sequence is a nucleotide sequence comprising the sequence as shown in SEQ ID NO: 362;
(71) the transposase-related sequence is an amino acid sequence comprising the sequence as shown in SEQ ID NO: 71 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 217, and the 3′ recognition sequence is a nucleotide sequence comprising the sequence as shown in SEQ ID NO: 363;
(72) the transposase-related sequence is an amino acid sequence comprising the sequence as shown in SEQ ID NO: 72 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 218, and the 3′ recognition sequence is a nucleotide sequence comprising the sequence as shown in SEQ ID NO: 364;
(73) the transposase-related sequence is an amino acid sequence comprising the sequence as shown in SEQ ID NO: 73 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 219, and the 3′ recognition sequence is a nucleotide sequence comprising the sequence as shown in SEQ ID NO: 365;
(74) the transposase-related sequence is an amino acid sequence comprising the sequence as shown in SEQ ID NO: 74 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 220, and the 3′ recognition sequence is a nucleotide sequence comprising the sequence as shown in SEQ ID NO: 366;
(75) the transposase-related sequence is an amino acid sequence comprising the sequence as shown in SEQ ID NO: 75 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 221, and the 3′ recognition sequence is a nucleotide sequence comprising the sequence as shown in SEQ ID NO: 367;
(76) the transposase-related sequence is an amino acid sequence comprising the sequence as shown in SEQ ID NO: 76 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 222, and the 3′ recognition sequence is a nucleotide sequence comprising the sequence as shown in SEQ ID NO: 368;
(77) the transposase-related sequence is an amino acid sequence comprising the sequence as shown in SEQ ID NO: 77 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 223, and the 3′ recognition sequence is a nucleotide sequence comprising the sequence as shown in SEQ ID NO: 369;
(78) the transposase-related sequence is an amino acid sequence comprising the sequence as shown in SEQ ID NO: 78 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 224, and the 3′ recognition sequence is a nucleotide sequence comprising the sequence as shown in SEQ ID NO: 370;
(79) the transposase-related sequence is an amino acid sequence comprising the sequence as shown in SEQ ID NO: 79 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 225, and the 3′ recognition sequence is a nucleotide sequence comprising the sequence as shown in SEQ ID NO: 371;
(80) the transposase-related sequence is an amino acid sequence comprising the sequence as shown in SEQ ID NO: 80 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 226, and the 3′ recognition sequence is a nucleotide sequence comprising the sequence as shown in SEQ ID NO: 372;
(81) the transposase-related sequence is an amino acid sequence comprising the sequence as shown in SEQ ID NO: 81 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 227, and the 3′ recognition sequence is a nucleotide sequence comprising the sequence as shown in SEQ ID NO: 373;
(82) the transposase-related sequence is an amino acid sequence comprising the sequence as shown in SEQ ID NO: 82 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 228, and the 3′ recognition sequence is a nucleotide sequence comprising the sequence as shown in SEQ ID NO: 374;
(83) the transposase-related sequence is an amino acid sequence comprising the sequence as shown in SEQ ID NO: 83 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 229, and the 3′ recognition sequence is a nucleotide sequence comprising the sequence as shown in SEQ ID NO: 375;
(84) the transposase-related sequence is an amino acid sequence comprising the sequence as shown in SEQ ID NO: 84 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 230, and the 3′ recognition sequence is a nucleotide sequence comprising the sequence as shown in SEQ ID NO: 376;
(85) the transposase-related sequence is an amino acid sequence comprising the sequence as shown in SEQ ID NO: 85 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 231, and the 3′ recognition sequence is a nucleotide sequence comprising the sequence as shown in SEQ ID NO: 377;
(86) the transposase-related sequence is an amino acid sequence comprising the sequence as shown in SEQ ID NO: 86 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 232, and the 3′ recognition sequence is a nucleotide sequence comprising the sequence as shown in SEQ ID NO: 378;
(87) the transposase-related sequence is an amino acid sequence comprising the sequence as shown in SEQ ID NO: 87 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 233, and the 3′ recognition sequence is a nucleotide sequence comprising the sequence as shown in SEQ ID NO: 379;
(88) the transposase-related sequence is an amino acid sequence comprising the sequence as shown in SEQ ID NO: 88 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 234, and the 3′ recognition sequence is a nucleotide sequence comprising the sequence as shown in SEQ ID NO: 380;
(89) the transposase-related sequence is an amino acid sequence comprising the sequence as shown in SEQ ID NO: 89 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 235, and the 3′ recognition sequence is a nucleotide sequence comprising the sequence as shown in SEQ ID NO: 381;
(90) the transposase-related sequence is an amino acid sequence comprising the sequence as shown in SEQ ID NO: 90 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 236, and the 3′ recognition sequence is a nucleotide sequence comprising the sequence as shown in SEQ ID NO: 382;
(91) the transposase-related sequence is an amino acid sequence comprising the sequence as shown in SEQ ID NO: 91 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 237, and the 3′ recognition sequence is a nucleotide sequence comprising the sequence as shown in SEQ ID NO: 383;
(92) the transposase-related sequence is an amino acid sequence comprising the sequence as shown in SEQ ID NO: 92 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 238, and the 3′ recognition sequence is a nucleotide sequence comprising the sequence as shown in SEQ ID NO: 384;
(93) the transposase-related sequence is an amino acid sequence comprising the sequence as shown in SEQ ID NO: 93 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 239, and the 3′ recognition sequence is a nucleotide sequence comprising the sequence as shown in SEQ ID NO: 385;
(94) the transposase-related sequence is an amino acid sequence comprising the sequence as shown in SEQ ID NO: 94 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 240, and the 3′ recognition sequence is a nucleotide sequence comprising the sequence as shown in SEQ ID NO: 386;
(95) the transposase-related sequence is an amino acid sequence comprising the sequence as shown in SEQ ID NO: 95 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 241, and the 3′ recognition sequence is a nucleotide sequence comprising the sequence as shown in SEQ ID NO: 387;
(96) the transposase-related sequence is an amino acid sequence comprising the sequence as shown in SEQ ID NO: 96 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 242, and the 3′ recognition sequence is a nucleotide sequence comprising the sequence as shown in SEQ ID NO: 388;
(97) the transposase-related sequence is an amino acid sequence comprising the sequence as shown in SEQ ID NO: 97 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 243, and the 3′ recognition sequence is a nucleotide sequence comprising the sequence as shown in SEQ ID NO: 389;
(98) the transposase-related sequence is an amino acid sequence comprising the sequence as shown in SEQ ID NO: 98 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 244, and the 3′ recognition sequence is a nucleotide sequence comprising the sequence as shown in SEQ ID NO: 390;
(99) the transposase-related sequence is an amino acid sequence comprising the sequence as shown in SEQ ID NO: 99 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 245, and the 3′ recognition sequence is a nucleotide sequence comprising the sequence as shown in SEQ ID NO: 391;
(100) the transposase-related sequence is an amino acid sequence comprising the sequence as shown in SEQ ID NO: 100 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 246, and the 3′ recognition sequence is a nucleotide sequence comprising the sequence as shown in SEQ ID NO: 392;
(101) the transposase-related sequence is an amino acid sequence comprising the sequence as shown in SEQ ID NO: 101 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 247, and the 3′ recognition sequence is a nucleotide sequence comprising the sequence as shown in SEQ ID NO: 393;
(102) the transposase-related sequence is an amino acid sequence comprising the sequence as shown in SEQ ID NO: 102 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 248, and the 3′ recognition sequence is a nucleotide sequence comprising the sequence as shown in SEQ ID NO: 394;
(103) the transposase-related sequence is an amino acid sequence comprising the sequence as shown in SEQ ID NO: 103 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 249, and the 3′ recognition sequence is a nucleotide sequence comprising the sequence as shown in SEQ ID NO: 395;
(104) the transposase-related sequence is an amino acid sequence comprising the sequence as shown in SEQ ID NO: 104 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 250, and the 3′ recognition sequence is a nucleotide sequence comprising the sequence as shown in SEQ ID NO: 396;
(105) the transposase-related sequence is an amino acid sequence comprising the sequence as shown in SEQ ID NO: 105 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 251, and the 3′ recognition sequence is a nucleotide sequence comprising the sequence as shown in SEQ ID NO: 397;
(106) the transposase-related sequence is an amino acid sequence comprising the sequence as shown in SEQ ID NO: 106 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 252, and the 3′ recognition sequence is a nucleotide sequence comprising the sequence as shown in SEQ ID NO: 398;
(107) the transposase-related sequence is an amino acid sequence comprising the sequence as shown in SEQ ID NO: 107 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 253, and the 3′ recognition sequence is a nucleotide sequence comprising the sequence as shown in SEQ ID NO: 399;
(108) the transposase-related sequence is an amino acid sequence comprising the sequence as shown in SEQ ID NO: 108 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 254, and the 3′ recognition sequence is a nucleotide sequence comprising the sequence as shown in SEQ ID NO: 400;
(109) the transposase-related sequence is an amino acid sequence comprising the sequence as shown in SEQ ID NO: 109 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 255, and the 3′ recognition sequence is a nucleotide sequence comprising the sequence as shown in SEQ ID NO: 401;
(110) the transposase-related sequence is an amino acid sequence comprising the sequence as shown in SEQ ID NO: 110 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 256, and the 3′ recognition sequence is a nucleotide sequence comprising the sequence as shown in SEQ ID NO: 402;
(111) the transposase-related sequence is an amino acid sequence comprising the sequence as shown in SEQ ID NO: 111 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 257, and the 3′ recognition sequence is a nucleotide sequence comprising the sequence as shown in SEQ ID NO: 403;
(112) the transposase-related sequence is an amino acid sequence comprising the sequence as shown in SEQ ID NO: 112 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 258, and the 3′ recognition sequence is a nucleotide sequence comprising the sequence as shown in SEQ ID NO: 404;
(113) the transposase-related sequence is an amino acid sequence comprising the sequence as shown in SEQ ID NO: 113 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 259, and the 3′ recognition sequence is a nucleotide sequence comprising the sequence as shown in SEQ ID NO: 405;
(114) the transposase-related sequence is an amino acid sequence comprising the sequence as shown in SEQ ID NO: 114 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 260, and the 3′ recognition sequence is a nucleotide sequence comprising the sequence as shown in SEQ ID NO: 406;
(115) the transposase-related sequence is an amino acid sequence comprising the sequence as shown in SEQ ID NO: 115 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 261, and the 3′ recognition sequence is a nucleotide sequence comprising the sequence as shown in SEQ ID NO: 407;
(116) the transposase-related sequence is an amino acid sequence comprising the sequence as shown in SEQ ID NO: 116 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 262, and the 3′ recognition sequence is a nucleotide sequence comprising the sequence as shown in SEQ ID NO: 408;
(117) the transposase-related sequence is an amino acid sequence comprising the sequence as shown in SEQ ID NO: 117 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 263, and the 3′ recognition sequence is a nucleotide sequence comprising the sequence as shown in SEQ ID NO: 409;
(118) the transposase-related sequence is an amino acid sequence comprising the sequence as shown in SEQ ID NO: 118 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 264, and the 3′ recognition sequence is a nucleotide sequence comprising the sequence as shown in SEQ ID NO: 410;
(119) the transposase-related sequence is an amino acid sequence comprising the sequence as shown in SEQ ID NO: 119 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 265, and the 3′ recognition sequence is a nucleotide sequence comprising the sequence as shown in SEQ ID NO: 411;
(120) the transposase-related sequence is an amino acid sequence comprising the sequence as shown in SEQ ID NO: 120 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 266, and the 3′ recognition sequence is a nucleotide sequence comprising the sequence as shown in SEQ ID NO: 412;
(121) the transposase-related sequence is an amino acid sequence comprising the sequence as shown in SEQ ID NO: 121 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 267, and the 3′ recognition sequence is a nucleotide sequence comprising the sequence as shown in SEQ ID NO: 413;
(122) the transposase-related sequence is an amino acid sequence comprising the sequence as shown in SEQ ID NO: 122 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 268, and the 3′ recognition sequence is a nucleotide sequence comprising the sequence as shown in SEQ ID NO: 414;
(123) the transposase-related sequence is an amino acid sequence comprising the sequence as shown in SEQ ID NO: 123 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 269, and the 3′ recognition sequence is a nucleotide sequence comprising the sequence as shown in SEQ ID NO: 415;
(124) the transposase-related sequence is an amino acid sequence comprising the sequence as shown in SEQ ID NO: 124 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 270, and the 3′ recognition sequence is a nucleotide sequence comprising the sequence as shown in SEQ ID NO: 416;
(125) the transposase-related sequence is an amino acid sequence comprising the sequence as shown in SEQ ID NO: 125 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 271, and the 3′ recognition sequence is a nucleotide sequence comprising the sequence as shown in SEQ ID NO: 417;
(126) the transposase-related sequence is an amino acid sequence comprising the sequence as shown in SEQ ID NO: 126 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 272, and the 3′ recognition sequence is a nucleotide sequence comprising the sequence as shown in SEQ ID NO: 418;
(127) the transposase-related sequence is an amino acid sequence comprising the sequence as shown in SEQ ID NO: 127 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 273, and the 3′ recognition sequence is a nucleotide sequence comprising the sequence as shown in SEQ ID NO: 419;
(128) the transposase-related sequence is an amino acid sequence comprising the sequence as shown in SEQ ID NO: 128 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 274, and the 3′ recognition sequence is a nucleotide sequence comprising the sequence as shown in SEQ ID NO: 420;
(129) the transposase-related sequence is an amino acid sequence comprising the sequence as shown in SEQ ID NO: 129 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 275, and the 3′ recognition sequence is a nucleotide sequence comprising the sequence as shown in SEQ ID NO: 421;
(130) the transposase-related sequence is an amino acid sequence comprising the sequence as shown in SEQ ID NO: 130 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 276, and the 3′ recognition sequence is a nucleotide sequence comprising the sequence as shown in SEQ ID NO: 422;
(131) the transposase-related sequence is an amino acid sequence comprising the sequence as shown in SEQ ID NO: 131 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 277, and the 3′ recognition sequence is a nucleotide sequence comprising the sequence as shown in SEQ ID NO: 423;
(132) the transposase-related sequence is an amino acid sequence comprising the sequence as shown in SEQ ID NO: 132 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 278, and the 3′ recognition sequence is a nucleotide sequence comprising the sequence as shown in SEQ ID NO: 424;
(133) the transposase-related sequence is an amino acid sequence comprising the sequence as shown in SEQ ID NO: 133 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 279, and the 3′ recognition sequence is a nucleotide sequence comprising the sequence as shown in SEQ ID NO: 425;
(134) the transposase-related sequence is an amino acid sequence comprising the sequence as shown in SEQ ID NO: 134 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 280, and the 3′ recognition sequence is a nucleotide sequence comprising the sequence as shown in SEQ ID NO: 426;
(135) the transposase-related sequence is an amino acid sequence comprising the sequence as shown in SEQ ID NO: 135 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 281, and the 3′ recognition sequence is a nucleotide sequence comprising the sequence as shown in SEQ ID NO: 427;
(136) the transposase-related sequence is an amino acid sequence comprising the sequence as shown in SEQ ID NO: 136 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 282, and the 3′ recognition sequence is a nucleotide sequence comprising the sequence as shown in SEQ ID NO: 428;
(137) the transposase-related sequence is an amino acid sequence comprising the sequence as shown in SEQ ID NO: 137 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 283, and the 3′ recognition sequence is a nucleotide sequence comprising the sequence as shown in SEQ ID NO: 429;
(138) the transposase-related sequence is an amino acid sequence comprising the sequence as shown in SEQ ID NO: 138 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 284, and the 3′ recognition sequence is a nucleotide sequence comprising the sequence as shown in SEQ ID NO: 430;
(139) the transposase-related sequence is an amino acid sequence comprising the sequence as shown in SEQ ID NO: 139 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 285, and the 3′ recognition sequence is a nucleotide sequence comprising the sequence as shown in SEQ ID NO: 431;
(140) the transposase-related sequence is an amino acid sequence comprising the sequence as shown in SEQ ID NO: 140 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 286, and the 3′ recognition sequence is a nucleotide sequence comprising the sequence as shown in SEQ ID NO: 432;
(141) the transposase-related sequence is an amino acid sequence comprising the sequence as shown in SEQ ID NO: 141 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 287, and the 3′ recognition sequence is a nucleotide sequence comprising the sequence as shown in SEQ ID NO: 433;
(142) the transposase-related sequence is an amino acid sequence comprising the sequence as shown in SEQ ID NO: 142 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 288, and the 3′ recognition sequence is a nucleotide sequence comprising the sequence as shown in SEQ ID NO: 434;
(143) the transposase-related sequence is an amino acid sequence comprising the sequence as shown in SEQ ID NO: 143 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 289, and the 3′ recognition sequence is a nucleotide sequence comprising the sequence as shown in SEQ ID NO: 435;
(144) the transposase-related sequence is an amino acid sequence comprising the sequence as shown in SEQ ID NO: 144 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 290, and the 3′ recognition sequence is a nucleotide sequence comprising the sequence as shown in SEQ ID NO: 436;
(145) the transposase-related sequence is an amino acid sequence comprising the sequence as shown in SEQ ID NO: 145 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 291, and the 3′ recognition sequence is a nucleotide sequence comprising the sequence as shown in SEQ ID NO: 437;
(146) the transposase-related sequence is an amino acid sequence comprising the sequence as shown in SEQ ID NO: 146 or a nucleic acid encoding the amino acid sequence; and the nucleic acid set comprises a 5′ recognition sequence and a 3′ recognition sequence, wherein the 5′ recognition sequence is a nucleotide sequence comprising the sequence as shown in SEQ ID NO: 292, and the 3′ recognition sequence is a nucleotide sequence comprising the sequence as shown in SEQ ID NO: 438; or
(147) a variant of any one of the aforementioned groups (1)-(146),
wherein the transposase-related sequence is the amino acid sequence of the variant of the transposase in each group or a nucleic acid sequence encoding the variant, and the variant has a variant sequence of the aforementioned transposase having a transposase activity selected from the following (i)-(iii):
(i) at least one of sequences obtained by performing deletion, substitution, insertion, or mutation of 1, 2, 3, 4, 5, 6, 7, 8, 9 or 10 amino acids on the amino acid sequence of the transposase in each group;
(ii) at least one of amino acid sequences having at least 70%, 80%, 90%, 95% or 99% identity to the amino acid sequence as shown in any one of SEQ ID NOs: 1-146; and
(iii) at least one of sequences obtained by further fusing the amino acid sequence as shown in any one of SEQ ID NOs: 1-146 with other sequences.

31. The composition according to claim 30, wherein the nucleic acid set further comprises a promoter and a poly(A) sequence.

32.-33. (canceled)

34. The composition according to claim 30, wherein the nucleic acid set further comprises an exogenous nucleic acid fragment.

35. The composition according to claim 34, wherein the exogenous nucleic acid fragment is operably inserted into the nucleic acid set through a polyclonal insertion site, and there may be one or more exogenous nucleic acid fragments, which may be the same or different; and a promoter can also be inserted to control the expression of the exogenous nucleic acid fragment.

36. The composition according to claim 35, wherein the exogenous nucleic acid fragment includes any gene of interest or any gene that is transposable.

37. The composition according to claim 65, wherein the gene of a natural functional protein includes a fluorescence-based reporter gene, a luciferase gene, or a resistance gene.

38-40. (canceled)

41. The composition according to claim 65, wherein the artificial chimeric gene includes a gene of a chimeric antigen receptor.

42. (canceled)

43. A recombinant vector, wherein the recombinant vector comprises the nucleic acid encoding the transposase according to claim 1.

44. The recombinant vector according to claim 43, wherein the recombinant vector includes a recombinant cloning vector, a recombinant eukaryotic expression plasmid, or a recombinant viral vector.

45.-46. (canceled)

47. A recombinant host cell, wherein the recombinant host cell comprises the transposase according to claim 1.

48. The recombinant host cell according to claim 47, wherein the recombinant host cell includes an animal cell, a plant cell, an algal cell, a fungal cell, a yeast cell, or a bacterial cell.

49.-50. (canceled)

51. A method for introducing an exogenous nucleic acid fragment into the genome of a host cell, wherein the method comprises: delivering the transposase according to claim 1 or the nucleic acid encoding the transposase according to claim 1 into a host cell.

52. A method for editing the genome of a host cell, wherein the method comprises: delivering the transposase according to claim 1 or the nucleic acid encoding the transposase according to claim 1 into a host cell.

53. (canceled)

54. The method according to claim 52, wherein the delivery method includes cationic liposome delivery, lipoid nanoparticulate delivery, cationic polymer delivery, vesicle-exosome delivery, gold nanoparticulate delivery, polypeptide and protein delivery, retrovirus delivery, lentivirus delivery, adenovirus delivery, adeno-associated virus delivery, electroporation, agrobacterium infection, or gene gun.

55.-62. (canceled)

63. A kit, wherein the kit comprises the transposase according to claim 1.

64. The nucleic acid set construct according to claim 22, wherein the exogenous nucleic acid fragment includes a gene of a natural functional protein, an artificial chimeric gene, or a gene of a non-coding RNA.

65. The composition according to claim 36, wherein the exogenous nucleic acid fragment includes a gene of a natural functional protein, an artificial chimeric gene, or a gene of a non-coding RNA.
